(19) United States
(12) Patent Application Publication
(10) Pub. No.: US 2017/0362654 A1
(43) Pub. Date: Dec. 21, 2017

(54) BIOMARKERS OF OOCYTE QUALITY

(71) Applicant: THE TRUSTEES OF PRINCETON UNIVERSITY, Princeton, NJ (US)

(72) Inventor: Coleen T. MURPHY, Princeton, NJ (US)

(21) Appl. No.: 15/533,746

(22) PCT Filed: Dec. 9, 2015
(86) PCT No.: PCT/US15/64836

§ 371 Date: Jun. 7, 2017

Related U.S. Application Data
(60) Provisional application No. 62/089,604, filed on Dec. 9, 2014.
(60) Provisional application No. 62/254,356, filed on Nov. 12, 2015.

(51) Int. Cl. C12Q 1/68; G01N 33/50; G01N 33/53

(52) U.S. Cl. CPC C12Q 1/6881; G01N 33/53; C12Q 1/6883; G01N 33/5091; C12Q 2600/158

(57) ABSTRACT

The present technology relates to biomarkers of oocyte quality and oocyte quality dealine, as well as methods in connection with such biomarkers, including method of determining the quality of an oocyte, kits for the same, and libraries, reproductive aging gene expression profiles and profile sets with information relating the same.

### CROSS REFERENCE TO RELATED APPLICATIONS

This application claims priority to U.S. Provisional Application No. 62/089,604 filed Dec. 9, 2014, and U.S. Provisional Application No. 62/254,356 filed Nov. 12, 2015, the contents of both of which are hereby incorporated by reference.

### STATEMENT REGARDING FEDERALLY SPONSORED RESEARCH OR DEVELOPMENT

This invention was made with government support under Grant No. OD004402 awarded by the National Institutes of Health. The government has certain rights in the invention.

### BACKGROUND

The present technology relates to biomarkers of oocyte quality and oocyte quality decline, as well as methods in connection with such biomarkers.

The functions of the female reproductive system are known to decline with age. One major factor in decreased fertility is the decline of the quality of the eggs (oocytes) produced by the ovaries as a woman ages. Decline of quality of oocytes and ovarian follicles generally begins sometime in the 30 s for most women and eventually leads to menopause, in most cases between ages 45 and 55.

In certain instances, it can be useful and desirable to determine the quality of an oocyte; for example, when a woman is seeking information about her level of fertility and chances of conception; or when a clinician seeks such information in association with assisted reproductive technologies (ART). Current methods are limited to the determination of oocyte quantity, not quality; furthermore, such methods are generally time consuming, expensive and require destruction of the oocyte itself. For a female subject who may be concerned about declining egg reserve and quality as she ages, a serious disadvantage is associated with any method or kit that requires the destruction of an oocyte in order to measure the subject's chances of conception. Further, since known methods are invasive, currently they can be performed only by medical personnel in a clinic or hospital setting; a woman cannot determine oocyte quality on her own. Thus, the current limitation of the technology to invasive methods has prevented the widespread adoption of diagnostic tests.

Thus, there is a need for methods and systems that can accurately predict or determine the quality of an oocyte, namely methods that are non-invasive, easy for patients to use, affordable and provide rapid and accurate results.

### SUMMARY OF THE DISCLOSED TECHNOLOGY

In certain embodiments, the present technology is directed to a method of determining the quality of an oocyte in the body of a human without disturbing or destroying the oocyte, the method comprising:

(a) obtaining a cell sample from a female subject, wherein the cell sample does not include the oocyte;

(b) measuring a characteristic of a gene or pathway indicative of oocyte quality in the cell sample; and

(c) predicting or determining the quality of the oocyte based on the characteristic of the gene or pathway.

In certain embodiments, the present technology is directed to a method of predicting the quality of an oocyte in the body of a mammal without disturbing or destroying the oocyte, the method comprising the steps of:

(a) obtaining a cell sample from the mammal, wherein the cell sample does not include the oocyte;

(b) conducting an RT-PCR assay or an ELISA assay on the cell sample using a primer for a gene known to be correlated with aging, and comparing the result with a known value obtained from a library of genes known to be correlated with decreased oocyte quality; and

(d) predicting the likelihood of oocyte viability based on (b).

In certain embodiments, the present technology is directed to a kit for predicting a woman's oocyte quality without the need for disturbing or destroying an oocyte, or of measuring a characteristic that correlates with the quality of an oocyte, the kit comprising:

(a) a collection container for collecting a cell sample obtained from the woman's body, wherein the cell sample does not include an oocyte;

(b) a testing assay comprising RT-PCR or ELISA, wherein the testing assay measures a characteristic of a gene, pathway or transcriptional profile characteristic of the cell sample, and wherein the characteristic indicates the likely quality of an oocyte; and

(c) a visual indicator visible to the woman, the visual indicator providing information regarding the predicted quality of the oocyte.

In certain embodiments, the present technology is directed to a method of producing a library of genes as markers of oocyte quality. Such a method may comprise the steps of correlating a test gene with a quantitative and measured characteristic of oocyte quality, listing the correlation in the library; comparing a measured characteristic of a gene provided by a patient with that listed in the library; and determining the quality of an oocyte of a patient based on the comparison. In certain embodiments, the technology herein contemplates a method of producing a library of genes as markers of oocyte quality, the method comprising the steps of:

(a) gathering expression data from cells of women in a particular age range;

(b) calculating an average gene expression for each gene at each age in the range by averaging the expression for that gene in a window of a given period of time;

(c) comparing the average gene expression of (b) to an “age vector” to indicate which genes change most with age; and

(d) calculating a FisherZ score, thereby identifying the genes at the tail ends of the distribution as indicators of biological age.

In certain embodiments, the present technology is directed to a reproductive aging gene expression profile; and a method of developing a reproductive aging gene expression profile and one or more candidate markers of reproductive success.

In certain embodiments, the present technology is directed to a method of producing a profile set as an indicator of oocyte quality. Such methods discussed herein may be done, for example, with any gene, pathway, or transcriptional profile, or with a library of the same.

### BRIEF DESCRIPTION OF THE FIGURES

FIG. 1a is a graph showing the prevalence of Down syndrome and infertility in a typical female human as a function of age. FIG. 1b is a graph showing the aging profile of oocytes in a typical female human as a function of age.

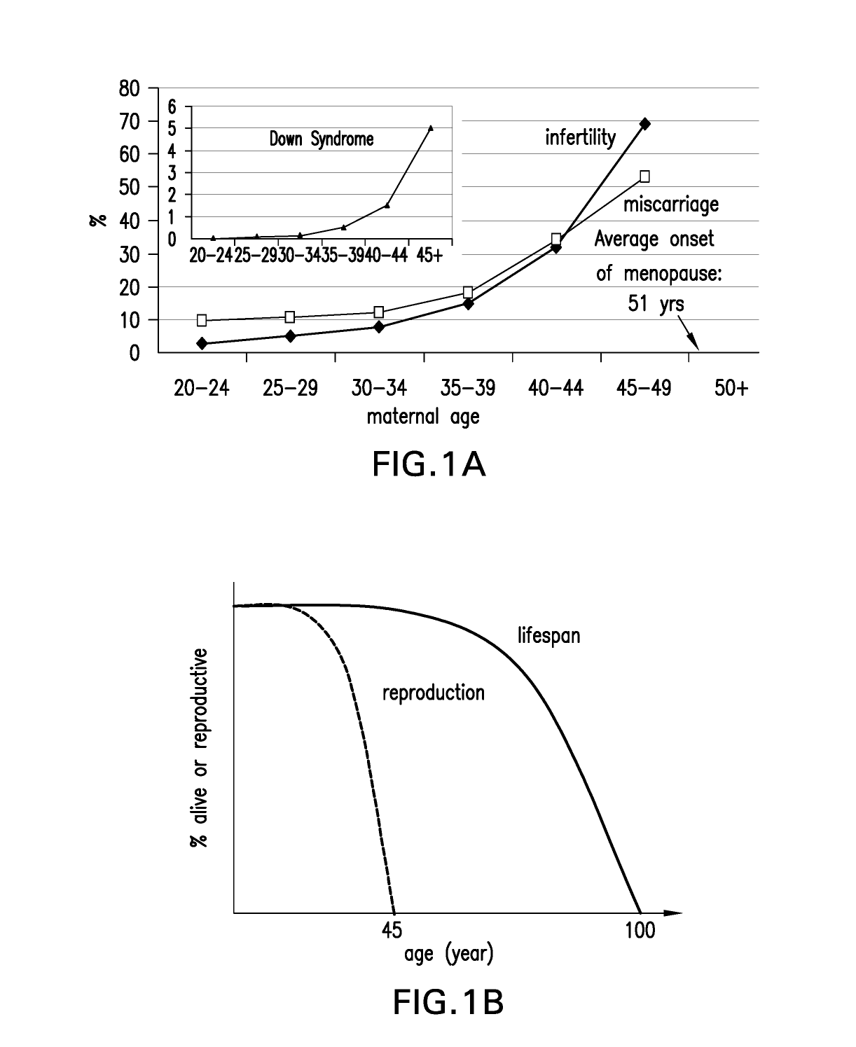

FIG. 2 shows age-dependent gene expression changes in blood from women aged 20 to 50, as a “heat map” which shows on a macro level relative changes.

### DETAILED DESCRIPTION

As used herein, “female subject” and “patient” are used interchangeably to refer to the individual whose oocyte quality is desired to be determined. As discussed herein, a “cell sample” refers to one or more cell from any part of the patient's body, which is desired to be tested in order to perform the diagnostic and scientific methods discussed herein. In various embodiments, a cell sample in accordance with the technology herein may be extracted from any of the following: blood, skin, hair, urine, saliva, sweat, vaginal secretion, any other fluid (including but not limited to intracellular or extracellular fluid, interstitial fluid, lymphatic fluid or transcellular fluid, cerebrospinal fluid, mucus or phlegm).

Oocyte quantity (the number of viable oocytes produced by a woman, the totality of which is determined long before the onset of puberty) and quality (the likelihood that the oocyte can be successfully fertilized and lead to pregnancy) are two factors of paramount importance in predicting fertility and the likelihood of carrying a pregnancy to term in a female. A woman's oocyte quality is generally predicted to decline after a certain age, until she reaches menopause and her ovaries stop releasing oocytes entirely. That is, with rising maternal age, and as a woman's oocytes age as well, a woman loses the ability to reproduce, and the children she may have are at greater risk for birth defects, chromosomal abnormalities, miscarriage and other problems. These defects start to arise when the woman is in her mid-30s and increase through the 40s, about a decade prior to menopause (the average age of menopause is 51), and are caused by declining oocyte quality.

Specifically, age-related reproductive decline is characterized by increasing levels of defects in oocyte chromosomal segregation, cell cycle arrest, and oocyte mitochondrial function and morphology, among other factors. Changes in gene expression correlate with these oocyte and egg quality changes, and have been shown in aging human and mouse oocytes. Our work in particular on C. elegans oocyte aging, discussed in greater detail below, has highlighted the evolutionary conservation of oocyte quality components. These components, particularly regulators of chromosome segregation fidelity, have been shown to be required for oocyte function and prevention of aneuploidy in worm and mammalian oocytes, and increased fidelity extends reproductive span.

However, despite the great concern about this problem, the exact age at which any particular woman might have problems is not known, and no diagnostic assay currently exists that can predict long-term age-related fertility status. Thus, a woman who is trying to conceive has limited options for determining the quality of an oocyte any given month or for predicting future success.

Current methods, such as endocrine tests of the ovarian reserve (oocyte number) predictors follicle stimulating hormone (FSH) and anti-mullerian hormone (AMH), have only immediate, short-term predictability of success with assisted reproductive technologies (ART) such as intrauterine insemination (IUI) in vitro fertilization (IVF), gamete intrafallopian tube transfer (GIFT) and zygote intrafallopian tube transfer (ZIFT). However, such tests cannot generally predict long-term prospective fertility. Similarly, known methods involving transvaginal ultrasound measurements of antral follicle count (AFC) focus on assessing oocyte number. However, oocyte quality, not quantity, is the limiting factor in most age-related fertility decline. In particular, aneuploidies (aberrations in chromosome numbers) and cell cycle arrest/maintenance failures can be a cause of infertility, birth defects, and miscarriage. Morphological assessments are too gross to identify other important measures of quality, such as the levels of particular maternal RNAs that regulate processes in the oocyte. Finally, current measures of oocyte quality require invasive approaches, and the assessment itself can destroy the oocyte in question, further limiting a woman's number of healthy oocytes and potentially requiring a larger pool of donated oocytes for treatment.

By contrast, bodily fluids such as blood and urine are easy to obtain, and in the case of blood, PBMCs (peripheral blood mononuclear cells) have been shown to provide information about the physiological state of other tissues, including ovarian cells. In fact, a recent study by Gielchinsky et al. in 2008 showed that PBMC expression analysis was able to identify candidate genes up- and down-regulated in women who delivered babies after spontaneous pregnancy at ?45 years of age. These candidate genes included markers of ovarian function, apoptosis, ubiquitination, energy production, and insulin/IGF-1 signaling—the same pathway that we showed extends reproductive span in C. elegans. While these factors are good candidates for age-related oocyte quality biomarkers in blood, our work can establish a timecourse for oocyte aging biomarkers in blood throughout the reproductive aging years, allowing us to identify and verify a set of biomarker genes to use for diagnostic purposes.

A diagnostic of oocyte quality, particularly a non-invasive measure, would be in high demand at several levels: clinics conducting assisted reproductive technology methods such as IVF would like this information before embarking on ART approaches to improve their success rates, egg-freezing companies could use such a diagnostic to predict the success of use of eggs they have frozen, and women could use the data to make informed decisions about their reproductive lives in a manner that is minimally invasive and highly convenient. Thus, in certain embodiments, the technology herein identifies, and permits health care workers or patients to identify biomarkers of oocyte quality in blood or other bodily fluids, as well as the development of a reliable long-term diagnostic of fertility.

Aging studies have been performed on Caenorhabditis elegans, a worm that has been developed as a model of reproductive aging for humans. As demonstrated by data from C. elegans and mice, certain genes in an organism are related to the quality of the oocytes of the organism.

In certain embodiments herein, the quality of an oocyte can be determined by, for example, measuring certain factors and comparing those factors with known values of oocytes of known age. Certain methods herein permit an investigator (including a health care worker or the subject herself) to determine or predict the quality of an oocyte in the body of a human without disturbing (e.g., touching, sampling, moving or altering) or destroying the oocyte itself, and without taking it out of the human's body, e.g., in vivo. Thus, in certain embodiments, the oocyte itself can be evaluated (or its characteristics determined and predicted) without harming the oocyte, and the oocyte can itself subsequently be fertilized and lead to a successful pregnancy.

In certain embodiments, the present technology relates to novel methods for assessing or predicting the quality of oocytes without the need for disturbing or destroying the oocytes themselves. These methods are based at least in part on the discovery that genes in C. elegans, which display changes as oocytes age, are the same genes as those found in mammals; further, even if no directly associated genes were located, mammalian genes with similar functions to the C. elegans genes were located. The C. elegans research also revealed data regarding the characteristics of gene expression in mutant C. elegans with long reproductive spans. Many of these genes are involved in chromosome segregation, spindle localization, chromosome organization, DNA damage response and repair, and mitochondrial processes such as ATP metabolism. These genes, therefore, are good biomarkers for oocyte quality from oocytes.

In certain embodiments, the present technology is directed to the exploitation of the non-invasive nature of blood biomarkers, or biomarkers from other bodily fluids. In certain embodiments, the present technology is directed to the development of single-gene markers as biomarkers; in other embodiments, the present technology is directed to profiles or multiple-gene markers, rather than single-gene markers, as biomarkers.

Table 1 shows pathways that Shown in the Table 1 is a summary of certain genes and pathways that have previously been identified as being of potential interest for human (H), mouse (M) and worm (W), in connection with applications of the present technology.

| | | Genes from oocyte array studies (homologs) | | |
|---|---|---|---|---|
| Gene Ontology | Gene | Worm (up | | |
| category | count | in sma-2) | Mouse | Human |
| Cell cycle | | | | |
| mitosis ^{HMW} | 8 | cyb-1(Ccnb2), | Ccnb2, Ccna2, | CCNA2, CCNG1, |
| | | cyb-3(Ccnb3), | Cdc16 | CDK7 |
| | | cdc-25.2(Cdc25a), | | |
| | | cki-1* | | |
| Chromosome | | | | |
| segregation, org'n | | | | |
| chromosome | 7 | smc-4(Smc4l1), klp-7, | Smc4l1, Nin, | Smc3l1, |
| segregation ^{HMW} | | frm-5 | Kif3b, Bub1 | BUB1B, BUB3 |
| spindle localization ^{M} | 4 | gad-1, mes-1, par-3 | Hook1, Nin, | |
| | | | Rnf19 | |
| spindle organization ^{M} | 3 | mbk-2, sur-6, goa-1 | Tuba2, Tubd1, | |
| | | | Pcnt2 | |
| chromosome | 12 | spr-5, nurf-1, hpl-1, | Hdac2, | |
| organization ^{MW} | | hil-2 | Morf4l2, | |
| | | | Rbbp7 | |
| DNA damage | | | | |
| response and repair | | | | |
| response to DNA | 4 | mlh-1(MLH1), clk-2, | Msh-3, Exo1, | MBD4 |
| damage stim. ^{M} | | pme-5, uev-2* | Shprh | (interacts with |
| | | | | MLH1), ATR, |
| | | | | NBS1 |
| Proteolytic pathway | | | | |
| proteolysis ^{HM} | 19 | ubc-1(Ube2a), | Ube2a, Ubc, | USP1, CTSC, |
| | | ubc-2(Ube2d1), ulp-1 | Usp1 | GRP58 |
| Energy pathway, | | | | |
| mitochondrial fn. | | | | |
| ATP metabolic process ^{M} | 7 | pmr-1(Atp2c1), (Atp6v1a), | Atp2c1, | |
| | | tat-5(Atp9b)vha-13 | Atp6v1d, Atp5b | |
| ATP binding ^{HMW} | 42 | pgp-7(Abcb11), mrp-2 | Abcb6, Abcf3, | ABCC4, |
| | | (Abcc3), | Cct2 | SMARCA5, SUV3 |
| | | psa-4(SMARCA5), | | |
| | | pdk-1, akt-2 | | |
| Cell signalling and | | | | |
| communication | | | | |
| intracellular signaling | 11 | cdc-42(RHO GTPase), | Rhoh, Kras2, | ATF1, CREB1, |
| cascade ^{HMW} | | vhp-1, sel-12 | Mek1 | CLK1 |
| cell-cell signaling ^{MW} | 5 | unc-18, ace1, cab-1 | Gja7, Shroom3, | |
| | | | Mmp2 | |
| Protein transport | | | | |
| protein transport ^{HMW} | 11 | arf-1.1(Arf1), arl-13 | Arf1, Arl4, Rab1 | ARF4, ARF6, |
| | | (Arl13b) | | RAB11a |
| | | rab-6.2 (Rab6) | | |
| Transcription | 19 | hlh-1, efI-1, spt-5 | Phtf1, Crsp6, | PHTF1, NFE2L2, |
| regulation ^{HMW} | | | Lhx8 | ElF2AK2 |
| Reproductive process | | | | |
| oogenesis ^{M} | 5 | hrp-1, goa-1, fem-3 | Nalp5, Padi5, | |
| | | | Nalp9a | |
| oyiposition ^{W} | 15 | unc-84, cki-1, mtm-3 | | |
| Other | | | | |
| cell death ^{MW} | 7 | ced-1, ced-8, crn-4 | Tnfaip8, | |
| | | | Mdm4, BcI2110 | |
| cell differentiation ^{W} | 24 | par-1, eor-2, !in-28 | | |
| cell adhesion ^{mw} | 6 | epi-1(Lama2), | Lama2, Cdh2, | |
| | | hmr-1(Cdh11), | Pcdhb17 | |
| | | cdh-3(Cdh23) | | |

In certain embodiments, the genes and pathways further include any of the following:

Genes in the IIS pathway: RHEB2, 14-3-3 beta/alpha, UDP-D-glucose cytosol, IGF-2, p90Rsk, MEK2 (MAP2K2), c-Myc, IRS-1, PtdIns(3,4,5)P3, PtdIns(4,5)P2, IKK (cat), Erk (MAPK1/3), Hamartin, c-Raf-1, AKT (PKB), PI3K cat class IA, ASK1 (MAP3K5), c-Raf-1, 14-3-3 epsilon, PTEN, 14-3-3 zeta/delta, IGF-1, PI3K reg class IA, p70 S6 kinase1, CREB1, GSK3 alpha/beta, Cyclin D, 3.1.3.67, MEK1 (MAP2K1), Bcl-XL, GRB2, PDK (PDPK1), mTOR, Caspase-9, FOXO3A, Tuberin, 2.7.1.137, Glycogen, I-kB, BAD, 4E-BP1, IGF-1 receptor, H-Ras, IBP, IKK-alpha, GYS1, 2.4.1.11, RPS6, She, SOS, NF-kB, Elk-1

Akt pathway (overlaps with IIS): MDM2, HGF receptor (Met), GYS1, 2.7.1.153, Bcl-XL, IKK-alpha, GAB1, Bax, Hamartin, PI3K cat class IA, BAD, IRS-1, PtdIns(3,4,5)P3, PtdIns(4,5)P2, 4E-BPI, mTOR, Caspase-9, FasL(TNFSF6), GSK3 alpha/beta, p21, NF-kB, IGF-1 receptor, RHEB2, PCNA, PI3K reg class IA, PTEN, PP2A catalytic, PDK (PDPK1), Cyclin D3, p53, 3.1.3.67, p27KIP1, AKT(PKB), Tuberin, RPS6, p70 S6 kinase1, c-Myc, HSP90, Cyclin D, IKK (cat), Bim, FOXO3A, I-kB

PTEN (overlaps with IIS): RHEB2, MEK1(MAP2K1), PtdIns(4,5)P2, Caspase-3, PDK (PDPK1), 3.1.3.67, ERK1/2, c-Jun, IGF-1, mTOR, PCNA, MEK2(MAP2K2), FOXO3A, p53, p21, FAK1, p130CAS, c-Cbl, Shc, GSK3 beta, BAD, Paxillin, EGF, PI3K reg class IA, H-Ras, MAGI-2, SOS, PTEN, c-Src, Tcf(Lef), alpha-5/beta-1 integrin, IRS-1, PI3K cat class IA, Beta-catenin, MAGI-3, EGFR, PtdIns(3,4,5)P3, IGF-1 receptor, Tuberin, ILK, GRB2, 2.7.1.137, AKT(PKB), c-Raf-1, MDM2, Caspase-9

Genes in the TGF-β pathway. p300, MSK1, SOS, NF-kB, TGF-beta receptor type I, SMAD7, p15, Elk-1, TGF-beta 1, SMAD4, Caveolin-1, ErbB2, IKK-beta, TSC-22, TIEG1, FKBP12, SMURF2, APC/hCDH1 complex, FAST-1/2, IKK-alpha, ERK1/2, SMAD3, TAK1(MAP3K7), PAIL ER81, MEKK4(MAP3K4), NFKBIA, GADD45 beta, H-Ras, Anaphase-promoting complex (APC), c-Raf-1, XIAP, TGF-beta receptor type II, SMURF1, CBP, p21, MEK6(MAP2K6), Sno-N, MEK2(MAP2K2), YY1, MEK3(MAP2K3), SARA, SP1, Shc, Importin (karyopherin)-beta, SMAD2, TABT, Ski, p38 MAPK, MEK1(MAP2K1)

FOXO transcription factors: FOXO1, FOXO3a

Genes in the Apoptosis pathways: ACVR1, BCL2L1, BIRC1, CAPN2, CAPNS1, CCNG1, CD47, CD81, CD99, CFLAR, CULT, DAD1, DGKA, DPP4, FST, GZMA, HLA-G, HSPA8, HSPD1, IGF1R, IL15, IL1A, IL2RG, IRS2, ITGB3, MAD2L1, MAP2K1, MAX, NRAS, ODC1, PDGFA, PECAM1, PGRMC1, PRKAR2B, PROS1, PTPN13, RBBP7, RPS3, SERPINB2, SKI, TEGT, TOP1, TPM1, YWHAQ, ZFF148

Genes in the Ubiquitination pathways: CAPN2, CAST, CD47, CFLAR, CLTC, CUL1, ESPL1, FLNA, HSPA8, IRS2, MAD2L1, MAP2K1, SIRT2, SKI, SNRPN, TOP1, UBQLN4, YWHAB, YWHAQ

Genes in the Energy Production pathways: ACVR1, BCL2L1, BIRC1, GSS, HLA-G, HSPA8, HSPD1, IGF1R, IRS2, KATNB1, MAP2K1, PGK1, PMS2, PRKD2, SLC25A3, SNRPN, TCF8, TOP1, TPM1

In certain embodiments, the gene or pathway indicative of oocyte quality is chosen from a gene expressed in connection with one or more of the following categories (reflecting those listed in Table 1): cell cycle (e.g., mitosis); chromosome segregation or organization (e.g., chromosome segregation, spindle localization or chromosome organization); DNA damage response and repair (e.g., response to DNA damage stimulus); proteolytic pathway (e.g., proteolysis); energy pathway or mitochondrial function (e.g., ATP metabolic process or ATP binding); cell signaling and communication (e.g., intracellular signaling cascade or cell-cell signaling); protein transport; transcription regulation; reproductive process (e.g., oogenesis or oviposition); cell death; cell differentiation or cell adhesion.

Other genes have been found to be of interest—among them, genes of chromatin structure, DNA methylation and genome stability. In particular, genes that have been shown to be associated with both ovarian function and general aging have shown promise in the methods, kits and libraries of the present technology. These include SERPINB2 (serpin peptidase inhibitor, Glade B, member 2, also known as PAI-2); IGFIR (insulin-like growth factor 1 receptor); PIK3CB (phosphoinositide-3-kinase, catalytic, beta polypeptide), IRS2 (insulin receptor substrate 2), HSPA8, HSPD1 and HSP60. Two genetic pathways in particular have been found herein to be significant in the regulation of reproductive aging. Specifically, it has previously been determined that TGF-β and insulin/IGF-1 (IIS) signaling pathways regulate reproductive aging cell non-autonomously. That is, these pathways regulate reproductive aging systemically, outside of the oocyte itself rather than by signaling just inside the oocyte. The function of these signaling pathways outside of oocytes (that is, in other areas of the organism's system) can determine the rate of reproductive aging, and analysis of a systemic tissue such as blood or urine for the activity of these pathways can provide useful data, including one or more biological indicators of reproductive aging of the oocytes.

In certain embodiments, the present technology is directed to transcriptional analysis of peripheral blood mononuclear cells (PMBCs). RNA sequencing is used to achieve greater sensitivity and depth. Biomarkers of reproductive age and reproductive success have been identified. The focus is on the most significant genes associated with reproductive status, regardless of whether the gene function is known or not, thus removing bias in selection. By using a panel of the genes most significantly associated with reproductive success, and then testing the expression of these genes for their predictive power, a diagnostic with a high correlation with outcome has been created. Additionally, analysis of the systemic effects on reproduction can inform subsequent approaches for treatments. Genes that have been shown to be downregulated include members of the insulin/IGF-1 (IIS) pathway (IGFAR, IRS2), and increased SIRT2, a co-regulator of the IIS pathway, is associated with late reproductive success. Therefore, previous studies of the C. elegans reproductive span correlates exactly with these observations in women, that one of the most important regulators of reproductive span appears to be the IIS pathway. Members of the TGF-β pathway can also be systemic markers of reproductive success. IIS can influence both lifespan and reproductive span, while TGF-β has an effect specifically on reproductive span. Thus, IIS differences in PBMCs are thought also to act as a diagnostic for longevity.

Thus, in certain embodiments, the technology herein is directed to methods for assessing a woman's likely oocyte quality, chances of conception, comprising developing a quantitative score that offers information regarding chances of conception or otherwise a measure of likelihood of success of an assisted reproductive procedure. In certain embodiments, the score can be calculated as follows: A sample's gene expression values can be obtained, and then how well the genes match a particular age can be determined in one of several ways, including but not limited to the following: (1) by Pearson correlation with a) the set of genes or pathways in the profile, if that is what is available by the gene set qRT-PCR assay, or b) by the Pearson correlation with the entire gene expression profile when the sample's total mRNA-sequence is assessed; or (2) by counting the genes with most extreme FisherZ scores more heavily (weighting) that includes the average expression of all the genes in the set.

The latter could give a single score that depends most heavily on the set of genes already evaluated. The former would likely allow for a better match of the whole transcriptome by age. In certain embodiments, both scores can be used to assess the score of the sample.

In certain embodiments, a “reproductive age” (as opposed to an actual age) of a given individual can be calculated as follows:

A pre-calculated age-correlation-score Zᵢ for each gene i (for a total of 19,600 genes in the genome) is based on public gene-expression data from blood from females. (These are the “fisherz” scores reported in all the analyses results.)

Given a new gene-expression sample with normalized expression levels eᵢ for each gene i, the “reproductive-age” aᵣₑₚ of that sample equals the weighted average of all the genes measured in the sample (N), weighted by the ‘age-correlation’ scores of those genes, in Equation (I) below:

$${a}_{rep} = \frac{{\sum }_{t = 1}^{N} {z}_{i} \cdot {e}_{i}}{{\sum }_{t = 1}^{N} {z}_{i}} \qquad ( I )$$

In certain embodiments, the present technology is directed to a kit comprising any one or more of the following: (a) a collection container for collecting a cell sample; and (b) an assay that measures a characteristic of a gene or pathway related to oocyte quality. In use, a medical professional, or the patient herself, may collect the cell sample, subject it to the assay and use the results to predict the patient's oocyte quality. For example, in certain embodiments the kit can predict a woman's likelihood of conceiving, a woman's “reproductive age” (as described later herein) or the likelihood that ovulation will result in a viable oocyte in any given month. Any of these can even be packaged in conjunction with other tools used by women trying to conceive, including but not limited to ovulation predictor kits, tools for measuring body temperature (such as basal body temperature thermometers), tools for measuring or evaluating mucus signs and other physically manifested indicators of fertility and fertile windows.

In various other embodiments, the kit comprises:

(a) a collection container for collecting a cell sample obtained from the woman's body;

(b) a testing assay that measures a characteristic of a gene, pathway or transcriptional profile characteristic of the cell sample, wherein the characteristic indicates the likely quality of an oocyte; and

(c) a visual indicator visible to the woman, the visual indicator providing information regarding the likely quality of the oocyte.

The visual indicator can be, for example, a color-coded indicator showing a binary result such as one color for an above average score, and another for a below average score.

In certain embodiments, the testing assay can be, for example, an RT-PCR assay on blood using the primers for the best genes. In the case of other samples such as urine or other bodily fluids, an assay can include metabolomics to assess metabolites or hormones, such as, for example, an enzyme-linked immunosorbent (ELISA) assay. In other embodiments, a colorimetric test could work in the event that the assay is matter of determining the levels of relatively few (e.g., just one, two or three, or in various embodiments fewer than about 10, fewer than about 25 or fewer than about 50) genes, metabolites or hormones. For example, in certain embodiments, the results of the assay could be displayed as one or more lines that would indicate the score. In certain embodiments, different lines could present different data points or results (for example, different colors or configurations, with the combination providing a score or other type of quantitative or qualitative result).

In certain embodiments, such a kit could be made commercially available for at-home use, and could include information taken from a library of genes and pathways generated in connection with certain embodiments herein.

In certain embodiments, the kit will include primers to amplify genes in this set (genes with FisherZ scores above 2 and below −2; see Table 2) from blood. In certain embodiments, any or all of the sample, or any characteristic thereof, can be assigned a score that is equal to the average of the expression level of all the genes in the array weighted by the age-correlation (FisherZ) score of each of the genes. This score conveys the expected oocyte viability for a woman compared to an average for women of the same age.

In certain embodiments, the present technology is directed to a method for the production of a library of genes, or a reproductive aging gene expression profile, as markers of oocyte quality. Such a method may comprise the steps of correlating a test gene with a quantitative and measured characteristic of oocyte quality, listing the correlation in the library; comparing a measured characteristic of a gene provided by a patient with that listed in the library; and determining the quality of an oocyte of a patient based on the comparison.

For example, in certain embodiments, a library is produced through any of the following steps: First, list certain genes known or thought to relate to factors such as ovarian function and general aging. Next, obtain samples of oocytes known to be from women of certain ages (for example, age 20, age 25, age 30 and the like) and measure one or more of the characteristics of those oocytes to obtain baseline values. Next, when a patient desires the assay, a cell sample is taken from the patient, the one or more characteristics can be determined by running the assay on the patient's cell sample and comparing the values to those of the library. For example, a patient may be 35 years old but may have oocytes that are typical of a 25 year old or a 40 year old, as determined by a method of the present technology, using the library generated according to this embodiment. This is valuable information that the patient can acquire without the need for invasive testing or destruction of her oocytes, and in certain embodiments, is obtained by implementing a method or kit in accordance with the embodiments herein. In certain embodiments, a yearly clinical assessment of oocyte age could allow a clinician or woman to determine the rate of change of oocyte quality, yet another indicator that could be useful for diagnostics and for advising patients.

In certain embodiments, a library can be created and used to establish a “profile set” for a user—that is, a set of one or more genes, pathways or transcriptional profiles that indicates various characteristics of a user's oocyte. A candidate gene expression assay can be developed based on the information obtained from bodily fluids such as blood, thus identifying biomarkers that correlate with oocyte quality and pregnancy success.

In one exemplary method, a library was made by the following steps:

(1) gathering expression data from samples (whole blood and PBMCs) from women in a particular age range (in certain embodiments, aged 20 to 50, but not so limited). An average gene expression for each gene at each age was calculated by averaging the expression for that gene in a given range of time (e.g., a 2 year window, in overlapping (sliding) windows). This allowed the inclusion of about 30 to about 90 samples in each year. In certain embodiments, an average of about 70 to about 80 GEO female blood samples were used for each 2 year sliding window; for each gene, the Spearmann correlation of the average gene expression to the age vector was determined, and then sorted by the FisherZ score; a score above 2 (top 5%) was used to generate a set of significantly changed age-dependent genes, all candidate biomarkers.

(2) The gene expression data were compared to an “age vector” to indicate which genes changed most with age. Since most genes do not have an age-related change in expression, the data are normally distributed. The FisherZ score makes it possible to find the genes at the tail ends of the distribution; those genes are most changed with age, and thus are the best indicators of biological age. Such genes are included in the library, and can be used to compare with data acquired from the test subject.

For the samples of women aged 25 to 50, in certain embodiments only samples from women who have no known fertility defect (i.e., presence at IVF clinic is due to sperm or other partner issue) can be used to establish a “young” profile. The expression profiles can then be sorted according to age and further segregated by pregnancy success/failure and other cytological information, and PCA can be performed to identify the genes best correlated with pregnancy success. Using qRT-PCR, the top genes most associated with pregnancy outcome can be tested on a separate sample set prior to IVF (or other ART treatment) to verify predictive power of the gene set. The most predictive genes can be used to develop a diagnostic tool that can be used in clinics after a simple blood draw or urine collection. The clinical procedure RT-PCR can be performed on RNA from blood samples using the mix of primers of the best candidate genes.

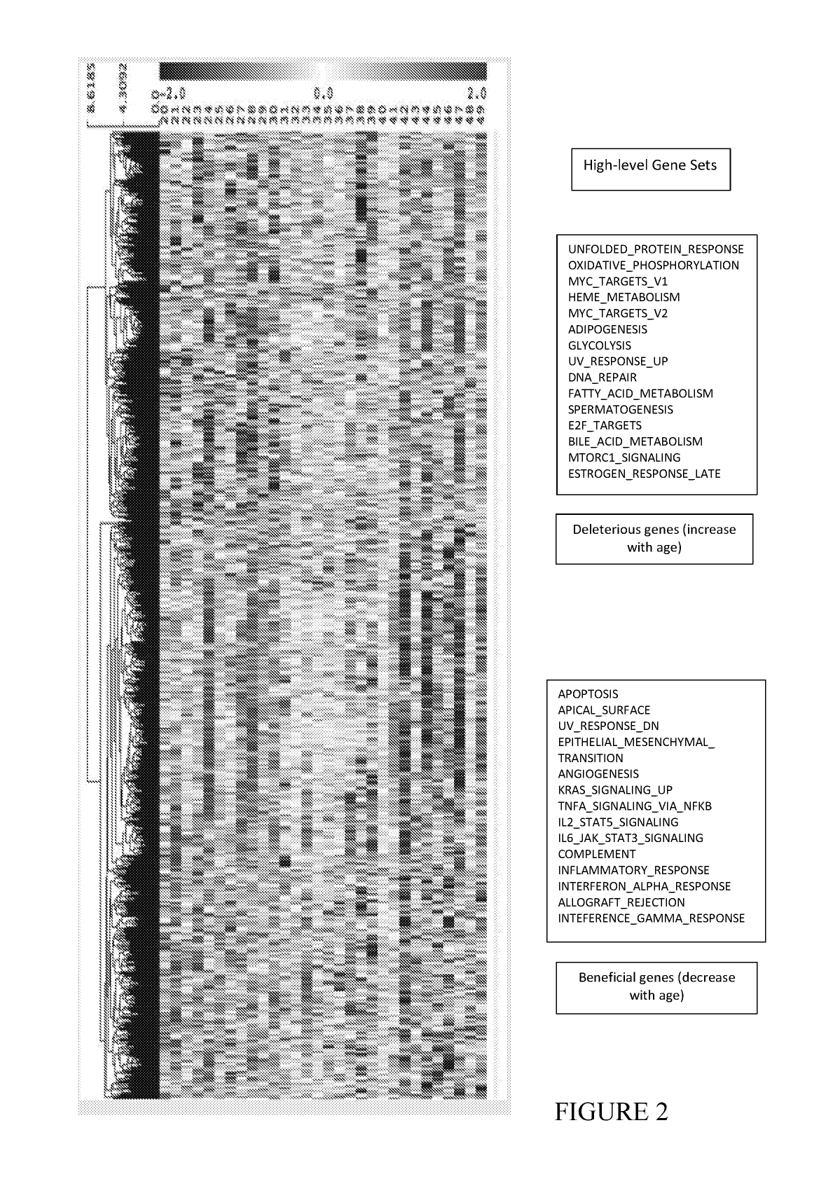

FIG. 2 shows a heat map of the results showing gene trees. Tables 2 and 3 below shows data from FIG. 2 in numerical form. Table 2 shows the results for ages 20-33; Table 3 shows the results for ages 34-40.

TABLE 2

| gene | symbol | name | fisherz | 20 | 21 |
|---|---|---|---|---|---|
| 56904 | SH3GLB2 | SH3-domain | 3.67 | −0.38 | −0.45 |
| | | GRB2-like | | | |
| | | endophilin B2 | | | |
| 6464 | SHC1 | SHC (Src | 3.56 | −0.45 | −1.44 |
| | | homology 2 | | | |
| | | domain | | | |
| | | containing) | | | |
| | | transforming | | | |
| | | protein 1 | | | |
| 79087 | ALG12 | ALG12, alpha- | 3.45 | −0.66 | −0.84 |
| | | 1,6- | | | |
| | | mannosyltransferase | | | |
| 101928190 | LOC101928190 | uncharacterized | 3.43 | −1.81 | −1.56 |
| | | LOC101928190 | | | |
| 3416 | IDE | insulin- | 3.17 | −0.90 | −0.60 |
| | | degrading | | | |
| | | enzyme | | | |
| 8001 | GLRA3 | glycine receptor, | 3.16 | −0.68 | −1.27 |
| | | alpha 3 | | | |
| 3727 | JUND | jun D proto- | 3.13 | −0.39 | −1.05 |
| | | oncogene | | | |
| 10945 | KDELR1 | KDEL (Lys- | 3.12 | −0.05 | −1.56 |
| | | Asp-Glu-Leu) | | | |
| | | endoplasmic | | | |
| | | reticulum | | | |
| | | protein retention | | | |
| | | receptor 1 | | | |
| 5025 | P2RX4 | purinergic | 3.09 | −0.52 | −0.03 |
| | | receptor P2X, | | | |
| | | ligand-gated ion | | | |
| | | channel, 4 | | | |
| 29920 | PYCR2 | pyrroline-5- | 3.07 | −0.44 | −0.06 |
| | | carboxylate | | | |
| | | reductase | | | |
| | | family, member 2 | | | |
| 101929248 | LOC101929248 | uncharacterized | 3.01 | −0.18 | −0.66 |
| | | LOC101929248 | | | |
| 23646 | PLD3 | phospholipase D | 2.98 | −0.35 | −1.18 |
| | | family, member 3 | | | |
| 10280 | SIGMAR1 | sigma non- | 2.97 | −0.26 | −0.98 |
| | | opioid | | | |
| | | intracellular | | | |
| | | receptor 1 | | | |
| 140699 | MROH8 | maestro heat- | 2.96 | −1.96 | −1.29 |
| | | like repeat | | | |
| | | family member 8 | | | |
| 4669 | NAGLU | N- | 2.94 | 1.04 | −1.05 |
| | | acetylglucosaminidase, | | | |
| | | alpha | | | |
| 8341 | HIST1H2BN | histone cluster | 2.91 | −0.20 | −1.26 |
| | | 1, H2bn | | | |
| 2519 | FUCA2 | fucosidase, | 2.90 | −2.72 | −1.16 |
| | | alpha-L-2, | | | |
| | | plasma | | | |
| 8985 | PLOD3 | procollagen- | 2.88 | 0.48 | −0.96 |
| | | lysine, 2- | | | |
| | | oxoglutarate 5- | | | |
| | | dioxygenase 3 | | | |
| 1690 | COCH | cochlin | 2.87 | −0.79 | −2.11 |
| 5111 | ZDHHC9 | zinc finger, | 2.86 | 0.19 | −0.88 |
| | | DHHC-type | | | |
| | | containing 9 | | | |
| 55907 | CMAS | cytidine | 2.85 | −0.20 | −0.12 |
| | | monophosphate | | | |
| | | N- | | | |
| | | acetylneuraminic | | | |
| | | acid synthetase | | | |
| 144811 | LACC1 | laccase | 2.84 | 0.65 | −2.69 |
| | | (multicopper | | | |
| | | oxidoreductase) | | | |
| | | domain | | | |
| | | containing 1 | | | |
| 92421 | CHMP4C | charged | 2.84 | −1.75 | 0.40 |
| | | multivesicular | | | |
| | | body protein 4C | | | |
| 23475 | QPRT | quinolinate | 2.83 | 0.03 | −0.54 |
| | | phosphoribosyltransferase | | | |
| 54913 | RPP25 | ribonuclease | 2.77 | −1.16 | −1.29 |
| | | P/MRP 25 kDa | | | |
| | | subunit | | | |
| 4645 | MYO5B | myosin VB | 2.76 | −1.61 | −0.94 |
| 9917 | FAM20B | family with | 2.74 | −0.21 | −0.82 |
| | | sequence | | | |
| | | similarity 20, | | | |
| | | member B | | | |
| 57213 | SPRYD7 | SPRY domain | 2.73 | 0.00 | −1.85 |
| | | containing 7 | | | |
| 113829 | SLC35A4 | solute carrier | 2.73 | −0.37 | −0.56 |
| | | family 35, | | | |
| | | member A4 | | | |
| 100128164 | LOC100128164 | four and a half | 2.72 | −1.16 | −0.96 |
| | | LIM domains 1 | | | |
| | | pseudogene | | | |
| 26284 | ERAL1 | Era-like 1 2S | 2.70 | 0.75 | −0.67 |
| | | mitochondrial | | | |
| | | rRNA | | | |
| | | chaperone 1 | | | |
| 64847 | SPATA20 | spermatogenesis | 2.70 | 1.79 | −1.47 |
| | | associated 20 | | | |
| 55974 | SLC50A1 | solute carrier | 2.69 | −0.45 | −0.88 |
| | | family 50 (sugar | | | |
| | | efflux | | | |
| | | transporter), | | | |
| | | member 1 | | | |
| 64405 | CDH22 | cadherin 22, | 2.68 | −0.95 | −1.41 |
| | | type 2 | | | |
| 6510 | SLC1A5 | solute carrier | 2.68 | −0.54 | −1.66 |
| | | family 1 (neutral | | | |
| | | amino acid | | | |
| | | transporter), | | | |
| | | member 5 | | | |
| 196463 | PLBD2 | phospholipase B | 2.67 | −0.48 | −1.13 |
| | | domain | | | |
| | | containing 2 | | | |
| 741 | ZNHIT2 | zinc finger, | 2.67 | −2.15 | 0.74 |
| | | HIT-type | | | |
| | | containing 2 | | | |
| 23568 | ARL2BP | ADP- | 2.66 | 0.53 | 0.31 |
| | | ribosylation | | | |
| | | factor-like 2 | | | |
| | | binding protein | | | |
| 79144 | PPDPF | pancreatic | 2.66 | 0.02 | −1.75 |
| | | progenitor cell | | | |
| | | differentiation | | | |
| | | and proliferation | | | |
| | | factor | | | |
| 91319 | DERL3 | derlin 3 | 2.66 | −0.64 | −0.55 |
| 79654 | HECTD3 | HECT domain | 2.64 | 0.28 | −2.10 |
| | | containing E3 | | | |
| | | ubiquitin protein | | | |
| | | ligase 3 | | | |
| 115950 | ZNF653 | zinc finger | 2.63 | −0.11 | −0.32 |
| | | protein 653 | | | |
| 1374 | CPT1A | carnitine | 2.63 | −0.46 | −1.45 |
| | | palmitoyltransferase | | | |
| | | 1A (liver) | | | |
| 81533 | ITFG1 | integrin alpha | 2.62 | −2.21 | −1.14 |
| | | FG-GAP repeat | | | |
| | | containing 1 | | | |
| 201931 | TMEM192 | transmembrane | 2.60 | −1.17 | −1.00 |
| | | protein 192 | | | |
| 7979 | SHFM1 | split hand/foot | 2.60 | −0.03 | 0.14 |
| | | malformation | | | |
| | | (ectrodactyly) | | | |
| | | type 1 | | | |
| 159296 | NKX2-3 | NK2 homeobox 3 | 2.59 | 0.18 | −0.81 |
| 51102 | MECR | mitochondrial | 2.59 | 0.73 | −0.13 |
| | | trans-2-enoyl- | | | |
| | | CoA reductase | | | |
| 8729 | GBF1 | golgi brefeldin | 2.59 | −0.82 | −0.51 |
| | | A resistant | | | |
| | | guanine | | | |
| | | nucleotide | | | |
| | | exchange factor 1 | | | |
| 118460 | EXOSC6 | exosome | 2.58 | −0.87 | 0.01 |
| | | component 6 | | | |
| 79586 | CHPF | chondroitin | 2.57 | −1.64 | −0.31 |
| | | polymerizing | | | |
| | | factor | | | |
| 2052 | EPHX1 | epoxide | 2.54 | 0.15 | −2.10 |
| | | hydrolase 1, | | | |
| | | microsomal | | | |
| | | (xenobiotic) | | | |
| 84893 | FBXO18 | F-box protein, | 2.53 | −1.99 | 0.08 |
| | | helicase, 18 | | | |
| 54726 | OTUD4 | OTU domain | 2.53 | −0.35 | 0.31 |
| | | containing 4 | | | |
| 813 | CALU | calumenin | 2.53 | −0.42 | 0.21 |
| 54431 | DNAJC10 | DnaJ (Hsp40) | 2.52 | 0.67 | −1.18 |
| | | homolog, | | | |
| | | subfamily C, | | | |
| | | member 10 | | | |
| 9612 | NCOR2 | nuclear receptor | 2.52 | −0.79 | −1.22 |
| | | corepressor 2 | | | |
| 9829 | DNAJC6 | DnaJ (Hsp40) | 2.51 | −1.56 | 0.25 |
| | | homolog, | | | |
| | | subfamily C, | | | |
| | | member 6 | | | |
| 79036 | KXD1 | KxDL motif | 2.51 | −1.30 | −0.97 |
| | | containing 1 | | | |
| 284361 | EMC10 | ER membrane | 2.51 | 0.21 | −0.27 |
| | | protein complex | | | |
| | | subunit 10 | | | |
| 131118 | DNAJC19 | DnaJ (Hsp40) | 2.50 | −0.13 | −0.23 |
| | | homolog, | | | |
| | | subfamily C, | | | |
| | | member 19 | | | |
| 644809 | C15orf56 | chromosome 15 | 2.50 | −2.50 | −1.68 |
| | | open reading | | | |
| | | frame 56 | | | |
| 2119 | ETV5 | ets variant 5 | 2.50 | 0.74 | −2.06 |
| 5833 | PCYT2 | phosphate | 2.50 | −0.95 | −0.34 |
| | | cytidylyltransferase | | | |
| | | 2, | | | |
| | | ethanolamine | | | |
| 922 | CD5L | CD5 molecule- | 2.49 | 0.26 | −1.47 |
| | | like | | | |
| 9261 | MAPKAPK2 | mitogen- | 2.49 | −0.65 | −0.96 |
| | | activated protein | | | |
| | | kinase-activated | | | |
| | | protein kinase 2 | | | |
| 57190 | SEPN1 | selenoprotein N, 1 | 2.49 | −0.88 | −0.59 |
| 79644 | SRD5A3 | steroid 5 alpha- | 2.48 | 1.23 | −0.67 |
| | | reductase 3 | | | |
| 79058 | ASPSCR1 | alveolar soft part | 2.48 | −0.54 | 0.00 |
| | | sarcoma | | | |
| | | chromosome | | | |
| | | region, | | | |
| | | candidate 1 | | | |
| 7832 | BTG2 | BTG family, | 2.47 | −0.51 | −0.67 |
| | | member 2 | | | |
| 11070 | TMEM115 | transmembrane | 2.47 | 0.00 | −0.03 |
| | | protein 115 | | | |
| 10847 | SRCAP | Snf2-related | 2.46 | −1.45 | −0.63 |
| | | CREBBP | | | |
| | | activator protein | | | |
| 4597 | MVD | mevalonate | 2.46 | −1.41 | −1.21 |
| | | (diphospho) | | | |
| | | decarboxylase | | | |
| 100506696 | PCAT6 | prostate cancer | 2.45 | 0.45 | −0.79 |
| | | associated | | | |
| | | transcript 6 | | | |
| | | (non-protein | | | |
| | | coding) | | | |
| 100507459 | LOC100507459 | uncharacterized | 2.45 | −0.67 | −2.06 |
| | | LOC100507459 | | | |
| 100130613 | CXorf64 | chromosome X | 2.45 | −0.70 | −0.39 |
| | | open reading | | | |
| | | frame 64 | | | |
| 5081 | PAX7 | paired box 7 | 2.45 | −0.18 | −0.61 |
| 100240728 | LOC100240728 | uncharacterized | 2.44 | 0.45 | −0.36 |
| | | LOC100240728 | | | |
| 26229 | B3GAT3 | beta-1,3- | 2.44 | −0.09 | 0.75 |
| | | glucuronyltransferase 3 | | | |
| | | (glucuronosyltransferase | | | |
| | | I) | | | |
| 950 | SCARB2 | scavenger | 2.44 | 0.11 | −1.84 |
| | | receptor class B, | | | |
| | | member 2 | | | |
| 161882 | ZFPM1 | zinc finger | 2.44 | 0.78 | −1.19 |
| | | protein, FOG | | | |
| | | family member 1 | | | |
| 5034 | P4HB | prolyl 4- | 2.43 | −0.03 | −0.65 |
| | | hydroxylase, | | | |
| | | beta polypeptide | | | |
| 123355 | LRRC28 | leucine rich | 2.43 | −3.02 | −0.37 |
| | | repeat | | | |
| | | containing 28 | | | |
| 6734 | SRPR | signal | 2.43 | −0.95 | −0.99 |
| | | recognition | | | |
| | | particle receptor | | | |
| | | (docking | | | |
| | | protein) | | | |
| 9911 | TMCC2 | transmembrane | 2.43 | −0.31 | −1.12 |
| | | and coiled-coil | | | |
| | | domain family 2 | | | |
| 222642 | TSPO2 | translocator | 2.42 | −0.04 | −0.31 |
| | | protein 2 | | | |
| 3643 | INSR | insulin receptor | 2.42 | −1.47 | −1.69 |
| 85462 | FHDC1 | FH2 domain | 2.41 | −1.86 | −1.61 |
| | | containing 1 | | | |
| 149175 | MANEAL | mannosidase, | 2.40 | 0.96 | −1.03 |
| | | endo-alpha-like | | | |
| 1819 | DRG2 | developmentally | 2.40 | −0.30 | 0.61 |
| | | regulated GTP | | | |
| | | binding protein 2 | | | |
| 10370 | CITED2 | Cbp/p300- | 2.39 | −0.55 | −1.11 |
| | | interacting | | | |
| | | transactivator, | | | |
| | | with Glu/Asp- | | | |
| | | rich carboxy- | | | |
| | | terminal | | | |
| | | domain, 2 | | | |
| 25792 | CIZ1 | CDKN1A | 2.39 | −0.51 | −0.14 |
| | | interacting zinc | | | |
| | | finger protein 1 | | | |
| 4072 | EPCAM | epithelial cell | 2.39 | −0.61 | −0.92 |
| | | adhesion | | | |
| | | molecule | | | |
| 84447 | SYVN1 | synovial | 2.39 | −0.16 | −1.58 |
| | | apoptosis | | | |
| | | inhibitor 1, | | | |
| | | synoviolin | | | |
| 527 | ATP6V0C | ATPase, H+ | 2.39 | −1.34 | −1.27 |
| | | transporting, | | | |
| | | lysosomal | | | |
| | | 16 kDa, V0 | | | |
| | | subunit c | | | |
| 10629 | TAF6L | TAF6-like RNA | 2.38 | −0.68 | −0.53 |
| | | polymerase II, | | | |
| | | p300/CBP- | | | |
| | | associated factor | | | |
| | | (PCAF)- | | | |
| | | associated | | | |
| | | factor, 65 kDa | | | |
| 51150 | SDF4 | stromal cell | 2.38 | −0.19 | −0.15 |
| | | derived factor 4 | | | |
| 57143 | ADCK1 | aarF domain | 2.37 | −0.37 | −0.83 |
| | | containing | | | |
| | | kinase 1 | | | |
| 682 | BSG | basigin | 2.37 | −0.21 | −1.33 |
| 10139 | ARFRP1 | ADP- | 2.37 | −1.43 | 0.38 |
| | | ribosylation | | | |
| | | factor related | | | |
| | | protein 1 | | | |
| 203238 | CCDC171 | coiled-coil | 2.37 | 0.06 | −0.78 |
| | | domain | | | |
| | | containing 171 | | | |
| 54805 | CNNM2 | cyclin M2 | 2.37 | −0.64 | 0.23 |
| 1200 | TPP1 | tripeptidyl | 2.37 | 0.19 | −2.22 |
| | | peptidase I | | | |
| 2271 | FH | fumarate | 2.37 | −0.93 | −0.36 |
| | | hydratase | | | |
| 376412 | RNF126P1 | ring finger | 2.37 | −0.06 | −0.52 |
| | | protein 126 | | | |
| | | pseudogene 1 | | | |
| 4924 | NUCB1 | nucleobindin 1 | 2.37 | −1.22 | −1.33 |
| 55132 | LARP1B | La | 2.37 | 0.00 | 0.03 |
| | | ribonucleoprotein | | | |
| | | domain | | | |
| | | family, member | | | |
| | | 1B | | | |
| 9650 | MTFR1 | mitochondrial | 2.37 | 0.28 | −0.07 |
| | | fission regulator 1 | | | |
| 29803 | REPIN1 | replication | 2.36 | −0.05 | −2.01 |
| | | initiator 1 | | | |
| 4507 | MTAP | methylthioadenosine | 2.36 | −0.50 | 0.16 |
| | | phosphorylase | | | |
| 816 | CAMK2B | calcium/calmodulin- | 2.36 | −0.96 | −1.38 |
| | | dependent | | | |
| | | protein kinase II | | | |
| | | beta | | | |
| 120379 | PIH1D2 | PIH1 domain | 2.36 | 0.24 | −0.66 |
| | | containing 2 | | | |
| 91289 | LMF2 | lipase | 2.36 | −0.15 | −1.01 |
| | | maturation | | | |
| | | factor 2 | | | |
| 10905 | MAN1A2 | mannosidase, | 2.35 | −0.52 | −0.40 |
| | | alpha, class 1A, | | | |
| | | member 2 | | | |
| 25930 | PTPN23 | protein tyrosine | 2.35 | −0.79 | −1.96 |
| | | phosphatase, | | | |
| | | non-receptor | | | |
| | | type 23 | | | |
| 57648 | KIAA1522 | KIAA1522 | 2.35 | −1.31 | −1.46 |
| 7249 | TSC2 | tuberous | 2.35 | −0.03 | −0.57 |
| | | sclerosis 2 | | | |
| 55151 | TMEM38B | transmembrane | 2.34 | 0.29 | −0.25 |
| | | protein 38B | | | |
| 160728 | SLC5A8 | solute carrier | 2.33 | −1.06 | −1.61 |
| | | family 5 | | | |
| | | (sodium/monocarboxylate | | | |
| | | cotransporter), | | | |
| | | member 8 | | | |
| 148418 | SAMD13 | sterile alpha | 2.33 | −0.78 | 0.86 |
| | | motif domain | | | |
| | | containing 13 | | | |
| 646762 | LOC646762 | uncharacterized | 2.32 | −0.60 | −1.03 |
| | | LOC646762 | | | |
| 79109 | MAPKAP1 | mitogen- | 2.32 | −0.93 | −0.74 |
| | | activated protein | | | |
| | | kinase | | | |
| | | associated | | | |
| | | protein 1 | | | |
| 81037 | CLPTM1L | CLPTM1-like | 2.31 | −0.28 | −0.13 |
| 51523 | CXXC5 | CXXC finger | 2.29 | 0.91 | −1.19 |
| | | protein 5 | | | |
| 55322 | C5orf22 | chromosome 5 | 2.29 | −0.14 | −1.05 |
| | | open reading | | | |
| | | frame 22 | | | |
| 100996671 | LOC100996671 | uncharacterized | 2.29 | −1.07 | 0.25 |
| | | LOC100996671 | | | |
| 55222 | LRRC20 | leucine rich | 2.29 | 0.14 | 0.15 |
| | | repeat | | | |
| | | containing 20 | | | |
| 55624 | POMGNT1 | protein O-linked | 2.29 | 0.42 | −0.59 |
| | | mannose N- | | | |
| | | acetylglucosaminyltransferase 1 | | | |
| | | (beta 1,2-) | | | |
| 100505983 | LOC100505983 | uncharacterized | 2.28 | −0.30 | −1.40 |
| | | LOC100505983 | | | |
| 3069 | HDLBP | high density | 2.28 | 0.02 | −0.50 |
| | | lipoprotein | | | |
| | | binding protein | | | |
| 10959 | TMED2 | transmembrane | 2.28 | −0.64 | 0.50 |
| | | emp24 domain | | | |
| | | trafficking | | | |
| | | protein 2 | | | |
| 6598 | SMARCB1 | SWI/SNF | 2.28 | −0.03 | −1.04 |
| | | related, matrix | | | |
| | | associated, actin | | | |
| | | dependent | | | |
| | | regulator of | | | |
| | | chromatin, | | | |
| | | subfamily b, | | | |
| | | member 1 | | | |
| 60412 | EXOC4 | exocyst complex | 2.27 | −0.97 | −0.75 |
| | | component 4 | | | |
| 54982 | CLN6 | ceroid- | 2.27 | −0.22 | −0.05 |
| | | lipofuscinosis, | | | |
| | | neuronal 6, late | | | |
| | | infantile, variant | | | |
| 91752 | ZNF804A | zinc finger | 2.27 | −0.60 | −0.96 |
| | | protein 804A | | | |
| 113178 | SCAMP4 | secretory carrier | 2.26 | −0.63 | −0.57 |
| | | membrane | | | |
| | | protein 4 | | | |
| 10 | NAT2 | N- | 2.26 | −2.14 | −0.97 |
| | | acetyltransferase | | | |
| | | 2 (arylamine N- | | | |
| | | acetyltransferase) | | | |
| 375 | ARF1 | ADP- | 2.26 | −0.93 | 0.03 |
| | | ribosylation | | | |
| | | factor 1 | | | |
| 5589 | PRKCSH | protein kinase C | 2.26 | −0.64 | −1.59 |
| | | substrate 80K-H | | | |
| 9136 | RRP9 | ribosomal RNA | 2.26 | −0.92 | 0.08 |
| | | processing 9, | | | |
| | | small subunit | | | |
| | | (SSU) | | | |
| | | processome | | | |
| | | component, | | | |
| | | homolog (yeast) | | | |
| 23193 | GANAB | glucosidase, | 2.26 | −0.20 | 0.04 |
| | | alpha; neutral | | | |
| | | AB | | | |
| 1798 | DPAGT1 | dolichyl- | 2.25 | 0.46 | −0.97 |
| | | phosphate | | | |
| | | (UDP-N- | | | |
| | | acetylglucosamine) | | | |
| | | N- | | | |
| | | acetylglucosaminephosphotransferase 1 | | | |
| | | (GlcNAc-1-P | | | |
| | | transferase) | | | |
| 85440 | DOCK7 | dedicator of | 2.25 | 0.62 | −1.10 |
| | | cytokinesis 7 | | | |
| 100505794 | LOC100505794 | uncharacterized | 2.24 | −1.88 | −0.15 |
| | | LOC100505794 | | | |
| 1173 | AP2M1 | adaptor-related | 2.24 | 0.00 | −0.87 |
| | | protein complex | | | |
| | | 2, mu 1 subunit | | | |
| 124540 | MSI2 | musashi RNA- | 2.24 | −0.83 | −0.24 |
| | | binding protein 2 | | | |
| 8372 | HYAL3 | hyaluronoglucosaminidase 3 | 2.24 | −0.32 | 0.20 |
| 64755 | C16orf58 | chromosome 16 | 2.24 | 0.94 | −0.41 |
| | | open reading | | | |
| | | frame 58 | | | |
| 79102 | RNF26 | ring finger | 2.24 | −0.02 | −0.85 |
| | | protein 26 | | | |
| 10963 | STIP1 | stress-induced- | 2.24 | −1.07 | −0.30 |
| | | phosphoprotein 1 | | | |
| 375387 | NRROS | negative | 2.24 | 0.14 | −0.08 |
| | | regulator of | | | |
| | | reactive oxygen | | | |
| | | species | | | |
| 972 | CD74 | CD74 molecule, | 2.23 | 1.04 | −3.36 |
| | | major | | | |
| | | histocompatibility | | | |
| | | complex, class | | | |
| | | II invariant | | | |
| | | chain | | | |
| 54676 | GTPBP2 | GTP binding | 2.23 | 0.44 | −1.39 |
| | | protein 2 | | | |
| 192286 | HIGD2A | HIG1 hypoxia | 2.23 | 1.25 | −0.69 |
| | | inducible | | | |
| | | domain family, | | | |
| | | member 2A | | | |
| 3295 | HSD17B4 | hydroxysteroid | 2.23 | −0.22 | −1.28 |
| | | (17-beta) | | | |
| | | dehydrogenase 4 | | | |
| 80852 | GRIP2 | glutamate | 2.23 | −1.32 | −0.75 |
| | | receptor | | | |
| | | interacting | | | |
| | | protein 2 | | | |
| 1298 | COL9A2 | collagen, type | 2.22 | −1.39 | −2.02 |
| | | IX, alpha 2 | | | |
| 2004 | ELK3 | ELK3, ETS- | 2.22 | 0.12 | −0.69 |
| | | domain protein | | | |
| | | (SRF accessory | | | |
| | | protein 2) | | | |
| 10279 | PRSS1 | protease, serine, | 2.22 | 0.08 | 0.44 |
| | | 16 (thymus) | | | |
| 151790 | WDR49 | WD repeat | 2.22 | −1.01 | −1.82 |
| | | domain 49 | | | |
| 399664 | MEX3D | mex-3 RNA | 2.21 | −1.01 | 0.55 |
| | | binding family | | | |
| | | member D | | | |
| 5664 | PSEN2 | presenilin 2 | 2.21 | 1.22 | −1.57 |
| | | (Alzheimer | | | |
| | | disease 4) | | | |
| 51614 | ERGIC3 | ERGIC and | 2.21 | 0.53 | −0.17 |
| | | golgi 3 | | | |
| 79178 | THTPA | thiamine | 2.21 | 0.37 | −0.69 |
| | | triphosphatase | | | |
| 51726 | DNAJB11 | DnaJ (Hsp40) | 2.20 | −0.15 | 0.16 |
| | | homolog, | | | |
| | | subfamily B, | | | |
| | | member 11 | | | |
| 3425 | IDUA | iduronidase, | 2.20 | 0.16 | 0.01 |
| | | alpha-L- | | | |
| 1642 | DDB1 | damage-specific | 2.20 | 0.72 | −0.92 |
| | | DNA binding | | | |
| | | protein 1, | | | |
| | | 127 kDa | | | |
| 101928770 | LOC101928770 | uncharacterized | 2.19 | −1.77 | −0.87 |
| | | LOC101928770 | | | |
| 11015 | KDELR3 | KDEL (Lys- | 2.19 | 0.74 | −1.45 |
| | | Asp-Glu-Leu) | | | |
| | | endoplasmic | | | |
| | | reticulum | | | |
| | | protein retention | | | |
| | | receptor 3 | | | |
| 35 | ACADS | acyl-CoA | 2.19 | −0.36 | −0.45 |
| | | dehydrogenase, | | | |
| | | C-2 to C-3 short | | | |
| | | chain | | | |
| 64798 | DEPTOR | DEP domain | 2.19 | 0.99 | −0.91 |
| | | containing | | | |
| | | MTOR- | | | |
| | | interacting | | | |
| | | protein | | | |
| 23457 | ABCB9 | ATP-binding | 2.19 | −0.90 | −0.60 |
| | | cassette, sub- | | | |
| | | family B | | | |
| | | (MDR/TAP), | | | |
| | | member 9 | | | |
| 56132 | PCDHB3 | protocadherin | 2.19 | −1.40 | 0.21 |
| | | beta 3 | | | |
| 23400 | ATP13A2 | ATPase type | 2.19 | 0.69 | 0.78 |
| | | 13A2 | | | |
| 3980 | LIG3 | ligase III, DNA, | 2.19 | −1.66 | 0.65 |
| | | ATP-dependent | | | |
| 5690 | PSMB2 | proteasome | 2.19 | −0.48 | −1.31 |
| | | (prosome, | | | |
| | | macropain) | | | |
| | | subunit, beta | | | |
| | | type, 2 | | | |
| 7917 | BAG6 | BCL2- | 2.19 | 0.61 | −1.07 |
| | | associated | | | |
| | | athanogene 6 | | | |
| 1739 | DLG1 | discs, large | 2.18 | −0.19 | 0.09 |
| | | homolog 1 | | | |
| | | (Drosophila) | | | |
| 2837 | UTS2R | urotensin 2 | 2.18 | 0.03 | −1.24 |
| | | receptor | | | |
| 83746 | L3MBTL2 | l(3)mbt-like 2 | 2.18 | −0.32 | 0.57 |
| | | (Drosophila) | | | |
| 5962 | RDX | radixin | 2.18 | 1.15 | −0.45 |
| 9956 | HS3ST2 | heparan sulfate | 2.18 | 0.52 | −0.59 |
| | | (glucosamine) | | | |
| | | 3-O- | | | |
| | | sulfotransferase 2 | | | |
| 162968 | ZNF497 | zinc finger | 2.18 | −0.39 | −0.58 |
| | | protein 497 | | | |
| 4192 | MDK | midkine (neurite | 2.18 | 0.14 | −1.17 |
| | | growth- | | | |
| | | promoting factor | | | |
| | | 2) | | | |
| 54919 | HEATR2 | HEAT repeat | 2.18 | −0.66 | −0.12 |
| | | containing 2 | | | |
| 10972 | TMED10 | transmembrane | 2.17 | 0.29 | −0.15 |
| | | emp24-like | | | |
| | | trafficking | | | |
| | | protein 10 | | | |
| | | (yeast) | | | |
| 23061 | TBC1D9B | TBC1 domain | 2.17 | 0.25 | −1.61 |
| | | family, member | | | |
| | | 9B (with GRAM | | | |
| | | domain) | | | |
| 3792 | KEL | Kell blood | 2.17 | −1.53 | −0.95 |
| | | group, metallo- | | | |
| | | endopeptidase | | | |
| 2063 | NR2F6 | nuclear receptor | 2.17 | 0.08 | −1.78 |
| | | subfamily 2, | | | |
| | | group F, | | | |
| | | member 6 | | | |
| 55177 | RMDN3 | regulator of | 2.17 | −0.02 | −0.72 |
| | | microtubule | | | |
| | | dynamics 3 | | | |
| 11147 | HHLA3 | HERV-H LTR- | 2.17 | −0.01 | −0.94 |
| | | associating 3 | | | |
| 57486 | NLN | neurolysin | 2.17 | −0.76 | −2.12 |
| | | (metallopeptidase | | | |
| | | M3 family) | | | |
| 30814 | PLA2G2E | phospholipase | 2.16 | −1.73 | −0.14 |
| | | A2, group IIE | | | |
| 57414 | RHBDD2 | rhomboid | 2.16 | −0.83 | 0.47 |
| | | domain 2 | | | |
| | | containing 2 | | | |
| 84836 | ABHD14B | abhydrolase | 2.16 | 0.25 | −0.70 |
| | | domain | | | |
| | | containing 14B | | | |
| 8516 | ITGA8 | integrin, alpha 8 | 2.16 | −1.05 | −0.45 |
| 89866 | SEC16B | SEC16 homolog | 2.16 | 0.34 | −1.11 |
| | | B (S. cerevisiae) | | | |
| 10491 | CRTAP | cartilage | 2.16 | −0.42 | −1.22 |
| | | associated | | | |
| | | protein | | | |
| 11047 | ADRM1 | adhesion | 2.16 | −0.86 | −0.75 |
| | | regulating | | | |
| | | molecule 1 | | | |
| 28971 | AAMDC | adipogenesis | 2.16 | −0.57 | −1.44 |
| | | associated, | | | |
| | | Mth938 domain | | | |
| | | containing | | | |
| 8078 | USP5 | ubiquitin | 2.16 | −0.20 | −1.15 |
| | | specific | | | |
| | | peptidase 5 | | | |
| | | (isopeptidase T) | | | |
| 37 | ACADVL | acyl-CoA | 2.16 | 0.77 | −1.27 |
| | | dehydrogenase, | | | |
| | | very long chain | | | |
| 145508 | CEP128 | centrosomal | 2.15 | −1.44 | −0.13 |
| | | protein 128 kDa | | | |
| 49861 | CLDN20 | claudin 20 | 2.15 | −1.53 | −0.99 |
| 5905 | RANGAP1 | Ran GTPase | 2.15 | 0.36 | 0.13 |
| | | activating | | | |
| | | protein 1 | | | |
| 7542 | ZFPL1 | zinc finger | 2.15 | −0.61 | −0.83 |
| | | protein-like 1 | | | |
| 9562 | MINPP1 | multiple | 2.15 | 0.75 | −0.88 |
| | | inositol- | | | |
| | | polyphosphate | | | |
| | | phosphatase 1 | | | |
| 79095 | C9orf16 | chromosome 9 | 2.15 | −0.91 | −1.40 |
| | | open reading | | | |
| | | frame 16 | | | |
| 8815 | BANF1 | barrier to | 2.15 | −0.86 | −0.94 |
| | | autointegration | | | |
| | | factor 1 | | | |
| 54867 | TMEM214 | transmembrane | 2.15 | −0.36 | −1.15 |
| | | protein 214 | | | |
| 6721 | SREBF2 | sterol regulatory | 2.15 | −0.66 | −1.09 |
| | | element binding | | | |
| | | transcription | | | |
| | | factor 2 | | | |
| 23133 | PHF8 | PHD finger | 2.15 | −2.33 | −0.20 |
| | | protein 8 | | | |
| 113675 | SDSL | serine | 2.14 | −0.45 | −1.26 |
| | | dehydratase-like | | | |
| 129303 | TMEM150A | transmembrane | 2.14 | −1.19 | −0.27 |
| | | protein 150A | | | |
| 57658 | CALCOCO1 | calcium binding | 2.14 | −0.87 | −2.39 |
| | | and coiled-coil | | | |
| | | domain 1 | | | |
| 162 | AP1B1 | adaptor-related | 2.14 | 0.16 | −2.22 |
| | | protein complex | | | |
| | | 1, beta 1 subunit | | | |
| 2873 | GPS1 | G protein | 2.14 | −0.39 | 0.88 |
| | | pathway | | | |
| | | suppressor 1 | | | |
| 64978 | MRPL38 | mitochondrial | 2.14 | −1.05 | 0.55 |
| | | ribosomal | | | |
| | | protein L38 | | | |
| 220323 | OAF | OAF homolog | 2.14 | 1.31 | −1.11 |
| | | (Drosophila) | | | |
| 6006 | RHCE | Rh blood group, | 2.14 | −0.69 | −0.91 |
| | | CcEe antigens | | | |
| 389812 | LCN15 | lipocalin 15 | 2.14 | 0.12 | −1.10 |
| 2628 | GATM | glycine | 2.13 | −0.78 | −2.26 |
| | | amidinotransferase | | | |
| | | (L- | | | |
| | | arginine:glycine | | | |
| | | amidinotransferase) | | | |
| 6509 | SLC1A4 | solute carrier | 2.13 | −1.52 | 0.41 |
| | | family 1 | | | |
| | | (glutamate/neutral | | | |
| | | amino acid | | | |
| | | transporter), | | | |
| | | member 4 | | | |
| 3888 | KRT82 | keratin 82 | 2.13 | −0.47 | −0.10 |
| 5211 | PFKL | phosphofructokinase, | 2.13 | 0.14 | −1.68 |
| | | liver | | | |
| 55080 | TAPBPL | TAP binding | 2.13 | −0.22 | −1.22 |
| | | protein-like | | | |
| 2139 | EYA2 | eyes absent | 2.13 | −1.64 | 0.08 |
| | | homolog 2 | | | |
| | | (Drosophila) | | | |
| 2030 | SLC29A1 | solute carrier | 2.12 | 0.75 | −0.96 |
| | | family 29 | | | |
| | | (equilibrative | | | |
| | | nucleoside | | | |
| | | transporter), | | | |
| | | member 1 | | | |
| 56005 | C19orf10 | chromosome 19 | 2.12 | −0.77 | −0.18 |
| | | open reading | | | |
| | | frame 10 | | | |
| 6414 | SEPP1 | selenoprotein P, | 2.12 | 1.63 | −1.72 |
| | | plasma, 1 | | | |
| 5184 | PEPD | peptidase D | 2.12 | −0.14 | −1.08 |
| 643783 | USP46- | USP46 antisense | 2.12 | 0.35 | −1.04 |
| | AS1 | RNA 1 | | | |
| 101928185 | LOC101928185 | uncharacterized | 2.12 | −1.31 | −1.64 |
| | | LOC101928185 | | | |
| 22883 | CLSTN1 | calsyntenin 1 | 2.12 | −0.33 | −1.22 |
| 25800 | SLC39A6 | solute carrier | 2.12 | 0.89 | −1.29 |
| | | family 39 (zinc | | | |
| | | 6 transporter), | | | |
| | | member 6 | | | |
| 54681 | P4HTM | prolyl 4- | 2.12 | 1.14 | −1.11 |
| | | hydroxylase, | | | |
| | | transmembrane | | | |
| | | (endoplasmic | | | |
| | | reticulum) | | | |
| 644150 | WIPF3 | WAS/WASL | 2.12 | −1.29 | 0.15 |
| | | interacting | | | |
| | | protein family, | | | |
| | | member 3 | | | |
| 9048 | ARTN | artemin | 2.12 | 0.12 | −1.89 |
| 3054 | HCFC1 | host cell factor | 2.11 | 0.43 | −0.62 |
| | | C1 (VP16- | | | |
| | | accessory | | | |
| | | protein) | | | |
| 115098 | CCDC124 | coiled-coil | 2.11 | −1.85 | 0.10 |
| | | domain | | | |
| | | containing 124 | | | |
| 55558 | PLXNA3 | plexin A3 | 2.11 | 0.92 | −0.78 |
| 675 | BRCA2 | breast cancer 2, | 2.11 | 0.63 | −0.16 |
| | | early onset | | | |
| 9777 | TM9SF4 | transmembrane | 2.11 | 1.49 | −1.09 |
| | | 9 superfamily | | | |
| | | protein member 4 | | | |
| 83707 | TRPT1 | tRNA | 2.11 | −0.04 | −0.15 |
| | | phosphotransferase 1 | | | |
| 55334 | SLC39A9 | solute carrier | 2.10 | −1.47 | −2.10 |
| | | family 39, | | | |
| | | member 9 | | | |
| 9793 | CKAP5 | cytoskeleton | 2.10 | 0.88 | −0.71 |
| | | associated | | | |
| | | protein 5 | | | |
| 56834 | GPR137 | G protein- | 2.10 | 0.20 | −1.10 |
| | | coupled receptor | | | |
| | | 137 | | | |
| 25921 | ZDHHC5 | zinc finger, | 2.10 | 0.57 | −1.11 |
| | | DHHC-type | | | |
| | | containing 5 | | | |
| 6520 | SLC3A2 | solute carrier | 2.10 | −0.83 | 0.35 |
| | | family 3 (amino | | | |
| | | acid transparter | | | |
| | | heavy chain), | | | |
| | | member 2 | | | |
| 728591 | CCDC169 | coiled-coil | 2.10 | 0.35 | −1.49 |
| | | domain | | | |
| | | containing 169 | | | |
| 7415 | VCP | valosin | 2.10 | −0.60 | −0.61 |
| | | containing | | | |
| | | protein | | | |
| 130827 | TMEM182 | transmembrane | 2.09 | 0.93 | −1.07 |
| | | protein 182 | | | |
| 9620 | CELSR1 | cadherin, EGF | 2.09 | −0.77 | −0.90 |
| | | LAG seven-pass | | | |
| | | G-type receptor 1 | | | |
| 55335 | NIPSNAP3B | nipsnap | 2.09 | 0.69 | 0.11 |
| | | homolog 3B (C. elegans) | | | |
| 84988 | PPP1R16A | protein | 2.08 | −0.45 | −0.84 |
| | | phosphatase 1, | | | |
| | | regulatory | | | |
| | | subunit 16A | | | |
| 100192378 | ZFHX4- | ZFHX4 | 2.08 | −2.06 | −0.64 |
| | AS1 | antisense RNA1 | | | |
| 442524 | DPY19L2P3 | DPY19L2 | 2.08 | −1.30 | −0.58 |
| | | pseudogene 3 | | | |
| 51661 | FKBP7 | FK506 binding | 2.08 | −0.88 | −0.66 |
| | | protein 7 | | | |
| 84922 | FIZ1 | FLT3- | 2.08 | −0.46 | 0.44 |
| | | interacting zinc | | | |
| | | finger 1 | | | |
| 84954 | MPND | MPN domain | 2.08 | −0.90 | −1.51 |
| | | containing | | | |
| 245972 | ATP6V0D2 | ATPase, H+ | 2.08 | −1.56 | −0.90 |
| | | transporting, | | | |
| | | lysosomal | | | |
| | | 38 kDa, V0 | | | |
| | | subunit d2 | | | |
| 4316 | MMP7 | matrix | 2.08 | 0.13 | −0.58 |
| | | metallopeptidase | | | |
| | | 7 (matrilysin, | | | |
| | | uterine) | | | |
| 55157 | DARS2 | aspartyl-tRNA | 2.08 | −0.09 | −0.44 |
| | | synthetase 2, | | | |
| | | mitochondrial | | | |
| 7466 | WFS1 | Wolfram | 2.08 | −0.39 | −1.04 |
| | | syndrome 1 | | | |
| | | (wolframin) | | | |
| 79365 | BHLHE41 | basic helix-loop- | 2.08 | −0.44 | −0.14 |
| | | helix family, | | | |
| | | member e41 | | | |
| 10066 | SCAMP2 | secretory carrier | 2.08 | 0.95 | −0.99 |
| | | membrane | | | |
| | | protein 2 | | | |
| 203068 | TUBB | tubulin, beta | 2.08 | 0.35 | −0.93 |
| | | class I | | | |
| 5780 | PTPN9 | protein tyrosine | 2.07 | −0.81 | −1.50 |
| | | phosphatase, | | | |
| | | non-receptor | | | |
| | | type 9 | | | |
| 285627 | LOC285627 | uncharacterized | 2.07 | −0.60 | −1.20 |
| | | LOC285627 | | | |
| 2058 | EPRS | glutamyl-prolyl- | 2.07 | 1.25 | −0.56 |
| | | tRNA | | | |
| | | synthetase | | | |
| 1737 | DLAT | dihydrolipoamide | 2.06 | 0.55 | −0.03 |
| | | S- | | | |
| | | acetyltransferase | | | |
| 22924 | MAPRE3 | microtubule- | 2.06 | −1.75 | −1.32 |
| | | associated | | | |
| | | protein, RP/EB | | | |
| | | family, member 3 | | | |
| 6005 | RHAG | Rh-associated | 2.06 | 0.29 | −0.86 |
| | | glycoprotein | | | |
| 5286 | PIK3C2A | phosphatidylinositol- | 2.06 | 0.32 | −0.71 |
| | | 4- | | | |
| | | phosphate 3- | | | |
| | | kinase, catalytic | | | |
| | | subunit type 2 | | | |
| | | alpha | | | |
| 11226 | GALNT6 | UDP-N-acetyl- | 2.06 | −0.01 | −0.06 |
| | | alpha-D- | | | |
| | | galactosamine:polypeptide | | | |
| | | N- | | | |
| | | acetylgalactosaminyltransferase | | | |
| | | 6 (GalNAc-T6) | | | |
| 2762 | GMDS | GDP-mannose | 2.06 | −0.47 | −0.69 |
| | | 4,6-dehydratase | | | |
| 8908 | GYG2 | glycogenin 2 | 2.06 | −1.16 | −0.54 |
| 10693 | CCT6B | chaperonin | 2.06 | −0.80 | 0.01 |
| | | containing | | | |
| | | TCP1, subunit | | | |
| | | 6B (zeta 2) | | | |
| 199953 | TMEM201 | transmembrane | 2.06 | 0.96 | −0.49 |
| | | protein 201 | | | |
| 2760 | GM2A | GM2 | 2.06 | −0.83 | −2.47 |
| | | ganglioside | | | |
| | | activator | | | |
| 8295 | TRRAP | transformation/transcription | 2.06 | −0.95 | 0.47 |
| | | domain- | | | |
| | | associated | | | |
| | | protein | | | |
| 1822 | ATN1 | atrophin 1 | 2.06 | 0.34 | −1.85 |
| 55328 | RNLS | renalase, FAD- | 2.06 | −1.82 | −1.29 |
| | | dependent | | | |
| | | amine oxidase | | | |
| 2069 | EREG | epiregulin | 2.05 | 0.33 | −1.08 |
| 4329 | ALDH6A1 | aldehyde | 2.05 | −0.73 | −1.24 |
| | | dehydrogenase 6 | | | |
| | | family, member | | | |
| | | A1 | | | |
| 537 | ATP6AP1 | ATPase, H+ | 2.05 | −0.44 | −0.63 |
| | | transporting, | | | |
| | | lysosomal | | | |
| | | accessory | | | |
| | | protein 1 | | | |
| 64764 | CREB3L2 | cAMP | 2.05 | 0.14 | −0.79 |
| | | responsive | | | |
| | | element binding | | | |
| | | protein 3-like 2 | | | |
| 811 | CALR | calreticulin | 2.05 | −0.14 | −0.89 |
| 9779 | TBC1D5 | TBC1 domain | 2.05 | −1.68 | −2.31 |
| | | family, member 5 | | | |
| 114815 | SORCS1 | sortilin-related | 2.05 | 0.76 | −1.44 |
| | | VPS10 domain | | | |
| | | containing | | | |
| | | receptor 1 | | | |
| 29927 | SEC61A1 | Sec61 alpha 1 | 2.05 | 0.85 | −0.38 |
| | | subunit (S. cerevisiae) | | | |
| 6007 | RHD | Rh blood group, | 2.05 | −0.65 | −0.49 |
| | | D antigen | | | |
| 5192 | PEX10 | peroxisomal | 2.05 | 0.46 | −0.02 |
| | | biogenesis | | | |
| | | factor 10 | | | |
| 125875 | CLDND2 | claudin domain | 2.04 | −0.14 | −0.57 |
| | | containing 2 | | | |
| 10882 | C1QL1 | complement | 2.04 | −1.07 | −0.41 |
| | | component 1, q | | | |
| | | subcomponent- | | | |
| | | like 1 | | | |
| 192683 | SCAMP5 | secretory carrier | 2.04 | −1.38 | −0.77 |
| | | membrane | | | |
| | | protein 5 | | | |
| 8028 | MLLT10 | myeloid/lymphoid | 2.04 | −0.77 | −0.35 |
| | | or mixed- | | | |
| | | lineage | | | |
| | | leukemia | | | |
| | | (trithorax | | | |
| | | homolog, | | | |
| | | Drosophila); | | | |
| | | translocated to, | | | |
| | | 10 | | | |
| 150368 | FAM109B | family with | 2.04 | −2.05 | −0.51 |
| | | sequence | | | |
| | | similarity 109, | | | |
| | | member B | | | |
| 84206 | MEX3B | mex-3 RNA | 2.04 | 0.32 | −0.13 |
| | | binding family | | | |
| | | member B | | | |
| 101929288 | LOC101929288 | uncharacterized | 2.04 | 1.28 | −1.48 |
| | | LOC101929288 | | | |
| 826 | CAPNS1 | calpain, small | 2.04 | 0.59 | −1.22 |
| | | subunit 1 | | | |
| 83440 | ADPGK | ADP-dependent | 2.04 | −0.02 | −0.60 |
| | | glucokinase | | | |
| 1128 | CHRM1 | cholinergic | 2.03 | −1.51 | −0.76 |
| | | receptor, | | | |
| | | muscarinic 1 | | | |
| 160287 | LDHAL6A | lactate | 2.03 | −2.65 | −1.74 |
| | | dehydrogenase | | | |
| | | A-like 6A | | | |
| 412 | STS | steroid sulfatase | 2.03 | −1.76 | −1.65 |
| | | (microsomal), | | | |
| | | isozyme S | | | |
| 64215 | DNAJC1 | DnaJ (Hsp40) | 2.03 | 1.51 | −1.51 |
| | | homolog, | | | |
| | | subfamily C, | | | |
| | | member 1 | | | |
| 7343 | UBTF | upstream | 2.03 | −0.53 | −0.11 |
| | | binding | | | |
| | | transcription | | | |
| | | factor, RNA | | | |
| | | polymerase I | | | |
| 25840 | METTL7A | methyltransferase | 2.03 | −0.61 | −2.47 |
| | | like 7A | | | |
| 55315 | SLC29A3 | solute carrier | 2.03 | −0.28 | −1.38 |
| | | family 29 | | | |
| | | (equilibrative | | | |
| | | nucleoside | | | |
| | | transporter), | | | |
| | | member 3 | | | |
| 100507513 | LOC100507513 | uncharacterized | 2.03 | −2.70 | −0.19 |
| | | LOC100507513 | | | |
| 55704 | CCDC88A | coiled-coil | 2.03 | 0.69 | −1.77 |
| | | domain | | | |
| | | containing 88A | | | |
| 57604 | KIAA1456 | KIAA1 456 | 2.03 | −0.70 | −0.30 |
| 10449 | ACAA2 | acetyl-CoA | 2.03 | −1.02 | 0.19 |
| | | acyltransferase 2 | | | |
| 1727 | CYB5R3 | cytochrome b5 | 2.03 | −0.54 | −2.22 |
| | | reductase 3 | | | |
| 9569 | GTF2IRD1 | GTF2I repeat | 2.03 | 0.34 | −1.07 |
| | | domain | | | |
| | | containing 1 | | | |
| 10009 | ZBTB33 | zinc finger and | 2.02 | 0.14 | −0.17 |
| | | BTB domain | | | |
| | | containing 33 | | | |
| 5188 | PET112 | PET112 | 2.02 | 0.13 | 0.24 |
| | | homolog (yeast) | | | |
| 2132 | EXT2 | exostosin | 2.02 | −0.14 | −0.75 |
| | | glycosyltransferase 2 | | | |
| 2239 | GPC4 | glypican 4 | 2.02 | −0.72 | −2.04 |
| 56927 | GPR108 | G protein- | 2.02 | −1.22 | −0.51 |
| | | coupled receptor | | | |
| | | 108 | | | |
| 2950 | GSTP1 | glutathione S- | 2.02 | 0.65 | −1.37 |
| | | transferase pi 1 | | | |
| 342538 | NACA2 | nascent | 2.02 | −0.31 | −0.61 |
| | | polypeptide- | | | |
| | | associated | | | |
| | | complex alpha | | | |
| | | subunit 2 | | | |
| 55218 | EXD2 | exonuclease 3′- | 2.02 | −0.54 | −0.04 |
| | | 5′ domain | | | |
| | | containing 2 | | | |
| 151827 | LRRC34 | leucine rich | 2.01 | 0.17 | −1.19 |
| | | repeat | | | |
| | | containing 34 | | | |
| 64689 | GORASP1 | golgi | 2.01 | 0.31 | −0.64 |
| | | reassembly | | | |
| | | stacking protein | | | |
| | | 1, 65 kDa | | | |
| 10324 | KLHL41 | ketch-like | 2.01 | −1.94 | −0.23 |
| | | family member | | | |
| | | 41 | | | |
| 1314 | COPA | coatomer | 2.01 | 0.86 | −0.79 |
| | | protein complex, | | | |
| | | subunit alpha | | | |
| 54928 | IMPAD1 | inositol | 2.01 | −0.51 | −0.54 |
| | | monophosphatase | | | |
| | | domain | | | |
| | | containing 1 | | | |
| 8971 | Hl FX | Hl histone | 2.01 | 0.81 | −0.77 |
| | | family, member X | | | |
| 79832 | QSER1 | glutamine and | 2.01 | 0.76 | −2.38 |
| | | serine rich 1 | | | |
| 10150 | MBNL2 | muscleblind-like | 2.00 | 0.94 | −0.70 |
| | | splicing | | | |
| | | regulator 2 | | | |
| 8744 | TNFSF9 | tumor necrosis | 2.00 | −0.11 | −0.84 |
| | | factor (ligand) | | | |
| | | superfamily, | | | |
| | | member 9 | | | |
| 23287 | AGTPBP1 | ATP/GTP | −2.00 | −1.57 | 0.97 |
| | | binding protein 1 | | | |
| 606553 | C8orf49 | chromosome 8 | −2.00 | 2.76 | 0.90 |
| | | open reading | | | |
| | | frame 49 | | | |
| 103 | ADAR | adenosine | −2.01 | −0.46 | 0.24 |
| | | deaminase, | | | |
| | | RNA-specific | | | |
| 55509 | BATF3 | basic leucine | −2.01 | 2.57 | −0.14 |
| | | zipper | | | |
| | | transcription | | | |
| | | factor, ATF-like 3 | | | |
| 729614 | FLJ37453 | uncharacterized | −2.01 | −0.39 | 1.58 |
| | | LOC729614 | | | |
| 9241 | NOG | noggin | −2.01 | 0.41 | 2.02 |
| 26118 | WSB1 | WD repeat and | −2.01 | 1.08 | 0.29 |
| | | SOCS box | | | |
| | | containing 1 | | | |
| 64400 | AKTIP | AKT interacting | −2.01 | −0.63 | 1.59 |
| | | protein | | | |
| 283237 | TTC9C | tetratricopeptide | −2.01 | 2.06 | 1.25 |
| | | repeat domain | | | |
| | | 9C | | | |
| 51155 | HN1 | hematological | −2.01 | −0.85 | 0.39 |
| | | and neurological | | | |
| | | expressed 1 | | | |
| 18 | ABAT | 4-aminobutyrate | −2.01 | −1.10 | 0.52 |
| | | aminotransferase | | | |
| 22990 | PCNX | pecanex | −2.01 | −1.17 | 0.95 |
| | | homolog | | | |
| | | (Drosophila) | | | |
| 23347 | SMCHD1 | structural | −2.01 | −1.02 | 0.76 |
| | | maintenance of | | | |
| | | chromosomes | | | |
| | | flexible hinge | | | |
| | | domain | | | |
| | | containing 1 | | | |
| 83988 | NCALD | neurocalcin | −2.01 | 1.58 | 0.85 |
| | | delta | | | |
| 3428 | IFI16 | interferon, | −2.02 | −0.62 | 0.35 |
| | | gamma- | | | |
| | | inducible | | | |
| | | protein 16 | | | |
| 3700 | ITIH4 | inter-alpha- | −2.02 | −2.31 | 1.74 |
| | | trypsin inhibitor | | | |
| | | heavy chain | | | |
| | | family, member 4 | | | |
| 64859 | NABP1 | nucleic acid | −2.02 | 0.63 | −0.50 |
| | | binding protein 1 | | | |
| 55192 | DNAJC17 | DnaJ (Hsp40) | −2.02 | 0.76 | 2.14 |
| | | homolog, | | | |
| | | subfamily C, | | | |
| | | member 17 | | | |
| 101928869 | LOC101928869 | uncharacterized | −2.02 | −0.94 | 0.98 |
| | | LOC101928869 | | | |
| 10513 | APPBP2 | amyloid beta | −2.02 | 0.18 | 1.66 |
| | | precursor | | | |
| | | protein | | | |
| | | (cytoplasmic | | | |
| | | tail) binding | | | |
| | | protein 2 | | | |
| 11009 | IL24 | interleukin 24 | −2.02 | 1.97 | 0.89 |
| 169355 | IDO2 | indoleamine 2,3- | −2.02 | 1.52 | 0.85 |
| | | dioxygenase 2 | | | |
| 10109 | ARPC2 | actin related | −2.02 | 0.24 | 0.98 |
| | | protein 2/3 | | | |
| | | complex, | | | |
| | | subunit 2, | | | |
| | | 34 kDa | | | |
| 9903 | KLHL21 | kelch-like | −2.02 | −1.20 | 1.23 |
| | | family member | | | |
| | | 21 | | | |
| 23150 | FRMD4B | FERM domain | −2.03 | 1.12 | 0.47 |
| | | containing 4B | | | |
| 10865 | ARID5A | AT rich | −2.03 | 0.52 | 1.51 |
| | | interactive | | | |
| | | domain 5A | | | |
| | | (MRF1-like) | | | |
| 5165 | PDK3 | pyruvate | −2.03 | −0.54 | 0.91 |
| | | dehydrogenase | | | |
| | | kinase, isozyme 3 | | | |
| 57403 | RAB22A | RAB22A, | −2.03 | −0.08 | 2.12 |
| | | member RAS | | | |
| | | oncogene family | | | |
| 22900 | CARD8 | caspase | −2.03 | 0.52 | 0.73 |
| | | recruitment | | | |
| | | domain family, | | | |
| | | member 8 | | | |
| 80345 | ZSCAN16 | zinc finger and | −2.03 | 0.80 | 0.98 |
| | | SCAN domain | | | |
| | | containing 16 | | | |
| 10964 | IFI44L | interferon- | −2.03 | −0.11 | 1.45 |
| | | induced protein | | | |
| | | 44-like | | | |
| 27040 | LAT | linker for | −2.03 | 0.98 | 2.49 |
| | | activation of T | | | |
| | | cells | | | |
| 91010 | FMNL3 | formin-like 3 | −2.03 | 1.02 | 1.86 |
| 29065 | ASAP1- | ASAP1 intronic | −2.04 | −0.30 | −0.18 |
| | IT1 | transcript 1 | | | |
| | | (non-protein | | | |
| | | coding) | | | |
| 84078 | KBTBD7 | kelch repeat and | −2.04 | −1.19 | 0.90 |
| | | BTB (POZ) | | | |
| | | domain | | | |
| | | containing 7 | | | |
| 101927910 | LOC101927910 | keratin- | −2.04 | −0.31 | 2.24 |
| | | associated | | | |
| | | protein 5-5-like | | | |
| 51380 | CSAD | cysteine sulfinic | −2.04 | −0.09 | −0.06 |
| | | acid | | | |
| | | decarboxylase | | | |
| 10289 | EIF1B | eukaryotic | −2.04 | −0.24 | 0.83 |
| | | translation | | | |
| | | initiation factor | | | |
| | | 1B | | | |
| 10296 | MAEA | macrophage | −2.04 | −0.68 | 0.90 |
| | | erythroblast | | | |
| | | attacher | | | |
| 114224 | PRO2852 | uncharacterized | −2.04 | 0.11 | −0.51 |
| | | protein | | | |
| | | PRO2852 | | | |
| 2153 | F5 | coagulation | −2.04 | −1.17 | 0.70 |
| | | factor V | | | |
| | | (proaccelerin, | | | |
| | | labile factor) | | | |
| 55825 | PECR | peroxisomal | −2.04 | −0.87 | 0.84 |
| | | trans-2-enoyl- | | | |
| | | CoA reductase | | | |
| 6503 | SLA | Src-like-adaptor | −2.04 | −0.29 | 1.61 |
| 7913 | DEK | DEK oncogene | −2.04 | 1.72 | 0.60 |
| 30834 | ZNRD1 | zinc ribbon | −2.04 | −0.10 | 2.21 |
| | | domain | | | |
| | | containing 1 | | | |
| 26999 | CYFIP2 | cytoplasmic | −2.05 | −1.41 | 2.03 |
| | | FMR1 | | | |
| | | interacting | | | |
| | | protein 2 | | | |
| 286437 | LOC286437 | uncharacterized | −2.05 | −1.23 | 1.77 |
| | | LOC286437 | | | |
| 4277 | MICB | MHC class I | −2.05 | 0.44 | 0.88 |
| | | polypeptide- | | | |
| | | related sequence B | | | |
| 5698 | PSMB9 | proteasome | −2.05 | 0.22 | 0.77 |
| | | (prosome, | | | |
| | | macropain) | | | |
| | | subunit, beta | | | |
| | | type, 9 | | | |
| 4318 | MMP9 | matrix | −2.05 | 0.13 | 0.27 |
| | | metallopeptidase | | | |
| | | 9 (gelatinase B, | | | |
| | | 92 kDa | | | |
| | | gelatinase, | | | |
| | | 92 kDa type IV | | | |
| | | collagenase) | | | |
| 10616 | RBCK1 | RanBP-type and | −2.06 | 1.31 | −0.02 |
| | | C3HC4-type | | | |
| | | zinc finger | | | |
| | | containing 1 | | | |
| 23012 | STK38L | serine/threonine | −2.06 | −0.51 | 0.07 |
| | | kinase 38 like | | | |
| 100129361 | LOC100129361 | chromosome X | −2.06 | 0.66 | 1.07 |
| | | open reading | | | |
| | | frame 69-like | | | |
| 10742 | RAI2 | retinoic acid | −2.06 | −1.54 | 0.78 |
| | | induced 2 | | | |
| 196264 | MPZL3 | myelin protein | −2.06 | −0.75 | 1.46 |
| | | zero-like 3 | | | |
| 27197 | GPR82 | G protein- | −2.06 | 1.47 | −0.05 |
| | | coupled receptor | | | |
| | | 82 | | | |
| 7529 | YWHAB | tyrosine 3- | −2.06 | 0.12 | 1.88 |
| | | monooxygenase/ | | | |
| | | tryptophan 5- | | | |
| | | monooxygenase | | | |
| | | activation | | | |
| | | protein, beta | | | |
| | | polypeptide | | | |
| 94235 | GNG8 | guanine | −2.06 | 0.86 | 1.60 |
| | | nucleotide | | | |
| | | binding protein | | | |
| | | (G protein), | | | |
| | | gamma 8 | | | |
| 352961 | HCG26 | HLA complex | −2.06 | 0.17 | 1.72 |
| | | group 26 (non- | | | |
| | | protein coding) | | | |
| 49856 | WRAP73 | WD repeat | −2.06 | 1.00 | 0.94 |
| | | containing, | | | |
| | | antisense to | | | |
| | | TP73 | | | |
| 9448 | MAP4K4 | mitogen- | −2.06 | −0.65 | 0.97 |
| | | activated protein | | | |
| | | kinase kinase | | | |
| | | kinase kinase 4 | | | |
| 9525 | VPS4B | vacuolar protein | −2.06 | −0.29 | 0.95 |
| | | sorting 4 | | | |
| | | homolog B (S. cerevisiae) | | | |
| 100505576 | LINC00672 | long intergenic | −2.06 | −0.31 | 0.81 |
| | | non-protein | | | |
| | | coding RNA | | | |
| | | 672 | | | |
| 151556 | GPR155 | G protein- | −2.06 | −0.84 | 1.11 |
| | | coupled receptor | | | |
| | | 155 | | | |
| 284613 | CYB561D1 | cytochrome | −2.06 | −0.29 | 1.00 |
| | | b561 family, | | | |
| | | member D1 | | | |
| 286223 | C9orf47 | chromosome 9 | −2.06 | −0.06 | −0.46 |
| | | open reading | | | |
| | | frame 47 | | | |
| 57061 | HYMAI | hydatidiform | −2.06 | −0.54 | −1.51 |
| | | mole associated | | | |
| | | and imprinted | | | |
| | | (non-protein | | | |
| | | coding) | | | |
| 9520 | NPEPPS | aminopeptidase | −2.07 | −0.18 | 0.60 |
| | | puromycin | | | |
| | | sensitive | | | |
| 202299 | C5orf27 | chromosome 5 | −2.07 | 0.22 | 0.55 |
| | | open reading | | | |
| | | frame 27 | | | |
| 2015 | EMR1 | egf-like module | −2.07 | 0.71 | −0.20 |
| | | containing, | | | |
| | | mucin-like, | | | |
| | | hormone | | | |
| | | receptor-like 1 | | | |
| 57763 | ANKRA2 | ankyrin repeat, | −2.07 | 1.06 | 1.86 |
| | | family A | | | |
| | | (RFXANK- | | | |
| | | like), 2 | | | |
| 54778 | RNF111 | ring finger | −2.07 | 0.08 | 0.96 |
| | | protein 111 | | | |
| 762 | CA4 | carbonic | −2.07 | −1.35 | 0.56 |
| | | anhydrase IV | | | |
| 192668 | CYS1 | cystin 1 | −2.08 | 0.29 | 0.23 |
| 24146 | CLDN15 | claudin 15 | −2.08 | 0.77 | 1.30 |
| 54843 | SYTL2 | synaptotagmin- | −2.08 | 1.28 | 1.88 |
| | | like 2 | | | |
| 55374 | TMCO6 | transmembrane | −2.08 | 0.23 | 0.72 |
| | | and coiled-coil | | | |
| | | domains 6 | | | |
| 79603 | CERS4 | ceramide | −2.08 | −0.79 | 1.36 |
| | | synthase 4 | | | |
| 3577 | CXCR1 | chemokine | −2.08 | −0.87 | 0.16 |
| | | (C—X—C motif) | | | |
| | | receptor 1 | | | |
| 10385 | BTN2A2 | butyrophilin, | −2.08 | 2.47 | 0.07 |
| | | subfamily 2, | | | |
| | | member A2 | | | |
| 79792 | GSDMD | gasdermin D | −2.08 | 1.86 | −0.51 |
| 84674 | CARD6 | caspase | −2.09 | −0.39 | 0.54 |
| | | recruitment | | | |
| | | domain family, | | | |
| | | member 6 | | | |
| 51131 | PHF11 | PHD finger | −2.09 | −0.26 | 1.37 |
| | | protein 11 | | | |
| 5966 | REL | v-rel avian | −2.09 | 1.12 | 1.40 |
| | | reticuloendotheliosis | | | |
| | | viral | | | |
| | | oncogene | | | |
| | | homolog | | | |
| 9733 | SART3 | squamous cell | −2.09 | 2.19 | 0.91 |
| | | carcinoma | | | |
| | | antigen | | | |
| | | recognized by T | | | |
| | | cells 3 | | | |
| 10328 | EMC8 | ER membrane | −2.09 | 0.55 | 1.57 |
| | | protein complex | | | |
| | | subunit 8 | | | |
| 255231 | MCOLN2 | mucolipin 2 | −2.09 | 0.84 | 0.52 |
| 25801 | GCA | grancalcin, EF- | −2.09 | 0.50 | −0.38 |
| | | hand calcium | | | |
| | | binding protein | | | |
| 6352 | CCL5 | chemokine (C-C | −2.09 | 0.66 | 2.04 |
| | | motif) ligand 5 | | | |
| 54811 | ZNF562 | zinc finger | −2.09 | 0.02 | 0.03 |
| | | protein 562 | | | |
| 59348 | ZNF350 | zinc finger | −2.09 | −0.38 | 1.17 |
| | | protein 350 | | | |
| 9967 | THRAP3 | thyroid hormone | −2.09 | 0.61 | 0.79 |
| | | receptor | | | |
| | | associated | | | |
| | | protein 3 | | | |
| 2533 | FYB | FYN binding | −2.10 | −0.93 | 1.62 |
| | | protein | | | |
| 28985 | MCTS1 | malignant T cell | −2.10 | 1.15 | 1.28 |
| | | amplified | | | |
| | | sequence 1 | | | |
| 55234 | SMU1 | smu-1 | −2.10 | −0.47 | 1.24 |
| | | suppressor of | | | |
| | | mec-8 and unc- | | | |
| | | 52 homolog (C. elegans) | | | |
| 1301 | COL11A1 | collagen, type | −2.10 | 1.40 | −0.09 |
| | | XI, alpha 1 | | | |
| 84803 | AGPAT9 | 1-acylglycerol- | −2.10 | 0.07 | 0.36 |
| | | 3-phosphate O- | | | |
| | | acyltransferase 9 | | | |
| 100506779 | BZRAP1- | BZRAP1 | −2.10 | −0.30 | 1.43 |
| | AS1 | antisense RNA 1 | | | |
| 197135 | PATL2 | protein | −2.10 | −0.32 | 1.45 |
| | | associated with | | | |
| | | topoisomerase II | | | |
| | | homolog 2 | | | |
| | | (yeast) | | | |
| 3614 | IMPDH1 | IMP (inosine 5′- | −2.10 | −0.75 | −0.34 |
| | | monophosphate) | | | |
| | | dehydrogenase 1 | | | |
| 80781 | COL18A1 | collagen, type | −2.10 | 0.13 | 0.76 |
| | | XVIII, alpha 1 | | | |
| 5586 | PKN2 | protein kinase | −2.11 | 0.30 | 0.71 |
| | | N2 | | | |
| 79415 | C17orf62 | chromosome 17 | −2.11 | 0.76 | −0.67 |
| | | open reading | | | |
| | | frame 62 | | | |
| 8893 | EIF2B5 | eukaryotic | −2.11 | 1.21 | 1.89 |
| | | translation | | | |
| | | initiation factor | | | |
| | | 2B, subunit 5 | | | |
| | | epsilon, 82 kDa | | | |
| 79736 | TEFM | transcription | −2.11 | 1.33 | 0.37 |
| | | elongation | | | |
| | | factor, | | | |
| | | mitochondrial | | | |
| 5770 | PTPN1 | protein tyrosine | −2.11 | 2.04 | −0.16 |
| | | phosphatase, | | | |
| | | non-receptor | | | |
| | | type 1 | | | |
| 9360 | PPIG | peptidylprolyl | −2.11 | 0.97 | 1.56 |
| | | isomerase G | | | |
| | | (cyclophilin G) | | | |
| 29940 | DSE | dermatan sulfate | −2.11 | 0.87 | 0.10 |
| | | epimerase | | | |
| 7150 | TOP1 | topoisomerase | −2.11 | 2.04 | −0.62 |
| | | (DNA) I | | | |
| 84964 | ALKBH6 | alkB, alkylation | −2.11 | 1.92 | −0.46 |
| | | repair homolog | | | |
| | | 6 (E. coli) | | | |
| 10614 | HEXIM1 | hexamethylene | −2.11 | 0.25 | 1.20 |
| | | bis-acetamide | | | |
| | | inducible 1 | | | |
| 147923 | ZNF420 | zinc finger | −2.12 | 0.88 | 1.70 |
| | | protein 420 | | | |
| 284029 | LINC00324 | long intergenic | −2.12 | −1.08 | 1.66 |
| | | non-protein | | | |
| | | coding RNA | | | |
| | | 324 | | | |
| 54952 | TRNAU1AP | tRNA | −2.12 | −0.15 | 1.42 |
| | | selenocysteine 1 | | | |
| | | associated | | | |
| | | protein 1 | | | |
| 57476 | GRAMD1B | GRAM domain | −2.12 | −0.38 | 0.44 |
| | | containing 1B | | | |
| 8745 | ADAM23 | ADAM | −2.12 | 2.31 | 0.05 |
| | | metallopeptidase | | | |
| | | domain 23 | | | |
| 22928 | SEPHS2 | selenophosphate | −2.12 | 0.67 | 0.44 |
| | | synthetase 2 | | | |
| 25880 | TMEM186 | transmembrane | −2.12 | 1.09 | 1.51 |
| | | protein 186 | | | |
| 28638 | TRBC2 | T cell receptor | −2.12 | −0.78 | 2.11 |
| | | beta constant 2 | | | |
| 29933 | GPR132 | G protein- | −2.12 | 0.04 | 1.76 |
| | | coupled receptor | | | |
| | | 132 | | | |
| 30009 | TBX21 | T-box 21 | −2.12 | 0.92 | 0.97 |
| 3554 | ILIR1 | interleukin 1 | −2.12 | 0.44 | 0.16 |
| | | receptor, type I | | | |
| 8821 | INPP4B | inositol | −2.12 | 1.75 | 1.83 |
| | | polyphosphate- | | | |
| | | 4-phosphatase, | | | |
| | | type II, 105 kDa | | | |
| 10943 | MSL3 | male-specific | −2.12 | −0.77 | 1.06 |
| | | lethal 3 homolog | | | |
| | | (Drosophila) | | | |
| 57724 | EPG5 | ectopic P- | −2.12 | −0.14 | 0.63 |
| | | granules | | | |
| | | autophagy | | | |
| | | protein 5 | | | |
| | | homolog (C. elegans) | | | |
| 100506282 | LOC100506282 | uncharacterized | −2.13 | 1.67 | 0.76 |
| | | LOC100506282 | | | |
| 147699 | PPM1N | protein | −2.13 | 0.16 | 0.53 |
| | | phosphatase, | | | |
| | | Mg2+/Mn2+ | | | |
| | | dependent, 1 N | | | |
| | | (putative) | | | |
| 3002 | GZMB | granzyme B | −2.13 | 0.76 | 0.87 |
| | | (granzyme 2, | | | |
| | | cytotoxic T- | | | |
| | | lymphocyte- | | | |
| | | associated serine | | | |
| | | esterase 1) | | | |
| 389320 | C5orf48 | chromosome 5 | −2.13 | 0.37 | 1.37 |
| | | open reading | | | |
| | | frame 48 | | | |
| 9914 | ATP2C2 | ATPase, Ca++ | −2.13 | 0.23 | 1.12 |
| | | transporting, | | | |
| | | type 2C, | | | |
| | | member 2 | | | |
| 10804 | GJB6 | gap junction | −2.13 | −1.12 | 1.65 |
| | | protein, beta 6, | | | |
| | | 30 kDa | | | |
| 51439 | FAM8A1 | family with | −2.13 | −0.89 | 1.07 |
| | | sequence | | | |
| | | similarity 8, | | | |
| | | member A1 | | | |
| 814 | CAMK4 | calcium/calmodulin- | −2.13 | −0.47 | 2.19 |
| | | dependent | | | |
| | | protein kinase | | | |
| | | IV | | | |
| 5272 | SERPINB9 | serpin peptidase | −2.13 | −0.34 | 0.40 |
| | | inhibitor, clade | | | |
| | | B (ovalbumin), | | | |
| | | member 9 | | | |
| 8428 | STK24 | serine/threonine | −2.13 | −1.09 | 1.59 |
| | | kinase 24 | | | |
| 23048 | FNBP1 | formin binding | −2.14 | 1.36 | 0.52 |
| | | protein 1 | | | |
| 344558 | SH3RF3 | SH3 domain | −2.14 | −0.73 | 1.32 |
| | | containing ring | | | |
| | | finger 3 | | | |
| 79078 | C1orf50 | chromosome 1 | −2.14 | 0.08 | 1.02 |
| | | open reading | | | |
| | | frame 50 | | | |
| 81606 | LBH | limb bud and | −2.14 | 0.57 | 2.63 |
| | | heart | | | |
| | | development | | | |
| 64377 | CHST8 | carbohydrate | −2.14 | 0.58 | −0.51 |
| | | (N- | | | |
| | | acetylgalactosamine | | | |
| | | 4-0) | | | |
| | | sulfotransferase 8 | | | |
| 57501 | KIAA1257 | KIAA1257 | −2.14 | −0.10 | 0.47 |
| 5991 | RFX3 | regulatory factor | −2.14 | 1.47 | 1.11 |
| | | X, 3 (influences | | | |
| | | HLA class II | | | |
| | | expression) | | | |
| 90075 | ZNF30 | zinc finger | −2.14 | 1.82 | 1.22 |
| | | protein 30 | | | |
| 375341 | C3orf62 | chromosome 3 | −2.14 | −0.26 | 0.66 |
| | | open reading | | | |
| | | frame 62 | | | |
| 100130548 | LOC100130548 | uncharacterized | −2.15 | −0.64 | 1.52 |
| | | LOC100130548 | | | |
| 114786 | XKR4 | XK, Kell blood | −2.15 | 0.58 | −0.23 |
| | | group complex | | | |
| | | subunit-related | | | |
| | | family, member 4 | | | |
| 150084 | IGSF5 | immunoglobulin | −2.15 | 0.75 | −0.16 |
| | | superfamily, | | | |
| | | member 5 | | | |
| 55012 | PPP2R3C | protein | −2.15 | −0.91 | 0.93 |
| | | phosphatase 2, | | | |
| | | regulatory | | | |
| | | subunit B″, | | | |
| | | gamma | | | |
| 147525 | LINC00526 | long intergenic | −2.15 | −0.23 | 1.84 |
| | | non-protein | | | |
| | | coding RNA | | | |
| | | 526 | | | |
| 54557 | SGTB | small glutamine- | −2.15 | −0.86 | 0.84 |
| | | rich | | | |
| | | tetratricopeptide | | | |
| | | repeat (TPR)- | | | |
| | | containing, beta | | | |
| 56267 | CCBL2 | cysteine | −2.15 | 0.21 | 1.69 |
| | | conjugate-beta | | | |
| | | lyase 2 | | | |
| 51176 | LEF1 | lymphoid | −2.15 | −1.11 | 2.08 |
| | | enhancer- | | | |
| | | binding factor 1 | | | |
| 10468 | FST | follistatin | −2.15 | −0.19 | 2.02 |
| 23258 | DENND5A | DENN/MADD | −2.15 | −0.31 | 0.08 |
| | | domain | | | |
| | | containing 5A | | | |
| 90768 | MGC45800 | uncharacterized | −2.15 | −0.26 | 1.01 |
| | | LOC90768 | | | |
| 134957 | STXBP5 | syntaxin binding | −2.16 | 0.06 | 0.64 |
| | | protein 5 | | | |
| | | (tomosyn) | | | |
| 80342 | TRAF3IP3 | TRAF3 | −2.16 | −0.07 | 1.61 |
| | | interacting | | | |
| | | protein 3 | | | |
| 91351 | DDX60L | DEAD (Asp- | −2.16 | −0.99 | 0.41 |
| | | Glu-Ala-Asp) | | | |
| | | box polypeptide | | | |
| | | 60-like | | | |
| 29916 | SNX11 | sorting nexin 11 | −2.16 | −0.18 | −0.11 |
| 8519 | IFITM1 | interferon | −2.16 | −0.67 | 1.50 |
| | | induced | | | |
| | | transmembrane | | | |
| | | protein 1 | | | |
| 2113 | ETS1 | v-ets avian | −2.16 | −0.80 | 1.75 |
| | | erythroblastosis | | | |
| | | virus E26 | | | |
| | | oncogene | | | |
| | | homolog 1 | | | |
| 55770 | EXOC2 | exocyst complex | −2.16 | 1.87 | 1.84 |
| | | component 2 | | | |
| 91833 | WDR20 | WD repeat | −2.16 | −0.21 | 1.84 |
| | | domain 20 | | | |
| 117177 | RAB3IP | RAB3A | −2.16 | −0.62 | 0.78 |
| | | interacting | | | |
| | | protein | | | |
| 546 | ATRX | alpha | −2.16 | 0.55 | 0.60 |
| | | thalassemia/mental | | | |
| | | retardation | | | |
| | | syndrome X- | | | |
| | | linked | | | |
| 4253 | CTAGE5 | CTAGE family, | −2.17 | 0.57 | 0.77 |
| | | member 5 | | | |
| 23214 | XPO6 | exportin 6 | −2.17 | −1.37 | 1.00 |
| 53347 | UBASH3A | ubiquitin | −2.17 | −1.35 | 2.45 |
| | | associated and | | | |
| | | SH3 domain | | | |
| | | containing A | | | |
| 56888 | KCMF1 | potassium | −2.17 | 0.00 | 0.23 |
| | | channel | | | |
| | | modulatory | | | |
| | | factor 1 | | | |
| 777 | CACNA1E | calcium channel, | −2.17 | −0.20 | 0.28 |
| | | voltage- | | | |
| | | dependent, R | | | |
| | | type, alpha 1E | | | |
| | | subunit | | | |
| 100216545 | KMT2E- | KMT2E | −2.17 | 0.71 | 2.04 |
| | AS1 | antisense RNA1 | | | |
| | | (head to head) | | | |
| 8879 | SGPL1 | sphingosine-1- | −2.17 | 1.73 | −0.25 |
| | | phosphate lyase 1 | | | |
| 9759 | HDAC4 | histone | −2.17 | −2.02 | 1.08 |
| | | deacetylase 4 | | | |
| 26578 | OSTF1 | osteoclast | −2.18 | −0.80 | 0.56 |
| | | stimulating | | | |
| | | factor 1 | | | |
| 4050 | LTB | lymphotoxin | −2.18 | −1.06 | 1.52 |
| | | beta (TNF | | | |
| | | superfamily, | | | |
| | | member 3) | | | |
| 4236 | MFAP1 | microfibrillar- | −2.18 | 0.60 | 0.85 |
| | | associated | | | |
| | | protein 1 | | | |
| 54491 | FAM105A | family with | −2.18 | −0.93 | 1.49 |
| | | sequence | | | |
| | | similarity 105, | | | |
| | | member A | | | |
| 55847 | CISD1 | CDGSH iron | −2.18 | 0.64 | 1.22 |
| | | sulfur domain 1 | | | |
| 100132707 | PAXIP1- | PAXIP1 | −2.19 | 0.24 | 0.73 |
| | AS2 | antisense RNA 2 | | | |
| 55884 | WSB2 | WD repeat and | −2.19 | 0.99 | −0.17 |
| | | SOCS box | | | |
| | | containing 2 | | | |
| 83607 | AMMECR1L | AMMECR1-like | −2.19 | 1.07 | 1.60 |
| 9252 | RPS6KA5 | ribosomal | −2.19 | −0.18 | 0.95 |
| | | protein S6 | | | |
| | | kinase, 90 kDa, | | | |
| | | polypeptide 5 | | | |
| 55119 | PRPF38B | pre-mRNA | −2.19 | 1.71 | 1.08 |
| | | processing | | | |
| | | factor 38B | | | |
| 57520 | HECW2 | HECT, C2 and | −2.19 | 0.30 | 0.43 |
| | | WW domain | | | |
| | | containing E3 | | | |
| | | ubiquitin protein | | | |
| | | ligase 2 | | | |
| 79818 | ZNF552 | zinc finger | −2.19 | −1.20 | 1.23 |
| | | protein 552 | | | |
| 8320 | EOMES | eomesodermin | −2.19 | 0.15 | 1.44 |
| 2792 | GNGT1 | guanine | −2.19 | 1.41 | −0.14 |
| | | nucleotide | | | |
| | | binding protein | | | |
| | | (G protein), | | | |
| | | gamma | | | |
| | | transducing | | | |
| | | activity | | | |
| | | polypeptide 1 | | | |
| 54910 | SEMA4C | sema domain, | −2.19 | 1.21 | 0.99 |
| | | immunoglobulin | | | |
| | | domain (Ig), | | | |
| | | transmembrane | | | |
| | | domain (TM) | | | |
| | | and short | | | |
| | | cytoplasmic | | | |
| | | domain, | | | |
| | | (semaphorin) 4C | | | |
| 55114 | ARHGAP17 | Rho GTPase | −2.19 | 0.18 | −0.27 |
| | | activating | | | |
| | | protein 17 | | | |
| 56253 | CRTAM | cytotoxic and | −2.19 | 0.83 | 1.31 |
| | | regulatory T cell | | | |
| | | molecule | | | |
| 65082 | VPS33A | vacuolar protein | −2.19 | −0.65 | 1.18 |
| | | sorting 33 | | | |
| | | homolog A (S. cerevisiae) | | | |
| 84719 | LINC00260 | long intergenic | −2.20 | 1.45 | 1.51 |
| | | non-protein | | | |
| | | coding RNA | | | |
| | | 260 | | | |
| 253558 | LCLAT1 | lysocardiolipin | −2.20 | 0.62 | 0.95 |
| | | acyltransferase 1 | | | |
| 6777 | STAT5B | signal | −2.20 | −0.54 | 1.15 |
| | | transducer and | | | |
| | | activator of | | | |
| | | transcription 5B | | | |
| 80025 | PANK2 | pantothenate | −2.20 | −0.45 | 0.85 |
| | | kinase 2 | | | |
| 84662 | GLIS2 | GLIS family | −2.20 | 0.95 | 0.42 |
| | | zinc finger 2 | | | |
| 89890 | KBTBD6 | kelch repeat and | −2.20 | 0.03 | 1.98 |
| | | BTB (POZ) | | | |
| | | domain | | | |
| | | containing 6 | | | |
| 10390 | CEPT1 | choline/ethanolamine | −2.20 | 0.35 | 1.23 |
| | | phosphotransferase 1 | | | |
| 2841 | GPR18 | G protein- | −2.20 | 1.15 | 2.42 |
| | | coupled receptor | | | |
| | | 18 | | | |
| 3904 | LAIR2 | leukocyte- | −2.20 | 0.84 | 0.21 |
| | | associated | | | |
| | | immunoglobulin- | | | |
| | | like receptor 2 | | | |
| 140460 | ASB7 | ankyrin repeat | −2.20 | 0.07 | 1.03 |
| | | and SOCS box | | | |
| | | containing 7 | | | |
| 9605 | VPS9D1 | VPS9 domain | −2.20 | −0.23 | 0.95 |
| | | containing 1 | | | |
| 283349 | RASSF3 | Ras association | −2.21 | −0.83 | 0.96 |
| | | (RalGDS/AF-6) | | | |
| | | domain family | | | |
| | | member 3 | | | |
| 51246 | SHISA5 | shisa family | −2.21 | −1.42 | 0.99 |
| | | member 5 | | | |
| 148268 | ZNF570 | zinc finger | −2.21 | 1.26 | 2.25 |
| | | protein 570 | | | |
| 26589 | MRPL46 | mitochondrial | −2.21 | 1.15 | 1.68 |
| | | ribosomal | | | |
| | | protein L46 | | | |
| 7049 | TGFBR3 | transforming | −2.21 | 1.01 | 2.02 |
| | | growth factor, | | | |
| | | beta receptor III | | | |
| 2791 | GNG11 | guanine | −2.21 | −0.39 | −0.02 |
| | | nucleotide | | | |
| | | binding protein | | | |
| | | (G protein), | | | |
| | | gamma 11 | | | |
| 51455 | REV1 | REV1, | −2.21 | 1.24 | 1.77 |
| | | polymerase | | | |
| | | (DNA directed) | | | |
| 1178 | CLC | Charcot-Leyden | −2.22 | 0.06 | 0.48 |
| | | crystal galectin | | | |
| 79153 | GDPD3 | glycerophosphodiester | −2.22 | −0.34 | 0.91 |
| | | phosphodiesterase | | | |
| | | domain | | | |
| | | containing 3 | | | |
| 101928524 | LOC101928524 | uncharacterized | −2.22 | −0.67 | 0.78 |
| | | LOC101928524 | | | |
| 11064 | CNTRL | centriolin | −2.22 | 0.66 | −0.56 |
| 23429 | RYBP | RING1 and | −2.22 | 1.35 | 0.64 |
| | | YY1 binding | | | |
| | | protein | | | |
| 5170 | PDPK1 | 3- | −2.22 | −0.83 | 0.31 |
| | | phosphoinositide | | | |
| | | dependent | | | |
| | | protein kinase-1 | | | |
| 10677 | AVIL | advillin | −2.23 | −0.85 | 0.84 |
| 3772 | KCNJ15 | potassium | −2.23 | −1.08 | 0.27 |
| | | inwardly- | | | |
| | | rectifying | | | |
| | | channel, | | | |
| | | subfamily J, | | | |
| | | member 15 | | | |
| 128061 | C1orf131 | chromosome 1 | −2.23 | 0.81 | 1.10 |
| | | open reading | | | |
| | | frame 131 | | | |
| 50856 | CLEC4A | C-type lectin | −2.23 | −0.08 | −0.28 |
| | | domain family | | | |
| | | 4, member A | | | |
| 6198 | RPS6KB1 | ribosomal | −2.23 | 2.01 | 1.73 |
| | | protein S6 | | | |
| | | kinase, 70 kDa, | | | |
| | | polypeptide 1 | | | |
| 101927588 | LOC101927588 | uncharacterized | −2.24 | −0.02 | 0.56 |
| | | LOC101927588 | | | |
| 2000 | ELF4 | E74-like factor 4 | −2.24 | 0.13 | 0.14 |
| | | (ets domain | | | |
| | | transcription | | | |
| | | factor) | | | |
| 493753 | COA5 | cytochrome c | −2.24 | 0.79 | 1.30 |
| | | oxidase | | | |
| | | assembly factor 5 | | | |
| 23081 | KDM4C | lysine (K)- | −2.24 | 1.09 | 0.42 |
| | | specific | | | |
| | | demethylase 4C | | | |
| 4773 | NFATC2 | nuclear factor of | −2.24 | 0.57 | 1.16 |
| | | activated T- | | | |
| | | cells, | | | |
| | | cytoplasmic, | | | |
| | | calcineurin- | | | |
| | | dependent 2 | | | |
| 10178 | TENM1 | teneurin | −2.25 | −0.87 | 0.62 |
| | | transmembrane | | | |
| | | protein 1 | | | |
| 4974 | OMG | oligodendrocyte | −2.25 | −1.43 | 0.63 |
| | | myelin | | | |
| | | glycoprotein | | | |
| 83860 | TAF3 | TAF3 RNA | −2.25 | 0.03 | 1.69 |
| | | polymerase II, | | | |
| | | TATA box | | | |
| | | binding protein | | | |
| | | (TBP)- | | | |
| | | associated | | | |
| | | factor, 140 kDa | | | |
| 91612 | CHURC1 | churchill | −2.25 | −1.11 | 1.06 |
| | | domain | | | |
| | | containing 1 | | | |
| 2178 | FANCE | Fanconi anemia, | −2.26 | −0.72 | 0.82 |
| | | complementation | | | |
| | | group E | | | |
| 55303 | GIMAP4 | GTPase, IMAP | −2.26 | −0.40 | 1.33 |
| | | family member 4 | | | |
| 221002 | RASGEF1A | RasGEF domain | −2.26 | −0.14 | 0.69 |
| | | family, member | | | |
| | | 1A | | | |
| 3930 | LBR | lamin B receptor | −2.26 | 0.12 | 0.71 |
| 55471 | NDUFAF7 | NADH | −2.26 | 1.20 | 1.05 |
| | | dehydrogenase | | | |
| | | (ubiquinone) | | | |
| | | complex I, | | | |
| | | assembly factor 7 | | | |
| 9354 | UBE4A | ubiquitination | −2.26 | 0.81 | 0.66 |
| | | factor E4A | | | |
| 83700 | JAM3 | junctional | −2.26 | −0.33 | 1.46 |
| | | adhesion | | | |
| | | molecule 3 | | | |
| 9140 | ATG12 | autophagy | −2.26 | 1.06 | 0.85 |
| | | related 12 | | | |
| 23526 | HMHA1 | histocompatibility | −2.27 | 0.14 | 1.43 |
| | | (minor) HA-1 | | | |
| 23590 | PDSS1 | prenyl | −2.27 | 0.01 | 0.69 |
| | | (decaprenyl) | | | |
| | | diphosphate | | | |
| | | synthase, | | | |
| | | subunit 1 | | | |
| 9270 | ITGB1BP1 | integrin beta 1 | −2.27 | 1.68 | 0.62 |
| | | binding protein 1 | | | |
| 10627 | MYL12A | myosin, light | −2.27 | −0.87 | 1.16 |
| | | chain 12A, | | | |
| | | regulatory, non- | | | |
| | | sarcomeric | | | |
| 26048 | ZNF500 | zinc finger | −2.27 | 3.47 | 1.42 |
| | | protein 500 | | | |
| 917 | CD3G | CD3g molecule, | −2.27 | −0.29 | 2.30 |
| | | gamma (CD3- | | | |
| | | TCR complex) | | | |
| 94039 | ZNF101 | zinc finger | −2.27 | −0.72 | 1.69 |
| | | protein 101 | | | |
| 4215 | MAP3K3 | mitogen- | −2.28 | −0.81 | 1.06 |
| | | activated protein | | | |
| | | kinase kinase | | | |
| | | kinase 3 | | | |
| 10663 | CXCR6 | chemokine | −2.28 | 0.37 | 1.78 |
| | | (C—X—C motif) | | | |
| | | receptor 6 | | | |
| 10308 | ZNF267 | zinc finger | −2.28 | 0.38 | 0.74 |
| | | protein 267 | | | |
| 8527 | DGKD | diacylglycerol | −2.28 | 0.75 | 1.41 |
| | | kinase, delta | | | |
| | | 130 kDa | | | |
| 926 | CD8B | CD8b molecule | −2.28 | −0.83 | 2.69 |
| 9619 | ABCG1 | ATP-binding | −2.28 | 0.94 | 1.01 |
| | | cassette, sub- | | | |
| | | family G | | | |
| | | (WHITE), | | | |
| | | member 1 | | | |
| 5998 | RGS3 | regulator of G- | −2.29 | −0.40 | 0.51 |
| | | protein signaling 3 | | | |
| 132625 | ZFP42 | ZFP42 zinc | −2.29 | 0.10 | 1.45 |
| | | finger protein | | | |
| 88455 | ANKRD13A | ankyrin repeat | −2.30 | −0.39 | 0.13 |
| | | domain 13A | | | |
| 3604 | TNFRSF9 | tumor necrosis | −2.30 | −0.59 | 0.42 |
| | | factor receptor | | | |
| | | superfamily, | | | |
| | | member 9 | | | |
| 100507398 | INTS6- | INTS6 antisense | −2.30 | 1.02 | 1.07 |
| | AS1 | RNA 1 | | | |
| 58517 | RBM25 | RNA binding | −2.30 | 2.48 | 1.59 |
| | | motif protein 25 | | | |
| 51192 | CKLF | chemokine-like | −2.31 | −0.45 | 0.59 |
| | | factor | | | |
| 2177 | FANCD2 | Fanconi anemia, | −2.31 | 1.14 | 1.23 |
| | | complementation | | | |
| | | group D2 | | | |
| 57097 | PARP11 | poly (ADP- | −2.31 | −0.19 | 0.77 |
| | | ribose) | | | |
| | | polymerase | | | |
| | | family, member | | | |
| | | 11 | | | |
| 11214 | AKAP13 | A kinase | −2.31 | 0.37 | 0.68 |
| | | (PRKA) anchor | | | |
| | | protein 13 | | | |
| 4600 | MX2 | myxovirus | −2.31 | −1.58 | 0.05 |
| | | (influenza virus) | | | |
| | | resistance 2 | | | |
| | | (mouse) | | | |
| 84255 | SLC37A3 | solute carrier | −2.31 | −0.59 | 0.93 |
| | | family 37, | | | |
| | | member 3 | | | |
| 11216 | AKAP10 | A kinase | −2.32 | 0.82 | 1.04 |
| | | (PRKA) anchor | | | |
| | | protein 10 | | | |
| 162417 | NAGS | N- | −2.32 | 0.09 | 1.46 |
| | | acetylglutamate | | | |
| | | synthase | | | |
| 101928617 | LOC101928617 | uncharacterized | −2.32 | −0.30 | 0.64 |
| | | LOC101928617 | | | |
| 10617 | STAMBP | STAM binding | −2.32 | 1.84 | −0.17 |
| | | protein | | | |
| 1326 | MAP3K8 | mitogen- | −2.32 | 0.72 | 0.63 |
| | | activated protein | | | |
| | | kinase kinase | | | |
| | | kinase 8 | | | |
| 23315 | SLC9A8 | solute carrier | −2.32 | −1.35 | 0.88 |
| | | family 9, | | | |
| | | subfamily A | | | |
| | | (NHE8, cation | | | |
| | | proton antiporter | | | |
| | | 8), member 8 | | | |
| 10791 | VAMP5 | vesicle- | −2.32 | −0.11 | 1.36 |
| | | associated | | | |
| | | membrane | | | |
| | | protein 5 | | | |
| 8718 | TNFRSF25 | tumor necrosis | −2.32 | −0.24 | 3.91 |
| | | factor receptor | | | |
| | | superfamily, | | | |
| | | member 25 | | | |
| 51571 | FAM49B | family with | −2.33 | −0.68 | 0.75 |
| | | sequence | | | |
| | | similarity 49, | | | |
| | | member B | | | |
| 283521 | LINC00282 | long intergenic | −2.33 | −2.30 | 1.78 |
| | | non-protein | | | |
| | | coding RNA | | | |
| | | 282 | | | |
| 51122 | COMMD2 | COMM domain | −2.33 | −0.09 | 1.53 |
| | | containing 2 | | | |
| 5372 | PMM1 | phosphomanno | −2.33 | 0.16 | 1.25 |
| | | mutase 1 | | | |
| 9830 | TRIM14 | tripartite motif | −2.33 | −0.29 | 1.44 |
| | | containing 14 | | | |
| 314 | AOC2 | amine oxidase, | −2.33 | −0.52 | 0.23 |
| | | copper | | | |
| | | containing 2 | | | |
| | | (retina-specific) | | | |
| 51761 | ATP8A2 | ATPase, | −2.33 | 0.45 | 1.61 |
| | | aminophospholipid | | | |
| | | transporter, | | | |
| | | class I, type 8A, | | | |
| | | member 2 | | | |
| 493861 | EID3 | EP300 | −2.34 | 0.84 | 1.87 |
| | | interacting | | | |
| | | inhibitor of | | | |
| | | differentiation 3 | | | |
| 6672 | SP100 | SP100 nuclear | −2.34 | −0.15 | 0.33 |
| | | antigen | | | |
| 55578 | SUPT20H | suppressor of Ty | −2.34 | −0.88 | 2.05 |
| | | 20 homolog (S. cerevisiae) | | | |
| 159 | ADSS | adenylosuccinate | −2.34 | 1.28 | 1.03 |
| | | synthase | | | |
| 9683 | N4BP1 | NEDD4 binding | −2.34 | −0.82 | 0.93 |
| | | protein 1 | | | |
| 26287 | ANKRD2 | ankyrin repeat | −2.35 | 0.96 | −1.11 |
| | | domain 2 | | | |
| | | (stretch | | | |
| | | responsive | | | |
| | | muscle) | | | |
| 57147 | SCYL3 | SCY1-like 3 (S. cerevisiae) | −2.35 | 0.41 | 1.20 |
| 1808 | DPYSL2 | dihydropyrimidinase- | −2.35 | 0.81 | −0.69 |
| | | like 2 | | | |
| 23355 | VPS8 | vacuolar protein | −2.35 | −0.37 | 0.18 |
| | | sorting 8 | | | |
| | | homolog (S. cerevisiae) | | | |
| 8115 | TCL1A | T-cell | −2.35 | 0.02 | 0.58 |
| | | leukemia/lymphoma | | | |
| | | 1A | | | |
| 94120 | SYTL3 | synaptotagmin- | −2.35 | 0.66 | 1.11 |
| | | like 3 | | | |
| 22950 | SLC4A1AP | solute carrier | −2.35 | −0.93 | 1.36 |
| | | family 4 (anion | | | |
| | | exchanger), | | | |
| | | member 1, | | | |
| | | adaptor protein | | | |
| 27342 | RABGEF1 | RAB guanine | −2.35 | −0.93 | 1.58 |
| | | nucleotide | | | |
| | | exchange factor | | | |
| | | (GEF)1 | | | |
| 64926 | RASAL3 | RAS protein | −2.35 | 0.01 | 0.96 |
| | | activator like 3 | | | |
| 100507331 | ZSWIM8- | ZSWIM8 | −2.36 | 0.57 | 1.26 |
| | AS1 | antisense RNA 1 | | | |
| 4068 | SH2D1A | SH2 domain | −2.36 | 1.66 | 1.54 |
| | | containing 1A | | | |
| 60481 | ELOVL5 | ELOVL fatty | −2.36 | 1.02 | 0.79 |
| | | acid elongase 5 | | | |
| 2634 | GBP2 | guanylate | −2.36 | 0.00 | 0.80 |
| | | binding protein | | | |
| | | 2, interferon- | | | |
| | | inducible | | | |
| 55824 | PAG1 | phosphoprotein | −2.36 | −1.03 | 1.59 |
| | | associated with | | | |
| | | glycosphingolipid | | | |
| | | microdomains 1 | | | |
| 79074 | C2orf49 | chromosome 2 | −2.36 | 1.44 | 0.56 |
| | | open reading | | | |
| | | frame 49 | | | |
| 220930 | ZEB1- | ZEB1 antisense | −2.36 | 1.06 | 0.36 |
| | AS1 | RNA 1 | | | |
| 64421 | DCLRE1C | DNA cross-link | −2.36 | 0.49 | 0.61 |
| | | repair 1C | | | |
| 64121 | RRAGC | Ras-related GTP | −2.37 | 0.32 | 0.40 |
| | | binding C | | | |
| 137835 | TMEM71 | transmembrane | −2.37 | −0.41 | 1.11 |
| | | protein 71 | | | |
| 57674 | RNF213 | ring finger | −2.37 | −0.91 | 0.53 |
| | | protein 213 | | | |
| 728392 | LOC728392 | uncharacterized | −2.37 | −0.94 | 0.78 |
| | | LOC728392 | | | |
| 329 | BIRC2 | baculoviral IAP | −2.38 | 1.72 | 1.94 |
| | | repeat | | | |
| | | containing 2 | | | |
| 55279 | ZNF654 | zinc finger | −2.38 | 0.07 | 1.10 |
| | | protein 654 | | | |
| 7107 | GPR137B | G protein- | −2.38 | 0.57 | 0.21 |
| | | coupled receptor | | | |
| | | 137B | | | |
| 9416 | DDX23 | DEAD (Asp- | −2.38 | 0.21 | 0.21 |
| | | Glu-Ala-Asp) | | | |
| | | box polypeptide | | | |
| | | 23 | | | |
| 137964 | AGPAT6 | 1-acylglycerol- | −2.38 | 1.36 | 0.99 |
| | | 3-phosphate O- | | | |
| | | acyltransferase 6 | | | |
| 3431 | SP110 | SP110 nuclear | −2.38 | −0.53 | 0.24 |
| | | body protein | | | |
| 4140 | MARK3 | MAP/microtubule | −2.38 | 1.19 | 0.52 |
| | | affinity- | | | |
| | | regulating | | | |
| | | kinase 3 | | | |
| 59269 | HIVEP3 | human | −2.38 | 0.24 | 0.53 |
| | | immunodeficiency | | | |
| | | virus type I | | | |
| | | enhancer | | | |
| | | binding protein 3 | | | |
| 7846 | TUBA1A | tubulin, alpha 1a | −2.38 | 0.54 | 0.04 |
| 100652740 | C16orf98 | chromosome 16 | −2.38 | 0.57 | 1.13 |
| | | open reading | | | |
| | | frame 98 | | | |
| 23398 | PPWD1 | peptidylprolyl | −2.38 | 0.94 | 1.67 |
| | | isomerase | | | |
| | | domain and WD | | | |
| | | repeat | | | |
| | | containing 1 | | | |
| 158747 | MOSPD2 | motile sperm | −2.39 | −0.04 | 0.20 |
| | | domain | | | |
| | | containing 2 | | | |
| 285512 | FAM13A- | FAM13A | −2.39 | 0.05 | 0.98 |
| | AS1 | antisense RNA 1 | | | |
| 3937 | LCP2 | lymphocyte | −2.39 | 0.47 | 1.08 |
| | | cytosolic protein | | | |
| | | 2 (SH2 domain | | | |
| | | containing | | | |
| | | leukocyte | | | |
| | | protein of | | | |
| | | 76 kDa) | | | |
| 730051 | ZNF814 | zinc finger | −2.39 | 0.33 | 1.83 |
| | | protein 814 | | | |
| 9320 | TRIP12 | thyroid hormone | −2.39 | 1.30 | 0.94 |
| | | receptor | | | |
| | | interactor 12 | | | |
| 6932 | TCF7 | transcription | −2.39 | −0.88 | 2.43 |
| | | factor 7 (T-cell | | | |
| | | specific, HMG- | | | |
| | | box) | | | |
| 79722 | ANKRD55 | ankyrin repeat | −2.39 | −0.59 | 1.57 |
| | | domain 55 | | | |
| 115362 | GBP5 | guanylate | −2.39 | −0.13 | 0.78 |
| | | binding protein 5 | | | |
| 29909 | GPR171 | G protein- | −2.39 | −0.05 | 2.07 |
| | | coupled receptor | | | |
| | | 171 | | | |
| 440503 | PLIN5 | perilipin 5 | −2.40 | −0.13 | −0.04 |
| 80183 | KIAA0226L | KIAA0226-like | −2.40 | −0.11 | −0.47 |
| 80709 | AKNA | AT-hook | −2.40 | −0.40 | 1.34 |
| | | transcription | | | |
| | | factor | | | |
| 10562 | OLFM4 | olfactomedin 4 | −2.40 | −0.72 | 1.37 |
| 64780 | MICAL1 | microtubule | −2.41 | 0.90 | −0.01 |
| | | associated | | | |
| | | monooxygenase, | | | |
| | | calponin and | | | |
| | | LIM domain | | | |
| | | containing 1 | | | |
| 155038 | GIMAP8 | GTPase, IMAP | −2.41 | −0.08 | 1.03 |
| | | family member 8 | | | |
| 1783 | DYNC1L12 | dynein, | −2.41 | 0.29 | 0.55 |
| | | cytoplasmic 1, | | | |
| | | light | | | |
| | | intermediate | | | |
| | | chain 2 | | | |
| 100287569 | LINC00173 | long intergenic | −2.42 | 0.79 | 0.50 |
| | | non-protein | | | |
| | | coding RNA | | | |
| | | 173 | | | |
| 5696 | PSMB8 | proteasome | −2.42 | 1.75 | 1.55 |
| | | (prosome, | | | |
| | | macropain) | | | |
| | | subunit, beta | | | |
| | | type, 8 | | | |
| 11066 | SNRNP35 | small nuclear | −2.42 | −0.97 | 0.66 |
| | | ribonucleoprotein | | | |
| | | 35 kDa | | | |
| | | (U11/U12) | | | |
| 122553 | TRAPPC6B | trafficking | −2.42 | 1.80 | 0.59 |
| | | protein particle | | | |
| | | complex 6B | | | |
| 50484 | RRM2B | ribonucleotide | −2.43 | 0.68 | 0.40 |
| | | reductase M2 B | | | |
| | | (TP53 | | | |
| | | inducible) | | | |
| 9847 | C2CD5 | C2 calcium- | −2.43 | −0.31 | 1.91 |
| | | dependent | | | |
| | | domain | | | |
| | | containing 5 | | | |
| 129285 | PPP1R21 | protein | −2.43 | 0.25 | 0.06 |
| | | phosphatase 1, | | | |
| | | regulatory | | | |
| | | subunit 21 | | | |
| 9794 | MAML1 | mastermind-like | −2.43 | −0.49 | 1.65 |
| | | 1 (Drosophila) | | | |
| 84309 | NUDT16L1 | nudix | −2.43 | 0.68 | 1.62 |
| | | (nucleoside | | | |
| | | diphosphate | | | |
| | | linked moiety | | | |
| | | X)-type motif | | | |
| | | 16-like 1 | | | |
| 65258 | MPPE1 | metallophosphoesterase 1 | −2.43 | 0.07 | 0.99 |
| 5562 | PRKAA1 | protein kinase, | −2.44 | 0.73 | 0.77 |
| | | AMP-activated, | | | |
| | | alpha 1 catalytic | | | |
| | | subunit | | | |
| 123036 | TC2N | tandem C2 | −2.44 | −0.21 | 2.02 |
| | | domains, | | | |
| | | nuclear | | | |
| 84166 | NLRC5 | NLR family, | −2.45 | −0.28 | 1.58 |
| | | CARD domain | | | |
| | | containing 5 | | | |
| 253143 | PRR14L | proline rich 14- | −2.45 | 0.27 | −0.17 |
| | | like | | | |
| 1236 | CCR7 | chemokine (C-C | −2.45 | 0.54 | 2.19 |
| | | motif) receptor 7 | | | |
| 266747 | RGL4 | ral guanine | −2.46 | −1.20 | 1.65 |
| | | nucleotide | | | |
| | | dissociation | | | |
| | | stimulator-like 4 | | | |
| 25988 | HINFP | histone H4 | −2.46 | 1.72 | 0.72 |
| | | transcription | | | |
| | | factor | | | |
| 6515 | SLC2A3 | solute carrier | −2.46 | −0.20 | 0.92 |
| | | family 2 | | | |
| | | (facilitated | | | |
| | | glucose | | | |
| | | transporter), | | | |
| | | member 3 | | | |
| 90592 | ZNF700 | zinc finger | −2.46 | −0.40 | 0.59 |
| | | protein 700 | | | |
| 6867 | TACC1 | transforming, | −2.47 | 0.01 | 1.16 |
| | | acidic coiled- | | | |
| | | coil containing | | | |
| | | protein 1 | | | |
| 23041 | MON2 | MON2 homolog | −2.47 | 0.67 | 1.20 |
| | | (S. cerevisiae) | | | |
| 100289230 | LOC100289230 | uncharacterized | −2.47 | 0.61 | 2.09 |
| | | LOC100289230 | | | |
| 57169 | ZNFX1 | zinc finger, | −2.48 | −0.54 | 0.80 |
| | | NFX1-type | | | |
| | | containing 1 | | | |
| 1105 | CHD1 | chromodomain | −2.48 | 0.28 | 1.87 |
| | | helicase DNA | | | |
| | | binding protein 1 | | | |
| 943 | TNFRSF8 | tumor necrosis | −2.48 | 1.74 | −0.67 |
| | | factor receptor | | | |
| | | superfamily, | | | |
| | | member 8 | | | |
| 3275 | PRMT2 | protein arginine | −2.48 | −0.49 | 1.32 |
| | | methyltransferase 2 | | | |
| 8851 | CDK5R1 | cyclin- | −2.49 | −0.76 | 2.06 |
| | | dependent | | | |
| | | kinase 5, | | | |
| | | regulatory | | | |
| | | subunit 1 (p35) | | | |
| 168537 | GIMAP7 | GTPase, IMAP | −2.49 | 0.04 | 1.78 |
| | | family member 7 | | | |
| 256236 | NAPSB | napsin B | −2.49 | 1.72 | −0.85 |
| | | aspartic | | | |
| | | peptidase, | | | |
| | | pseudogene | | | |
| 26235 | FBXL4 | F-box and | −2.50 | 1.20 | −0.11 |
| | | leucine-rich | | | |
| | | repeat protein 4 | | | |
| 9363 | RAB33A | RAB33A, | −2.50 | 0.55 | 2.57 |
| | | member RAS | | | |
| | | oncogene family | | | |
| 729683 | LOC729683 | uncharacterized | −2.50 | 0.55 | 1.35 |
| | | LOC729683 | | | |
| 388969 | C2orf68 | chromosome 2 | −2.51 | 0.77 | 1.38 |
| | | open reading | | | |
| | | frame 68 | | | |
| 4818 | NKG7 | natural killer | −2.51 | 0.44 | 1.69 |
| | | cell group 7 | | | |
| | | sequence | | | |
| 93953 | ACRC | acidic repeat | −2.51 | 0.38 | 1.86 |
| | | containing | | | |
| 28982 | FLVCR1 | feline leukemia | −2.51 | 1.33 | 0.57 |
| | | virus subgroup | | | |
| | | C cellular | | | |
| | | receptor 1 | | | |
| 84911 | ZNF382 | zinc finger | −2.52 | 1.69 | 1.32 |
| | | protein 382 | | | |
| 66036 | MTMR9 | myotubularin | −2.52 | −0.10 | 1.66 |
| | | related protein 9 | | | |
| 9750 | FAM65B | family with | −2.52 | −0.37 | 1.14 |
| | | sequence | | | |
| | | similarity 65, | | | |
| | | member B | | | |
| 9938 | ARHGAP25 | Rho GTPase | −2.52 | 0.29 | 0.14 |
| | | activating | | | |
| | | protein 25 | | | |
| 120425 | AMICA1 | adhesion | −2.52 | −0.42 | 0.68 |
| | | molecule, | | | |
| | | interacts with | | | |
| | | CXADR antigen 1 | | | |
| 6840 | SVIL | supervillin | −2.53 | −0.64 | 0.50 |
| 26268 | FBXO9 | F-box protein 9 | −2.53 | 0.69 | 1.35 |
| 55206 | SBNO1 | strawberry notch | −2.53 | −0.54 | 1.57 |
| | | homolog 1 | | | |
| | | (Drosophila) | | | |
| 355 | FAS | Fas cell surface | −2.54 | 0.43 | 0.39 |
| | | death receptor | | | |
| 50615 | IL21R | interleukin 21 | −2.54 | 0.35 | 1.97 |
| | | receptor | | | |
| 221178 | SPATA13 | spermatogenesis | −2.54 | −2.61 | 1.22 |
| | | associated 13 | | | |
| 11104 | KATNA1 | katanin p60 | −2.54 | 0.20 | 2.21 |
| | | (ATPase | | | |
| | | containing) | | | |
| | | subunit A1 | | | |
| 1117 | CHI3L2 | chitinase 3-like 2 | −2.54 | 0.52 | 1.23 |
| 374969 | CCDC23 | coiled-coil | −2.54 | −1.26 | 1.25 |
| | | domain | | | |
| | | containing 23 | | | |
| 6285 | S100B | S100 calcium | −2.55 | 1.40 | 1.92 |
| | | binding protein B | | | |
| 387357 | THEMIS | thymocyte | −2.55 | −0.15 | 1.81 |
| | | selection | | | |
| | | associated | | | |
| 2960 | GTF2E1 | general | −2.56 | 0.13 | 0.92 |
| | | transcription | | | |
| | | factor IIE, | | | |
| | | polypeptide 1, | | | |
| | | alpha 56 kDa | | | |
| 100129550 | LOC100129550 | uncharacterized | −2.56 | −0.15 | 0.93 |
| | | LOC100129550 | | | |
| 54520 | CCDC93 | coiled-coil | −2.56 | −1.12 | 2.30 |
| | | domain | | | |
| | | containing 93 | | | |
| 10302 | SNAPC5 | small nuclear | −2.56 | 0.07 | 1.15 |
| | | RNA activating | | | |
| | | complex, | | | |
| | | polypeptide 5, | | | |
| | | 19 kDa | | | |
| 50939 | IMPG2 | interphotoreceptor | −2.56 | 1.12 | 1.76 |
| | | matrix | | | |
| | | proteoglycan 2 | | | |
| 8802 | SUCLG1 | succinate-CoA | −2.56 | 2.64 | 1.54 |
| | | ligase, alpha | | | |
| | | subunit | | | |
| 23332 | CLASP1 | cytoplasmic | −2.57 | 1.26 | 2.20 |
| | | linker associated | | | |
| | | protein 1 | | | |
| 203328 | SUSD3 | sushi domain | −2.57 | −0.72 | 2.46 |
| | | containing 3 | | | |
| 126231 | ZNF573 | zinc finger | −2.58 | 0.65 | 1.46 |
| | | protein 573 | | | |
| 6993 | DYNLT1 | dynein, light | −2.58 | −0.49 | 1.03 |
| | | chain, Tctex- | | | |
| | | type 1 | | | |
| 58500 | ZNF250 | zinc finger | −2.59 | −0.24 | 0.92 |
| | | protein 250 | | | |
| 10507 | SEMA4D | sema domain, | −2.59 | −1.16 | 1.46 |
| | | immunoglobulin | | | |
| | | domain (Ig), | | | |
| | | transmembrane | | | |
| | | domain (TM) | | | |
| | | and short | | | |
| | | cytoplasmic | | | |
| | | domain, | | | |
| | | (semaphorin) | | | |
| | | 4D | | | |
| 170482 | CLEC4C | C-type lectin | −2.59 | −0.58 | 0.36 |
| | | domain family | | | |
| | | 4, member C | | | |
| 3004 | GZMM | granzyme M | −2.59 | 1.40 | 2.20 |
| | | (lymphocyte | | | |
| | | met-ase 1) | | | |
| 64895 | PAPOLG | poly(A) | −2.59 | 1.43 | 0.78 |
| | | polymerase | | | |
| | | gamma | | | |
| 7850 | IL1R2 | interleukin 1 | −2.59 | 0.45 | 0.05 |
| | | receptor, type II | | | |
| 93594 | TBC1D31 | TBC1 domain | −2.59 | 0.76 | 0.44 |
| | | family, member | | | |
| | | 31 | | | |
| 23215 | PRRC2C | proline-rich | −2.60 | −0.05 | 1.60 |
| | | coiled-coil 2C | | | |
| 80196 | RNF34 | ring finger | −2.60 | 0.73 | 1.11 |
| | | protein 34, E3 | | | |
| | | ubiquitin protein | | | |
| | | ligase | | | |
| 284415 | VSTM1 | V-set and | −2.60 | −0.29 | 0.72 |
| | | transmembrane | | | |
| | | domain | | | |
| | | containing 1 | | | |
| 56882 | CDC42SE1 | CDC42 small | −2.60 | −0.64 | 1.52 |
| | | effector 1 | | | |
| 6508 | SLC4A3 | solute carrier | −2.60 | 0.54 | 0.57 |
| | | family 4 (anion | | | |
| | | exchanger), | | | |
| | | member 3 | | | |
| 79663 | HSPBAP1 | HSPB (heat | −2.61 | −0.64 | 0.48 |
| | | shock 27 kDa) | | | |
| | | associated | | | |
| | | protein 1 | | | |
| 474344 | GIMAP6 | GTPase, IMAP | −2.61 | 0.08 | 1.31 |
| | | family member 6 | | | |
| 100505746 | ITGB2- | ITGB2 antisense | −2.62 | 0.00 | 0.48 |
| | AS1 | RNA 1 | | | |
| 1293 | COL6A3 | collagen type | −2.62 | 1.08 | 0.33 |
| | | VI, alpha 3 | | | |
| 10294 | DNAJA2 | DnaJ (Hsp40) | −2.62 | −0.42 | 0.80 |
| | | homolog, | | | |
| | | subfamily A, | | | |
| | | member 2 | | | |
| 3091 | HIF1A | hypoxia | −2.62 | 0.89 | 0.98 |
| | | inducible factor | | | |
| | | 1, alpha subunit | | | |
| | | (basic helix- | | | |
| | | loop-helix | | | |
| | | transcription | | | |
| | | factor) | | | |
| 79573 | TTC13 | tetratricopeptide | −2.63 | −0.03 | 1.77 |
| | | repeat domain | | | |
| | | 13 | | | |
| 9208 | LRRFIP1 | leucine rich | −2.63 | 1.90 | 0.55 |
| | | repeat (in FLII) | | | |
| | | interacting | | | |
| | | protein 1 | | | |
| 29116 | MYLIP | myosin | −2.63 | 0.05 | 1.36 |
| | | regulatory light | | | |
| | | chain interacting | | | |
| | | protein | | | |
| 92370 | ACPL2 | acid | −2.63 | 0.23 | 1.78 |
| | | phosphatase-like 2 | | | |
| 9546 | APBA3 | amyloid beta | −2.64 | 0.44 | 0.47 |
| | | (A4) precursor | | | |
| | | protein-binding, | | | |
| | | family A, | | | |
| | | member 3 | | | |
| 253018 | HCG27 | HLA complex | −2.64 | −0.10 | 1.10 |
| | | group 27 (non- | | | |
| | | protein coding) | | | |
| 10096 | ACTR3 | ARP3 actin- | −2.65 | 1.04 | −0.12 |
| | | related protein 3 | | | |
| | | homolog (yeast) | | | |
| 84138 | SLC7A6OS | solute carrier | −2.65 | 0.77 | 0.59 |
| | | family 7, | | | |
| | | member 6 | | | |
| | | opposite strand | | | |
| 8440 | NCK2 | NCK adaptor | −2.65 | 0.72 | 1.76 |
| | | protein 2 | | | |
| 7277 | TUBA4A | tubulin, alpha 4a | −2.65 | −0.26 | 1.61 |
| 8269 | TMEM187 | transmembrane | −2.66 | 0.48 | 0.64 |
| | | protein 187 | | | |
| 8807 | IL18RAP | interleukin 18 | −2.66 | −0.44 | 1.01 |
| | | receptor | | | |
| | | accessory | | | |
| | | protein | | | |
| 1235 | CCR6 | chemokine (C-C | −2.67 | 1.38 | 2.29 |
| | | motif) receptor 6 | | | |
| 7059 | THBS3 | thrombospondin 3 | −2.67 | 0.99 | 0.61 |
| 54971 | BANP | BTG3 | −2.67 | −0.13 | 1.08 |
| | | associated | | | |
| | | nuclear protein | | | |
| 57677 | ZFP14 | ZFP14 zinc | −2.68 | 1.28 | 1.43 |
| | | finger protein | | | |
| 6890 | TAP1 | transporter 1, | −2.68 | 0.01 | 1.23 |
| | | ATP-binding | | | |
| | | cassette, sub- | | | |
| | | family B | | | |
| | | (MDR/TAP) | | | |
| 22944 | KIN | KIN, antigenic | −2.69 | 0.44 | 1.04 |
| | | determinant of | | | |
| | | recA protein | | | |
| | | homolog | | | |
| | | (mouse) | | | |
| 26512 | INTS6 | integrator | −2.69 | 0.30 | 0.91 |
| | | complex subunit 6 | | | |
| 92797 | HELB | helicase (DNA) B | −2.69 | 0.69 | 0.21 |
| 5481 | PPID | peptidylprolyl | −2.70 | 0.46 | 0.93 |
| | | isomerase D | | | |
| 22897 | CEP164 | centrosomal | −2.71 | −0.13 | 0.44 |
| | | protein 164 kDa | | | |
| 3620 | IDO1 | indoleamine 2,3- | −2.71 | 0.37 | 0.85 |
| | | dioxygenase 1 | | | |
| 64766 | S100PBP | S100P binding | −2.71 | 1.02 | 0.38 |
| | | protein | | | |
| 8809 | IL18R1 | interleukin 18 | −2.71 | −0.63 | 1.28 |
| | | receptor 1 | | | |
| 116984 | ARAP2 | ArfGAP with | −2.71 | 0.12 | 1.76 |
| | | RhoGAP | | | |
| | | domain, ankyrin | | | |
| | | repeat and PH | | | |
| | | domain 2 | | | |
| 440823 | MIAT | myocardial | −2.72 | 1.47 | 1.42 |
| | | infarction | | | |
| | | associated | | | |
| | | transcript (non- | | | |
| | | protein coding) | | | |
| 59340 | HRH4 | histamine | −2.72 | 1.07 | 0.24 |
| | | receptor H4 | | | |
| 197259 | MLKL | mixed lineage | −2.73 | 0.14 | −0.03 |
| | | kinase domain- | | | |
| | | like | | | |
| 23670 | TMEM2 | transmembrane | −2.73 | −0.89 | 1.36 |
| | | protein 2 | | | |
| 643314 | KIAA0754 | KIAA0754 | −2.73 | 1.42 | −0.20 |
| 28526 | TRDC | T cell receptor | −2.73 | 0.84 | 1.43 |
| | | delta constant | | | |
| 55096 | EBLN2 | endogenous | −2.74 | 0.08 | 1.21 |
| | | Bornavirus-like | | | |
| | | nucleoprotein 2 | | | |
| 2796 | GNRH1 | gonadotropin- | −2.74 | 1.82 | 0.79 |
| | | releasing | | | |
| | | hormone 1 | | | |
| | | (luteinizing- | | | |
| | | releasing | | | |
| | | hormone) | | | |
| 54509 | RHOF | ras homolog | −2.74 | 2.02 | 1.91 |
| | | family member | | | |
| | | F (in filopodia) | | | |
| 11120 | BTN2A1 | butyrophilin, | −2.75 | 0.47 | 1.81 |
| | | subfamily 2, | | | |
| | | member A1 | | | |
| 84869 | CBR4 | carbonyl | −2.75 | 1.20 | 0.13 |
| | | reductase 4 | | | |
| 28991 | COMMD5 | COMM domain | −2.76 | 0.53 | 0.53 |
| | | containing 5 | | | |
| 154141 | MBOAT1 | membrane | −2.79 | 0.40 | 0.42 |
| | | bound O- | | | |
| | | acyltransferase | | | |
| | | domain | | | |
| | | containing 1 | | | |
| 3659 | IRF1 | interferon | −2.79 | 0.33 | 1.23 |
| | | regulatory factor 1 | | | |
| 154007 | SNRNP48 | small nuclear | −2.80 | 0.64 | 0.71 |
| | | ribonucleoprotein | | | |
| | | 48 kDa | | | |
| | | (U11/U12) | | | |
| 116835 | HSPA12B | heat shock 70 kD | −2.81 | 0.73 | 0.95 |
| | | protein 12B | | | |
| 9488 | PIGB | phosphatidylinositol | −2.82 | −0.03 | 1.02 |
| | | glycan | | | |
| | | anchor | | | |
| | | biosynthesis, | | | |
| | | class B | | | |
| 100293516 | ZNF587B | zinc finger | −2.83 | −0.05 | 0.73 |
| | | protein 587B | | | |
| 9217 | VAPB | VAMP (vesicle- | −2.84 | 0.11 | 1.97 |
| | | associated | | | |
| | | membrane | | | |
| | | protein)- | | | |
| | | associated | | | |
| | | protein B and C | | | |
| 10538 | BATF | basic leucine | −2.84 | 0.90 | 0.63 |
| | | zipper | | | |
| | | transcription | | | |
| | | factor, ATF-like | | | |
| 6935 | ZEB1 | zinc finger E- | −2.87 | 0.92 | 1.75 |
| | | box binding | | | |
| | | homeobox 1 | | | |
| 10443 | N4BP2L2 | NEDD4 binding | −2.88 | 0.79 | 1.38 |
| | | protein 2-like 2 | | | |
| 3837 | KPNB1 | karyopherin | −2.88 | 0.72 | 0.69 |
| | | (importin) beta 1 | | | |
| 81698 | LINC00597 | long intergenic | −2.88 | 0.51 | 0.98 |
| | | non-protein | | | |
| | | coding RNA | | | |
| | | 597 | | | |
| 2643 | GCH1 | GTP | −2.89 | 1.47 | 1.66 |
| | | cyclohydrolase 1 | | | |
| 57559 | STAMBPL1 | STAM binding | −2.89 | 1.12 | 1.44 |
| | | protein-like 1 | | | |
| 116842 | LEAP2 | liver expressed | −2.89 | 1.23 | 1.24 |
| | | antimicrobial | | | |
| | | peptide 2 | | | |
| 202 | AIM1 | absent in | −2.90 | −0.39 | 1.31 |
| | | melanoma 1 | | | |
| 9934 | P2RY14 | purinergic | −2.90 | 0.39 | 0.49 |
| | | receptor P2Y, | | | |
| | | G-protein | | | |
| | | coupled, 14 | | | |
| 100216546 | LINC01004 | long intergenic | −2.90 | 1.20 | 0.90 |
| | | non-protein | | | |
| | | coding RNA | | | |
| | | 1004 | | | |
| 9692 | KIAA0391 | KIAA0391 | −2.90 | 1.44 | 1.45 |
| 89845 | ABCC10 | ATP-binding | −2.92 | 1.26 | 0.32 |
| | | cassette, sub- | | | |
| | | family C | | | |
| | | (CFTR/MRP), | | | |
| | | member 10 | | | |
| 26034 | IPCEF1 | interaction | −2.92 | −0.35 | 1.30 |
| | | protein for | | | |
| | | cytohesin | | | |
| | | exchange factors 1 | | | |
| 10906 | TRAFD1 | TRAF-type zinc | −2.93 | 0.59 | −0.14 |
| | | finger domain | | | |
| | | containing 1 | | | |
| 84811 | BUD13 | BUD13 | −2.94 | 0.68 | 2.47 |
| | | homolog (S. cerevisiae) | | | |
| 100527964 | LOC100527964 | uncharacterized | −2.96 | 2.19 | 0.20 |
| | | LOC100527964 | | | |
| 118426 | LOH12CR1 | loss of | −2.97 | 0.48 | 1.63 |
| | | heterozygosity, | | | |
| | | 12, | | | |
| | | chromosomal | | | |
| | | region 1 | | | |
| 54331 | GNG2 | guanine | −2.98 | −0.03 | 0.87 |
| | | nucleotide | | | |
| | | binding protein | | | |
| | | (G protein), | | | |
| | | gamma 2 | | | |
| 94081 | SFXN1 | sideroflexin 1 | −2.99 | 0.80 | 1.65 |
| 145474 | LOC145474 | uncharacterized | −2.99 | −0.36 | 0.65 |
| | | LOC145474 | | | |
| 84969 | TOX2 | TOX high | −3.00 | 0.40 | 2.29 |
| | | mobility group | | | |
| | | box family | | | |
| | | member 2 | | | |
| 4907 | NT5E | 5′-nucleotidase, | −3.02 | 1.48 | 0.94 |
| | | ecto (CD73) | | | |
| 51735 | RAPGEF6 | Rap guanine | −3.03 | 0.23 | 2.17 |
| | | nucleotide | | | |
| | | exchange factor | | | |
| | | (GEF) 6 | | | |
| 9953 | HS3ST3B1 | heparan sulfate | −3.03 | 0.28 | 1.28 |
| | | (glucosamine) | | | |
| | | 3-O- | | | |
| | | sulfotransferase | | | |
| | | 3B1 | | | |
| 23112 | TNRC6B | trinucleotide | −3.04 | −0.25 | 1.32 |
| | | repeat | | | |
| | | containing 6B | | | |
| 91526 | ANKRD44 | ankyrin repeat | −3.05 | 0.02 | 1.28 |
| | | domain 44 | | | |
| 101928017 | LOC101928017 | uncharacterized | −3.08 | 1.33 | 2.56 |
| | | LOC101928017 | | | |
| 84859 | LRCH3 | leucine-rich | −3.08 | 1.45 | 0.64 |
| | | repeats and | | | |
| | | calponin | | | |
| | | homology (CH) | | | |
| | | domain | | | |
| | | containing 3 | | | |
| 159013 | CXorf38 | chromosome X | −3.11 | −0.05 | 0.79 |
| | | open reading | | | |
| | | frame 38 | | | |
| 23208 | SYT11 | synaptotagmin | −3.19 | 2.10 | 0.96 |
| | | XI | | | |
| 101928649 | LOC101928649 | uncharacterized | −3.23 | 2.32 | 0.72 |
| | | LOC101928649 | | | |
| 85459 | KIAA1731 | KIAA1731 | −3.28 | 0.85 | 0.74 |
| 9617 | MTRF1 | mitochondrial | −3.31 | 0.72 | 1.67 |
| | | translational | | | |
| | | release factor 1 | | | |
| 56898 | BDH2 | 3- | −3.50 | 1.08 | 1.68 |
| | | hydroxybutyrate | | | |
| | | dehydrogenase, | | | |
| | | type 2 | | | |
| 387882 | C12orf75 | chromosome12 | −3.61 | 1.40 | 1.07 |
| | | open reading | | | |
| | | frame 75 | | | |
| 677769 | SCARNA17 | small Cajal | −3.64 | 1.73 | 0.35 |
| | | body-specific | | | |
| | | RNA17 | | | |

| gene | 22 | 23 | 24 | 25 | 26 | 27 | 28 | 29 | 30 | 31 | 32 | 33 |
|---|---|---|---|---|---|---|---|---|---|---|---|---|
| 56904 | −1.28 | −1.83 | −1.21 | −1.05 | −0.66 | −0.43 | −1.78 | −0.69 | −1.58 | 0.44 | 1.01 | −0.76 |
| 6464 | −0.81 | −1.13 | −1.00 | −1.03 | −0.56 | −1.20 | −0.33 | −0.28 | −1.31 | 0.34 | 0.51 | −0.13 |
| 79087 | −0.31 | −2.90 | −0.53 | −0.29 | −0.66 | −0.35 | −0.03 | −0.18 | −1.81 | −0.22 | 0.44 | −0.73 |
| 101928190 | −1.47 | −0.25 | −1.19 | −1.22 | −0.40 | −0.39 | −1.16 | −0.67 | 0.23 | 0.08 | −0.21 | −0.22 |
| 3416 | −0.59 | −0.45 | −1.01 | −1.58 | −0.51 | −0.95 | −1.91 | −0.71 | 0.15 | −0.62 | 0.07 | 0.00 |
| 8001 | −1.71 | −0.80 | −0.48 | −1.40 | −1.24 | 0.01 | −1.10 | −1.56 | −0.51 | 0.69 | −0.22 | −0.24 |
| 3727 | −0.07 | −1.14 | −1.52 | −0.73 | −1.41 | −1.82 | −1.51 | −0.20 | −1.07 | 1.02 | 0.47 | −0.41 |
| 10945 | 0.01 | −0.88 | −1.24 | −1.27 | −1.42 | −0.93 | −1.21 | −0.26 | −1.24 | 0.72 | 0.71 | −1.17 |
| 5025 | −0.87 | −0.79 | −1.00 | −1.24 | −0.59 | −0.49 | −1.65 | −0.52 | −1.23 | −0.24 | 1.11 | −0.76 |
| 29920 | −1.54 | −2.73 | −0.24 | −0.34 | 0.17 | −0.54 | −1.47 | −0.54 | −0.92 | −0.26 | 1.05 | 0.64 |
| 101929248 | −0.47 | −0.98 | −0.65 | −0.85 | −0.61 | −1.35 | −0.22 | −0.65 | −2.54 | −0.18 | −0.62 | 0.23 |
| 23646 | 0.25 | −1.68 | −1.32 | −0.48 | −0.76 | −0.64 | −1.76 | −0.22 | −1.69 | 0.35 | 0.61 | −0.46 |
| 10280 | −0.02 | −1.39 | −2.09 | −0.07 | −0.19 | −0.76 | −1.03 | −0.53 | −1.88 | 0.14 | 0.20 | 0.17 |
| 140699 | −0.57 | −1.14 | −1.47 | −0.35 | 0.30 | −0.45 | −0.69 | −0.25 | −0.56 | −0.93 | −0.15 | 0.62 |
| 4669 | −0.47 | −1.76 | −1.51 | −0.69 | −0.46 | −0.80 | −0.95 | −0.60 | −1.13 | 0.61 | −0.69 | −1.08 |
| 8341 | −1.79 | −0.53 | −0.66 | −2.02 | −0.06 | −0.55 | −1.55 | 0.83 | 0.33 | 0.10 | −1.31 | −0.85 |
| 2519 | −0.08 | −0.83 | −0.05 | −0.54 | −0.22 | −0.07 | −0.77 | −0.76 | −0.44 | −1.20 | 0.37 | −0.76 |
| 8985 | −0.65 | −0.90 | −1.85 | −0.41 | −0.45 | −1.61 | −1.23 | −1.48 | 0.02 | 1.31 | −0.16 | −0.18 |
| 1690 | 0.48 | −0.84 | −0.58 | −1.09 | −0.97 | −2.01 | −0.69 | −0.58 | 0.82 | −0.57 | −0.36 | −0.26 |
| 5111 | −0.47 | −0.20 | −1.68 | −0.68 | −2.12 | −1.33 | −0.96 | −0.36 | −0.56 | −0.61 | 0.86 | 0.09 |
| 55907 | −0.94 | −2.31 | −0.59 | 0.30 | −0.38 | −1.00 | −0.93 | −0.24 | −0.94 | −0.46 | −0.09 | −0.01 |
| 144811 | −0.27 | −1.17 | −0.87 | −0.18 | −1.12 | −0.19 | −0.07 | −1.80 | 0.53 | −1.35 | −0.94 | 0.83 |
| 92421 | −0.42 | −2.35 | −0.64 | −0.34 | −1.10 | 0.72 | −1.28 | −1.34 | −1.09 | −0.85 | 1.39 | −0.10 |
| 23475 | −0.22 | −0.75 | −1.34 | −0.29 | −0.98 | −1.44 | −1.67 | −1.38 | −0.50 | 0.13 | −0.15 | −1.58 |
| 54913 | 0.21 | −1.45 | −0.52 | −0.96 | −0.73 | 0.41 | −1.10 | −0.99 | −0.08 | −1.89 | 1.72 | 0.18 |
| 4645 | 0.39 | −0.57 | −1.59 | −1.36 | −0.92 | 0.78 | −1.14 | −0.16 | −0.26 | 0.48 | −0.44 | −0.56 |
| 9917 | −0.91 | −1.04 | −0.90 | −0.17 | −2.40 | −0.36 | −0.74 | −0.39 | −0.28 | −0.24 | 0.79 | 0.49 |
| 57213 | −0.11 | −0.01 | −1.02 | −0.38 | −1.40 | −1.66 | −0.89 | −0.57 | −0.24 | 0.64 | −0.12 | −0.12 |
| 113829 | −1.00 | −0.66 | −0.97 | −0.50 | −0.62 | −0.35 | −1.90 | −1.13 | −1.82 | 0.44 | 0.07 | 0.00 |
| 100128164 | −0.93 | −1.50 | −1.29 | −0.90 | −0.90 | −0.44 | −2.12 | −0.20 | 0.18 | 1.48 | −0.25 | 0.99 |
| 26284 | −0.65 | −1.15 | −1.48 | −0.12 | −1.63 | −1.48 | −1.39 | −1.07 | −0.54 | 0.46 | −0.15 | −0.42 |
| 64847 | −0.62 | −1.92 | −1.96 | −0.13 | −0.02 | −0.58 | −0.71 | −1.04 | −0.62 | −0.12 | −1.53 | −0.35 |
| 55974 | 0.39 | −1.37 | −1.53 | −0.83 | 0.39 | −0.73 | −1.67 | 0.03 | −1.45 | −0.84 | 0.27 | −0.77 |
| 64405 | −2.30 | 0.28 | −1.42 | 0.70 | −1.02 | −0.86 | −0.51 | −0.35 | −0.10 | −0.82 | −0.53 | 0.03 |
| 6510 | −0.02 | −0.23 | −1.46 | −1.54 | 0.84 | −1.10 | −1.53 | −1.06 | −1.39 | 0.33 | 0.39 | −0.85 |
| 196463 | −1.29 | −0.53 | 0.13 | −0.08 | −0.93 | −0.44 | 0.11 | −0.72 | −0.89 | −0.78 | −1.26 | −0.54 |
| 741 | −1.37 | −3.02 | 0.00 | 0.07 | −0.16 | 0.09 | −0.92 | −0.36 | −0.79 | −0.99 | −0.83 | −0.17 |
| 23568 | −1.77 | −0.16 | −1.18 | −0.81 | −2.57 | −0.51 | −1.35 | −1.08 | −0.44 | 0.18 | 0.40 | −0.47 |
| 79144 | −1.35 | −0.47 | −1.91 | −0.40 | −0.61 | −1.14 | −0.69 | −1.72 | 0.25 | 0.75 | 0.04 | −0.05 |
| 91319 | −0.94 | 1.17 | −0.93 | −1.11 | −0.59 | −1.45 | −1.01 | −0.61 | −1.22 | 0.69 | −0.01 | −0.98 |
| 79654 | −0.42 | −1.14 | −1.00 | −0.09 | −1.03 | −1.33 | −1.32 | −1.46 | 0.30 | 0.85 | −0.15 | 0.17 |
| 115950 | −1.94 | −0.21 | −0.26 | 0.09 | −1.18 | 0.55 | −0.81 | −2.66 | −0.81 | −0.28 | −0.64 | 0.54 |
| 1374 | −0.66 | −1.14 | 0.77 | −1.00 | −0.91 | −1.78 | −0.13 | −0.16 | 0.02 | −0.29 | −1.12 | −0.12 |
| 81533 | 0.04 | −1.03 | 0.34 | −0.28 | −2.54 | 0.09 | −0.12 | −0.45 | −1.29 | −0.70 | 0.74 | −0.35 |
| 201931 | −0.12 | −0.79 | −0.56 | −0.56 | −1.24 | −0.23 | −0.93 | −0.61 | −1.18 | −0.14 | 1.05 | 1.26 |
| 7979 | −0.54 | −1.85 | −1.60 | −0.34 | −0.51 | −0.74 | −2.66 | −0.05 | −1.07 | 0.47 | 0.57 | 0.10 |
| 159296 | −0.29 | −2.23 | −1.09 | −0.47 | −1.19 | −1.19 | −0.70 | −1.29 | −0.45 | −1.48 | 0.39 | 0.44 |
| 51102 | −0.55 | −1.70 | −2.25 | −0.26 | 0.01 | −0.88 | −1.89 | −1.05 | −1.02 | 0.73 | −0.27 | −0.01 |
| 8729 | −1.04 | −2.12 | −0.75 | −0.91 | 0.64 | −0.47 | −1.75 | −0.60 | −1.64 | 0.81 | 1.01 | −0.03 |
| 118460 | −0.27 | −1.68 | −0.53 | −0.13 | −0.67 | −1.68 | −0.83 | −0.05 | −2.45 | 1.05 | 0.82 | 0.02 |
| 79586 | −1.32 | −0.05 | −0.27 | 0.04 | −0.77 | −0.24 | −1.94 | −0.42 | 0.14 | 0.47 | 0.85 | −1.27 |
| 2052 | 0.57 | −0.57 | −1.45 | −0.72 | −1.12 | −0.34 | −1.22 | −1.19 | −0.72 | −0.45 | 1.06 | −0.72 |
| 84893 | 0.16 | −1.13 | −1.60 | −0.80 | 1.38 | −1.32 | −0.62 | −0.94 | −0.82 | −0.21 | −0.18 | −0.09 |
| 54726 | −0.91 | −1.46 | −0.26 | −0.32 | −0.48 | 0.01 | −0.61 | −1.37 | −0.16 | −2.12 | 0.27 | 0.69 |
| 813 | −0.41 | −0.68 | −1.55 | −0.84 | −0.35 | −0.55 | −1.41 | −0.79 | −0.81 | 0.15 | 0.89 | −0.93 |
| 54431 | −0.24 | −0.94 | −1.36 | −0.25 | −1.86 | −0.57 | −0.40 | −0.67 | 0.04 | −0.18 | −0.12 | 0.19 |
| 9612 | −1.40 | −0.81 | −0.67 | −1.56 | −0.53 | −2.17 | 0.00 | −0.71 | −0.15 | 1.43 | 0.81 | −0.51 |
| 9829 | −0.84 | −1.18 | 0.51 | −0.02 | −0.51 | 0.15 | −0.61 | 0.37 | 0.70 | −2.38 | −0.42 | −0.55 |
| 79036 | −0.91 | −1.36 | −1.06 | −0.27 | −0.70 | −0.22 | −1.02 | −0.22 | −0.39 | 1.03 | 0.00 | −0.92 |
| 284361 | −0.93 | −0.39 | −1.29 | −0.05 | −1.87 | −0.60 | −1.53 | −0.98 | −1.21 | 0.35 | 0.79 | −0.74 |
| 131118 | −0.21 | −1.71 | −1.73 | −0.31 | 0.25 | 0.06 | −1.88 | −0.71 | −0.67 | 0.06 | −0.02 | 0.91 |
| 644809 | 0.03 | −0.72 | −0.54 | −1.48 | 0.15 | −0.39 | −0.20 | −0.50 | −1.93 | −0.92 | 0.89 | 0.93 |
| 2119 | −0.40 | −0.52 | −1.29 | −0.50 | −1.03 | −0.50 | −0.28 | −0.92 | −2.05 | 0.97 | 0.94 | −1.10 |
| 5833 | 0.60 | −0.87 | −1.39 | −0.89 | −0.33 | −1.09 | −2.70 | −0.29 | −0.55 | 1.50 | −0.13 | −0.64 |
| 922 | 1.04 | −1.78 | −1.65 | −0.19 | −0.25 | −1.34 | −0.88 | 0.64 | −2.09 | −0.07 | −0.15 | −0.01 |
| 9261 | −0.37 | −0.46 | −0.25 | −1.13 | −1.38 | −0.95 | −0.90 | 0.00 | −2.42 | 1.26 | 1.47 | −1.44 |
| 57190 | 0.58 | −1.54 | −0.12 | −1.04 | −1.51 | −0.51 | 0.19 | −1.52 | 0.70 | −0.67 | 1.23 | −0.29 |
| 79644 | −0.27 | −0.81 | −1.01 | −0.45 | −2.99 | −0.84 | −0.84 | −0.75 | −0.73 | −0.14 | 0.74 | −0.03 |
| 79058 | −0.68 | −2.55 | −1.65 | −1.17 | 1.07 | −1.90 | −1.07 | −0.50 | −1.01 | 0.76 | −0.18 | −0.25 |
| 7832 | −0.75 | −0.80 | 1.75 | −0.97 | −1.72 | 0.60 | −0.34 | −0.58 | −1.79 | −1.22 | 0.20 | −0.75 |
| 11070 | −0.31 | −1.85 | −0.85 | −0.67 | −1.96 | 0.67 | −0.29 | −1.12 | −1.17 | −1.09 | 0.85 | −0.04 |
| 10847 | −1.00 | −0.84 | −0.94 | −0.74 | −0.27 | −0.99 | −1.16 | −0.22 | −0.68 | 2.32 | 0.59 | −0.72 |
| 4597 | −1.30 | −0.64 | 0.43 | −0.66 | 0.62 | −0.59 | −1.03 | −1.00 | −0.89 | −0.32 | −0.72 | −1.64 |
| 100506696 | −0.46 | −0.28 | −1.50 | −0.99 | −1.11 | −1.28 | −1.19 | −1.05 | −0.97 | 0.69 | 0.83 | −0.65 |
| 100507459 | 0.13 | −0.44 | −1.72 | −1.06 | 0.52 | 0.04 | −1.09 | −0.21 | −1.81 | 0.60 | 0.41 | −0.41 |
| 100130613 | −0.55 | −0.91 | −0.72 | −0.29 | −1.82 | −1.75 | −1.36 | −0.10 | −0.54 | 0.44 | 1.21 | 0.32 |
| 5081 | −0.05 | −2.10 | −1.71 | −0.98 | 0.31 | −0.49 | −0.67 | −0.54 | −2.58 | 0.31 | 0.21 | 0.68 |
| 100240728 | −0.61 | −1.48 | −1.29 | 0.18 | −0.53 | −1.28 | −1.80 | −0.35 | −1.66 | 1.60 | −1.14 | −0.16 |
| 26229 | −0.81 | −2.19 | −1.42 | −0.62 | −0.44 | −1.67 | −1.24 | −0.10 | −1.09 | 1.04 | −0.35 | −0.10 |
| 950 | −0.51 | 0.23 | −1.23 | −0.64 | −0.61 | 0.58 | −1.21 | −0.71 | −1.21 | −0.52 | 1.47 | −0.41 |
| 161882 | −0.40 | −0.48 | −1.15 | −1.45 | −1.12 | −0.53 | −0.79 | −1.05 | −1.85 | 1.20 | −0.43 | −0.39 |
| 5034 | 0.03 | −0.90 | −1.26 | −0.72 | −1.37 | −1.06 | −2.03 | −0.52 | −1.36 | 0.68 | 0.97 | −0.45 |
| 123355 | −0.11 | −1.46 | −0.65 | −1.18 | 0.74 | −0.82 | −0.18 | −0.86 | −0.79 | 0.71 | 0.54 | 0.02 |
| 6734 | −0.46 | −1.37 | −0.59 | −0.55 | −1.25 | −0.25 | −0.54 | −0.25 | −0.02 | −0.30 | 0.10 | 0.54 |
| 9911 | −0.50 | 0.14 | −0.95 | −1.31 | −0.60 | −1.53 | −1.37 | −0.86 | 0.64 | −0.61 | −0.20 | −0.91 |
| 222642 | −0.73 | −0.25 | 0.39 | −0.95 | −1.52 | −1.68 | −0.71 | −0.93 | 0.44 | −1.02 | −1.00 | −1.54 |
| 3643 | −0.40 | −0.21 | −1.22 | −0.76 | −1.70 | −1.17 | 0.68 | −0.88 | −0.92 | 1.24 | −0.15 | −0.62 |
| 85462 | −0.81 | −0.67 | 0.75 | 0.12 | −0.65 | 0.05 | −1.41 | −1.02 | 0.54 | −0.51 | −0.90 | −0.39 |
| 149175 | −1.02 | −0.86 | −0.89 | 0.07 | 0.22 | −1.72 | −1.95 | −0.40 | −1.95 | 0.41 | −0.35 | −0.10 |
| 1819 | −0.59 | −2.41 | −0.72 | −0.95 | 0.00 | −0.78 | −1.19 | −0.94 | −1.34 | −0.10 | 0.60 | −0.48 |
| 10370 | −0.62 | −1.04 | −0.27 | 0.09 | −1.87 | −0.74 | −0.30 | −1.07 | 0.04 | 0.42 | 0.80 | −0.26 |
| 25792 | −0.05 | −1.30 | −0.78 | −0.73 | −1.09 | −1.18 | −0.71 | −0.53 | −0.61 | 0.86 | 0.57 | −0.14 |
| 4072 | 0.35 | −1.47 | −1.29 | −0.52 | −0.06 | −0.55 | −1.07 | −0.51 | −1.51 | 0.54 | −0.64 | −0.97 |
| 84447 | −0.62 | −0.51 | −0.82 | −0.07 | −0.69 | −1.53 | −0.53 | −0.87 | −0.63 | 1.35 | −0.03 | −0.26 |
| 527 | −1.10 | −0.71 | 0.26 | −1.08 | −1.38 | −1.11 | −1.26 | −0.47 | −1.00 | 0.53 | 1.03 | −0.13 |
| 10629 | −0.44 | −0.46 | −1.55 | −1.87 | 0.86 | −1.13 | −2.13 | −1.10 | −0.15 | 0.89 | 0.86 | −0.60 |
| 51150 | 0.08 | −0.87 | −0.86 | −0.98 | −2.08 | −1.07 | −0.97 | −0.52 | −1.61 | 0.18 | 0.65 | 0.01 |
| 57143 | −0.99 | 0.37 | −2.09 | 0.44 | 0.53 | −2.14 | −0.98 | −1.30 | −0.25 | 1.34 | −0.33 | −0.70 |
| 682 | 0.61 | −1.96 | −0.72 | −1.18 | −0.56 | −0.79 | −1.33 | −0.03 | −0.85 | −0.05 | 0.29 | −1.11 |
| 10139 | −0.03 | −1.44 | −0.88 | −1.19 | 0.15 | −0.10 | −1.03 | −1.09 | −2.20 | 0.12 | 0.61 | 0.24 |
| 203238 | −0.07 | −1.31 | −2.56 | 0.15 | −0.77 | −1.35 | −0.24 | 0.80 | −1.81 | 0.39 | 0.47 | −1.01 |
| 54805 | −0.71 | −0.19 | −0.69 | −0.53 | −1.14 | −0.89 | −2.27 | −0.46 | −1.16 | 0.42 | −0.95 | −0.04 |
| 1200 | −0.27 | −1.56 | −0.55 | −0.04 | −0.17 | −1.95 | −1.26 | −0.68 | 0.97 | −0.16 | 0.11 | 0.49 |
| 2271 | −0.51 | −0.72 | −0.29 | 0.39 | −0.72 | 0.11 | −2.07 | −1.33 | −2.12 | −0.19 | 0.26 | −0.53 |
| 376412 | −0.30 | −0.59 | −1.49 | −0.81 | 0.10 | −1.53 | −1.99 | −0.59 | −1.49 | 1.02 | 0.36 | 0.28 |
| 4924 | 1.30 | −2.44 | −0.35 | −0.63 | −1.12 | −0.38 | −1.00 | 0.02 | −0.94 | 0.15 | 0.09 | −0.41 |
| 55132 | −0.14 | −0.36 | −0.74 | −0.76 | −1.46 | −1.45 | −1.07 | −1.06 | −0.86 | 0.31 | −0.92 | −0.11 |
| 9650 | −0.47 | −2.56 | −0.84 | −0.51 | −0.78 | −0.38 | −1.17 | −0.77 | −0.31 | −0.27 | −0.58 | −0.60 |
| 29803 | −0.66 | −0.23 | −1.24 | −0.42 | −0.37 | −1.50 | −1.76 | −0.78 | −1.36 | 1.43 | 0.63 | 0.34 |
| 4507 | −1.34 | −0.25 | −1.03 | −0.78 | −0.51 | −1.08 | −0.71 | −1.44 | −0.62 | 0.31 | 1.21 | 1.00 |
| 816 | −0.92 | −1.72 | −0.50 | −2.28 | −0.06 | −1.48 | −1.37 | 0.91 | 0.17 | 0.67 | 0.44 | −0.01 |
| 120379 | 0.42 | 0.09 | −0.74 | −2.12 | −2.85 | 0.20 | −1.88 | −0.51 | −0.61 | 0.50 | −0.07 | 0.03 |
| 91289 | −0.77 | 0.02 | −1.30 | −0.42 | 0.39 | −1.30 | −1.02 | −0.83 | −2.28 | 0.92 | −0.22 | −0.63 |
| 10905 | −0.47 | −1.17 | 0.50 | −1.56 | −1.18 | −0.65 | 0.10 | −0.93 | −0.18 | −0.28 | 0.54 | −0.14 |
| 25930 | −0.03 | −1.18 | −0.27 | 0.57 | −2.29 | −2.08 | −0.50 | −0.90 | 0.10 | 0.69 | 0.20 | 0.71 |
| 57648 | −0.14 | −0.35 | −0.88 | −0.42 | −0.20 | 0.17 | −1.18 | 0.01 | 0.41 | 0.19 | 0.45 | −0.22 |
| 7249 | 0.01 | −2.52 | −0.50 | −0.69 | −1.10 | −0.75 | −0.32 | −0.30 | −1.75 | 0.18 | 0.74 | −0.06 |
| 55151 | −0.17 | −0.96 | −0.14 | −0.33 | −1.17 | 0.40 | −1.88 | −0.70 | −0.12 | −1.22 | −0.12 | 0.04 |
| 160728 | −2.74 | −0.89 | 0.44 | 0.81 | −1.06 | −1.07 | −0.70 | −0.41 | −0.74 | 0.19 | 0.24 | −0.70 |
| 148418 | −0.47 | −1.41 | −0.78 | −1.09 | −0.57 | −1.40 | −1.27 | −1.02 | −0.98 | 1.80 | 1.04 | −1.05 |
| 646762 | −0.50 | 0.05 | −1.42 | −0.18 | −0.11 | −1.67 | −1.88 | −1.42 | 0.23 | 1.52 | 0.15 | −0.80 |
| 79109 | 0.05 | −0.62 | −0.40 | −0.58 | −1.97 | −0.53 | −0.98 | 0.65 | −2.58 | −0.26 | 0.40 | 0.20 |
| 81037 | −0.56 | 0.12 | −1.47 | −0.44 | −0.95 | −1.21 | −1.68 | −0.82 | −1.86 | 0.99 | 0.53 | −0.60 |
| 51523 | −0.86 | 0.69 | −0.77 | −0.96 | −1.13 | −1.08 | −1.31 | −1.02 | −0.68 | −0.33 | 0.23 | −0.98 |
| 55322 | −1.19 | −1.92 | −0.84 | −0.43 | 0.08 | −0.06 | −1.94 | −0.30 | −0.46 | 0.04 | 0.66 | 1.47 |
| 100996671 | 0.83 | −2.37 | −0.81 | −2.13 | −0.74 | −1.37 | −0.90 | 0.07 | −0.24 | 0.59 | −0.06 | −0.83 |
| 55222 | −0.15 | −0.38 | −2.38 | −1.07 | 0.40 | −0.33 | −1.72 | −0.20 | −1.69 | −0.75 | 0.47 | −1.05 |
| 55624 | 0.05 | −0.95 | −2.00 | −0.16 | −1.30 | −1.25 | −1.45 | −0.62 | −1.24 | 0.38 | 0.43 | 0.58 |
| 100505983 | −0.22 | −1.36 | −0.31 | −0.14 | 0.80 | −0.64 | −0.81 | −0.39 | 0.10 | −1.00 | −0.77 | −1.21 |
| 3069 | 0.20 | −0.05 | −1.09 | −0.89 | −0.54 | −1.77 | −1.65 | −0.48 | −1.57 | 1.64 | −0.17 | −0.39 |
| 10959 | −0.45 | −1.37 | −0.78 | −0.66 | −1.67 | −0.30 | −1.87 | −0.27 | −1.19 | 0.35 | 1.51 | 0.14 |
| 6598 | 0.23 | −1.33 | −1.85 | −0.82 | −0.83 | −1.68 | −0.75 | −0.49 | −0.64 | 1.09 | 0.22 | −1.02 |
| 60412 | 0.09 | −0.82 | −1.06 | −0.22 | 1.00 | 0.34 | −2.64 | −0.77 | −0.34 | −0.03 | 0.34 | −0.47 |
| 54982 | −1.13 | −0.14 | −0.97 | −0.85 | −0.43 | −0.81 | −1.53 | 0.19 | −2.33 | 0.61 | 0.38 | −0.88 |
| 91752 | −0.74 | −0.90 | −0.06 | −0.82 | 0.39 | −1.31 | −1.41 | −1.05 | 0.19 | −0.79 | 0.26 | −1.36 |
| 113178 | 0.58 | −2.16 | 0.01 | −0.48 | −1.48 | −0.91 | −0.98 | 0.52 | −1.27 | 0.00 | 0.41 | −0.64 |
| 10 | −2.02 | −1.50 | −0.51 | −0.82 | 0.00 | 1.29 | 0.07 | −0.19 | −0.94 | −0.06 | 0.31 | 0.49 |
| 375 | −0.04 | −1.44 | −0.75 | −0.71 | −0.31 | −0.59 | −1.57 | 0.25 | −1.27 | 0.54 | 1.30 | −1.11 |
| 5589 | 0.51 | −0.95 | −1.22 | −0.60 | −0.45 | −1.20 | −1.19 | 0.19 | −1.22 | 1.45 | 0.22 | −0.09 |
| 9136 | −1.22 | −1.47 | 0.45 | 0.16 | −0.13 | −0.18 | −1.11 | −0.12 | −2.21 | −1.46 | −0.45 | 0.16 |
| 23193 | −0.10 | −1.18 | −0.78 | −0.57 | −1.28 | −1.20 | −2.09 | −0.13 | −0.77 | 0.29 | 1.21 | −0.14 |
| 1798 | 0.23 | −0.18 | −2.01 | −0.74 | −0.59 | −1.17 | −1.80 | −0.78 | −0.66 | 0.56 | 0.23 | −0.25 |
| 85440 | 0.08 | −0.45 | −1.70 | −0.49 | −1.37 | −0.71 | −1.44 | −0.34 | −0.15 | −0.53 | −0.03 | 0.71 |
| 100505794 | −1.64 | −0.18 | 0.84 | 0.07 | −0.81 | −1.09 | 0.79 | −0.19 | −0.74 | −0.81 | 0.69 | −0.41 |
| 1173 | 0.19 | −0.83 | −0.58 | 0.01 | −1.24 | −0.72 | −1.26 | −0.17 | −1.41 | 0.52 | 0.41 | 0.49 |
| 124540 | 0.21 | −0.60 | −1.31 | −0.30 | −1.63 | −1.61 | −1.18 | −0.02 | −1.23 | 0.49 | 0.32 | 0.17 |
| 8372 | −0.55 | −0.71 | −2.60 | −0.63 | −0.10 | −1.86 | −0.78 | 0.12 | 1.45 | −0.18 | −1.76 | −0.41 |
| 64755 | −0.56 | −0.55 | −1.42 | −0.35 | −0.24 | −1.81 | −1.76 | −0.68 | −1.66 | 0.41 | 0.59 | −0.48 |
| 79102 | 0.30 | −1.43 | −0.70 | −1.38 | 0.39 | −1.46 | −2.44 | −0.21 | −1.16 | 1.19 | 0.28 | −0.77 |
| 10963 | 0.04 | −2.09 | −0.40 | −0.42 | −0.73 | −0.21 | −1.47 | 0.49 | −1.67 | −0.12 | 1.24 | −0.17 |
| 375387 | −0.66 | −0.87 | −0.01 | −0.17 | −0.11 | −1.28 | −0.50 | −0.60 | 0.24 | −1.54 | −0.83 | −0.65 |
| 972 | −0.16 | −0.28 | −1.43 | −0.11 | −0.01 | −0.70 | −1.47 | −0.12 | −0.90 | 0.20 | 0.38 | −0.74 |
| 54676 | −0.84 | −2.43 | −0.14 | −0.94 | 0.04 | −1.20 | −1.73 | −0.38 | −1.14 | −0.52 | −0.67 | 0.09 |
| 192286 | −1.21 | −0.95 | −1.64 | −0.10 | −0.35 | −0.96 | −1.57 | −0.62 | −0.45 | −0.12 | −0.06 | 0.30 |
| 3295 | 0.23 | −0.96 | −1.28 | −0.07 | −1.22 | −0.35 | −1.62 | −0.12 | −0.33 | −0.44 | 0.74 | 0.28 |
| 80852 | −2.48 | 0.46 | −0.23 | −1.00 | −0.28 | −0.56 | −1.60 | 0.29 | −0.25 | 0.99 | −0.45 | 0.32 |
| 1298 | −0.09 | −1.17 | −1.30 | 0.06 | −0.27 | −1.24 | 0.45 | 0.11 | −2.40 | 1.06 | −0.53 | −0.79 |
| 2004 | −0.62 | 1.45 | −0.81 | −0.93 | −0.98 | −0.84 | −1.47 | −1.45 | −0.39 | −2.20 | −0.39 | 0.73 |
| 10279 | −2.85 | −1.22 | −0.19 | −0.43 | 0.00 | 0.14 | −0.38 | −1.05 | −0.19 | −0.73 | −1.41 | 0.10 |
| 151790 | 0.21 | −0.42 | −0.58 | 0.23 | −0.53 | −0.55 | −0.87 | −0.58 | −1.29 | −0.51 | −0.23 | −0.55 |
| 399664 | −2.20 | 0.21 | −0.20 | −1.11 | 0.19 | −0.11 | −0.84 | −1.00 | −2.02 | 0.71 | 0.86 | −0.53 |
| 5664 | −0.49 | −0.06 | −0.67 | −0.95 | −1.10 | −0.55 | −0.82 | −1.52 | −0.69 | −0.48 | 0.70 | −1.26 |
| 51614 | −0.81 | −0.96 | −1.69 | −1.06 | −0.57 | −0.66 | −1.27 | −0.86 | −1.00 | 1.10 | 0.44 | −0.68 |
| 79178 | −0.20 | −0.91 | −0.99 | −0.52 | −0.45 | −0.70 | −1.55 | 0.06 | −2.35 | 0.14 | 0.23 | −0.52 |
| 51726 | −0.46 | −0.02 | −1.07 | −0.56 | −1.29 | −1.52 | −1.64 | −0.24 | −2.37 | 0.98 | 0.57 | −0.65 |
| 3425 | −0.59 | −0.24 | −1.67 | 0.48 | −1.44 | −1.45 | −1.37 | −1.23 | −1.32 | 0.23 | 0.19 | 0.15 |
| 1642 | −1.11 | −0.14 | −1.68 | −0.30 | 0.04 | −1.21 | −1.77 | −1.06 | −1.05 | 0.65 | −0.11 | −0.13 |
| 101928770 | 0.07 | −2.41 | 0.42 | −0.74 | −0.38 | −1.28 | 1.03 | −1.00 | −0.24 | 0.14 | −0.97 | 0.15 |
| 11015 | 0.13 | −0.90 | −1.05 | −1.01 | −0.66 | 0.40 | −1.50 | −1.03 | −1.40 | 0.20 | 1.30 | −0.43 |
| 35 | −0.31 | −0.92 | −1.80 | −1.34 | −0.11 | −1.72 | −0.57 | −0.59 | −1.68 | 1.63 | −0.30 | 0.76 |
| 64798 | −0.04 | 0.17 | −1.91 | −0.69 | −1.41 | −1.48 | −1.15 | −0.95 | −0.40 | 0.49 | 0.30 | −0.05 |
| 23457 | −1.70 | 0.23 | 0.95 | −0.75 | −0.57 | −2.38 | −2.52 | −0.49 | 0.58 | 0.06 | 1.04 | −0.19 |
| 56132 | −2.75 | −1.26 | −0.74 | 0.98 | −0.40 | 0.17 | 0.89 | −0.77 | −0.68 | −0.57 | −1.01 | −0.72 |
| 23400 | −0.24 | −0.55 | −0.61 | −0.38 | −1.52 | −1.24 | −1.72 | −1.52 | −1.07 | −1.03 | 0.51 | −0.12 |
| 3980 | 0.25 | −0.89 | −2.55 | −0.81 | −0.74 | 0.02 | −2.38 | −1.06 | 0.51 | 0.52 | 0.50 | −0.07 |
| 5690 | −0.93 | −0.06 | −1.09 | −0.42 | 0.34 | −2.37 | −1.23 | 0.14 | −0.81 | 1.55 | −1.12 | 0.64 |
| 7917 | −0.41 | −0.50 | −1.20 | −1.11 | −0.12 | −1.42 | −1.52 | −1.02 | −0.94 | 0.53 | 0.32 | −0.02 |
| 1739 | −0.49 | −0.98 | −0.97 | −0.58 | −1.71 | −1.05 | −1.23 | 0.06 | −1.57 | 0.23 | 0.49 | 1.12 |
| 2837 | −0.19 | 0.46 | −1.16 | −1.84 | 0.00 | −1.49 | −2.39 | 0.82 | 0.24 | −0.42 | −0.46 | 0.40 |
| 83746 | −1.37 | −0.75 | −2.66 | −0.21 | 1.28 | −0.65 | −2.26 | −0.13 | −1.23 | 0.41 | −0.08 | −0.23 |
| 5962 | 0.35 | −1.71 | −0.86 | −0.84 | −1.85 | −0.50 | −0.59 | 0.17 | −1.83 | −1.01 | −0.33 | −0.52 |
| 9956 | −1.02 | 0.24 | −1.50 | −0.94 | −0.36 | −0.32 | −0.42 | −0.53 | −2.99 | 0.42 | −0.16 | −0.13 |
| 162968 | −0.52 | −1.78 | −1.45 | 0.14 | −0.66 | 0.23 | 0.98 | −1.94 | −0.57 | −1.14 | 0.93 | −0.68 |
| 4192 | 0.55 | 0.08 | −0.98 | −0.23 | −1.37 | −0.57 | −1.05 | −1.01 | −2.53 | 0.54 | 1.26 | −0.99 |
| 54919 | 0.37 | −0.55 | −1.79 | −0.04 | −1.01 | 0.14 | −1.22 | −0.85 | −2.07 | −0.84 | 0.81 | −0.31 |
| 10972 | −0.89 | −0.25 | −0.98 | −0.40 | −1.46 | −0.19 | −1.53 | −0.52 | −0.83 | −0.04 | 0.35 | 0.62 |
| 23061 | −0.61 | 0.36 | −2.05 | −0.57 | 0.68 | −1.81 | −2.03 | −0.54 | −0.50 | 1.07 | 0.38 | −0.24 |
| 3792 | 0.95 | −0.63 | −1.58 | 0.55 | −0.34 | −0.72 | −0.74 | −1.03 | 0.51 | −1.59 | −0.61 | −0.82 |
| 2063 | −0.45 | −1.95 | −0.34 | −0.82 | −0.42 | −0.19 | −0.82 | −0.99 | −1.05 | 1.04 | −0.08 | −1.50 |
| 55177 | −0.89 | −0.07 | −2.03 | −0.04 | 0.26 | −0.51 | −1.87 | −1.07 | −0.42 | −0.46 | 0.51 | −0.67 |
| 11147 | 0.21 | −1.73 | −0.67 | −0.17 | 0.33 | 0.02 | −1.46 | −0.63 | −0.49 | −1.48 | −0.53 | −0.15 |
| 57486 | 0.41 | 0.16 | −0.80 | −1.04 | −0.54 | −1.81 | 0.10 | 0.08 | −0.39 | −0.68 | −0.02 | −0.44 |
| 30814 | −0.20 | −1.11 | −1.25 | −1.55 | −0.09 | 0.17 | −1.32 | 1.17 | −1.13 | 1.76 | 0.08 | 0.07 |
| 57414 | −0.68 | −0.87 | −0.95 | −0.83 | −0.40 | −0.34 | −1.04 | −0.51 | −1.61 | 1.09 | 1.76 | −0.93 |
| 84836 | −0.77 | 0.14 | −1.42 | −0.30 | −0.83 | −1.04 | −1.21 | −1.88 | −1.02 | 0.71 | 0.55 | 0.04 |
| 8516 | 0.93 | −1.32 | 0.38 | −1.48 | 0.19 | −0.48 | 0.22 | −1.34 | −0.59 | −0.80 | −0.71 | −0.91 |
| 89866 | −0.52 | −1.19 | −2.47 | −0.47 | −1.09 | −1.23 | −1.28 | −0.86 | 1.00 | 0.80 | 0.49 | −0.17 |
| 10491 | −1.46 | 1.30 | −0.37 | −0.39 | −1.44 | −1.15 | −0.34 | −1.17 | −0.38 | 0.86 | 0.22 | 0.63 |
| 11047 | −0.35 | −1.14 | −1.04 | −0.78 | 1.01 | −1.44 | −1.42 | −0.65 | −1.92 | 0.73 | 0.22 | −0.91 |
| 28971 | 0.87 | 0.17 | −2.19 | −0.58 | 0.06 | −1.82 | −1.72 | −1.03 | −1.00 | 1.01 | 0.39 | −0.43 |
| 8078 | −0.68 | −0.66 | −1.54 | −0.64 | 0.06 | −1.61 | −0.79 | −0.05 | 0.00 | 0.26 | 0.29 | 0.46 |
| 37 | 0.27 | −0.90 | −1.46 | −0.37 | −0.18 | −1.67 | −1.00 | −0.69 | −2.44 | 0.71 | 0.40 | −0.81 |
| 145508 | −2.43 | 0.86 | −0.27 | −0.22 | −1.14 | −0.22 | −0.88 | −1.73 | 0.49 | −0.87 | 0.38 | −0.08 |
| 49861 | −1.79 | −1.45 | −1.00 | −0.62 | −0.48 | 0.21 | −0.66 | −0.44 | 0.45 | 1.92 | 0.48 | −0.68 |
| 5905 | −0.26 | −0.19 | −1.82 | −0.41 | −0.21 | −1.01 | −1.46 | −0.07 | −2.74 | 0.95 | −0.20 | −0.18 |
| 7542 | 0.40 | −1.75 | 0.16 | −0.45 | −0.49 | −0.71 | −0.43 | −0.82 | −2.69 | −0.62 | 1.20 | −0.85 |
| 9562 | −1.41 | −1.06 | −0.10 | 0.68 | −1.68 | 0.34 | −0.44 | −0.28 | −0.58 | −1.52 | −0.25 | 0.18 |
| 79095 | −0.54 | −1.81 | −1.41 | −0.33 | 0.96 | −0.80 | −0.84 | −0.51 | −1.63 | 0.97 | 0.93 | −0.10 |
| 8815 | 0.62 | −1.77 | −0.92 | −0.97 | −0.73 | −0.13 | −1.25 | 0.19 | −0.20 | −0.05 | 0.57 | 0.37 |
| 54867 | −0.26 | −1.31 | −1.25 | −0.40 | −2.00 | −0.89 | −0.75 | −0.56 | 0.61 | 1.97 | −0.05 | 0.68 |
| 6721 | −0.41 | −0.84 | −1.28 | −0.71 | −0.20 | −1.67 | −0.98 | 0.44 | −1.03 | 1.08 | 1.63 | −1.16 |
| 23133 | −1.80 | 0.31 | −0.51 | −1.00 | −0.42 | −1.45 | −0.75 | −0.02 | −0.49 | 1.72 | −0.57 | 0.70 |
| 113675 | −0.08 | −0.65 | −0.86 | −0.59 | −0.23 | 0.05 | −1.80 | −0.22 | −1.55 | −0.74 | −0.31 | −0.39 |
| 129303 | 0.63 | −0.87 | −1.14 | −0.18 | −1.12 | −2.01 | −2.22 | −1.38 | 0.92 | −0.08 | 0.79 | 0.77 |
| 57658 | −1.31 | −0.60 | −0.22 | −0.57 | −0.06 | −1.37 | −1.12 | −0.15 | 0.04 | 2.19 | 0.40 | 0.59 |
| 162 | −0.01 | −0.65 | −1.04 | −0.46 | 0.39 | −1.24 | −0.53 | −0.45 | −2.53 | 0.87 | 0.24 | −1.00 |
| 2873 | 0.19 | −0.64 | −1.89 | −0.46 | 0.03 | −1.64 | −1.37 | −0.52 | −1.98 | −0.63 | 0.97 | −0.55 |
| 64978 | 0.51 | −2.16 | −0.49 | −0.44 | 0.02 | −0.43 | −1.89 | −0.71 | −1.57 | −0.49 | 1.44 | 0.29 |
| 220323 | 0.05 | −0.21 | −1.96 | −1.16 | −1.10 | −1.46 | −0.88 | −0.76 | −0.15 | 0.57 | −0.16 | −0.45 |
| 6006 | −0.21 | 0.44 | −0.98 | −0.96 | 0.11 | −0.35 | −0.32 | −2.07 | 1.25 | −0.63 | −1.58 | −0.47 |
| 389812 | −0.97 | −1.86 | −0.92 | −0.47 | 0.18 | −1.89 | −1.46 | 0.51 | −0.27 | 0.30 | −0.11 | 0.54 |
| 2628 | −0.51 | −0.24 | −0.77 | −0.41 | −0.50 | 0.76 | −1.29 | −0.12 | −0.63 | −0.91 | 0.26 | −0.80 |
| 6509 | 0.40 | 0.52 | −0.81 | −0.96 | −0.62 | −0.94 | −1.58 | −1.54 | −0.58 | 0.41 | 0.35 | −1.26 |
| 3888 | −0.62 | −0.26 | −1.61 | −1.47 | −0.07 | −2.05 | −0.47 | −0.53 | −0.82 | 2.32 | 0.46 | −0.22 |
| 5211 | −1.00 | 0.14 | −1.54 | −0.58 | 0.42 | −1.76 | −1.34 | −0.31 | −1.71 | 1.16 | 0.16 | −0.38 |
| 55080 | −1.01 | −0.64 | 0.06 | −0.50 | 0.32 | −0.05 | −0.43 | −1.30 | −1.03 | −0.22 | 0.34 | 1.22 |
| 2139 | 0.41 | −1.52 | −1.27 | −0.08 | 0.94 | 0.72 | −1.22 | −1.55 | −0.82 | −0.31 | −0.73 | −0.04 |
| 2030 | 0.08 | −1.26 | −1.36 | −0.97 | −0.26 | −1.64 | −0.75 | −0.98 | −0.28 | −0.02 | 0.19 | −1.69 |
| 56005 | −0.18 | 0.35 | −1.40 | −0.24 | −0.71 | −0.49 | −1.30 | −0.95 | −1.94 | 0.46 | 0.86 | −0.87 |
| 6414 | 0.86 | −1.04 | −1.09 | −0.68 | −1.59 | −0.49 | −0.89 | −0.68 | 0.14 | −1.32 | 0.19 | −0.45 |
| 5184 | −0.28 | 0.05 | −0.97 | −0.12 | −0.53 | −0.49 | −0.93 | −0.41 | −0.92 | 0.78 | 0.21 | 0.29 |
| 643783 | −0.30 | −0.73 | −0.39 | −1.05 | −0.58 | 0.11 | −0.85 | −2.07 | −0.01 | −0.15 | −0.59 | −0.69 |
| 101928185 | −0.15 | −1.37 | −0.63 | −0.59 | −1.98 | 0.77 | −0.44 | −1.62 | 1.00 | −0.18 | 2.19 | −0.74 |
| 22883 | −0.04 | 0.08 | −1.78 | 0.15 | −0.12 | −1.13 | −1.16 | −1.23 | −0.93 | 1.48 | 0.62 | 0.54 |
| 25800 | 0.19 | −1.18 | −1.06 | −0.44 | −2.67 | −0.39 | −0.57 | −0.39 | −1.73 | 0.50 | 0.69 | −0.05 |
| 54681 | −0.26 | −0.49 | −2.25 | −0.14 | −0.71 | −0.87 | −1.45 | −1.29 | −1.09 | 0.79 | 0.73 | 0.36 |
| 644150 | −0.20 | −1.36 | −0.25 | −1.42 | −1.20 | 0.56 | −1.50 | 0.02 | −0.58 | 0.38 | 1.10 | −0.66 |
| 9048 | −0.02 | −0.51 | −1.44 | −0.70 | −1.11 | −1.39 | −0.58 | −0.96 | 0.03 | 1.54 | −0.41 | −0.20 |
| 3054 | 0.29 | −0.46 | −1.57 | −0.46 | −0.74 | −1.34 | −2.37 | −1.10 | −1.16 | 1.18 | 0.15 | −0.53 |
| 115098 | −0.23 | −1.33 | −0.65 | −0.49 | −0.36 | 0.11 | −0.91 | −0.95 | −1.73 | 0.73 | −0.10 | −0.74 |
| 55558 | −0.16 | −0.98 | −1.98 | −0.76 | −0.43 | −1.30 | −1.51 | −0.78 | −0.45 | 0.20 | 0.59 | 0.09 |
| 675 | 0.83 | −0.67 | −0.79 | −0.77 | −1.18 | −1.71 | −0.78 | −1.13 | −1.61 | −0.06 | −0.15 | −1.28 |
| 9777 | −0.22 | −0.71 | −0.94 | −0.60 | −1.42 | −1.36 | −0.78 | −0.99 | −1.21 | −0.17 | 0.44 | −0.12 |
| 83707 | 0.66 | −0.32 | −0.96 | 0.26 | −1.13 | −0.12 | −1.86 | −1.51 | −1.91 | 0.78 | 0.09 | 0.07 |
| 55334 | 0.10 | −1.24 | 0.02 | −0.97 | 0.16 | −0.36 | −2.12 | 0.21 | −0.09 | 0.21 | 0.56 | −0.75 |
| 9793 | −0.58 | −0.79 | −2.20 | −0.63 | −0.99 | −1.59 | −1.83 | −1.05 | −0.61 | 0.33 | 0.21 | 0.14 |
| 56834 | −0.16 | −1.80 | −0.65 | −0.91 | 1.33 | −1.08 | −1.71 | −0.58 | −0.68 | −0.32 | 1.03 | −1.49 |
| 25921 | 0.18 | −0.80 | −1.37 | −0.94 | −0.61 | −1.61 | −1.67 | 0.08 | −2.40 | 0.82 | 1.06 | −0.75 |
| 6520 | −0.52 | −1.00 | −1.42 | −0.24 | −0.20 | −1.68 | −1.78 | −0.18 | −0.41 | 0.14 | 0.70 | −0.34 |
| 728591 | −0.25 | −2.19 | −1.03 | −0.77 | −0.31 | −2.56 | −0.93 | 0.44 | −0.52 | 1.26 | 0.29 | 0.26 |
| 7415 | 0.52 | −0.15 | −1.24 | −0.38 | −0.49 | −1.55 | −1.45 | −0.43 | −2.29 | 1.44 | 0.18 | −1.31 |
| 130827 | 0.22 | −0.27 | −1.98 | 0.13 | −1.18 | 0.68 | −2.05 | −1.40 | −0.24 | −0.42 | 0.11 | −0.11 |
| 9620 | −0.16 | −1.36 | −1.62 | −0.97 | −1.27 | 0.61 | 0.57 | −0.53 | −0.17 | −0.14 | 0.46 | −0.19 |
| 55335 | −1.05 | −1.02 | −0.39 | −0.77 | −0.14 | −1.00 | −0.52 | −1.05 | 0.17 | 0.33 | −0.47 | −0.28 |
| 84988 | 0.96 | −0.89 | −1.50 | 0.07 | −0.98 | −0.32 | −1.33 | 0.34 | −1.48 | −2.09 | 1.44 | −0.70 |
| 100192378 | −0.08 | −0.77 | −0.53 | −0.89 | 0.11 | −1.09 | −1.44 | 0.41 | 1.80 | −0.16 | 0.04 | −0.76 |
| 442524 | −1.03 | −1.65 | −0.06 | 0.00 | −1.42 | −0.34 | −0.81 | 0.06 | 1.36 | −0.63 | −0.05 | −0.84 |
| 51661 | 0.68 | −0.53 | −2.05 | −0.71 | 0.13 | −2.12 | −1.11 | −0.33 | −0.66 | 0.39 | 0.71 | −0.07 |
| 84922 | −0.63 | −1.48 | −0.47 | 0.27 | −1.56 | −0.92 | −1.93 | −0.11 | −1.40 | 0.08 | 1.24 | 0.55 |
| 84954 | 0.67 | −0.29 | −0.31 | −1.39 | −0.88 | −0.83 | −0.64 | −0.60 | −1.29 | 0.77 | 0.85 | −0.79 |
| 245972 | −1.17 | −0.90 | −1.01 | −0.54 | 1.67 | 0.72 | −0.73 | −1.14 | −1.10 | 2.12 | −0.40 | −0.49 |
| 4316 | −0.85 | −0.80 | −0.12 | −1.04 | 0.22 | −0.85 | −1.80 | 0.25 | −0.51 | 1.17 | −0.52 | −1.12 |
| 55157 | −0.11 | −1.26 | −0.46 | −1.05 | −0.51 | −1.34 | −1.89 | −0.78 | 0.46 | −0.81 | −1.38 | 0.79 |
| 7466 | −1.00 | −0.86 | −1.74 | 0.06 | 0.43 | 0.75 | −0.51 | −0.61 | −0.56 | 0.08 | 0.30 | −1.23 |
| 79365 | −1.16 | −0.08 | 0.05 | −0.90 | −0.83 | −0.38 | −0.80 | −0.68 | 0.48 | −0.30 | −0.18 | −1.83 |
| 10066 | −0.95 | −0.61 | −1.47 | −0.69 | 1.05 | −1.45 | −1.85 | −0.43 | −0.89 | 0.58 | 0.69 | −0.79 |
| 203068 | 0.23 | −0.33 | −2.04 | −0.26 | 0.25 | −1.53 | −1.88 | −0.53 | −1.11 | 0.26 | 0.37 | −0.66 |
| 5780 | −2.22 | −1.05 | −1.09 | −0.58 | 0.00 | −0.65 | −1.00 | 0.38 | 1.20 | 1.33 | −0.33 | 0.72 |
| 285627 | −1.77 | −1.12 | −1.14 | −1.16 | −1.29 | −1.59 | 1.73 | 1.59 | −0.91 | 1.41 | −0.14 | −0.38 |
| 2058 | −0.87 | 0.10 | −1.92 | 0.06 | −1.23 | −1.51 | −1.23 | −0.94 | −1.00 | 0.01 | −0.09 | −0.50 |
| 1737 | −0.10 | −1.31 | −0.80 | −0.29 | −1.99 | −0.31 | −1.56 | 0.19 | −0.58 | −0.82 | −0.05 | 0.58 |
| 22924 | 1.17 | −0.15 | 0.38 | −0.71 | −1.23 | −0.23 | −1.47 | −0.27 | −1.47 | −0.69 | 0.59 | −1.26 |
| 6005 | 0.24 | −0.48 | −1.01 | −1.18 | −1.11 | −1.20 | −0.26 | −0.87 | 0.90 | −1.20 | −1.47 | −1.03 |
| 5286 | −1.15 | −1.64 | −0.64 | −1.00 | −1.04 | −0.21 | −0.37 | −0.59 | −1.00 | 0.03 | 1.31 | 1.20 |
| 11226 | −1.72 | 0.35 | −0.68 | −1.38 | −0.95 | −0.33 | −0.02 | −0.95 | −0.32 | −0.54 | 0.45 | −0.30 |
| 2762 | 0.57 | 0.14 | −1.22 | −0.97 | −1.65 | −1.27 | −1.53 | −1.62 | −0.14 | 0.97 | 0.37 | −0.72 |
| 8908 | −0.28 | −0.02 | −1.42 | −0.38 | 0.32 | −1.32 | −0.94 | −1.34 | −1.44 | 1.24 | 1.45 | 0.28 |
| 10693 | −0.01 | 0.37 | −2.13 | −0.77 | −1.54 | −1.52 | −0.52 | −0.51 | −0.44 | 1.00 | 0.31 | −0.72 |
| 199953 | −0.44 | 0.07 | −1.27 | −0.22 | −0.53 | −1.57 | −1.59 | −1.06 | −1.95 | 0.05 | −0.05 | −0.50 |
| 2760 | −0.62 | −1.84 | 0.43 | 0.17 | −1.04 | 0.52 | −0.49 | −0.16 | −0.08 | −1.11 | 0.30 | −1.44 |
| 8295 | −0.18 | −1.13 | −1.01 | −0.82 | −0.91 | −1.02 | −1.77 | 0.63 | −0.62 | 0.38 | 0.59 | 0.06 |
| 1822 | −0.79 | −0.49 | −0.97 | −0.03 | 0.16 | −1.02 | −1.34 | −0.29 | −2.32 | 1.03 | 1.63 | −0.72 |
| 55328 | −1.06 | 1.34 | −0.21 | −0.33 | 0.71 | −1.28 | −2.18 | −2.27 | −0.14 | 0.91 | −0.29 | 0.76 |
| 2069 | −1.00 | −0.53 | 0.62 | −0.30 | −1.99 | 0.27 | −0.52 | −0.37 | 0.14 | −1.07 | −0.64 | 0.43 |
| 4329 | 0.51 | −0.52 | −1.68 | −0.34 | −1.07 | −1.27 | −1.02 | −0.75 | 1.43 | −0.14 | 0.25 | 0.69 |
| 537 | 0.30 | −0.83 | −0.17 | −0.66 | −1.80 | −0.72 | −0.99 | 0.01 | −2.03 | 0.38 | 1.24 | −1.43 |
| 64764 | −0.67 | 0.24 | −1.41 | −0.28 | 0.06 | −1.22 | −1.72 | −0.95 | −1.13 | 1.14 | −0.04 | −0.24 |
| 811 | 0.33 | −0.75 | −1.02 | −0.65 | −1.40 | −1.73 | −0.91 | −0.32 | −1.64 | 1.61 | 0.75 | −0.66 |
| 9779 | −1.07 | −0.28 | −0.56 | −0.45 | 0.87 | −1.17 | −1.16 | −0.75 | −0.74 | 1.42 | −0.18 | 0.54 |
| 114815 | 0.08 | −1.45 | −1.03 | 0.21 | −0.07 | −1.12 | −2.49 | −1.23 | 0.55 | 0.79 | −0.85 | 0.62 |
| 29927 | −0.14 | −0.62 | −1.01 | −0.41 | −0.78 | −0.95 | −1.19 | −0.23 | −2.17 | −0.18 | 0.62 | −0.99 |
| 6007 | 0.05 | −0.52 | 0.15 | −0.04 | −0.41 | −0.29 | −0.43 | 1.20 | −1.27 | −0.94 | −1.67 | −0.20 |
| 5192 | 0.03 | −0.69 | −1.31 | −1.10 | −0.21 | −0.63 | −1.40 | −1.44 | −1.37 | 0.00 | 0.02 | −0.26 |
| 125875 | −1.20 | −1.36 | −0.94 | 1.20 | −0.52 | −2.27 | −1.61 | −0.41 | −1.06 | 1.40 | 0.32 | 0.31 |
| 10882 | 0.12 | −0.68 | 1.53 | −2.31 | −0.49 | −0.46 | −1.90 | 0.23 | −0.81 | −0.39 | −0.73 | −0.03 |
| 192683 | −0.67 | −0.51 | −1.23 | −0.40 | 1.20 | −1.41 | −1.53 | −0.61 | 1.28 | 1.53 | 0.28 | 0.34 |
| 8028 | −0.59 | −0.34 | −0.03 | −0.41 | −1.91 | −0.70 | −0.92 | −0.78 | −1.75 | 0.00 | 0.39 | 0.32 |
| 150368 | −0.22 | −0.51 | −1.64 | −0.79 | 2.53 | 0.69 | −0.58 | −1.82 | −0.74 | −0.54 | 0.51 | −0.42 |
| 84206 | 0.08 | −1.12 | −0.84 | −0.63 | −1.18 | −1.68 | −1.75 | −0.56 | −1.05 | 0.44 | 0.65 | −0.93 |
| 101929288 | −0.72 | −0.69 | −1.90 | −0.51 | 0.48 | −0.39 | −0.37 | −0.77 | −0.73 | −0.43 | 1.18 | −0.22 |
| 826 | −0.28 | −1.17 | −0.41 | −0.58 | −0.49 | −0.98 | −1.05 | −0.41 | −0.81 | 0.56 | 0.39 | −0.04 |
| 83440 | −0.27 | −1.11 | −0.98 | −0.85 | −0.09 | −0.84 | −1.59 | −0.01 | −1.58 | 1.21 | 1.63 | 0.05 |
| 1128 | −0.39 | −1.54 | −1.81 | 0.21 | 0.16 | −2.12 | −0.69 | −0.41 | 0.70 | 0.97 | −0.80 | 0.89 |
| 160287 | 0.06 | −0.43 | 0.03 | −1.50 | 0.34 | −0.90 | −0.38 | 0.88 | 0.17 | 1.52 | −0.43 | −0.05 |
| 412 | −0.18 | −1.81 | 0.82 | −0.89 | 0.39 | 0.73 | 0.64 | −0.94 | −0.24 | −0.63 | −0.84 | 0.20 |
| 64215 | 0.09 | −0.31 | −1.81 | −0.25 | −1.24 | −1.03 | −1.72 | −0.20 | −0.01 | 0.04 | −0.02 | 0.19 |
| 7343 | −1.28 | −0.70 | −0.63 | −0.59 | 0.26 | −1.18 | −2.83 | 0.15 | −1.14 | 1.93 | 1.57 | −0.26 |
| 25840 | −0.33 | −0.25 | 0.09 | −0.10 | −0.81 | −0.75 | −1.86 | −0.40 | −0.04 | 1.14 | 0.65 | 0.04 |
| 55315 | −0.93 | 0.75 | −0.81 | −0.31 | −0.96 | 1.87 | −0.86 | −0.42 | 0.21 | −0.79 | 0.44 | −0.22 |
| 100507513 | −0.63 | 0.14 | −0.76 | −0.08 | −1.02 | −0.93 | −0.07 | −1.63 | 1.20 | −0.27 | 0.38 | −0.66 |
| 55704 | −0.57 | −1.65 | −0.54 | −0.10 | −1.07 | −2.00 | −0.06 | −0.13 | −1.38 | 0.49 | 0.56 | −0.85 |
| 57604 | −0.47 | −2.21 | −0.46 | 0.23 | −2.17 | −0.35 | 0.05 | −1.66 | 0.20 | 1.17 | 0.48 | −0.67 |
| 10449 | −0.82 | 0.15 | 0.45 | −2.21 | −1.40 | −0.69 | −1.52 | −0.28 | 0.20 | −0.35 | −0.94 | −0.91 |
| 1727 | −0.12 | −0.36 | −0.55 | −1.16 | 0.35 | −0.69 | −2.32 | −0.50 | −0.95 | 0.87 | 0.62 | −0.36 |
| 9569 | −0.12 | −1.84 | −2.20 | −0.34 | −0.82 | −0.24 | −0.72 | −0.16 | −1.93 | 1.36 | 0.32 | 0.33 |
| 10009 | −0.80 | −1.16 | −1.08 | −0.18 | −0.96 | −0.80 | −0.59 | −1.24 | 0.32 | 0.35 | 0.29 | 0.00 |
| 5188 | −0.42 | −3.01 | −1.09 | 0.55 | 0.26 | −0.06 | −2.33 | 0.25 | −0.43 | −0.53 | 0.25 | −0.38 |
| 2132 | −0.49 | −1.19 | 0.06 | 0.42 | −2.44 | −0.51 | 0.41 | −1.46 | 1.70 | −0.51 | 0.07 | 0.93 |
| 2239 | −0.62 | −0.72 | −1.45 | −1.88 | −0.30 | 0.56 | 0.00 | −0.65 | −0.13 | 0.61 | 1.59 | −0.09 |
| 56927 | 0.22 | −2.64 | −0.20 | −0.50 | −0.38 | 0.59 | 0.43 | −0.48 | −0.59 | −0.46 | 0.96 | −0.92 |
| 2950 | −0.42 | −1.24 | −1.53 | −0.25 | −0.04 | −0.52 | −1.24 | −0.17 | −1.67 | 0.19 | 0.49 | −0.84 |
| 342538 | −0.70 | −0.33 | −1.80 | 1.04 | −0.70 | −1.75 | −0.48 | −0.70 | −2.09 | 1.11 | −0.08 | 1.12 |
| 55218 | −0.98 | 0.35 | −1.78 | −0.68 | −0.02 | −1.07 | −2.27 | −0.41 | −0.07 | 0.83 | 0.78 | 0.28 |
| 151827 | 0.03 | −0.68 | −2.19 | −0.20 | −0.84 | −0.06 | −1.75 | −0.08 | −0.62 | 0.50 | 0.73 | −0.80 |
| 64689 | −0.05 | −1.44 | 0.10 | −0.65 | −1.71 | 0.34 | −0.40 | −0.98 | −1.40 | −0.45 | 0.37 | −0.81 |
| 10324 | −0.23 | −0.90 | −1.52 | 0.79 | 1.46 | 0.27 | 0.19 | −1.04 | −0.47 | −1.27 | −1.22 | −0.13 |
| 1314 | −0.71 | −1.48 | −0.79 | −1.03 | 0.16 | −0.59 | −1.60 | 0.16 | −1.99 | −0.28 | 1.25 | 0.04 |
| 54928 | −0.27 | −0.35 | −0.05 | −0.79 | −2.08 | −0.33 | 0.07 | −0.26 | −1.04 | −0.86 | 0.44 | −0.36 |
| 8971 | 0.07 | −0.49 | −1.86 | −0.38 | −0.69 | −1.67 | −1.08 | −0.99 | −1.20 | 1.07 | −0.15 | −0.43 |
| 79832 | −0.17 | −0.86 | −0.28 | −0.33 | −0.67 | −0.55 | −0.15 | −0.41 | −0.49 | −0.51 | 0.73 | 1.29 |
| 10150 | −0.95 | −1.27 | −1.00 | −0.38 | −1.14 | 0.64 | −1.05 | −0.17 | −0.74 | −0.42 | 0.85 | 0.01 |
| 8744 | −0.48 | 0.26 | −1.51 | −0.14 | −1.47 | −1.51 | −0.99 | −0.47 | −1.18 | 0.51 | 0.11 | −0.78 |
| 23287 | 1.22 | −0.26 | 2.13 | 0.44 | −0.11 | 0.13 | 1.42 | 1.05 | 0.79 | 0.77 | −0.25 | 0.62 |
| 606553 | 0.66 | 0.81 | 0.08 | 1.02 | 0.43 | −1.56 | −0.86 | 0.62 | 0.74 | −0.22 | 0.97 | −0.84 |
| 103 | 0.27 | 0.23 | 0.58 | 0.50 | 1.37 | 0.61 | 2.05 | 0.95 | 0.87 | −0.45 | 0.75 | 0.64 |
| 55509 | −0.02 | 0.69 | −0.21 | 0.45 | 0.60 | 1.32 | 1.27 | 0.90 | −1.19 | −1.38 | −0.01 | −0.22 |
| 729614 | 0.81 | −0.62 | 1.52 | 0.73 | 0.20 | 0.30 | 1.52 | 1.04 | 0.56 | 0.04 | −0.53 | 0.27 |
| 9241 | −0.87 | 2.11 | 0.05 | 0.74 | −0.47 | −0.65 | 0.35 | 0.07 | 1.78 | 1.04 | 0.16 | 1.41 |
| 26118 | 0.60 | −0.84 | 0.72 | 0.11 | −0.10 | 1.28 | 1.04 | 0.96 | 0.73 | 0.36 | 0.70 | 0.02 |
| 64400 | −0.03 | −0.16 | 1.10 | 0.53 | 0.44 | 1.81 | 1.33 | 1.01 | 1.39 | −0.44 | 0.28 | 1.03 |
| 283237 | −0.54 | 0.23 | 0.98 | 0.19 | 0.50 | 0.70 | 0.13 | 0.91 | −0.85 | −0.34 | 0.49 | 1.61 |
| 51155 | 0.68 | −0.19 | 1.52 | 0.82 | 0.58 | 0.64 | 0.87 | 1.25 | 0.22 | −0.07 | −0.03 | −0.24 |
| 18 | 0.31 | 0.33 | 1.74 | 0.49 | 0.10 | 1.28 | 1.60 | 0.61 | 0.95 | −0.86 | 0.15 | 0.58 |
| 22990 | 1.00 | −0.21 | 1.87 | 0.84 | −0.13 | 0.50 | 1.60 | 1.32 | 0.89 | 0.16 | 0.09 | 0.51 |
| 23347 | −0.33 | 0.55 | 1.55 | 0.38 | 0.49 | 0.74 | 1.69 | 0.44 | 1.37 | 0.40 | 0.26 | 0.27 |
| 83988 | −0.21 | 1.14 | −1.32 | 0.36 | 1.67 | 0.69 | 0.42 | 0.04 | 0.26 | 0.61 | −0.31 | 1.21 |
| 3428 | −0.23 | 0.76 | 0.89 | 0.80 | 1.81 | 1.23 | 0.86 | 0.99 | 1.04 | −0.54 | −0.22 | 0.79 |
| 3700 | 0.75 | 0.42 | 1.03 | 0.85 | 0.10 | 0.96 | 0.79 | 0.32 | 1.18 | −0.41 | 0.25 | 0.56 |
| 64859 | 1.06 | −0.95 | 1.92 | 0.65 | −0.52 | 1.36 | 1.76 | 1.07 | 0.79 | −0.98 | −0.24 | 0.27 |
| 55192 | −0.39 | −0.81 | 1.37 | −0.26 | 0.92 | 1.29 | 0.70 | 1.13 | 0.36 | −2.35 | 1.03 | 0.10 |
| 101928869 | −0.04 | 1.75 | 0.91 | 0.85 | 1.93 | 1.19 | 0.15 | −0.60 | −0.16 | 0.06 | 0.17 | 0.19 |
| 10513 | 0.01 | 0.67 | 1.59 | 0.67 | −0.63 | 1.67 | 1.45 | 0.81 | 0.31 | −0.53 | 0.18 | 0.91 |
| 11009 | −1.30 | 2.52 | −0.08 | −0.13 | 0.06 | −0.20 | 0.75 | −0.56 | 1.34 | 0.24 | −0.01 | 0.48 |
| 169355 | 2.06 | 1.60 | 0.56 | 1.18 | −0.24 | 0.98 | 0.94 | −0.33 | −0.81 | −1.63 | −0.34 | 0.14 |
| 10109 | −1.80 | 0.01 | 2.33 | 0.80 | 0.71 | 0.77 | 0.54 | 0.29 | 1.02 | 0.04 | −0.34 | 1.13 |
| 9903 | 0.43 | 0.47 | 2.17 | 1.10 | −0.11 | 0.89 | 1.64 | 0.08 | 0.63 | −0.97 | 0.08 | 1.22 |
| 23150 | 0.58 | 0.34 | 1.40 | 0.90 | −1.32 | 1.36 | 1.19 | 0.20 | 1.31 | −0.63 | 0.07 | 0.83 |
| 10865 | 1.21 | −0.41 | 0.75 | 0.12 | 0.34 | 0.22 | 1.51 | 1.44 | −1.16 | 0.65 | 0.52 | −0.43 |
| 5165 | −0.02 | 0.28 | 1.95 | 0.00 | 0.19 | 1.47 | 1.77 | 1.18 | 0.96 | −0.93 | 0.10 | −0.07 |
| 57403 | −0.13 | 0.51 | 2.05 | 0.52 | −0.33 | 1.09 | 1.44 | 0.70 | 1.31 | −0.64 | −0.45 | 0.29 |
| 22900 | −0.41 | −0.17 | 1.69 | 1.03 | 0.65 | 0.18 | 1.44 | 0.44 | 1.64 | −0.19 | −0.12 | 1.43 |
| 80345 | 0.44 | −0.40 | 1.15 | 0.61 | 0.78 | 0.37 | 1.82 | 1.31 | 0.13 | −0.62 | −1.06 | 1.05 |
| 10964 | −1.10 | 1.33 | 0.19 | 0.77 | 0.76 | 1.61 | 0.71 | 0.31 | 1.37 | −0.46 | −0.81 | 0.84 |
| 27040 | −0.63 | 2.03 | −1.09 | 1.40 | 0.58 | 0.63 | −0.26 | −0.09 | 0.57 | 0.41 | 0.15 | 1.44 |
| 91010 | −0.53 | 2.18 | 1.05 | 0.47 | 0.06 | 0.82 | 0.25 | 0.46 | 0.00 | −2.78 | 0.17 | 0.34 |
| 29065 | 0.47 | 0.25 | 1.51 | 1.33 | 0.21 | 0.72 | 1.55 | 1.02 | 1.40 | −0.68 | −0.21 | −0.34 |
| 84078 | 0.45 | −0.01 | 2.40 | 0.74 | −0.25 | 1.25 | 1.94 | 0.29 | 0.93 | −0.39 | 0.06 | 0.84 |
| 101927910 | 0.32 | 0.06 | 0.88 | 0.83 | 2.09 | 0.79 | −0.52 | 0.79 | 0.42 | 0.18 | −0.05 | −0.41 |
| 51380 | 0.73 | −0.40 | 1.68 | 0.42 | 0.27 | 1.05 | 1.50 | 1.50 | 0.48 | 0.11 | 0.31 | −0.12 |
| 10289 | 0.31 | 0.91 | 2.11 | 0.60 | −3.60 | −0.01 | 1.16 | 0.60 | 0.77 | −1.07 | −0.39 | 0.69 |
| 10296 | −0.33 | 0.38 | 0.97 | 0.18 | −0.05 | 0.73 | 0.88 | 1.42 | 0.80 | 1.48 | 0.27 | 0.65 |
| 114224 | 1.25 | 0.59 | 1.30 | 0.74 | 0.54 | 0.37 | 0.83 | 0.70 | 0.88 | 0.59 | −0.44 | 0.93 |
| 2153 | 0.31 | 0.23 | 1.78 | 0.32 | 0.84 | 1.13 | 1.30 | 0.99 | 1.49 | −0.18 | −0.35 | 0.03 |
| 55825 | 0.04 | −0.20 | 1.43 | 0.82 | 0.75 | 1.90 | 0.91 | 0.93 | 1.53 | −1.12 | 0.04 | 0.44 |
| 6503 | −0.56 | 1.07 | 1.09 | 0.58 | 0.45 | 0.79 | 1.27 | 0.61 | 0.40 | 0.20 | 1.09 | 0.51 |
| 7913 | 0.74 | 0.74 | 0.38 | 0.75 | −0.88 | −0.27 | 0.55 | 0.95 | 0.62 | −1.06 | −1.38 | 1.70 |
| 30834 | −0.49 | 1.36 | −0.23 | 0.88 | 1.26 | 1.21 | −0.55 | −0.36 | 0.45 | 0.30 | −0.01 | 0.31 |
| 26999 | −0.12 | 0.95 | 1.08 | 1.08 | 1.14 | 0.68 | 0.40 | 0.80 | 0.79 | −0.58 | −0.01 | 1.71 |
| 286437 | 0.04 | 0.27 | 1.68 | 0.85 | 0.55 | 1.37 | 1.97 | 0.57 | 0.98 | −0.83 | −0.43 | 0.89 |
| 4277 | −1.09 | 1.15 | 1.68 | 1.25 | 0.76 | −0.04 | 1.39 | −0.16 | 2.18 | −0.58 | −1.13 | 0.64 |
| 5698 | −0.85 | 1.13 | 0.20 | 0.73 | 3.00 | 1.24 | 0.60 | 0.14 | 0.18 | −1.15 | 0.05 | 0.24 |
| 4318 | 0.77 | −0.48 | 1.42 | 0.57 | −0.07 | 1.19 | 2.13 | 1.22 | 0.92 | −0.52 | 0.09 | −0.85 |
| 10616 | 1.30 | −0.93 | 0.33 | 0.37 | −0.37 | −0.11 | 0.63 | 1.17 | 1.32 | 0.23 | 0.23 | 0.83 |
| 23012 | 0.24 | −0.12 | 1.99 | 0.83 | 0.14 | 0.66 | 2.08 | 2.05 | 1.25 | −0.19 | 0.30 | −0.10 |
| 100129361 | 0.65 | −0.41 | 1.17 | 0.12 | −0.87 | 0.69 | 2.17 | 0.22 | 1.48 | −0.03 | −0.69 | 1.23 |
| 10742 | 0.22 | 1.27 | −0.11 | −0.07 | 0.50 | 2.51 | 1.59 | −0.28 | 1.29 | 0.20 | 0.53 | 1.14 |
| 196264 | −0.03 | 0.53 | 1.90 | 0.23 | 0.40 | 1.52 | 1.72 | 0.64 | 1.46 | −1.03 | −0.46 | 0.35 |
| 27197 | −0.56 | 0.52 | 1.68 | 0.11 | 0.86 | 1.19 | 0.76 | 0.01 | 1.50 | −1.24 | 0.30 | 0.62 |
| 7529 | −1.43 | 0.90 | 1.85 | 0.66 | −1.22 | 1.39 | 1.01 | 0.50 | 1.01 | 0.01 | 0.24 | 0.87 |
| 94235 | 0.38 | −0.99 | 1.29 | 0.27 | 1.86 | 1.27 | 1.57 | −0.33 | 0.76 | −1.95 | −0.40 | −1.23 |
| 352961 | −1.13 | 1.94 | 0.65 | 0.87 | 1.1 1 | 0.46 | 0.69 | −0.22 | −0.68 | 0.31 | 1.29 | 1.04 |
| 49856 | −0.34 | −1.11 | 2.08 | 1.20 | 0.31 | 1.03 | 0.65 | 1.78 | 0.14 | −0.41 | −0.03 | 0.28 |
| 9448 | 0.77 | −0.22 | 1.73 | 0.69 | −0.29 | 0.59 | 1.97 | 1.05 | 1.19 | 0.25 | −0.01 | 0.44 |
| 9525 | 0.05 | 1.17 | 1.85 | 1.07 | −0.99 | 0.07 | 1.39 | 0.89 | 1.57 | 0.61 | −0.67 | 1.62 |
| 100505576 | −1.00 | 0.50 | 2.03 | 0.82 | 1.04 | 1.15 | 1.21 | 0.48 | 1.14 | 0.14 | −0.51 | 0.25 |
| 151556 | −0.76 | 0.53 | 1.62 | 0.66 | 0.75 | 1.94 | 1.56 | 0.24 | 0.92 | −0.43 | 0.40 | 1.22 |
| 284613 | −0.95 | 0.94 | 1.30 | 1.37 | 1.91 | 0.38 | 0.31 | 0.18 | 0.03 | −0.32 | 0.27 | 1.14 |
| 286223 | −0.10 | 2.67 | 1.01 | 0.83 | 0.81 | 0.27 | 0.89 | −0.37 | 0.97 | −0.53 | −0.27 | −0.69 |
| 57061 | 0.71 | 1.37 | 1.32 | 1.18 | 1.46 | 0.26 | 0.77 | 1.33 | −1.18 | 0.40 | 0.72 | −0.91 |
| 9520 | 0.67 | −0.29 | 2.38 | 0.32 | −0.37 | 1.33 | 1.67 | 1.14 | 0.32 | −0.53 | 0.09 | −0.23 |
| 202299 | 1.88 | 0.47 | 0.12 | 0.22 | 1.70 | 0.34 | 0.42 | 1.23 | −0.34 | −2.27 | 0.04 | −0.76 |
| 2015 | 1.31 | 0.23 | 1.65 | 0.33 | −0.38 | 0.60 | 1.18 | 0.76 | 1.35 | −1.49 | −0.84 | 0.54 |
| 57763 | 0.48 | 0.65 | 0.70 | 1.43 | −0.90 | 0.38 | 0.55 | 0.90 | 0.97 | 0.05 | −0.81 | 1.86 |
| 54778 | −0.42 | 0.18 | 2.08 | 0.55 | −0.12 | 1.49 | 1.21 | 0.92 | 1.13 | −0.69 | 0.52 | 1.05 |
| 762 | 0.60 | 0.10 | 2.14 | −0.06 | 0.38 | 1.31 | 1.77 | 1.01 | 0.66 | −0.24 | 0.19 | −0.53 |
| 192668 | 1.30 | −0.16 | 0.38 | 0.06 | 2.18 | 1.76 | 0.63 | 0.93 | 1.55 | −1.64 | −0.80 | −1.50 |
| 24146 | −1.01 | 0.61 | 0.75 | 0.70 | −1.11 | 0.19 | 0.64 | 0.85 | 0.52 | −0.09 | 0.53 | 1.15 |
| 54843 | 0.17 | 1.23 | −0.47 | 0.27 | 0.31 | 1.88 | 0.79 | 0.52 | 0.71 | −0.62 | 0.25 | 1.08 |
| 55374 | 0.89 | −1.38 | 1.73 | 0.98 | 0.78 | 1.09 | 1.26 | 0.80 | 0.41 | −1.71 | 0.14 | 1.19 |
| 79603 | 0.39 | 0.24 | 1.41 | 0.56 | −0.14 | 2.37 | 1.73 | 0.22 | 0.87 | −1.87 | 0.41 | −0.18 |
| 3577 | 0.02 | 0.41 | 1.65 | 0.38 | 0.78 | 1.05 | 1.94 | 1.08 | 0.95 | 0.22 | 0.20 | −0.45 |
| 10385 | 0.09 | 1.37 | −0.04 | 1.81 | 0.77 | 1.33 | 1.48 | 0.17 | −0.19 | −0.89 | −0.75 | −0.27 |
| 79792 | 1.05 | 0.44 | −0.84 | 1.06 | 1.48 | −0.70 | 0.09 | 0.76 | −0.06 | 0.83 | 0.04 | −0.91 |
| 84674 | 0.60 | −0.10 | 1.92 | 0.49 | −0.08 | 0.89 | 1.17 | 1.17 | 1.35 | −0.49 | 0.06 | 0.21 |
| 51131 | −0.66 | 2.53 | 0.00 | 0.42 | 1.15 | 1.55 | 0.04 | −0.15 | 0.76 | −0.34 | 0.45 | 1.57 |
| 5966 | 1.10 | −0.25 | 2.05 | 0.05 | −1.56 | 0.88 | 0.99 | 0.70 | 0.06 | −1.99 | 0.25 | 0.28 |
| 9733 | −1.56 | 1.24 | 0.09 | 1.24 | −0.12 | 0.44 | 1.04 | 1.05 | −0.65 | 0.71 | 0.62 | 0.22 |
| 10328 | 0.44 | 1.46 | 0.72 | 1.45 | −0.45 | 0.42 | 1.19 | 0.22 | −0.96 | −0.08 | −0.14 | −0.22 |
| 255231 | −1.56 | 1.36 | −0.16 | 0.87 | 0.96 | 1.48 | 0.44 | 0.65 | −0.22 | −0.43 | −0.38 | 0.83 |
| 25801 | −0.16 | 0.75 | 1.99 | 0.56 | −0.33 | 0.66 | 0.95 | 1.23 | 1.93 | −0.35 | −0.04 | −0.34 |
| 6352 | −0.99 | 0.77 | 0.97 | 0.84 | 0.75 | 0.81 | 0.56 | −0.17 | 1.43 | −1.63 | −0.89 | 1.85 |
| 54811 | −0.48 | 2.78 | 0.89 | 0.42 | −0.23 | 0.04 | 0.69 | −0.38 | −0.41 | 1.33 | 0.16 | 0.03 |
| 59348 | 0.72 | 0.92 | 1.52 | −0.06 | −1.02 | 0.65 | 1.83 | 0.48 | 1.01 | −0.88 | 0.18 | 0.58 |
| 9967 | 0.50 | 0.34 | 1.38 | 0.42 | −0.18 | 0.82 | 1.13 | 1.04 | 0.01 | 0.19 | 0.42 | −1.21 |
| 2533 | −0.12 | 0.63 | 1.64 | 0.60 | 0.06 | 1.33 | 0.87 | 0.79 | 1.42 | −0.11 | 0.06 | 0.98 |
| 28985 | 0.01 | 0.87 | 0.08 | 1.86 | 0.77 | 0.38 | 2.11 | −0.30 | 0.53 | −0.45 | −0.71 | 1.60 |
| 55234 | 0.87 | 1.08 | 1.66 | 0.78 | −0.39 | −0.24 | 1.02 | 0.09 | 1.09 | 0.76 | 0.11 | 1.58 |
| 1301 | 0.56 | 0.58 | 0.52 | 0.09 | 1.35 | 1.68 | 0.40 | 1.30 | 0.14 | −1.27 | −0.59 | −1.00 |
| 84803 | 0.57 | 0.77 | 1.75 | 0.09 | −0.19 | 1.09 | 1.49 | 1.32 | 1.46 | −1.63 | −0.04 | 0.34 |
| 100506779 | −0.34 | 1.03 | 0.52 | 1.04 | 0.95 | −0.25 | 0.59 | 0.61 | 1.56 | 0.60 | −0.84 | 1.97 |
| 197135 | −0.85 | 0.86 | 0.54 | 0.70 | 1.70 | 1.12 | 1.12 | −0.13 | 1.75 | −1.20 | −0.31 | 1.55 |
| 3614 | 0.30 | 0.96 | 1.83 | 0.45 | 1.02 | 0.05 | 1.63 | 0.31 | 1.20 | 0.04 | −0.59 | 0.18 |
| 80781 | 0.35 | 0.34 | 0.80 | −0.26 | 1.12 | 1.50 | 1.39 | 0.87 | −0.60 | 0.01 | 1.40 | 0.88 |
| 5586 | 0.00 | 0.82 | 2.16 | 0.30 | −1.31 | 1.42 | 1.77 | 0.51 | −0.09 | 0.04 | 1.01 | −0.23 |
| 79415 | 0.50 | −0.90 | 0.99 | 1.16 | 0.88 | 0.65 | 0.73 | 1.39 | 0.74 | 0.29 | 0.33 | 0.22 |
| 8893 | 0.97 | 1.33 | −1.57 | 1.62 | 0.98 | −0.49 | 0.54 | −0.32 | 1.23 | 0.11 | −2.02 | 0.86 |
| 79736 | 0.40 | −0.29 | 1.26 | 0.56 | 0.09 | 1.61 | 0.35 | 0.66 | −0.24 | −1.38 | −0.24 | −0.37 |
| 5770 | 0.32 | 0.41 | 1.10 | 0.12 | 0.64 | 0.96 | 1.02 | 0.64 | 0.10 | −0.69 | 0.96 | −1.02 |
| 9360 | −0.15 | 0.55 | 1.10 | 0.39 | −0.08 | 0.54 | 1.34 | 0.98 | 0.83 | 1.00 | −0.98 | 0.10 |
| 29940 | 0.80 | 0.95 | 2.13 | −0.14 | −1.14 | 1.24 | 0.94 | 1.56 | 0.98 | −1.34 | 0.34 | −0.17 |
| 7150 | 0.33 | 2.22 | 0.31 | 0.27 | −0.34 | −0.25 | 1.11 | 0.48 | −0.64 | 0.16 | −0.13 | −0.72 |
| 84964 | 1.22 | 0.31 | −0.89 | 1.78 | 1.24 | −0.29 | −0.06 | 0.67 | 1.82 | −0.02 | −0.78 | 1.67 |
| 10614 | 0.02 | 1.76 | 1.99 | 0.77 | −1.31 | −0.09 | 1.62 | 0.85 | 0.60 | −1.08 | −0.71 | −0.06 |
| 147923 | 0.00 | 0.45 | 0.82 | 0.57 | 0.48 | 1.70 | 1.89 | −0.09 | 1.09 | −0.96 | 0.36 | 0.87 |
| 284029 | −0.49 | 0.56 | 1.66 | 0.65 | 0.91 | 1.60 | 1.44 | 0.87 | 1.52 | −0.73 | −0.02 | 0.54 |
| 54952 | 0.05 | −1.00 | 1.41 | 1.18 | 0.24 | 1.67 | 1.29 | 0.71 | 0.98 | −1.15 | −1.42 | 0.67 |
| 57476 | −0.25 | 1.90 | −0.34 | 0.80 | 1.04 | 0.08 | 1.05 | 1.07 | 1.17 | −0.65 | 0.31 | 0.53 |
| 8745 | 0.81 | −0.18 | −0.01 | 2.00 | −0.14 | 0.00 | 0.11 | 0.78 | 0.86 | −0.61 | 0.67 | −0.09 |
| 22928 | 0.29 | −0.55 | 2.68 | 0.68 | 0.38 | 0.74 | 0.84 | 0.96 | 0.53 | −0.67 | −1.04 | 0.61 |
| 25880 | −0.68 | 1.35 | −0.51 | 0.99 | 1.52 | 0.32 | 1.42 | 0.81 | 0.68 | 1.25 | −1.14 | 0.66 |
| 28638 | −1.29 | 1.68 | −0.07 | 0.91 | 1.16 | 0.90 | 0.34 | 0.41 | 1.54 | 0.06 | 0.48 | 1.13 |
| 29933 | 0.74 | −0.12 | 2.20 | −0.13 | −0.29 | 0.67 | 0.55 | 1.14 | −0.22 | −0.91 | 0.64 | −0.33 |
| 30009 | −1.29 | 1.57 | 0.87 | 1.50 | 1.10 | 0.29 | 0.91 | 0.31 | 1.27 | −0.98 | −0.42 | 1.09 |
| 3554 | 1.54 | −0.20 | 1.25 | 0.05 | 0.01 | 1.49 | 1.69 | 1.08 | 1.14 | −1.73 | 0.14 | −1.36 |
| 8821 | 0.31 | 1.81 | −0.27 | 0.79 | 0.11 | 0.92 | 0.10 | −0.38 | 1.87 | −1.03 | 0.37 | 1.31 |
| 10943 | 0.45 | −0.82 | 1.44 | 0.47 | 0.70 | 1.31 | 1.19 | 0.71 | 1.43 | 0.23 | 0.14 | 1.36 |
| 57724 | −0.20 | 0.61 | 1.42 | 0.76 | 0.56 | 0.27 | 1.57 | −0.17 | 1.99 | 0.49 | −0.19 | 1.02 |
| 100506282 | 1.07 | −0.07 | 0.74 | −0.53 | 0.01 | 0.89 | 1.95 | −1.25 | 0.53 | −1.01 | 0.05 | −0.26 |
| 147699 | −0.19 | 0.22 | 1.70 | 0.96 | 0.95 | 0.97 | 0.78 | 1.39 | 0.99 | −0.81 | 0.07 | −0.33 |
| 3002 | −1.46 | 1.40 | 0.57 | 0.71 | 0.54 | 0.99 | 1.01 | 0.60 | 1.91 | −1.01 | −0.19 | 1.05 |
| 389320 | 1.13 | 1.57 | 1.12 | 2.12 | 0.00 | −1.54 | 0.65 | −0.73 | −1.55 | 1.05 | −0.61 | 0.70 |
| 9914 | 0.78 | −0.40 | 1.76 | −0.16 | 0.77 | 1.89 | 1.04 | 1.22 | −0.06 | −0.19 | 0.43 | −1.13 |
| 10804 | 0.81 | 0.49 | 1.50 | 0.51 | −0.28 | 1.35 | 2.22 | 0.90 | 0.30 | −0.80 | 0.74 | −0.01 |
| 51439 | 0.42 | 0.69 | 1.97 | 0.43 | −0.38 | 1.46 | 1.59 | 0.98 | 1.18 | −0.17 | 0.32 | 0.55 |
| 814 | −1.77 | 0.95 | 0.46 | 1.09 | 1.10 | 1.19 | 1.02 | 0.51 | 1.22 | −0.06 | −0.03 | 1.23 |
| 5272 | −0.98 | 2.13 | 1.17 | 0.13 | 1.86 | 0.91 | 1.06 | 0.87 | 0.19 | −0.51 | −0.21 | 1.19 |
| 8428 | −0.29 | 1.84 | 1.70 | 0.16 | 1.13 | 0.66 | 0.00 | 0.54 | 0.98 | 0.51 | −0.54 | 1.42 |
| 23048 | 0.23 | 2.57 | −0.01 | 0.94 | 0.21 | −0.59 | 0.81 | 0.85 | −0.35 | 0.48 | −0.79 | 0.91 |
| 344558 | −0.41 | 0.74 | 0.36 | 0.16 | 0.16 | 1.04 | 2.22 | 0.64 | 1.22 | 0.82 | 0.42 | 0.44 |
| 79078 | −1.09 | 2.24 | 0.25 | 0.60 | 0.34 | 1.06 | 1.02 | −0.36 | −0.43 | 0.20 | −0.55 | 0.20 |
| 81606 | −1.26 | 2.52 | 0.10 | 0.75 | 0.87 | 0.65 | −0.09 | −0.51 | 0.85 | 0.30 | 0.08 | 1.25 |
| 64377 | 0.79 | 2.02 | 1.14 | −0.65 | 0.58 | 0.67 | 0.51 | 1.91 | 1.05 | −0.55 | 0.16 | −0.13 |
| 57501 | 0.78 | 1.58 | 0.16 | 0.24 | 1.80 | 0.46 | 0.96 | 0.62 | 0.91 | −0.83 | −0.51 | 0.22 |
| 5991 | 2.05 | −0.76 | 1.31 | −0.08 | −0.70 | 1.25 | 1.43 | 0.81 | −0.71 | −0.33 | 0.77 | −0.36 |
| 90075 | 0.52 | 1.22 | 0.04 | 1.13 | −0.22 | 0.09 | 0.17 | −0.46 | 1.92 | −0.28 | −0.05 | 0.73 |
| 375341 | 0.88 | −0.16 | 2.46 | 0.28 | −0.15 | 0.87 | 1.81 | 0.37 | 0.21 | −0.73 | 0.03 | 0.21 |
| 100130548 | 0.77 | −0.28 | 0.79 | −0.17 | 1.39 | 1.47 | −0.18 | 1.85 | 1.51 | −0.18 | −0.70 | 0.20 |
| 114786 | 0.36 | 0.18 | −0.33 | 0.18 | 0.15 | 1.43 | 1.42 | 0.86 | 2.80 | −1.10 | 0.01 | −0.43 |
| 150084 | 0.36 | 1.25 | 0.66 | −1.41 | 0.71 | 0.30 | −0.46 | 2.19 | 0.68 | 0.28 | 0.00 | 0.06 |
| 55012 | 0.14 | 0.42 | 2.15 | 0.26 | −0.14 | 1.51 | 1.48 | 1.12 | 0.75 | −0.90 | −0.32 | 0.57 |
| 147525 | −0.31 | −0.06 | 1.45 | 0.95 | 2.63 | 1.24 | −1.14 | 0.73 | 0.72 | −1.12 | 0.69 | 0.95 |
| 54557 | 0.17 | 0.59 | 2.07 | 0.35 | −0.05 | 1.50 | 1.79 | 0.93 | 0.64 | −0.54 | −0.02 | 0.72 |
| 56267 | 0.59 | 0.68 | 1.12 | 0.66 | 0.00 | 1.40 | 0.55 | 0.51 | −1.04 | −0.31 | −1.27 | 2.06 |
| 51176 | −1.07 | 0.94 | 0.73 | 1.19 | 0.97 | 1.09 | 1.34 | −0.10 | 1.68 | −0.17 | 0.40 | 1.23 |
| 10468 | 0.77 | 0.69 | 0.48 | 0.50 | 0.02 | 0.92 | 0.94 | 1.12 | −1.83 | −0.50 | 1.95 | −1.06 |
| 23258 | 0.68 | 0.33 | 2.08 | 0.61 | 0.09 | 0.54 | 1.76 | 1.08 | 0.95 | −0.33 | 0.11 | 0.44 |
| 90768 | 0.45 | 0.94 | 0.64 | 0.78 | 1.61 | 1.01 | 0.49 | 0.60 | 1.84 | −1.64 | −1.32 | −0.38 |
| 134957 | 0.98 | −0.56 | 2.12 | 0.46 | −0.53 | 0.73 | 1.82 | 0.91 | 1.02 | −0.17 | −0.11 | 0.44 |
| 80342 | −0.81 | 1.33 | 0.34 | 0.89 | 0.53 | 0.13 | 0.34 | 0.28 | 1.29 | 0.80 | −0.40 | 1.93 |
| 91351 | 0.33 | 0.10 | 1.95 | 0.83 | 0.14 | 1.02 | 1.47 | 0.92 | 1.37 | −0.12 | 0.11 | 0.83 |
| 29916 | 0.79 | −0.08 | 1.78 | 0.79 | 0.64 | 0.74 | 1.63 | 1.25 | −0.33 | −0.90 | −0.74 | −0.48 |
| 8519 | −1.06 | 0.89 | 1.30 | 0.22 | 1.44 | 1.02 | 0.77 | 0.61 | 1.56 | −0.27 | 0.36 | 0.81 |
| 2113 | −0.93 | 2.02 | 0.13 | 0.53 | 1.00 | 0.61 | 0.39 | 0.46 | 1.48 | 0.16 | 0.65 | 1.32 |
| 55770 | −0.31 | 0.60 | 0.06 | 0.36 | 0.01 | 0.40 | 1.04 | −0.23 | 0.99 | 0.29 | 0.76 | 1.44 |
| 91833 | 0.30 | 0.45 | 2.25 | 0.37 | −0.15 | 1.37 | 1.26 | 0.24 | 0.42 | −0.54 | 0.23 | 0.99 |
| 117177 | 0.40 | −0.60 | 1.24 | 0.82 | 0.82 | 1.56 | 1.51 | 0.97 | 1.34 | −1.48 | 0.31 | 0.76 |
| 546 | 0.60 | −0.01 | 1.70 | 0.81 | −0.48 | 0.46 | 1.21 | 0.85 | 0.12 | 0.44 | 0.92 | 0.15 |
| 4253 | −0.38 | −0.14 | 1.90 | 0.97 | 0.59 | 1.30 | 1.46 | 0.76 | 0.17 | −0.48 | 0.41 | 1.74 |
| 23214 | 0.31 | 0.36 | 1.22 | 0.42 | 0.52 | 0.44 | 1.66 | 1.25 | 0.88 | 0.49 | −0.29 | 0.40 |
| 53347 | 0.00 | 1.13 | 0.52 | 0.51 | 0.39 | 1.80 | 0.65 | 0.03 | 1.42 | 0.08 | −0.21 | 1.10 |
| 56888 | −1.20 | 0.83 | 1.90 | 0.98 | 0.93 | 1.15 | 0.21 | 0.71 | 1.29 | 0.74 | 1.09 | 0.34 |
| 777 | 2.32 | −0.64 | 1.29 | 0.70 | 0.27 | 0.87 | 1.67 | 0.84 | 0.60 | −0.91 | 0.25 | −0.91 |
| 100216545 | 0.78 | 0.22 | 1.92 | 1.46 | 0.35 | −0.38 | 2.02 | 0.78 | −1.40 | −0.32 | −1.32 | −0.63 |
| 8879 | 0.87 | 0.44 | 1.20 | 0.78 | −0.59 | 1.65 | 1.47 | 1.07 | −0.07 | −0.56 | 0.00 | 0.19 |
| 9759 | 0.56 | 0.72 | 2.01 | 0.65 | 0.21 | 0.45 | 1.28 | 0.07 | 1.02 | 0.52 | −0.27 | 0.34 |
| 26578 | 0.25 | 0.68 | 1.82 | 0.77 | −0.03 | 1.27 | 1.69 | 0.70 | 0.87 | −0.17 | 0.31 | −0.22 |
| 4050 | −1.14 | 1.24 | 0.75 | 1.10 | 0.97 | 1.15 | 1.11 | 0.84 | 1.04 | 0.10 | −0.02 | 0.83 |
| 4236 | −1.82 | 1.39 | 1.80 | 0.64 | 0.59 | 1.39 | 0.06 | 0.29 | 0.08 | 0.28 | −0.44 | 0.50 |
| 54491 | −0.02 | 0.58 | 1.64 | 0.86 | 0.89 | 0.86 | 0.49 | 0.77 | 1.34 | 0.12 | 0.11 | 1.09 |
| 55847 | 1.28 | 0.70 | 1.63 | 0.58 | −0.42 | 2.24 | 0.33 | 0.51 | 0.95 | −1.47 | −1.03 | 0.25 |
| 100132707 | −0.19 | −0.29 | 1.25 | 1.04 | 1.40 | 1.93 | 1.29 | −0.14 | 1.03 | −0.96 | −0.49 | 0.84 |
| 55884 | 0.61 | 0.84 | 1.93 | −0.23 | −0.12 | 1.56 | 2.12 | 0.32 | 0.51 | −1.73 | −0.19 | −0.43 |
| 83607 | −0.11 | 2.29 | 1.09 | 1.92 | −0.29 | −1.00 | 1.17 | 0.14 | 0.69 | 0.39 | −0.23 | 0.70 |
| 9252 | −0.58 | 0.79 | 1.76 | 0.63 | −0.51 | 1.63 | 2.18 | 0.01 | 0.87 | −0.60 | 0.79 | 1.59 |
| 55119 | 0.42 | −0.47 | 0.89 | 1.10 | −1.07 | 0.09 | 1.28 | 0.93 | 0.92 | −0.05 | 0.27 | 1.60 |
| 57520 | 1.06 | −0.19 | 1.25 | 0.23 | 0.17 | 1.25 | 2.16 | 0.89 | 1.30 | −1.12 | −0.70 | −0.46 |
| 79818 | −0.01 | 0.32 | 2.65 | 0.87 | −0.10 | 2.00 | 1.00 | 0.88 | 0.39 | −0.87 | −0.27 | 0.61 |
| 8320 | −0.76 | 0.99 | 0.59 | 0.27 | 1.15 | 1.49 | 0.52 | 0.78 | 1.25 | −0.66 | −0.29 | 2.05 |
| 2792 | 0.28 | 0.50 | 0.39 | 0.84 | 0.38 | −0.25 | 0.04 | −0.08 | 1.59 | 0.58 | −0.57 | −0.11 |
| 54910 | 0.63 | 2.60 | −0.10 | −0.02 | −1.29 | 0.13 | 0.44 | 0.65 | 0.36 | 0.67 | 0.85 | −0.63 |
| 55114 | −0.27 | 2.50 | 0.98 | 0.68 | 1.83 | 0.11 | 0.88 | −0.55 | 0.52 | 0.26 | −1.49 | −0.30 |
| 56253 | 0.15 | 1.02 | 0.85 | 0.39 | −0.53 | 1.13 | 1.34 | −0.02 | 1.25 | −0.01 | 0.41 | 1.86 |
| 65082 | −0.57 | 1.06 | 1.62 | 0.08 | −0.31 | 1.29 | 0.88 | 1.13 | 0.98 | −0.34 | −0.93 | 2.21 |
| 84719 | −0.68 | −1.18 | 1.53 | 0.94 | 0.46 | 1.00 | 1.54 | 1.14 | 0.61 | −0.14 | 0.05 | 0.66 |
| 253558 | 1.24 | 0.01 | 0.49 | 1.38 | 0.35 | −0.13 | 1.26 | 1.05 | 1.19 | −0.40 | 0.30 | 2.03 |
| 6777 | 0.74 | −0.50 | 1.64 | 0.39 | 0.78 | 0.54 | 1.17 | 1.41 | 0.11 | 0.29 | 0.21 | 0.33 |
| 80025 | −0.61 | 1.82 | 1.80 | 0.20 | 0.10 | 1.03 | 1.26 | 0.57 | 1.24 | −0.42 | −0.59 | −0.24 |
| 84662 | 0.03 | 0.61 | 1.80 | −0.94 | −0.33 | 1.78 | 2.12 | 1.20 | −0.62 | 0.32 | −0.66 | 0.02 |
| 89890 | 1.00 | −0.65 | 2.49 | 1.13 | −0.34 | 1.21 | 0.86 | 0.32 | −0.65 | −0.49 | 0.46 | 1.02 |
| 10390 | 0.57 | 0.67 | 1.39 | 0.54 | −0.69 | 1.68 | 0.96 | 0.88 | 1.49 | −0.90 | 0.27 | −0.72 |
| 2841 | −1.03 | 2.51 | 0.55 | 0.30 | −0.18 | 0.62 | 0.06 | −0.10 | 0.97 | −1.02 | −0.39 | 1.74 |
| 3904 | −0.44 | 0.77 | −0.42 | 0.80 | 2.51 | 0.13 | 0.95 | 1.18 | 0.65 | 0.84 | −1.06 | 1.43 |
| 140460 | 1.30 | −0.11 | 2.16 | 0.77 | −0.72 | 0.50 | 1.35 | 1.65 | 0.95 | −0.18 | −0.56 | 0.11 |
| 9605 | 0.68 | −1.02 | 1.87 | 0.88 | 1.02 | 0.03 | 1.54 | 1.51 | −0.21 | 1.08 | −0.21 | −0.15 |
| 283349 | 0.02 | 0.63 | 1.99 | 0.51 | −0.21 | 1.09 | 1.87 | 0.57 | 1.23 | −0.11 | −0.47 | 0.95 |
| 51246 | 0.13 | 0.60 | 0.57 | 0.51 | 2.19 | 0.73 | 1.63 | 0.37 | 0.08 | 0.50 | 0.06 | 0.81 |
| 148268 | 0.36 | 0.60 | 1.09 | 0.73 | 0.63 | 1.36 | −0.69 | 0.19 | 0.55 | 0.08 | 0.28 | −0.67 |
| 26589 | 0.71 | −0.10 | −0.28 | 2.06 | 1.41 | 1.27 | 0.48 | 0.64 | −1.12 | −0.65 | −0.67 | 0.72 |
| 7049 | −0.17 | 1.32 | 0.17 | 1.16 | 0.92 | 0.95 | 1.02 | −0.25 | 1.40 | −0.92 | −0.32 | 1.46 |
| 2791 | 0.39 | 0.42 | 1.64 | 0.34 | 1.17 | 1.26 | 2.19 | 1.17 | 1.24 | −1.01 | −0.53 | 0.44 |
| 51455 | −0.17 | 0.25 | 1.09 | 1.24 | −0.48 | 0.99 | 0.97 | 0.45 | 1.46 | −0.58 | 0.15 | 1.21 |
| 1178 | 0.09 | 0.20 | 1.78 | 0.51 | −0.13 | 0.64 | 1.87 | 0.98 | 2.19 | −0.60 | 0.32 | −0.25 |
| 79153 | 0.18 | −0.58 | 1.62 | 0.72 | 1.27 | 0.78 | 1.50 | 0.97 | 1.38 | −0.35 | −0.30 | 0.47 |
| 101928524 | 0.40 | 0.08 | 1.77 | 1.16 | −0.46 | 0.73 | 0.94 | 1.31 | 1.31 | −0.02 | −0.31 | 0.93 |
| 11064 | 1.20 | 0.12 | −0.51 | 1.36 | 1.59 | 0.01 | −0.03 | 0.05 | 0.79 | 1.94 | 0.51 | 0.43 |
| 23429 | 0.34 | 0.90 | 1.15 | 0.20 | −1.70 | 0.21 | 1.99 | 1.03 | 0.49 | 0.29 | 0.20 | 0.01 |
| 5170 | 0.56 | 0.68 | 1.76 | 0.38 | 0.14 | 0.81 | 2.13 | 0.73 | 1.11 | 0.69 | −0.52 | 0.06 |
| 10677 | −0.08 | 0.35 | 1.90 | 0.81 | 0.36 | 1.44 | 1.66 | 0.83 | 0.69 | 0.22 | 0.20 | 0.19 |
| 3772 | 0.28 | 0.14 | 2.03 | 0.52 | 0.51 | 1.10 | 1.60 | 0.82 | 1.35 | −0.24 | 0.07 | −0.32 |
| 128061 | −0.79 | 0.76 | 1.14 | 1.31 | 1.07 | 0.65 | 2.07 | 0.85 | 1.41 | −0.41 | −0.93 | 1.16 |
| 50856 | 0.35 | 1.13 | 1.69 | 0.48 | −0.23 | 1.62 | 1.62 | 1.15 | 1.23 | −1.44 | −0.54 | 0.24 |
| 6198 | 0.04 | 1.34 | 0.64 | 0.55 | −3.33 | 0.42 | 0.38 | 1.20 | 0.40 | 0.29 | −0.61 | 0.63 |
| 101927588 | 1.52 | 1.05 | 1.26 | 0.45 | 2.73 | 0.04 | −0.27 | −0.20 | 1.03 | −1.19 | 0.20 | −0.19 |
| 2000 | 0.01 | 1.08 | 2.17 | 0.66 | −0.34 | 0.03 | 1.33 | 0.91 | 1.07 | −0.17 | −0.57 | 0.68 |
| 493753 | 0.77 | 0.60 | 0.88 | 1.75 | −0.83 | 1.79 | 0.96 | 0.54 | 0.70 | −1.86 | −0.62 | 1.51 |
| 23081 | −1.06 | 1.82 | 1.08 | 1.58 | −0.25 | 0.68 | 1.35 | −0.19 | 1.59 | 0.01 | −0.37 | 1.24 |
| 4773 | −0.30 | 1.31 | 0.77 | 0.35 | −0.14 | 1.02 | 1.27 | −0.15 | 1.62 | −0.86 | 0.63 | 1.80 |
| 10178 | 0.49 | 0.94 | 1.45 | 1.09 | 0.56 | 0.80 | 1.72 | 1.02 | 1.43 | −0.28 | −0.48 | 0.71 |
| 4974 | 1.07 | 0.11 | 2.44 | 0.35 | 0.13 | −0.07 | 1.00 | 0.14 | 0.44 | 1.03 | 0.18 | 0.37 |
| 83860 | 1.03 | 0.98 | 1.57 | 0.03 | −0.62 | 0.37 | 1.25 | 0.73 | 1.36 | −1.88 | −1.19 | 0.73 |
| 91612 | 0.63 | 0.76 | 1.04 | 0.38 | 0.62 | 2.28 | 0.47 | −0.43 | 1.24 | 0.05 | −0.19 | 1.48 |
| 2178 | 0.67 | 0.55 | 1.26 | 2.07 | 1.92 | −0.26 | 1.78 | 0.19 | 1.00 | −0.18 | −0.95 | 0.29 |
| 55303 | −0.81 | 1.32 | 1.02 | 0.67 | 0.91 | 1.67 | 0.74 | 0.48 | 1.27 | −0.46 | 0.03 | 1.72 |
| 221002 | 0.68 | 0.33 | 1.15 | 0.77 | 0.59 | 0.99 | 1.68 | 0.12 | 1.29 | −0.88 | −0.21 | 1.82 |
| 3930 | 0.54 | 0.30 | 1.44 | 0.47 | 0.22 | 0.11 | 1.61 | 1.22 | 1.38 | −0.60 | −1.01 | 0.90 |
| 55471 | 0.71 | −1.10 | 1.68 | 1.02 | −0.60 | 1.18 | 1.09 | 1.62 | −0.08 | −0.28 | 0.03 | 1.16 |
| 9354 | 0.18 | 1.46 | 0.54 | 0.75 | 0.02 | 0.38 | 1.12 | 1.15 | −0.05 | 0.60 | −0.09 | 1.36 |
| 83700 | 1.10 | 0.50 | 0.45 | −0.11 | 0.81 | −0.48 | 1.17 | 0.99 | 1.41 | −0.76 | 0.43 | 1.16 |
| 9140 | 0.87 | −0.73 | 0.84 | 1.14 | 0.11 | 0.89 | 0.88 | 1.51 | 0.57 | −0.51 | 0.79 | 0.68 |
| 23526 | −0.76 | 1.54 | 0.94 | 0.81 | 0.93 | −0.64 | 1.23 | 0.37 | 0.55 | 0.58 | −1.06 | 1.41 |
| 23590 | 1.26 | −0.05 | 1.26 | 0.78 | 0.60 | −0.05 | 1.57 | 1.36 | 0.50 | −0.54 | 0.09 | −0.78 |
| 9270 | 0.35 | 0.84 | 0.38 | 1.22 | −1.18 | −0.41 | 1.27 | 0.87 | 0.46 | −0.44 | 0.06 | 1.13 |
| 10627 | 0.00 | 0.44 | 1.93 | 0.96 | −0.62 | 1.75 | 1.01 | −0.14 | 0.95 | −0.08 | −0.14 | 1.40 |
| 26048 | 0.12 | 0.24 | 0.49 | 1.43 | 1.06 | −0.63 | 0.73 | −0.32 | −0.65 | 1.04 | −0.25 | 0.28 |
| 917 | −1.44 | 1.55 | −0.41 | 0.92 | 1.54 | 1.57 | 0.96 | 0.01 | 1.21 | 0.31 | 0.74 | 1.00 |
| 94039 | −0.18 | 0.83 | 0.62 | 0.80 | 1.02 | 1.49 | 1.14 | 0.52 | 1.38 | 0.03 | −0.66 | 1.61 |
| 4215 | 0.08 | 0.86 | 1.75 | 0.38 | 0.31 | 0.62 | 2.04 | 0.84 | 0.33 | 0.39 | −0.56 | −0.11 |
| 10663 | −0.60 | 2.22 | 0.22 | 0.33 | 0.83 | 2.29 | 0.13 | −0.30 | 1.03 | −1.04 | 0.77 | 1.09 |
| 10308 | 0.86 | −0.09 | 1.79 | 0.22 | −0.93 | 1.12 | 1.95 | 1.16 | 1.19 | −0.74 | −0.57 | 0.04 |
| 8527 | 0.23 | 0.20 | 0.57 | 0.04 | 0.51 | −1.51 | 1.22 | 0.55 | −0.40 | 2.52 | −0.15 | 0.52 |
| 926 | −0.16 | 0.61 | 0.47 | 0.71 | 1.26 | 0.68 | 0.83 | 0.32 | 1.73 | −0.67 | 0.70 | 1.62 |
| 9619 | 0.91 | 1.62 | 0.12 | 0.71 | 0.50 | 1.60 | 1.43 | 0.88 | −0.69 | −1.34 | 0.63 | 0.66 |
| 5998 | 1.00 | 0.62 | 0.40 | 0.66 | 1.43 | 1.44 | 0.38 | 0.92 | −0.51 | 0.37 | −0.43 | 0.62 |
| 132625 | 0.56 | 1.09 | 1.45 | 0.38 | 0.01 | 0.27 | 0.66 | 1.12 | 1.63 | −0.10 | −0.30 | 0.31 |
| 88455 | 0.41 | 0.85 | 2.27 | 0.47 | 0.15 | 0.62 | 2.02 | 0.41 | 1.14 | −0.11 | −0.13 | 1.04 |
| 3604 | 0.11 | 0.10 | 1.56 | 0.22 | 0.88 | 2.06 | 1.57 | 1.63 | 0.00 | −0.42 | −0.06 | −0.08 |
| 100507398 | 1.97 | −0.25 | 1.82 | 0.07 | −0.32 | 0.82 | 0.17 | 0.71 | 0.47 | −0.82 | −1.01 | 1.65 |
| 58517 | 0.61 | 0.66 | 0.22 | 0.90 | −0.78 | −0.82 | 1.14 | 0.53 | −1.04 | 1.36 | 0.06 | −0.21 |
| 51192 | 0.36 | −0.18 | 1.98 | 0.69 | 0.41 | 0.96 | 1.55 | 1.30 | 1.40 | 0.22 | −0.50 | 0.29 |
| 2177 | 1.11 | 0.65 | 0.97 | 1.23 | −0.22 | −0.14 | 1.53 | 0.87 | 0.09 | −0.85 | −1.31 | −0.41 |
| 57097 | 0.11 | 0.50 | 0.85 | 0.54 | 0.62 | 0.51 | 0.58 | −0.07 | 2.33 | −1.58 | 0.72 | 1.28 |
| 11214 | 0.25 | 0.59 | 0.75 | 0.43 | 0.66 | 0.71 | 1.74 | 1.23 | 0.55 | 0.35 | 0.42 | 0.28 |
| 4600 | 0.01 | 1.21 | 1.31 | 0.41 | 1.55 | 0.72 | 1.01 | 0.83 | 1.26 | 0.04 | 0.03 | 0.38 |
| 84255 | 0.61 | −0.05 | 1.47 | 0.53 | 0.24 | 1.59 | 1.69 | 1.15 | 1.45 | −1.07 | 0.11 | 0.34 |
| 11216 | −0.62 | 0.19 | 1.36 | 0.45 | −0.89 | 1.30 | 0.18 | 0.87 | 0.61 | 0.07 | 0.80 | 1.27 |
| 162417 | 0.28 | 0.06 | 1.77 | 0.33 | 0.19 | 1.35 | 1.42 | 0.28 | −0.08 | −1.94 | −0.09 | 0.91 |
| 101928617 | −0.61 | 1.50 | 1.43 | 0.45 | 2.39 | 0.53 | 0.62 | 0.59 | 1.14 | 0.11 | −1.13 | 1.12 |
| 10617 | 1.19 | 0.41 | −0.15 | 0.84 | 0.50 | −0.06 | 0.63 | 1.59 | 0.03 | 0.90 | −1.13 | 1.28 |
| 1326 | 0.15 | 0.53 | 2.43 | 0.34 | −1.12 | 1.37 | 1.18 | 0.88 | 1.00 | −1.77 | 0.23 | −0.12 |
| 23315 | 0.60 | 0.19 | 1.76 | 0.60 | 0.89 | 0.67 | 1.35 | 0.90 | −0.11 | 0.73 | 0.17 | 0.38 |
| 10791 | −0.88 | 0.35 | 0.03 | 0.73 | 2.07 | 1.02 | 0.89 | 0.75 | 1.02 | −0.63 | 0.56 | 0.26 |
| 8718 | −0.69 | 0.85 | 0.36 | 1.17 | 0.14 | 1.06 | 0.42 | −0.22 | 0.55 | −0.19 | 0.23 | 1.01 |
| 51571 | 0.20 | 0.99 | 1.67 | 0.76 | 0.02 | 0.74 | 1.74 | 1.09 | 0.96 | 0.20 | −0.06 | 0.70 |
| 283521 | 0.05 | 1.12 | 0.70 | 0.42 | 1.45 | 0.85 | 0.92 | 0.74 | 1.41 | −0.07 | −0.18 | 1.04 |
| 51122 | 0.88 | 0.73 | 1.30 | 0.91 | 0.33 | 0.54 | 1.71 | 0.83 | 0.87 | −1.63 | −0.91 | 1.59 |
| 5372 | 0.70 | 0.87 | 1.51 | −0.30 | 0.03 | 1.53 | 1.61 | 0.65 | 0.66 | 0.40 | 0.84 | −0.63 |
| 9830 | −0.28 | 1.60 | 0.34 | 1.21 | 1.44 | 1.06 | −0.50 | 0.32 | 1.02 | −0.07 | 1.06 | 0.50 |
| 314 | 0.02 | 0.87 | 2.05 | 0.30 | 0.23 | 0.51 | 2.08 | 1.28 | 0.83 | 0.42 | −0.07 | −0.06 |
| 51761 | −1.13 | 1.10 | 0.25 | 0.58 | −0.01 | 0.67 | 0.45 | 0.84 | 3.18 | 0.18 | −0.06 | −0.39 |
| 493861 | 0.88 | 0.96 | −0.75 | 1.10 | 0.37 | 0.11 | 1.50 | 0.27 | 0.40 | −0.55 | 0.44 | 1.12 |
| 6672 | 0.13 | 0.53 | 1.85 | 0.28 | 0.38 | 1.94 | 1.21 | 0.99 | 0.92 | −0.22 | 0.13 | 0.07 |
| 55578 | −0.26 | −0.67 | 0.83 | 0.70 | 0.55 | 0.67 | 1.37 | 1.08 | 1.45 | 0.14 | 0.60 | 1.26 |
| 159 | 0.02 | 0.98 | 1.82 | 0.16 | −1.09 | 0.25 | 0.00 | 0.37 | 1.31 | 1.43 | 0.31 | 0.48 |
| 9683 | −0.13 | 0.83 | 1.89 | 0.35 | 0.69 | 1.18 | 1.74 | 0.67 | 1.27 | −0.16 | −0.02 | 0.27 |
| 26287 | 2.34 | 0.73 | 0.27 | −0.10 | 0.70 | 0.88 | 0.95 | 0.53 | 1.32 | −0.41 | 0.70 | 0.07 |
| 57147 | 0.45 | −0.84 | 1.60 | 0.94 | 0.45 | 1.55 | 1.49 | 1.10 | 0.58 | −0.29 | −0.29 | 0.92 |
| 1808 | 0.07 | 1.64 | 0.89 | 0.96 | 0.15 | 0.52 | 1.13 | 0.85 | 1.00 | −0.53 | −0.26 | 1.02 |
| 23355 | 1.13 | −0.35 | 1.71 | 0.76 | 0.83 | 1.12 | 1.25 | 1.07 | 0.16 | −0.04 | 0.80 | 0.19 |
| 8115 | −1.16 | 1.01 | 1.89 | −0.31 | 1.19 | 1.25 | 1.15 | 0.86 | 1.26 | −0.42 | −0.68 | −0.61 |
| 94120 | −0.17 | 1.37 | 1.68 | 0.14 | −0.31 | 1.89 | 1.29 | 0.21 | 0.72 | −0.94 | 0.11 | 1.11 |
| 22950 | 0.15 | 0.63 | 1.64 | 1.21 | 0.76 | 1.55 | 1.81 | 0.87 | 0.90 | −1.41 | −1.03 | 0.58 |
| 27342 | 0.45 | 0.16 | 2.11 | 0.06 | −0.21 | 1.34 | 1.20 | 1.26 | 1.38 | −0.14 | 0.34 | 0.14 |
| 64926 | 0.27 | 1.48 | 0.15 | 0.69 | 0.94 | −1.20 | 0.52 | 0.77 | 1.39 | 0.60 | −0.20 | 1.57 |
| 100507331 | 1.67 | 0.99 | 0.83 | 0.89 | −0.67 | −0.38 | 0.68 | 0.17 | −0.24 | 1.46 | −0.53 | −0.02 |
| 4068 | 0.32 | 1.15 | −0.38 | 0.44 | −0.19 | 0.42 | 1.40 | 0.95 | 1.39 | −0.63 | −0.42 | 1.64 |
| 60481 | −0.43 | 1.66 | 1.09 | 0.23 | −0.45 | 1.43 | −0.01 | 0.72 | 0.06 | 1.43 | −0.25 | 0.41 |
| 2634 | −0.31 | 1.17 | 1.10 | 0.29 | 1.72 | 1.38 | 1.24 | 0.69 | 1.45 | −0.97 | 0.08 | 0.36 |
| 55824 | −0.87 | 1.01 | 1.35 | 0.89 | 0.88 | 0.78 | 1.87 | 0.94 | 1.37 | 0.01 | −0.14 | 0.95 |
| 79074 | 0.88 | 0.01 | 1.34 | 0.51 | −0.92 | 0.49 | 1.07 | 0.88 | 0.72 | −0.71 | −0.15 | 1.07 |
| 220930 | 1.60 | −0.60 | 1.48 | −0.06 | 0.33 | 0.26 | 2.02 | 1.50 | 0.91 | −0.30 | −0.09 | 0.65 |
| 64421 | 0.27 | 0.78 | 1.57 | 0.87 | 0.15 | 0.12 | 1.70 | 0.38 | 1.38 | −0.35 | −0.41 | 0.79 |
| 64121 | −0.50 | 1.60 | 2.31 | 0.25 | 0.49 | 1.30 | 1.49 | 1.24 | −0.35 | −0.52 | −0.20 | 0.12 |
| 137835 | 0.62 | 0.67 | 1.50 | 0.48 | −0.21 | 0.62 | 1.64 | 0.32 | 1.77 | −0.46 | −0.75 | 1.12 |
| 57674 | 0.09 | 0.75 | 0.98 | 0.66 | 0.81 | 1.43 | 1.18 | 1.14 | 1.29 | −1.06 | 0.98 | 0.24 |
| 728392 | 1.08 | 0.94 | 0.69 | −0.12 | 0.59 | 1.80 | 1.62 | 0.62 | 0.60 | 0.17 | −0.26 | 1.61 |
| 329 | 0.71 | 0.38 | 0.64 | 0.85 | −1.56 | 0.82 | 1.38 | 0.85 | 0.41 | −0.11 | −1.01 | 1.38 |
| 55279 | 0.14 | 0.32 | 2.40 | 0.68 | −0.06 | 1.46 | 1.26 | 0.80 | 1.11 | −0.85 | −0.02 | 0.71 |
| 7107 | −0.70 | 0.82 | 1.49 | 0.72 | 0.40 | 2.26 | 1.48 | 0.57 | 1.00 | −1.92 | −0.21 | 0.14 |
| 9416 | 0.77 | −0.06 | 0.23 | 1.52 | −0.15 | 0.69 | 1.76 | 1.16 | −0.29 | 0.75 | −0.61 | 0.98 |
| 137964 | 0.64 | 2.56 | 0.12 | 0.97 | −1.16 | −0.65 | 0.42 | 1.16 | −2.19 | 0.86 | 0.70 | 0.57 |
| 3431 | −0.78 | 0.86 | 1.19 | 0.66 | 1.43 | 1.56 | 1.06 | 0.55 | 1.33 | −0.27 | 0.16 | 1.14 |
| 4140 | 0.47 | 0.60 | 1.73 | 0.75 | 0.26 | −0.47 | 1.08 | 1.16 | −0.18 | 0.35 | −0.66 | 0.53 |
| 59269 | 0.70 | 0.58 | 0.60 | 1.36 | 1.52 | 0.02 | 1.56 | 1.60 | 1.17 | −1.21 | −0.51 | 0.45 |
| 7846 | 0.66 | 1.03 | 0.56 | 0.63 | 0.04 | 0.74 | 0.87 | 0.79 | 0.77 | 0.19 | −0.39 | −0.33 |
| 100652740 | 0.08 | 0.30 | 0.97 | 1.47 | 0.59 | 0.24 | 0.29 | 0.92 | 1.69 | 0.07 | −0.62 | −0.89 |
| 23398 | 0.73 | 0.21 | 0.44 | 1.35 | −1.17 | 0.10 | 0.47 | 0.70 | 0.67 | 0.97 | −0.42 | 2.08 |
| 158747 | 0.26 | −0.04 | 2.04 | 0.63 | 0.13 | 1.51 | 1.61 | 1.18 | 1.23 | −0.74 | 0.10 | 0.42 |
| 285512 | 0.11 | 0.28 | 1.69 | 0.75 | 0.26 | 0.30 | 2.21 | 0.39 | 1.10 | −0.19 | −0.46 | 0.95 |
| 3937 | −0.27 | 0.84 | 0.90 | 0.75 | −0.13 | 0.47 | 0.49 | 0.50 | 1.43 | −0.03 | 0.81 | 1.85 |
| 730051 | 0.68 | 1.09 | 1.57 | 0.62 | −0.82 | −0.93 | 0.66 | 0.22 | 1.04 | 0.32 | −0.14 | 1.42 |
| 9320 | −0.17 | 1.55 | 1.08 | 0.07 | −2.00 | 0.47 | 0.79 | 0.86 | 0.14 | 0.43 | 0.46 | 0.76 |
| 6932 | −1.09 | 1.37 | 0.50 | 0.73 | 0.44 | 1.17 | 0.61 | 0.47 | 1.06 | 0.39 | 0.39 | 1.63 |
| 79722 | −0.41 | 0.88 | 1.17 | 0.74 | 0.27 | 1.50 | 1.30 | 0.56 | 1.77 | −0.89 | 0.27 | 0.12 |
| 115362 | −0.29 | 1.49 | −0.02 | 1.26 | 1.46 | 1.19 | 0.55 | −0.05 | 1.99 | −0.51 | 0.69 | 1.24 |
| 29909 | −0.56 | 0.88 | 1.28 | 0.65 | 0.90 | 1.66 | 0.88 | 0.47 | 1.80 | −1.36 | 0.24 | 1.33 |
| 440503 | 0.83 | 0.33 | 2.05 | 0.14 | 0.45 | 0.59 | 0.93 | 1.69 | 0.32 | 1.11 | −0.18 | −0.34 |
| 80183 | 0.26 | 0.27 | 1.91 | 0.48 | 0.38 | 1.01 | 1.58 | 1.33 | 1.23 | −0.22 | −0.40 | −0.76 |
| 80709 | −0.21 | 0.67 | 0.92 | 0.61 | 0.58 | 0.59 | 1.01 | 0.60 | 1.04 | 0.99 | 0.16 | 1.28 |
| 10562 | 0.89 | −0.39 | 2.05 | 0.92 | 0.49 | 0.72 | 1.00 | 1.20 | 1.78 | −1.10 | 0.02 | −0.06 |
| 64780 | 1.48 | 0.26 | 0.63 | 1.04 | −0.26 | −0.09 | 2.00 | 1.38 | 0.06 | 1.25 | 0.02 | −0.14 |
| 155038 | −1.06 | 1.56 | 1.12 | 0.57 | 0.95 | 1.41 | 1.01 | 0.72 | 0.50 | −0.21 | 0.33 | 1.28 |
| 1783 | 0.93 | 0.38 | 1.95 | 0.70 | −1.11 | 1.71 | 1.64 | 0.68 | −0.45 | −0.12 | 0.68 | 0.07 |
| 100287569 | 0.39 | 0.32 | 1.40 | 0.59 | 0.71 | −0.45 | 1.00 | 1.34 | 0.79 | −0.50 | −0.35 | 0.59 |
| 5696 | 0.44 | 1.12 | 0.03 | 1.78 | 0.93 | 0.89 | −0.08 | 0.22 | −2.47 | −0.20 | 0.67 | 0.03 |
| 11066 | 1.09 | −0.04 | 1.16 | 0.44 | 1.21 | 0.14 | 0.91 | 1.70 | 1.01 | 0.41 | 0.62 | −0.32 |
| 122553 | 0.36 | −0.63 | 1.39 | 0.56 | −0.93 | 0.77 | 0.80 | 1.42 | 0.31 | 0.49 | −0.09 | 1.37 |
| 50484 | 0.71 | 0.41 | 2.16 | 0.69 | −0.45 | 1.31 | 1.81 | 1.13 | −0.29 | −0.26 | 0.13 | 0.17 |
| 9847 | 0.18 | 0.82 | 1.07 | 1.59 | 0.11 | 1.41 | 0.76 | 0.81 | 0.31 | −0.69 | 0.01 | 1.15 |
| 129285 | 0.41 | −0.58 | 1.41 | 0.55 | 0.54 | 0.61 | 0.82 | 0.98 | 0.85 | 0.04 | 0.54 | 2.65 |
| 9794 | −0.21 | 0.86 | 2.10 | 0.43 | 0.18 | 0.45 | 1.39 | 1.23 | 0.21 | 0.26 | −0.07 | 1.37 |
| 84309 | 1.06 | 1.08 | 0.92 | 1.33 | −1.60 | 0.08 | 1.20 | −0.35 | −0.09 | 0.78 | −0.73 | 0.06 |
| 65258 | 0.10 | 0.91 | 2.01 | 0.10 | −1.04 | 1.16 | 2.05 | 0.81 | 0.84 | −0.12 | −0.49 | 0.44 |
| 5562 | 0.85 | −0.31 | 2.07 | 0.30 | −0.88 | 0.95 | 1.03 | 0.81 | 1.47 | 0.02 | 0.12 | 1.35 |
| 123036 | −1.10 | 1.32 | 0.58 | 0.73 | 0.34 | 1.88 | 0.62 | 0.25 | 1.61 | −1.02 | 0.44 | 1.77 |
| 84166 | 0.03 | 1.45 | 0.08 | 1.27 | 1.65 | −0.26 | 0.73 | 0.73 | 1.25 | 0.76 | 0.07 | 0.89 |
| 253143 | 0.16 | 0.30 | 1.74 | 1.33 | 1.03 | 0.73 | 1.99 | 0.97 | 0.09 | −0.11 | 0.01 | −0.40 |
| 1236 | −0.91 | 1.10 | −0.17 | 0.59 | 1.24 | 0.65 | 0.70 | 0.96 | 1.18 | −0.18 | 0.29 | 1.06 |
| 266747 | 0.35 | 0.48 | 1.77 | 0.88 | 0.42 | 0.75 | 1.35 | 0.73 | 1.32 | −0.25 | −0.07 | 0.66 |
| 25988 | −0.63 | 0.89 | 0.80 | 1.02 | −0.37 | 0.31 | 0.61 | 0.39 | 0.38 | 0.39 | 0.75 | 0.64 |
| 6515 | 0.47 | 0.65 | 1.85 | 0.11 | −0.06 | 0.41 | 1.51 | 1.55 | 1.39 | −0.40 | −0.12 | −0.35 |
| 90592 | 1.21 | −0.05 | 1.09 | 1.02 | 0.46 | 0.73 | 1.68 | 1.49 | −0.92 | 0.64 | −0.39 | 1.35 |
| 6867 | 0.49 | 0.94 | 2.45 | 0.30 | −0.47 | 0.69 | 1.84 | 0.32 | 0.56 | −0.40 | −1.37 | −0.74 |
| 23041 | 0.77 | −1.09 | 0.76 | 0.48 | −0.39 | 0.74 | 1.89 | 0.63 | 1.64 | −0.08 | 0.66 | 1.60 |
| 100289230 | 0.17 | 0.81 | 1.13 | 0.29 | −1.19 | 1.80 | 1.95 | 0.86 | 0.19 | −2.01 | 0.16 | 0.13 |
| 57169 | 0.09 | 0.55 | 1.50 | 0.33 | 0.83 | 1.63 | 1.61 | 0.13 | 1.45 | −0.63 | 0.03 | 0.98 |
| 1105 | 0.72 | 0.97 | 1.24 | 0.49 | −1.30 | 0.11 | 1.55 | 0.86 | 0.95 | −0.32 | 0.12 | 0.77 |
| 943 | 0.47 | 0.19 | 1.34 | 0.36 | 1.00 | 0.25 | 1.11 | 2.05 | −0.21 | −0.35 | 0.17 | −1.44 |
| 3275 | −0.31 | 0.48 | 1.06 | 0.41 | 0.95 | 1.11 | 0.74 | 0.81 | 0.07 | 1.71 | 0.51 | 1.21 |
| 8851 | −0.36 | 0.57 | 2.04 | −0.04 | 0.72 | 1.27 | 0.56 | 0.68 | 1.06 | −0.27 | 0.48 | 0.78 |
| 168537 | −1.22 | 1.09 | 0.82 | 1.02 | 0.81 | 1.09 | 1.16 | 0.58 | 1.38 | 0.38 | −0.37 | 1.66 |
| 256236 | 0.59 | 0.85 | 0.80 | 0.33 | 1.03 | 0.91 | 0.55 | 1.48 | 1.29 | −1.55 | −0.27 | 0.15 |
| 26235 | 1.21 | −0.26 | 1.84 | 0.70 | −0.39 | 1.09 | 2.06 | 0.36 | 1.17 | −1.42 | −0.11 | 0.64 |
| 9363 | −0.44 | 1.27 | 1.45 | −0.19 | −0.26 | 1.01 | 0.60 | 0.92 | −0.10 | −0.16 | 1.03 | −0.53 |
| 729683 | −0.65 | 0.87 | 1.30 | 0.21 | 1.98 | 0.50 | 0.61 | −0.72 | 0.38 | 1.28 | 0.16 | 1.17 |
| 388969 | −0.40 | 1.18 | 1.54 | 0.74 | −1.94 | 0.39 | 1.23 | 0.79 | −0.18 | 1.07 | −0.04 | 1.74 |
| 4818 | −0.12 | 0.57 | 0.76 | 1.05 | 0.49 | 0.11 | 0.45 | 0.90 | 2.00 | −0.75 | 0.16 | 1.11 |
| 93953 | 0.00 | −0.35 | 1.64 | 1.34 | −0.42 | 0.96 | 1.36 | 0.82 | 1.36 | −0.55 | −0.15 | 0.92 |
| 28982 | 0.26 | 1.27 | 1.28 | 1.42 | −0.60 | 0.68 | 1.61 | −0.22 | −0.74 | −0.24 | −0.61 | 1.83 |
| 84911 | 0.47 | −0.60 | 0.73 | 0.60 | 0.16 | 1.09 | 1.40 | −0.04 | 2.50 | −0.92 | −0.46 | 0.44 |
| 66036 | −0.12 | 1.79 | 1.40 | 0.57 | −0.19 | 0.54 | 2.24 | 0.42 | 1.37 | −0.11 | −0.52 | 1.38 |
| 9750 | −0.34 | 0.82 | 1.48 | 0.74 | 0.49 | 0.52 | 0.91 | 0.47 | 1.42 | 0.37 | −0.08 | 1.47 |
| 9938 | −0.26 | 1.05 | 1.22 | 0.74 | 0.82 | 0.41 | 1.80 | 0.81 | 0.70 | 0.64 | 0.06 | 0.58 |
| 120425 | 0.50 | 0.77 | 1.52 | 0.76 | 0.58 | 1.25 | 1.31 | 1.33 | 0.85 | −0.58 | −0.15 | 0.79 |
| 6840 | 0.29 | 0.86 | 1.20 | 0.59 | 0.19 | 0.74 | 1.52 | 0.40 | 1.72 | −0.21 | 0.39 | 0.62 |
| 26268 | 0.19 | 1.31 | 1.98 | 1.07 | −0.61 | 1.10 | 1.08 | −0.30 | 1.30 | −0.88 | 0.14 | 0.52 |
| 55206 | 0.30 | 0.70 | 2.42 | 0.60 | −0.30 | 0.70 | 2.19 | 0.42 | 0.46 | 0.47 | −0.23 | 0.26 |
| 355 | 0.75 | −0.19 | 1.88 | 0.04 | 0.15 | 2.14 | 1.10 | 0.82 | 1.07 | −1.25 | −0.22 | 0.34 |
| 50615 | −0.81 | 1.49 | 1.00 | 0.22 | 0.90 | 1.16 | 0.94 | 0.97 | 0.88 | −1.86 | 0.04 | −0.03 |
| 221178 | 0.40 | 1.14 | 1.04 | 0.40 | 0.98 | 1.33 | 0.19 | 1.08 | 1.15 | 0.26 | 0.44 | 1.42 |
| 11104 | 0.15 | −0.35 | 1.56 | 1.05 | 1.98 | 0.36 | 1.03 | 1.12 | −0.19 | 0.52 | −1.14 | 0.36 |
| 1117 | −0.94 | 1.73 | 0.09 | 0.77 | 1.18 | 2.05 | −0.83 | 1.49 | −0.09 | 0.11 | 0.27 | 0.61 |
| 374969 | 0.52 | 0.95 | 1.62 | 0.39 | 0.72 | 1.64 | 1.69 | 0.74 | 0.50 | −0.87 | −0.15 | 0.19 |
| 6285 | 0.66 | 1.66 | 0.22 | −0.29 | 0.23 | 0.47 | 2.40 | −0.14 | 1.60 | 0.08 | −0.53 | 0.23 |
| 387357 | −0.25 | 0.42 | 0.58 | 0.88 | 0.38 | 1.49 | 1.05 | 0.38 | 1.75 | −0.66 | 0.32 | 2.20 |
| 2960 | 0.46 | 0.05 | 1.75 | 0.60 | 0.45 | 1.98 | 1.10 | 0.63 | 0.93 | −0.86 | 0.37 | 0.93 |
| 100129550 | 0.40 | 0.98 | 2.25 | 0.42 | −0.51 | 0.93 | 1.92 | 0.54 | 0.95 | 0.15 | −0.23 | −0.07 |
| 54520 | 0.56 | 1.00 | 1.95 | 0.57 | 0.20 | 0.83 | 1.02 | 0.45 | 1.01 | 0.46 | −0.36 | 0.04 |
| 10302 | 0.47 | 1.39 | 0.92 | 0.94 | 2.08 | 1.20 | 0.86 | −0.13 | 0.27 | −0.68 | −0.41 | 1.09 |
| 50939 | 0.25 | 0.88 | 1.11 | 1.52 | 0.97 | −0.52 | 0.34 | −0.48 | 0.70 | 0.57 | −1.29 | −0.37 |
| 8802 | 0.65 | 0.50 | 1.05 | 1.24 | −2.63 | 1.10 | 0.94 | 0.44 | −0.40 | −0.45 | 0.01 | −0.50 |
| 23332 | 0.71 | 0.25 | 1.48 | 1.22 | −0.75 | 0.32 | 1.97 | 0.41 | −1.34 | −0.45 | 0.38 | 0.22 |
| 203328 | −0.31 | 0.84 | 0.94 | 0.92 | 0.73 | 1.34 | 1.12 | 0.86 | 0.63 | −1.00 | −0.47 | 0.74 |
| 126231 | −0.25 | 2.14 | 0.29 | 1.06 | 0.34 | 0.75 | 1.21 | −0.48 | 0.66 | 0.36 | 0.13 | 1.63 |
| 6993 | 0.82 | 0.55 | 1.52 | −0.09 | 0.35 | 2.13 | 1.40 | 0.11 | 1.40 | −0.55 | 0.33 | 0.40 |
| 58500 | 1.21 | 1.15 | 2.33 | 0.54 | −0.27 | 0.72 | 0.78 | 0.11 | −0.64 | 0.90 | −0.60 | 0.86 |
| 10507 | −0.04 | 1.02 | 1.83 | 0.47 | 0.44 | 0.19 | 1.52 | 0.86 | 0.83 | 0.35 | 0.01 | 0.98 |
| 170482 | 0.78 | 1.64 | 0.77 | 0.99 | −0.35 | 2.06 | 0.48 | 0.57 | 2.67 | −0.61 | −0.90 | −0.37 |
| 3004 | −1.17 | 0.69 | −0.20 | 1.10 | 1.03 | 1.29 | −0.34 | 0.50 | 0.91 | 0.71 | 1.02 | 1.57 |
| 64895 | −0.61 | 0.09 | 1.82 | 0.86 | 0.06 | 1.01 | 1.49 | 1.11 | 0.78 | 0.00 | −0.08 | 0.87 |
| 7850 | 0.37 | 0.31 | 1.82 | 0.25 | 0.17 | 0.98 | 1.74 | 1.34 | 0.74 | −0.71 | −0.03 | −0.58 |
| 93594 | −0.83 | 1.85 | −0.23 | 1.06 | 0.91 | 0.06 | 1.25 | 0.46 | 1.95 | 0.16 | −0.09 | 1.96 |
| 23215 | 0.21 | 1.43 | 0.54 | 0.85 | 0.29 | −0.17 | 1.41 | 0.17 | −0.20 | 1.44 | 0.15 | −0.23 |
| 80196 | −0.35 | 0.42 | 1.89 | 0.77 | 0.07 | 1.69 | 1.03 | 1.17 | 1.06 | −0.58 | 0.14 | 0.84 |
| 284415 | 0.08 | 0.88 | 2.09 | 1.00 | 0.02 | −0.40 | 1.54 | 1.87 | 1.64 | −0.13 | −0.06 | −1.12 |
| 56882 | 0.06 | 0.75 | 2.04 | 0.57 | −0.17 | 0.59 | 2.13 | 0.25 | 0.42 | 0.57 | 0.11 | −0.13 |
| 6508 | 1.27 | 1.67 | 0.97 | 0.51 | 0.51 | 1.58 | 1.36 | 0.27 | −0.80 | −0.44 | 0.23 | 0.78 |
| 79663 | 0.55 | 0.94 | 2.44 | 0.67 | −0.06 | 1.07 | 1.48 | 0.26 | 1.56 | −0.03 | −0.58 | 0.72 |
| 474344 | −1.13 | 1.63 | 0.77 | 0.91 | 0.90 | 1.05 | 0.46 | 0.33 | 0.94 | 0.17 | −0.10 | 1.94 |
| 100505746 | 0.26 | 1.02 | 0.80 | 1.13 | 0.83 | 0.59 | 1.35 | 1.54 | 1.32 | −0.57 | −0.63 | 1.22 |
| 1293 | 1.42 | 0.70 | 0.20 | 0.95 | 0.85 | 1.33 | 1.54 | 0.96 | 1.16 | −0.57 | −0.23 | −0.15 |
| 10294 | 0.61 | 0.81 | 1.92 | 0.29 | −2.02 | 1.93 | 0.68 | 0.58 | 1.76 | −0.19 | 0.13 | 1.26 |
| 3091 | 0.92 | 0.47 | 1.45 | 0.07 | 0.04 | 0.63 | 1.66 | 1.04 | 0.98 | −2.00 | −0.71 | 0.26 |
| 79573 | −0.49 | 0.89 | 1.51 | 1.47 | 0.14 | 0.83 | 0.66 | 0.81 | 0.93 | −0.70 | −0.33 | 2.34 |
| 9208 | 0.36 | 0.68 | 1.13 | 0.63 | −1.15 | 0.13 | 1.25 | 1.10 | −0.10 | 0.95 | −0.35 | 0.52 |
| 29116 | 0.05 | 2.07 | 0.69 | 0.37 | −0.43 | 0.03 | 1.95 | 0.12 | 0.83 | −0.33 | 0.11 | 1.50 |
| 92370 | 0.72 | 0.55 | 1.14 | 0.71 | 0.13 | 1.12 | 1.67 | 1.06 | 0.80 | 0.04 | −0.29 | 1.12 |
| 9546 | −0.42 | 0.93 | 1.19 | 0.64 | 1.81 | 0.58 | 1.19 | 0.26 | −1.08 | 0.38 | 0.71 | 0.90 |
| 253018 | −0.40 | 0.37 | 1.54 | 0.33 | 0.70 | 1.54 | 2.03 | 0.29 | 1.51 | 0.00 | −0.62 | 0.35 |
| 10096 | −0.18 | 0.65 | 1.15 | 1.29 | −0.25 | 0.38 | 0.69 | 1.65 | 1.38 | −0.20 | −0.45 | 0.82 |
| 84138 | −0.11 | 1.94 | 0.71 | 0.61 | 0.92 | 1.42 | 0.62 | 0.53 | −0.60 | −0.02 | −1.03 | 0.89 |
| 8440 | −1.44 | 1.15 | 0.75 | 0.32 | 1.35 | 0.51 | 1.57 | 0.37 | 1.51 | 0.32 | −0.17 | 1.03 |
| 7277 | −0.14 | 0.59 | 1.42 | 0.67 | 1.32 | 0.97 | 0.98 | 0.59 | 1.01 | −0.39 | −0.49 | 0.50 |
| 8269 | 0.72 | 0.93 | 1.95 | 0.42 | 0.54 | 1.96 | −0.12 | 0.60 | −0.65 | −0.21 | 0.84 | 0.28 |
| 8807 | −0.16 | 0.55 | 1.98 | 0.82 | 0.56 | 0.78 | 1.65 | 1.12 | 1.70 | −0.51 | −0.05 | 0.59 |
| 1235 | −0.68 | 1.67 | 1.50 | 0.33 | 0.37 | 1.55 | 0.12 | −0.25 | 0.85 | −0.46 | −0.84 | 0.85 |
| 7059 | 1.95 | 0.89 | 1.47 | −0.02 | 1.14 | −0.06 | −0.22 | 0.71 | −0.72 | 0.00 | 0.33 | −0.07 |
| 54971 | −0.42 | 1.75 | 2.08 | 0.38 | 0.58 | 0.76 | 1.60 | 0.63 | 0.70 | 0.64 | −0.99 | −0.06 |
| 57677 | 1.02 | 0.18 | 0.05 | 0.35 | −0.01 | 0.40 | 2.20 | 0.17 | 0.56 | 0.74 | −0.57 | 0.90 |
| 6890 | 0.39 | 0.26 | 0.79 | 0.72 | 0.98 | 1.34 | 1.71 | 0.64 | 0.63 | −1.03 | −0.17 | 0.66 |
| 22944 | 0.09 | −0.32 | 1.94 | 0.73 | 0.00 | 1.44 | 1.28 | 1.19 | 1.12 | −0.05 | −0.18 | 1.23 |
| 26512 | 0.00 | 0.49 | 1.72 | 0.26 | −0.96 | 1.66 | 1.82 | 1.78 | 0.92 | 0.02 | −0.55 | 1.06 |
| 92797 | 1.09 | −0.03 | 1.19 | 1.08 | −0.37 | 0.13 | 1.17 | 1.27 | 2.08 | 0.29 | 0.15 | 0.28 |
| 5481 | 0.38 | −0.13 | 1.29 | 0.80 | −0.54 | 1.49 | 0.89 | 1.07 | −0.15 | −0.44 | −0.16 | 1.25 |
| 22897 | 0.20 | 1.63 | −0.43 | 0.51 | 3.52 | 0.05 | 0.28 | 0.09 | 0.02 | 1.28 | −0.28 | 1.15 |
| 3620 | −0.36 | 0.77 | 1.28 | 0.63 | 0.56 | 1.92 | 1.06 | 0.66 | 1.37 | −1.58 | 0.08 | 0.03 |
| 64766 | 0.93 | 0.83 | 0.37 | 1.77 | 0.42 | −0.57 | 0.49 | 1.17 | 0.48 | 1.12 | −0.57 | 1.49 |
| 8809 | 0.39 | 0.19 | 1.61 | 0.57 | 0.17 | 1.19 | 1.66 | 1.13 | 1.69 | −0.60 | 0.21 | 0.52 |
| 116984 | −0.12 | 1.32 | 0.97 | 1.27 | 1.02 | 0.99 | 1.41 | 1.06 | 1.03 | −1.31 | −0.37 | 1.42 |
| 440823 | −0.05 | 0.90 | 0.56 | 0.24 | 0.70 | 0.92 | −0.15 | 1.56 | 1.36 | 0.18 | 0.96 | 0.39 |
| 59340 | −0.36 | −0.17 | 1.40 | 0.32 | 1.80 | 0.61 | 1.00 | 1.12 | 1.55 | 0.73 | 0.56 | 0.03 |
| 197259 | −0.41 | 0.04 | 2.12 | 0.83 | 1.57 | 1.26 | 0.65 | 0.65 | 1.09 | −0.49 | −0.28 | 1.28 |
| 23670 | 0.25 | 0.73 | 1.86 | 0.32 | 0.66 | 1.42 | 1.25 | 0.79 | 1.14 | −0.51 | 0.44 | 0.65 |
| 643314 | 1.32 | 0.38 | 0.60 | 0.37 | −0.32 | 0.79 | 1.39 | 1.01 | 0.50 | 0.14 | 0.09 | 0.93 |
| 28526 | −0.85 | 1.74 | 0.35 | 1.07 | 0.55 | 0.93 | 0.70 | −0.11 | 1.52 | 0.75 | −0.26 | 1.46 |
| 55096 | 0.85 | 1.02 | 1.94 | 0.13 | −0.08 | 1.22 | 1.64 | 0.56 | 0.61 | −0.58 | −0.16 | 0.11 |
| 2796 | −0.16 | 0.76 | 0.78 | 0.24 | 1.70 | 0.70 | 0.46 | 0.48 | −0.44 | 0.63 | 0.13 | 1.22 |
| 54509 | −0.18 | 1.61 | 0.96 | 0.22 | 0.15 | 0.33 | −0.36 | 1.34 | 0.29 | −1.91 | −0.14 | −0.07 |
| 11120 | −1.10 | 0.74 | 1.59 | 1.22 | 1.25 | 0.54 | 1.62 | 0.07 | 0.36 | 0.70 | 0.07 | 0.37 |
| 84869 | 0.50 | 0.27 | 0.41 | 1.51 | 0.07 | 1.46 | 1.79 | 1.91 | 0.17 | 0.75 | −0.61 | 0.70 |
| 28991 | 0.04 | 0.57 | 2.23 | 0.61 | 0.06 | 1.64 | 1.49 | 0.50 | −0.44 | −0.56 | −0.06 | 0.31 |
| 154141 | 1.28 | −0.32 | 1.71 | 0.48 | 0.68 | 0.97 | 1.43 | 0.93 | 0.11 | 0.16 | 0.55 | −0.53 |
| 3659 | 0.46 | 0.85 | 1.63 | 0.44 | 0.41 | 0.87 | 2.23 | −0.13 | 1.69 | −0.78 | −0.51 | 0.22 |
| 154007 | 0.84 | −0.19 | 1.65 | 0.32 | 0.02 | 0.39 | 1.85 | 1.53 | 1.44 | −0.29 | −0.70 | 0.17 |
| 116835 | 0.79 | 0.78 | −0.15 | −0.17 | 2.37 | 0.99 | 0.77 | 0.54 | 1.12 | 0.12 | 0.26 | −0.34 |
| 9488 | 0.43 | 0.80 | 1.28 | 0.77 | 1.28 | −0.25 | 2.06 | 0.67 | −0.14 | 0.41 | −0.12 | 0.82 |
| 100293516 | 1.59 | 0.62 | 0.76 | 1.92 | 0.19 | 0.23 | 1.07 | 0.75 | 0.57 | 1.62 | −0.80 | 1.85 |
| 9217 | 0.05 | 1.47 | 1.18 | 0.82 | 0.73 | 1.49 | 1.54 | −0.05 | 0.49 | 0.58 | −0.07 | 0.14 |
| 10538 | 1.05 | −0.09 | 0.04 | 1.11 | 0.93 | 1.13 | 1.13 | 1.29 | −0.07 | 0.25 | −0.27 | 0.46 |
| 6935 | 0.62 | 0.40 | 0.57 | 0.10 | −0.09 | 0.26 | 1.22 | 1.16 | 0.67 | 0.42 | 0.47 | 0.38 |
| 10443 | 0.19 | 0.33 | 0.96 | 1.05 | 0.11 | 0.71 | 1.52 | 0.69 | 0.92 | −0.06 | 0.56 | 1.76 |
| 3837 | −0.05 | 1.52 | 1.10 | 1.48 | 0.98 | 0.29 | 0.83 | 1.02 | 0.88 | 0.28 | 0.41 | 0.01 |
| 81698 | 0.58 | 0.50 | 1.27 | −0.17 | 1.15 | 1.10 | 1.20 | 0.94 | −0.19 | −0.26 | 0.57 | −0.32 |
| 2643 | 0.50 | 0.99 | 1.44 | −0.26 | 0.30 | 0.89 | 0.61 | −0.21 | 1.51 | −2.32 | −0.02 | 1.22 |
| 57559 | −0.87 | 1.17 | 0.28 | 0.80 | 1.87 | 1.24 | 0.52 | 0.84 | 0.96 | −0.53 | −0.33 | 1.18 |
| 116842 | −0.18 | 1.22 | 1.47 | 1.24 | −0.34 | 1.56 | 0.50 | 0.64 | 1.46 | −0.72 | −0.53 | 1.59 |
| 202 | 0.01 | 1.52 | 1.00 | 0.75 | 0.90 | 0.73 | 0.66 | 0.40 | 1.37 | 0.03 | −0.21 | 1.09 |
| 9934 | 0.14 | 0.61 | 1.54 | 0.87 | 0.14 | 1.44 | 2.04 | 0.82 | 0.82 | −1.17 | 0.24 | 0.64 |
| 100216546 | 0.72 | 0.98 | 1.37 | 0.09 | −0.33 | 1.60 | 1.27 | 1.30 | 1.61 | −0.28 | −0.58 | 0.24 |
| 9692 | 0.30 | 1.02 | 0.38 | −0.56 | 2.06 | 0.68 | 0.52 | 1.27 | 0.32 | 0.38 | −0.74 | 0.68 |
| 89845 | 0.22 | 1.66 | 0.08 | 1.44 | 1.47 | 0.53 | 0.21 | 1.07 | −0.51 | 0.40 | −0.76 | 1.25 |
| 26034 | −0.60 | 1.24 | 1.25 | 0.80 | 1.40 | 1.07 | 1.25 | 0.92 | 1.34 | −0.11 | −0.16 | 1.08 |
| 10906 | 0.29 | 0.91 | 1.62 | 0.23 | 1.86 | 1.01 | 1.12 | 1.38 | 0.13 | 0.05 | 0.27 | −0.50 |
| 84811 | 0.33 | 0.74 | 1.40 | 1.66 | 0.12 | −0.13 | 1.56 | 1.04 | 0.52 | 0.12 | −0.11 | 0.46 |
| 100527964 | 0.70 | 0.47 | 0.30 | 1.34 | 0.37 | −0.34 | −0.21 | 1.15 | 0.84 | 1.60 | −0.37 | 1.50 |
| 118426 | 0.15 | 1.01 | 0.95 | 0.81 | 0.19 | 1.80 | 1.21 | 1.04 | 1.61 | −0.72 | 0.09 | 0.21 |
| 54331 | −0.04 | 0.27 | 1.33 | 0.71 | 0.62 | 0.50 | 1.62 | 1.42 | 1.14 | 0.13 | 0.26 | 0.49 |
| 94081 | 0.60 | 0.43 | 1.31 | 1.26 | −0.58 | 1.19 | 0.68 | 1.33 | 0.42 | −0.71 | 0.21 | 0.72 |
| 145474 | 0.87 | 1.04 | 1.28 | 0.06 | 0.15 | 1.37 | 2.52 | 1.11 | 1.12 | −0.57 | −0.27 | 0.00 |
| 84969 | 0.74 | 0.73 | 0.71 | 0.64 | 0.33 | 1.83 | 1.20 | 0.92 | 0.57 | −0.79 | −0.46 | −0.03 |
| 4907 | −0.61 | 2.13 | 0.02 | 0.50 | −0.38 | 0.88 | 0.65 | −0.17 | 1.47 | 1.62 | 0.16 | 0.74 |
| 51735 | −0.04 | 1.08 | 1.90 | 0.65 | −0.13 | 1.37 | 1.58 | 0.38 | 1.25 | −0.35 | −0.08 | 0.48 |
| 9953 | 0.36 | 1.18 | 1.23 | 1.47 | 0.26 | 0.98 | 1.12 | 0.86 | 0.96 | −0.19 | 0.40 | 1.56 |
| 23112 | −0.24 | 1.51 | 1.77 | 0.55 | 0.67 | 1.02 | 1.48 | 0.19 | 0.40 | 1.01 | 0.23 | 0.65 |
| 91526 | 0.18 | 0.97 | 1.90 | 1.07 | 0.55 | 1.12 | 0.46 | 1.14 | 0.65 | −0.09 | −0.04 | 1.26 |
| 101928017 | −0.17 | 1.87 | 0.03 | 0.21 | 0.43 | 1.92 | 0.24 | 0.61 | 0.89 | −0.88 | −0.07 | −0.43 |
| 84859 | −0.48 | 0.28 | 1.08 | 1.43 | 1.70 | 0.97 | 0.72 | 0.52 | 0.21 | 0.59 | 0.52 | 1.69 |
| 159013 | −0.09 | 0.87 | 2.04 | 1.26 | 0.08 | 0.73 | 1.49 | 1.14 | 0.74 | −0.06 | −0.34 | 1.06 |
| 23208 | 0.61 | 1.08 | 1.11 | 1.25 | 0.52 | 0.54 | 0.41 | 1.02 | 0.94 | −1.17 | −0.67 | 1.05 |
| 101928649 | 1.25 | −0.40 | 1.41 | 1.37 | 0.14 | −0.38 | 1.40 | 1.81 | −0.35 | −0.41 | −1.01 | 0.57 |
| 85459 | 0.76 | 0.54 | 1.00 | 0.69 | −0.64 | 0.10 | 2.21 | 1.43 | 0.35 | 0.59 | −0.59 | −0.20 |
| 9617 | 0.54 | −0.11 | 0.92 | 1.44 | 0.33 | 0.80 | 1.02 | 0.78 | 0.74 | −0.78 | −0.53 | 2.48 |
| 56898 | 1.59 | 0.83 | 0.88 | 0.66 | 0.29 | 0.73 | −0.24 | 1.14 | 0.11 | 0.60 | −1.18 | 0.62 |
| 387882 | 1.33 | 1.38 | 1.03 | 0.70 | 0.51 | 0.55 | 0.32 | 0.55 | 1.23 | −0.65 | −0.91 | 1.27 |
| 677769 | 0.50 | 2.09 | 0.27 | 0.72 | 0.83 | 1.44 | 0.65 | 1.55 | 0.36 | 0.16 | −0.28 | −0.25 |

TABLE 3

| gene | symbol | name | fisherz | 34 | 35 | 36 |
|---|---|---|---|---|---|---|
| 56904 | SH3GLB2 | SH3-domain | 3.67 | 0.86 | 0.03 | 0.37 |
| | | GRB2-like | | | | |
| | | endophilin B2 | | | | |
| 6464 | SHC1 | SHC (Src | 3.56 | −0.43 | −1.06 | −1.09 |
| | | homology 2 | | | | |
| | | domain | | | | |
| | | containing) | | | | |
| | | transforming | | | | |
| | | protein 1 | | | | |
| 79087 | ALG12 | ALG12, | 3.45 | 1.14 | −0.24 | 0.59 |
| | | alpha-1,6- | | | | |
| | | mannosyltransferase | | | | |
| 101928190 | LOC101928190 | uncharacterized | 3.43 | 1.24 | 0.71 | 0.74 |
| | | LOC101928190 | | | | |
| 3416 | IDE | insulin- | 3.17 | 0.76 | 0.80 | −1.08 |
| | | degrading | | | | |
| | | enzyme | | | | |
| 8001 | GLRA3 | glycine | 3.16 | −0.21 | 1.30 | 0.32 |
| | | receptor, | | | | |
| | | alpha 3 | | | | |
| 3727 | JUND | jun D proto- | 3.13 | 0.20 | −0.25 | 0.01 |
| | | oncogene | | | | |
| 10945 | KDELR1 | KDEL (Lys- | 3.12 | 0.44 | −0.09 | −0.61 |
| | | Asp-Glu-Leu) | | | | |
| | | endoplasmic | | | | |
| | | reticulum | | | | |
| | | protein | | | | |
| | | retention | | | | |
| | | receptor 1 | | | | |
| 5025 | P2RX4 | purinergic | 3.09 | 0.96 | −0.42 | 0.15 |
| | | receptor P2X, | | | | |
| | | ligand-gated | | | | |
| | | ion channel, 4 | | | | |
| 29920 | PYCR2 | pyrroline-5- | 3.07 | −0.04 | 0.00 | 0.49 |
| | | carboxylate | | | | |
| | | reductase | | | | |
| | | family, | | | | |
| | | member 2 | | | | |
| 101929248 | LOC101929248 | uncharacterized | 3.01 | 0.19 | −0.17 | −0.18 |
| | | LOC101929248 | | | | |
| 23646 | PLD3 | phospholipase | 2.98 | 0.65 | −0.64 | 0.06 |
| | | D family, | | | | |
| | | member 3 | | | | |
| 10280 | SIGMAR1 | sigma non- | 2.97 | 0.22 | 0.01 | 0.14 |
| | | opioid | | | | |
| | | intracellular | | | | |
| | | receptor 1 | | | | |
| 140699 | MROH8 | maestro heat- | 2.96 | 0.32 | 1.56 | −0.41 |
| | | like repeat | | | | |
| | | family | | | | |
| | | member 8 | | | | |
| 4669 | NAGLU | N- | 2.94 | 0.38 | −0.30 | −0.61 |
| | | acetylglucosaminidase, | | | | |
| | | alpha | | | | |
| 8341 | HIST1H2BN | histone cluster | 2.91 | 0.70 | −0.94 | 0.44 |
| | | 1, H2bn | | | | |
| 2519 | FUCA2 | fucosidase, | 2.90 | 0.53 | −0.11 | 0.73 |
| | | alpha-L-2, | | | | |
| | | plasma | | | | |
| 8985 | PLOD3 | procollagen- | 2.88 | −0.01 | −0.40 | −0.24 |
| | | lysine, 2- | | | | |
| | | oxoglutarate | | | | |
| | | 5-dioxygenase 3 | | | | |
| 1690 | COCH | cochlin | 2.87 | 1.54 | 0.02 | 0.18 |
| 51114 | ZDHHC9 | zinc finger, | 2.86 | −0.02 | 0.17 | 0.23 |
| | | DHHC-type | | | | |
| | | containing 9 | | | | |
| 55907 | CMAS | cytidine | 2.85 | 1.20 | −0.01 | 0.04 |
| | | monophosphate | | | | |
| | | N- | | | | |
| | | acetylneuraminic | | | | |
| | | acid | | | | |
| | | synthetase | | | | |
| 144811 | LACC1 | laccase | 2.84 | 0.36 | −0.91 | 0.83 |
| | | (multicopper | | | | |
| | | oxidoreductase) | | | | |
| | | domain | | | | |
| | | containing 1 | | | | |
| 92421 | CHMP4C | charged | 2.84 | 0.78 | −0.20 | −0.06 |
| | | multivesicular | | | | |
| | | body protein | | | | |
| | | 4C | | | | |
| 23475 | QPRT | quinolinate | 2.83 | 0.37 | 0.89 | −0.26 |
| | | phosphoribosyltransferase | | | | |
| 54913 | RPP25 | ribonuclease | 2.77 | −0.43 | 0.75 | −0.35 |
| | | P/MRP 25 kDa | | | | |
| | | subunit | | | | |
| 4645 | MYO5B | myosin VB | 2.76 | −0.18 | −0.49 | −1.23 |
| 9917 | FAM20B | family with | 2.74 | 0.12 | −0.53 | 0.50 |
| | | sequence | | | | |
| | | similarity 20, | | | | |
| | | member B | | | | |
| 57213 | SPRYD7 | SPRY domain | 2.73 | −1.19 | −0.63 | −0.47 |
| | | containing 7 | | | | |
| 113829 | SLC35A4 | solute carrier | 2.73 | 0.87 | 1.35 | −0.10 |
| | | family 35, | | | | |
| | | member A4 | | | | |
| 100128164 | LOC100128164 | four and a half | 2.72 | 0.42 | 0.07 | 0.32 |
| | | LIM domains | | | | |
| | | 1 pseudogene | | | | |
| 26284 | ERAL1 | Era-like 12S | 2.70 | −0.05 | 0.90 | 0.35 |
| | | mitochondrial | | | | |
| | | rRNA | | | | |
| | | chaperone 1 | | | | |
| 64847 | SPATA20 | spermatogenesis | 2.70 | −0.42 | 0.01 | 0.46 |
| | | associated | | | | |
| | | 20 | | | | |
| 55974 | SLC50A1 | solute carrier | 2.69 | 0.40 | 0.27 | 0.44 |
| | | family 50 | | | | |
| | | (sugar efflux | | | | |
| | | transporter), | | | | |
| | | member 1 | | | | |
| 64405 | CDH22 | cadherin 22, | 2.68 | −0.66 | 0.02 | 0.78 |
| | | type 2 | | | | |
| 6510 | SLC1A5 | solute carrier | 2.68 | 0.25 | 1.07 | 0.13 |
| | | family 1 | | | | |
| | | (neutral amino | | | | |
| | | acid | | | | |
| | | transporter), | | | | |
| | | member 5 | | | | |
| 196463 | PLBD2 | phospholipase | 2.67 | −1.07 | −0.43 | 0.96 |
| | | B domain | | | | |
| | | containing 2 | | | | |
| 741 | ZNHIT2 | zinc finger, | 2.67 | 0.67 | 0.08 | 0.56 |
| | | HIT-type | | | | |
| | | containing 2 | | | | |
| 23568 | ARL2BP | ADP- | 2.66 | −0.14 | 0.26 | −0.22 |
| | | ribosylation | | | | |
| | | factor-like 2 | | | | |
| | | binding | | | | |
| | | protein | | | | |
| 79144 | PPDPF | pancreatic | 2.66 | 0.31 | 0.19 | −0.47 |
| | | progenitor cell | | | | |
| | | differentiation | | | | |
| | | and | | | | |
| | | proliferation | | | | |
| | | factor | | | | |
| 91319 | DERL3 | derlin 3 | 2.66 | −0.62 | 0.03 | −0.15 |
| 79654 | HECTD3 | HECT domain | 2.64 | 0.18 | −0.01 | 0.20 |
| | | containing E3 | | | | |
| | | ubiquitin | | | | |
| | | protein ligase 3 | | | | |
| 115950 | ZNF653 | zinc finger | 2.63 | 0.27 | 0.87 | 0.00 |
| | | protein 653 | | | | |
| 1374 | CPT1A | carnitine | 2.63 | 0.10 | −0.31 | −0.13 |
| | | palmitoyltransferase | | | | |
| | | 1A | | | | |
| | | (liver) | | | | |
| 81533 | ITFG1 | integrin alpha | 2.62 | 0.64 | −0.23 | 0.72 |
| | | FG-GAP | | | | |
| | | repeat | | | | |
| | | containing 1 | | | | |
| 201931 | TMEM192 | transmembrane | 2.60 | 0.09 | 0.73 | −0.14 |
| | | protein 192 | | | | |
| 7979 | SHFM1 | split hand/foot | 2.60 | 1.12 | −0.09 | −0.10 |
| | | malformation | | | | |
| | | (ectrodactyly) | | | | |
| | | type 1 | | | | |
| 159296 | NKX2-3 | NK2 | 2.59 | 0.50 | 1.03 | 1.28 |
| | | homeobox 3 | | | | |
| 51102 | MECR | mitochondrial | 2.59 | 0.16 | 0.07 | 0.17 |
| | | trans-2-enoyl- | | | | |
| | | CoA | | | | |
| | | reductase | | | | |
| 8729 | GBF1 | golgi | 2.59 | 1.24 | −0.79 | 0.52 |
| | | brefeldin A | | | | |
| | | resistant | | | | |
| | | guanine | | | | |
| | | nucleotide | | | | |
| | | exchange | | | | |
| | | factor 1 | | | | |
| 118460 | EXOSC6 | exosome | 2.58 | 0.83 | −0.62 | 0.09 |
| | | component 6 | | | | |
| 79586 | CHPF | chondroitin | 2.57 | 0.08 | −0.01 | −0.27 |
| | | polymerizing | | | | |
| | | factor | | | | |
| 2052 | EPHX1 | epoxide | 2.54 | 0.28 | −0.62 | −0.70 |
| | | hydrolase 1, | | | | |
| | | microsomal | | | | |
| | | (xenobiotic) | | | | |
| 84893 | FBXO18 | F-box protein, | 2.53 | 0.06 | −0.86 | 0.82 |
| | | helicase, 18 | | | | |
| 54726 | OTUD4 | OTU domain | 2.53 | −0.54 | 0.74 | −0.10 |
| | | containing 4 | | | | |
| 813 | CALU | calumenin | 2.53 | 0.77 | −0.57 | −0.59 |
| 54431 | DNAJC10 | DnaJ (Hsp40) | 2.52 | −0.14 | −0.64 | −0.26 |
| | | homolog, | | | | |
| | | subfamily C, | | | | |
| | | member 10 | | | | |
| 9612 | NCOR2 | nuclear | 2.52 | 0.79 | 0.41 | 1.31 |
| | | receptor | | | | |
| | | corepressor 2 | | | | |
| 9829 | DNAJC6 | DnaJ (Hsp40) | 2.51 | −1.46 | −1.60 | 0.51 |
| | | homolog, | | | | |
| | | subfamily C, | | | | |
| | | member 6 | | | | |
| 79036 | KXD1 | KxDL motif | 2.51 | 1.09 | 1.96 | 0.12 |
| | | containing 1 | | | | |
| 284361 | EMC10 | ER membrane | 2.51 | 0.39 | −0.02 | −0.10 |
| | | protein | | | | |
| | | complex | | | | |
| | | subunit 10 | | | | |
| 131118 | DNAJC19 | DnaJ (Hsp40) | 2.50 | −0.86 | −0.69 | −0.12 |
| | | homolog, | | | | |
| | | subfamily C, | | | | |
| | | member 19 | | | | |
| 644809 | C15orf56 | chromosome | 2.50 | 1.27 | 0.33 | −0.45 |
| | | 15 open | | | | |
| | | reading frame | | | | |
| | | 56 | | | | |
| 2119 | ETV5 | ets variant 5 | 2.50 | −0.01 | −0.98 | 0.28 |
| 5833 | PCYT2 | phosphate | 2.50 | 0.56 | −0.12 | −0.20 |
| | | cytidylyltransferase | | | | |
| | | 2, | | | | |
| | | ethanolamine | | | | |
| 922 | CD5L | CD5 | 2.49 | −0.83 | −0.01 | −0.54 |
| | | molecule-like | | | | |
| 9261 | MAPKAPK2 | mitogen- | 2.49 | 0.73 | 0.00 | −0.02 |
| | | activated | | | | |
| | | protein | | | | |
| | | kinase- | | | | |
| | | activated | | | | |
| | | protein kinase 2 | | | | |
| 57190 | SEPN1 | selenoprotein | 2.49 | 0.30 | −2.37 | −0.29 |
| | | N, 1 | | | | |
| 79644 | SRD5A3 | steroid 5 | 2.48 | 0.19 | −0.30 | −1.11 |
| | | alpha- | | | | |
| | | reductase 3 | | | | |
| 79058 | ASPSCR1 | alveolar soft | 2.48 | 1.16 | −0.14 | −0.09 |
| | | part sarcoma | | | | |
| | | chromosome | | | | |
| | | region, | | | | |
| | | candidate 1 | | | | |
| 7832 | BTG2 | BTG family, | 2.47 | 0.09 | −0.36 | −0.33 |
| | | member 2 | | | | |
| 11070 | TMEM115 | transmembrane | 2.47 | 0.04 | 0.53 | 0.48 |
| | | protein 115 | | | | |
| 10847 | SRCAP | Snf2-related | 2.46 | 0.51 | 0.46 | −1.01 |
| | | CREBBP | | | | |
| | | activator | | | | |
| | | protein | | | | |
| 4597 | MVD | mevalonate | 2.46 | 0.97 | 0.72 | 0.94 |
| | | (diphospho) | | | | |
| | | decarboxylase | | | | |
| 100506696 | PCAT6 | prostate | 2.45 | 0.60 | 0.46 | −0.56 |
| | | cancer | | | | |
| | | associated | | | | |
| | | transcript 6 | | | | |
| | | (non-protein | | | | |
| | | coding) | | | | |
| 100507459 | LOC100507459 | uncharacterized | 2.45 | −0.40 | 0.61 | 0.49 |
| | | LOC100507459 | | | | |
| 100130613 | CXorf64 | chromosome | 2.45 | 0.41 | −0.67 | −0.83 |
| | | X open | | | | |
| | | reading frame | | | | |
| | | 64 | | | | |
| 5081 | PAX7 | paired box 7 | 2.45 | 0.92 | 1.45 | −0.72 |
| 100240728 | LOC100240728 | uncharacterized | 2.44 | −0.30 | 0.84 | −0.05 |
| | | LOC100240728 | | | | |
| 26229 | B3GAT3 | beta-1,3- | 2.44 | 0.41 | 0.51 | −0.10 |
| | | glucuronyltransferase 3 | | | | |
| | | (glucuronosyltransferase | | | | |
| | | I) | | | | |
| 950 | SCARB2 | scavenger | 2.44 | 0.02 | −0.02 | −0.25 |
| | | receptor class | | | | |
| | | B, member 2 | | | | |
| 161882 | ZFPM1 | zinc finger | 2.44 | −0.12 | 0.46 | 0.89 |
| | | protein, FOG | | | | |
| | | family | | | | |
| | | member 1 | | | | |
| 5034 | P4HB | prolyl 4- | 2.43 | 0.81 | 0.23 | −0.36 |
| | | hydroxylase, | | | | |
| | | beta | | | | |
| | | polypeptide | | | | |
| 123355 | LRRC28 | leucine rich | 2.43 | 0.93 | 0.57 | 0.85 |
| | | repeat | | | | |
| | | containing 28 | | | | |
| 6734 | SRPR | signal | 2.43 | 1.45 | −0.69 | 0.11 |
| | | recognition | | | | |
| | | particle | | | | |
| | | receptor | | | | |
| | | (docking | | | | |
| | | protein) | | | | |
| 9911 | TMCC2 | transmembrane | 2.43 | −0.95 | −0.25 | 0.73 |
| | | and coiled- | | | | |
| | | coil domain | | | | |
| | | family 2 | | | | |
| 222642 | TSPO2 | translocator | 2.42 | −0.73 | −0.46 | 0.66 |
| | | protein 2 | | | | |
| 3643 | INSR | insulin | 2.42 | 0.82 | 0.91 | −0.72 |
| | | receptor | | | | |
| 85462 | FHDC1 | FH2 domain | 2.41 | −1.49 | −0.58 | 0.18 |
| | | containing 1 | | | | |
| 149175 | MANEAL | mannosidase, | 2.40 | −0.21 | 1.02 | −0.57 |
| | | endo-alpha- | | | | |
| | | like | | | | |
| 1819 | DRG2 | developmentally | 2.40 | 0.10 | −0.07 | 0.40 |
| | | regulated | | | | |
| | | GTP binding | | | | |
| | | protein 2 | | | | |
| 10370 | CITED2 | Cbp/p300- | 2.39 | 0.05 | −1.13 | −0.29 |
| | | interacting | | | | |
| | | transactivator, | | | | |
| | | with Glu/Asp- | | | | |
| | | rich carboxy- | | | | |
| | | terminal | | | | |
| | | domain, 2 | | | | |
| 25792 | CIZ1 | CDKN1A | 2.39 | 0.29 | 0.30 | −0.16 |
| | | interacting | | | | |
| | | zinc finger | | | | |
| | | protein 1 | | | | |
| 4072 | EPCAM | epithelial cell | 2.39 | −0.86 | −0.35 | 0.43 |
| | | adhesion | | | | |
| | | molecule | | | | |
| 84447 | SYVN1 | synovial | 2.39 | 0.55 | −0.02 | −1.26 |
| | | apoptosis | | | | |
| | | inhibitor 1, | | | | |
| | | synoviolin | | | | |
| 527 | ATP6V0C | ATPase, H+ | 2.39 | 0.82 | 0.46 | 0.25 |
| | | transporting, | | | | |
| | | lysosomal | | | | |
| | | 16 kDa, V0 | | | | |
| | | subunit c | | | | |
| 10629 | TAF6L | TAF6-like | 2.38 | 0.86 | 0.07 | 0.15 |
| | | RNA | | | | |
| | | polymerase II, | | | | |
| | | p300/CBP- | | | | |
| | | associated | | | | |
| | | factor | | | | |
| | | (PCAF)- | | | | |
| | | associated | | | | |
| | | factor, 65 kDa | | | | |
| 51150 | SDF4 | stromal cell | 2.38 | 0.64 | −0.15 | −0.38 |
| | | derived factor 4 | | | | |
| 57143 | ADCK1 | aarF domain | 2.37 | −0.13 | −0.14 | −0.63 |
| | | containing | | | | |
| | | kinase 1 | | | | |
| 682 | BSG | basigin | 2.37 | 1.54 | −0.94 | 0.51 |
| 10139 | ARFRP1 | ADP- | 2.37 | 0.91 | −0.06 | −0.20 |
| | | ribosylation | | | | |
| | | factor related | | | | |
| | | protein 1 | | | | |
| 203238 | CCDC171 | coiled-coil | 2.37 | 0.55 | −0.41 | −0.27 |
| | | domain | | | | |
| | | containing | | | | |
| | | 171 | | | | |
| 54805 | CNNM2 | cyclin M2 | 2.37 | −0.82 | 0.70 | 0.73 |
| 1200 | TPP1 | tripeptidyl | 2.37 | 0.44 | −1.00 | 0.97 |
| | | peptidase I | | | | |
| 2271 | FH | fumarate | 2.37 | 1.67 | 1.85 | −0.14 |
| | | hydratase | | | | |
| 376412 | RNF126P1 | ring finger | 2.37 | 0.37 | −0.03 | 0.02 |
| | | protein 126 | | | | |
| | | pseudogene 1 | | | | |
| 4924 | NUCB1 | nucleobindin 1 | 2.37 | 1.68 | −0.36 | 0.75 |
| 55132 | LARP1B | La | 2.37 | 0.33 | −0.97 | −0.07 |
| | | ribonucleoprotein | | | | |
| | | domain | | | | |
| | | family, | | | | |
| | | member 1B | | | | |
| 9650 | MTFR1 | mitochondrial | 2.37 | 0.55 | −1.00 | 0.41 |
| | | fission | | | | |
| | | regulator 1 | | | | |
| 29803 | REPIN1 | replication | 2.36 | 0.71 | 0.46 | −1.02 |
| | | initiator 1 | | | | |
| 4507 | MTAP | methylthioadenosine | 2.36 | −0.31 | −0.03 | 0.15 |
| | | phosphorylase | | | | |
| 816 | CAMK2B | calcium/calmodulin- | 2.36 | 0.87 | 0.30 | −0.16 |
| | | dependent | | | | |
| | | protein kinase | | | | |
| | | II beta | | | | |
| 120379 | PIH1D2 | PIH1 domain | 2.36 | −0.30 | 0.35 | −0.39 |
| | | containing 2 | | | | |
| 91289 | LMF2 | lipase | 2.36 | 0.26 | 0.57 | −0.74 |
| | | maturation | | | | |
| | | factor 2 | | | | |
| 10905 | MAN1A2 | mannosidase, | 2.35 | −0.51 | 0.22 | 0.09 |
| | | alpha, class | | | | |
| | | 1A, member 2 | | | | |
| 25930 | PTPN23 | protein | 2.35 | 0.19 | −0.75 | 0.81 |
| | | tyrosine | | | | |
| | | phosphatase, | | | | |
| | | non-receptor | | | | |
| | | type 23 | | | | |
| 57648 | KIAA1522 | KIAA1522 | 2.35 | −0.40 | 0.20 | −0.30 |
| 7249 | TSC2 | tuberous | 2.35 | 1.12 | 0.34 | −0.58 |
| | | sclerosis 2 | | | | |
| 55151 | TMEM38B | transmembrane | 2.34 | −0.35 | −1.27 | −0.84 |
| | | protein 38B | | | | |
| 160728 | SLC5A8 | solute carrier | 2.33 | 0.23 | 1.90 | 0.52 |
| | | family 5 | | | | |
| | | (sodium/mono | | | | |
| | | carboxylate | | | | |
| | | cotransporter), | | | | |
| | | member 8 | | | | |
| 148418 | SAMD13 | sterile alpha | 2.33 | −0.47 | −1.02 | 0.15 |
| | | motif domain | | | | |
| | | containing 13 | | | | |
| 646762 | LOC646762 | uncharacterized | 2.32 | −0.74 | 0.22 | 0.58 |
| | | LOC646762 | | | | |
| 79109 | MAPKAP1 | mitogen- | 2.32 | 1.94 | 1.42 | −0.40 |
| | | activated | | | | |
| | | protein kinase | | | | |
| | | associated | | | | |
| | | protein 1 | | | | |
| 81037 | CLPTM1L | CLPTM1-like | 2.31 | −0.21 | 0.34 | −0.22 |
| 51523 | CXXC5 | CXXC finger | 2.29 | −0.06 | 0.22 | −0.21 |
| | | protein 5 | | | | |
| 55322 | C5orf22 | chromosome | 2.29 | 0.39 | 0.87 | −0.82 |
| | | 5 open | | | | |
| | | reading frame | | | | |
| | | 22 | | | | |
| 100996671 | LOC100996671 | uncharacterized | 2.29 | 0.80 | −0.60 | 0.34 |
| | | LOC100996671 | | | | |
| 55222 | LRRC20 | leucine rich | 2.29 | 0.23 | −0.12 | 0.22 |
| | | repeat | | | | |
| | | containing 20 | | | | |
| 55624 | POMGNT1 | protein O- | 2.29 | −0.06 | 0.70 | −0.71 |
| | | linked | | | | |
| | | mannose N- | | | | |
| | | acetylglucosaminyltransferase | | | | |
| | | 1 (beta 1,2-) | | | | |
| 100505983 | LOC100505983 | uncharacterized | 2.28 | −1.93 | −0.81 | 0.07 |
| | | LOC100505983 | | | | |
| 3069 | HDLBP | high density | 2.28 | −0.16 | 0.12 | −0.65 |
| | | lipoprotein | | | | |
| | | binding | | | | |
| | | protein | | | | |
| 10959 | TMED2 | transmembrane | 2.28 | 0.83 | 0.00 | −0.50 |
| | | emp24 | | | | |
| | | domain | | | | |
| | | trafficking | | | | |
| | | protein 2 | | | | |
| 6598 | SMARCB1 | SWI/SNF | 2.28 | 1.36 | 0.07 | −0.79 |
| | | related, matrix | | | | |
| | | associated, | | | | |
| | | actin | | | | |
| | | dependent | | | | |
| | | regulator of | | | | |
| | | chromatin, | | | | |
| | | subfamily b, | | | | |
| | | member 1 | | | | |
| 60412 | EXOC4 | exocyst | 2.27 | 1.43 | −0.21 | 0.74 |
| | | complex | | | | |
| | | component 4 | | | | |
| 54982 | CLN6 | ceroid- | 2.27 | −0.12 | 1.03 | −0.50 |
| | | lipofuscinosis, | | | | |
| | | neuronal 6, | | | | |
| | | late infantile, | | | | |
| | | variant | | | | |
| 91752 | ZNF804A | zinc finger | 2.27 | 1.42 | −0.14 | 0.19 |
| | | protein 804A | | | | |
| 113178 | SCAMP4 | secretory | 2.26 | 1.06 | −0.85 | 0.25 |
| | | carrier | | | | |
| | | membrane | | | | |
| | | protein 4 | | | | |
| 10 | NAT2 | N- | 2.26 | 0.44 | −0.98 | 1.14 |
| | | acetyltransferase 2 | | | | |
| | | (arylamine N- | | | | |
| | | acetyltransferase) | | | | |
| 375 | ARF1 | ADP- | 2.26 | 1.14 | 0.27 | 0.39 |
| | | ribosylation | | | | |
| | | factor 1 | | | | |
| 5589 | PRKCSH | protein kinase | 2.26 | 0.76 | −0.28 | −0.42 |
| | | C substrate | | | | |
| | | 80K-H | | | | |
| 9136 | RRP9 | ribosomal | 2.26 | 0.58 | 1.04 | 0.45 |
| | | RNA | | | | |
| | | processing 9, | | | | |
| | | small subunit | | | | |
| | | (SSU) | | | | |
| | | processome | | | | |
| | | component, | | | | |
| | | homolog | | | | |
| | | (yeast) | | | | |
| 23193 | GANAB | glucosidase, | 2.26 | 0.68 | −0.80 | −0.03 |
| | | alpha; neutral | | | | |
| | | AB | | | | |
| 1798 | DPAGT1 | dolichyl- | 2.25 | 0.11 | 0.64 | −1.27 |
| | | phosphate | | | | |
| | | (UDP-N- | | | | |
| | | acetylglucosamine) | | | | |
| | | N- | | | | |
| | | acetylglucosaminephosphotransferase 1 | | | | |
| | | (GlcNAc-1-P | | | | |
| | | transferase) | | | | |
| 85440 | DOCK7 | dedicator of | 2.25 | 0.22 | −0.02 | −0.61 |
| | | cytokinesis 7 | | | | |
| 100505794 | LOC100505794 | uncharacterized | 2.24 | −0.85 | −1.84 | −0.01 |
| | | LOC100505794 | | | | |
| 1173 | AP2M1 | adaptor- | 2.24 | 0.69 | 0.34 | 0.20 |
| | | related protein | | | | |
| | | complex 2, | | | | |
| | | mu 1 subunit | | | | |
| 124540 | MSI2 | musashi | 2.24 | 0.42 | 0.40 | 0.33 |
| | | RNA-binding | | | | |
| | | protein 2 | | | | |
| 8372 | HYAL3 | hyaluronoglucosaminidase 3 | 2.24 | −0.17 | 0.81 | −0.39 |
| 64755 | C16orf58 | chromosome | 2.24 | 0.26 | −0.77 | −0.46 |
| | | 16 open | | | | |
| | | reading frame | | | | |
| | | 58 | | | | |
| 79102 | RNF26 | ring finger | 2.24 | 0.59 | 0.25 | −0.41 |
| | | protein 26 | | | | |
| 10963 | STIP1 | stress- | 2.24 | 1.54 | 0.24 | 0.38 |
| | | induced- | | | | |
| | | phosphoprotein 1 | | | | |
| 375387 | NRROS | negative | 2.24 | 0.14 | −0.62 | 0.52 |
| | | regulator of | | | | |
| | | reactive | | | | |
| | | oxygen | | | | |
| | | species | | | | |
| 972 | CD74 | CD74 | 2.23 | 1.35 | 0.06 | −0.03 |
| | | molecule, | | | | |
| | | major | | | | |
| | | histocompatibility | | | | |
| | | complex, | | | | |
| | | class II | | | | |
| | | invariant | | | | |
| | | chain | | | | |
| 54676 | GTPBP2 | GTP binding | 2.23 | 1.23 | 0.20 | 1.63 |
| | | protein 2 | | | | |
| 192286 | HIGD2A | HIG1 hypoxia | 2.23 | 0.13 | −0.59 | 0.61 |
| | | inducible | | | | |
| | | domain | | | | |
| | | family, | | | | |
| | | member 2A | | | | |
| 3295 | HSD17B4 | hydroxysteroid | 2.23 | 0.58 | .25 | −0.54 |
| | | (17-beta) | | | | |
| | | dehydrogenase 4 | | | | |
| 80852 | GRIP2 | glutamate | 2.23 | −1.75 | 0.47 | 0.38 |
| | | receptor | | | | |
| | | interacting | | | | |
| | | protein 2 | | | | |
| 1298 | COL9A2 | collagen, type | 2.22 | 0.82 | 0.86 | 0.11 |
| | | IX, alpha 2 | | | | |
| 2004 | ELK3 | ELK3, ETS- | 2.22 | −0.85 | 0.96 | −0.24 |
| | | domain | | | | |
| | | protein (SRF | | | | |
| | | accessory | | | | |
| | | protein 2) | | | | |
| 10279 | PRSS16 | protease, | 2.22 | 0.07 | 0.07 | −0.15 |
| | | serine, 16 | | | | |
| | | (thymus) | | | | |
| 151790 | WDR49 | WD repeat | 2.22 | 0.39 | 0.65 | −0.20 |
| | | domain 49 | | | | |
| 399664 | MEX3D | mex-3 RNA | 2.21 | −0.89 | 0.64 | −0.40 |
| | | binding | | | | |
| | | family | | | | |
| | | member D | | | | |
| 5664 | PSEN2 | presenilin 2 | 2.21 | 0.07 | −0.24 | 0.53 |
| | | (Alzheimer | | | | |
| | | disease 4) | | | | |
| 51614 | ERGIC3 | ERGIC and | 2.21 | 0.14 | −0.32 | −0.36 |
| | | golgi 3 | | | | |
| 79178 | THTPA | thiamine | 2.21 | 0.18 | 1.08 | 0.08 |
| | | triphosphatase | | | | |
| 51726 | DNAJB11 | DnaJ (Hsp40) | 2.20 | 0.26 | 0.41 | −0.49 |
| | | homolog, | | | | |
| | | subfamily B, | | | | |
| | | member 11 | | | | |
| 3425 | IDUA | iduronidase, | 2.20 | −0.08 | −0.06 | −0.61 |
| | | alpha-L- | | | | |
| 1642 | DDB1 | damage- | 2.20 | −0.46 | 0.93 | −0.32 |
| | | specific DNA | | | | |
| | | binding | | | | |
| | | protein 1, | | | | |
| | | 127 kDa | | | | |
| 101928770 | LOC101928770 | uncharacterized | 2.19 | 0.42 | −0.39 | 1.97 |
| | | LOC101928770 | | | | |
| 11015 | KDELR3 | KDEL (Lys- | 2.19 | 0.54 | −0.29 | −1.29 |
| | | Asp-Glu-Leu) | | | | |
| | | endoplasmic | | | | |
| | | reticulum | | | | |
| | | protein | | | | |
| | | retention | | | | |
| | | receptor 3 | | | | |
| 35 | ACADS | acyl-CoA | 2.19 | 1.08 | −0.33 | 0.02 |
| | | dehydrogenase, C-2 to C-3 | | | | |
| | | short chain | | | | |
| 64798 | DEPTOR | DEP domain | 2.19 | −0.97 | −0.54 | −0.53 |
| | | containing | | | | |
| | | MTOR- | | | | |
| | | interacting | | | | |
| | | protein | | | | |
| 23457 | ABCB9 | ATP-binding | 2.19 | −0.12 | 1.07 | −0.70 |
| | | cassette, sub- | | | | |
| | | family B | | | | |
| | | (MDR/TAP), | | | | |
| | | member 9 | | | | |
| 56132 | PCDHB3 | protocadherin | 2.19 | 0.22 | −0.59 | 1.11 |
| | | beta 3 | | | | |
| 23400 | ATP13A2 | ATPase type | 2.19 | −0.18 | −0.61 | 0.41 |
| | | 13A2 | | | | |
| 3980 | LIG3 | ligase III, | 2.19 | −0.46 | 0.56 | 0.18 |
| | | DNA, ATP- | | | | |
| | | dependent | | | | |
| 5690 | PSMB2 | proteasome | 2.19 | 0.25 | 0.78 | −0.61 |
| | | (prosome, | | | | |
| | | macropain) | | | | |
| | | subunit, beta | | | | |
| | | type, 2 | | | | |
| 7917 | BAG6 | BCL2- | 2.19 | 0.11 | 0.58 | −0.49 |
| | | associated | | | | |
| | | athanogene 6 | | | | |
| 1739 | DLG1 | discs, large | 2.18 | 0.29 | −0.28 | 0.06 |
| | | homolog 1 | | | | |
| | | (Drosophila) | | | | |
| 2837 | UTS2R | urotensin 2 | 2.18 | 1.12 | −0.79 | −1.10 |
| | | receptor | | | | |
| 83746 | L3MBTL2 | l(3)mbt-like 2 | 2.18 | 0.03 | 0.30 | −0.18 |
| | | (Drosophila) | | | | |
| 5962 | RDX | radixin | 2.18 | 0.90 | −0.78 | −0.53 |
| 9956 | HS3ST2 | heparan | 2.18 | 0.67 | 0.26 | −0.67 |
| | | sulfate | | | | |
| | | (glucosamine) | | | | |
| | | 3-O- | | | | |
| | | sulfotransferase 2 | | | | |
| 162968 | ZNF497 | zinc finger | 2.18 | 0.06 | 0.30 | −0.31 |
| | | protein 497 | | | | |
| 4192 | MDK | midkine | 2.18 | −0.74 | −0.74 | 0.03 |
| | | (neurite | | | | |
| | | growth- | | | | |
| | | promoting | | | | |
| | | factor 2) | | | | |
| 54919 | HEATR2 | HEAT repeat | 2.18 | 0.58 | 0.20 | −1.69 |
| | | containing 2 | | | | |
| 10972 | TMED10 | transmembrane | 2.17 | 0.24 | 0.15 | −0.92 |
| | | emp24-like | | | | |
| | | trafficking | | | | |
| | | protein 10 | | | | |
| | | (yeast) | | | | |
| 23061 | TBC1D9B | TBC1 domain | 2.17 | −0.48 | 0.44 | −0.23 |
| | | family, | | | | |
| | | member 9B | | | | |
| | | (with GRAM | | | | |
| | | domain) | | | | |
| 3792 | KEL | Kell blood | 2.17 | −0.27 | −1.35 | 1.11 |
| | | group, | | | | |
| | | metallo- | | | | |
| | | endopeptidase | | | | |
| 2063 | NR2F6 | nuclear | 2.17 | −0.44 | 0.57 | 0.74 |
| | | receptor | | | | |
| | | subfamily 2, | | | | |
| | | group F, | | | | |
| | | member 6 | | | | |
| 55177 | RMDN3 | regulator of | 2.17 | −0.50 | −0.29 | −0.19 |
| | | microtubule | | | | |
| | | dynamics 3 | | | | |
| 11147 | HHLA3 | HERV-H | 2.17 | 0.24 | −0.04 | 0.74 |
| | | LTR- | | | | |
| | | associating 3 | | | | |
| 57486 | NLN | neurolysin | 2.17 | 1.46 | 1.23 | −0.04 |
| | | (metallopeptidase | | | | |
| | | M3 | | | | |
| | | family) | | | | |
| 30814 | PLA2G2E | phospholipase | 2.16 | 0.03 | −0.89 | 0.15 |
| | | A2, group IIE | | | | |
| 57414 | RHBDD2 | rhomboid | 2.16 | −0.01 | −0.77 | −0.70 |
| | | domain | | | | |
| | | containing 2 | | | | |
| 84836 | ABHD14B | abhydrolase | 2.16 | −0.24 | 0.04 | −0.02 |
| | | domain | | | | |
| | | containing | | | | |
| | | 14B | | | | |
| 8516 | ITGA8 | integrin, alpha 8 | 2.16 | −0.45 | −0.84 | −0.31 |
| 89866 | SEC16B | SEC16 | 2.16 | −0.12 | 0.36 | 1.15 |
| | | homolog B (S. cerevisiae) | | | | |
| 10491 | CRTAP | cartilage | 2.16 | −0.69 | 0.36 | 0.47 |
| | | associated | | | | |
| | | protein | | | | |
| 11047 | ADRM1 | adhesion | 2.16 | −0.45 | 1.09 | 0.59 |
| | | regulating | | | | |
| | | molecule 1 | | | | |
| 28971 | AAMDC | adipogenesis | 2.16 | 0.19 | 0.18 | 0.52 |
| | | associated, | | | | |
| | | Mth938 | | | | |
| | | domain | | | | |
| | | containing | | | | |
| 8078 | USP5 | ubiquitin | 2.16 | 0.09 | 0.52 | −0.61 |
| | | specific | | | | |
| | | peptidase 5 | | | | |
| | | (isopeptidase | | | | |
| | | T) | | | | |
| 37 | ACADVL | acyl-CoA | 2.16 | 0.58 | −0.03 | −0.12 |
| | | dehydrogenase, | | | | |
| | | very long | | | | |
| | | chain | | | | |
| 145508 | CEP128 | centrosomal | 2.15 | −0.23 | 0.18 | −0.41 |
| | | protein | | | | |
| | | 128 kDa | | | | |
| 49861 | CLDN20 | claudin 20 | 2.15 | 0.38 | 0.63 | −0.14 |
| 5905 | RANGAP1 | Ran GTPase | 2.15 | 0.08 | 0.44 | 0.11 |
| | | activating | | | | |
| | | protein 1 | | | | |
| 7542 | ZFPL1 | zinc finger | 2.15 | 1.34 | 0.05 | 1.09 |
| | | protein-like 1 | | | | |
| 9562 | MINPP1 | multiple | 2.15 | −0.37 | −0.97 | −0.14 |
| | | inositol- | | | | |
| | | polyphosphate | | | | |
| | | phosphatase 1 | | | | |
| 79095 | C9orf16 | chromosome | 2.15 | 0.05 | 0.44 | 0.56 |
| | | 9 open | | | | |
| | | reading frame | | | | |
| | | 16 | | | | |
| 8815 | BANF1 | barrier to | 2.15 | 1.25 | 0.40 | 0.35 |
| | | autointegration | | | | |
| | | factor 1 | | | | |
| 54867 | TMEM214 | transmembrane | 2.15 | 1.05 | 0.54 | −0.11 |
| | | protein 214 | | | | |
| 6721 | SREBF2 | sterol | 2.15 | 1.52 | −1.11 | 0.63 |
| | | regulatory | | | | |
| | | element | | | | |
| | | binding | | | | |
| | | transcription | | | | |
| | | factor 2 | | | | |
| 23133 | PHF8 | PHD finger | 2.15 | −0.14 | 0.85 | −1.47 |
| | | protein 8 | | | | |
| 113675 | SDSL | serine | 2.14 | 0.18 | 0.46 | 0.40 |
| | | dehydratase- | | | | |
| | | like | | | | |
| 129303 | TMEM150A | transmembrane | 2.14 | 0.67 | −0.67 | 0.33 |
| | | protein | | | | |
| | | 150A | | | | |
| 57658 | CALCOCO1 | calcium | 2.14 | −0.17 | −0.13 | −0.05 |
| | | binding and | | | | |
| | | coiled-coil | | | | |
| | | domain 1 | | | | |
| 162 | AP1B1 | adaptor- | 2.14 | 0.94 | 1.04 | −0.67 |
| | | related protein | | | | |
| | | complex 1, | | | | |
| | | beta 1 subunit | | | | |
| 2873 | GPS1 | G protein | 2.14 | 0.97 | 0.03 | −0.43 |
| | | pathway | | | | |
| | | suppressor 1 | | | | |
| 64978 | MRPL38 | mitochondrial | 2.14 | 0.70 | −0.93 | −0.17 |
| | | ribosomal | | | | |
| | | protein L38 | | | | |
| 220323 | OAF | OAF homolog | 2.14 | −0.95 | −0.74 | 0.44 |
| | | (Drosophila) | | | | |
| 6006 | RHCE | Rh blood | 2.14 | −1.22 | −1.53 | 1.05 |
| | | group, CcEe | | | | |
| | | antigens | | | | |
| 389812 | LCN15 | lipocalin 15 | 2.14 | −0.38 | 0.83 | 0.01 |
| 2628 | GATM | glycine | 2.13 | 1.24 | 0.66 | −0.73 |
| | | amidinotransferase | | | | |
| | | (L- | | | | |
| | | arginine:glycine | | | | |
| | | amidinotransferase) | | | | |
| 6509 | SLC1A4 | solute carrier | 2.13 | −0.32 | −0.44 | −1.07 |
| | | family 1 | | | | |
| | | (glutamate/neutral | | | | |
| | | amino | | | | |
| | | acid | | | | |
| | | transporter), | | | | |
| | | member 4 | | | | |
| 3888 | KRT82 | keratin 82 | 2.13 | −0.02 | 0.75 | −0.44 |
| 5211 | PFKL | phosphofructo | 2.13 | 0.63 | 1.37 | −0.29 |
| | | kinase, liver | | | | |
| 55080 | TAPBPL | TAP binding | 2.13 | 0.69 | 0.59 | 0.34 |
| | | protein-like | | | | |
| 2139 | EYA2 | eyes absent | 2.13 | −0.64 | −0.46 | −0.24 |
| | | homolog 2 | | | | |
| | | (Drosophila) | | | | |
| 2030 | SLC29A1 | solute carrier | 2.12 | 0.37 | 0.86 | −0.34 |
| | | family 29 | | | | |
| | | (equilibrative | | | | |
| | | nucleoside | | | | |
| | | transporter), | | | | |
| | | member 1 | | | | |
| 56005 | C19orf10 | chromosome | 2.12 | 0.10 | 0.03 | −0.14 |
| | | 19 open | | | | |
| | | reading frame | | | | |
| | | 10 | | | | |
| 6414 | SEPP1 | selenoprotein | 2.12 | −0.17 | −1.23 | −0.28 |
| | | P, plasma, 1 | | | | |
| 5184 | PEPD | peptidase D | 2.12 | 0.58 | 1.17 | −0.10 |
| 643783 | USP46- | USP46 | 2.12 | 0.11 | −0.67 | 0.36 |
| | AS1 | antisense | | | | |
| | | RNA 1 | | | | |
| 101928185 | LOC101928185 | uncharacterized | 2.12 | 0.62 | 1.07 | −0.29 |
| | | LOC101928185 | | | | |
| 22883 | CLSTN1 | calsyntenin 1 | 2.12 | −0.88 | 0.33 | −1.64 |
| 25800 | SLC39A6 | solute carrier | 2.12 | 1.32 | −0.10 | −0.45 |
| | | family 39 | | | | |
| | | (zinc | | | | |
| | | transporter), | | | | |
| | | member 6 | | | | |
| 54681 | P4HTM | prolyl 4- | 2.12 | −0.37 | −0.25 | −0.91 |
| | | hydroxylase, | | | | |
| | | transmembrane | | | | |
| | | (endoplasmic | | | | |
| | | reticulum) | | | | |
| 644150 | WIPF3 | WAS/WASL | 2.12 | 0.18 | −1.20 | −0.10 |
| | | interacting | | | | |
| | | protein | | | | |
| | | family, | | | | |
| | | member 3 | | | | |
| 9048 | ARTN | artemin | 2.12 | 1.14 | 0.34 | −0.47 |
| 3054 | HCFC1 | host cell | 2.11 | −0.38 | 0.24 | 0.51 |
| | | factor C1 | | | | |
| | | (VP16- | | | | |
| | | accessory | | | | |
| | | protein) | | | | |
| 115098 | CCDC124 | coiled-coil | 2.11 | −0.46 | 0.69 | 1.79 |
| | | domain | | | | |
| | | containing | | | | |
| | | 124 | | | | |
| 55558 | PLXNA3 | plexin A3 | 2.11 | 0.55 | −0.45 | −0.92 |
| 675 | BRCA2 | breast cancer | 2.11 | 0.23 | 0.24 | 0.06 |
| | | 2, early onset | | | | |
| 9777 | TM9SF4 | transmembrane 9 | 2.11 | 0.69 | 0.39 | −0.54 |
| | | superfamily | | | | |
| | | protein | | | | |
| | | member 4 | | | | |
| 83707 | TRPT1 | tRNA | 2.11 | 0.84 | 0.89 | −0.54 |
| | | phosphotransferase 1 | | | | |
| 55334 | SLC39A9 | solute carrier | 2.10 | 1.31 | 1.47 | −0.86 |
| | | family 39, | | | | |
| | | member 9 | | | | |
| 9793 | CKAP5 | cytoskeleton | 2.10 | 0.83 | 0.75 | 0.01 |
| | | associated | | | | |
| | | protein 5 | | | | |
| 56834 | GPR137 | G protein- | 2.10 | 0.97 | −1.57 | 1.00 |
| | | coupled | | | | |
| | | receptor 137 | | | | |
| 25921 | ZDHHC5 | zinc finger, | 2.10 | 1.06 | 1.03 | −0.01 |
| | | DHHC-type | | | | |
| | | containing 5 | | | | |
| 6520 | SLC3A2 | solute carrier | 2.10 | 0.59 | 0.18 | −0.52 |
| | | family 3 | | | | |
| | | (amino acid | | | | |
| | | transporter | | | | |
| | | heavy chain), | | | | |
| | | member 2 | | | | |
| 728591 | CCDC169 | coiled-coil | 2.10 | −0.73 | 0.49 | 0.38 |
| | | domain | | | | |
| | | containing | | | | |
| | | 169 | | | | |
| 7415 | VCP | valosin | 2.10 | 0.38 | 0.37 | −0.38 |
| | | containing | | | | |
| | | protein | | | | |
| 130827 | TMEM182 | transmembrane | 2.09 | −0.70 | 0.39 | −0.81 |
| | | protein 182 | | | | |
| 9620 | CELSR1 | cadherin, EGF | 2.09 | 0.64 | −1.29 | 0.72 |
| | | LAG seven- | | | | |
| | | pass G-type | | | | |
| | | receptor 1 | | | | |
| 55335 | NIPSNAP3B | nipsnap | 2.09 | 0.24 | −0.35 | 0.20 |
| | | homolog 3B | | | | |
| | | (C. elegans) | | | | |
| 84988 | PPP1R16A | protein | 2.08 | 0.17 | 0.65 | −0.18 |
| | | phosphatase | | | | |
| | | 1, regulatory | | | | |
| | | subunit 16A | | | | |
| 100192378 | ZFHX4- | ZFHX4 | 2.08 | −0.02 | −0.09 | 0.21 |
| | AS1 | antisense | | | | |
| | | RNA 1 | | | | |
| 442524 | DPY19L2P3 | DPY19L2 | 2.08 | −0.04 | 0.55 | −0.86 |
| | | pseudogene 3 | | | | |
| 51661 | FKBP7 | FK506 | 2.08 | 0.48 | −0.50 | 0.03 |
| | | binding | | | | |
| | | protein 7 | | | | |
| 84922 | FIZ1 | FLT3- | 2.08 | 0.63 | −0.74 | 0.21 |
| | | interacting | | | | |
| | | zinc finger 1 | | | | |
| 84954 | MPND | MPN domain | 2.08 | 0.62 | 1.05 | −0.42 |
| | | containing | | | | |
| 245972 | ATP6V0D2 | ATPase, H+ | 2.08 | 0.45 | 0.17 | −0.66 |
| | | transporting, | | | | |
| | | lysosomal | | | | |
| | | 38 kDa, V0 | | | | |
| | | subunit d2 | | | | |
| 4316 | MMP7 | matrix | 2.08 | 0.46 | 0.54 | −1.74 |
| | | metallopeptidase 7 | | | | |
| | | (matrilysin, | | | | |
| | | uterine) | | | | |
| 55157 | DARS2 | aspartyl- | 2.08 | 0.58 | 1.19 | 0.48 |
| | | tRNA | | | | |
| | | synthetase 2, | | | | |
| | | mitochondrial | | | | |
| 7466 | WFS1 | Wolfram | 2.08 | 0.26 | −0.65 | 0.19 |
| | | syndrome 1 | | | | |
| | | (wolframin) | | | | |
| 79365 | BHLHE41 | basic helix- | 2.08 | 0.26 | −0.23 | −0.71 |
| | | loop-helix | | | | |
| | | family, | | | | |
| | | member e41 | | | | |
| 10066 | SCAMP2 | secretory | 2.08 | 0.03 | 0.23 | 0.02 |
| | | carrier | | | | |
| | | membrane | | | | |
| | | protein 2 | | | | |
| 203068 | TUBB | tubulin, beta | 2.08 | 0.67 | 0.41 | −0.39 |
| | | class I | | | | |
| 5780 | PTPN9 | protein | 2.07 | 1.31 | 0.61 | −1.20 |
| | | tyrosine | | | | |
| | | phosphatase, | | | | |
| | | non-receptor | | | | |
| | | type 9 | | | | |
| 285627 | LOC285627 | uncharacterized | 2.07 | 0.41 | 0.40 | 0.33 |
| | | LOC285627 | | | | |
| 2058 | EPRS | glutamyl- | 2.07 | 0.11 | 1.90 | −0.12 |
| | | prolyl-tRNA | | | | |
| | | synthetase | | | | |
| 1737 | DLAT | dihydrolipoamide | 2.06 | 0.30 | 0.31 | −1.50 |
| | | S- | | | | |
| | | acetyltransferase | | | | |
| 22924 | MAPRE3 | microtubule- | 2.06 | 1.73 | −0.26 | −0.87 |
| | | associated | | | | |
| | | protein, | | | | |
| | | RP/EB | | | | |
| | | family, | | | | |
| | | member 3 | | | | |
| 6005 | RHAG | Rh-associated | 2.06 | −0.62 | −0.69 | 0.67 |
| | | glycoprotein | | | | |
| 5286 | PIK3C2A | phosphatidylinositol- | 2.06 | 0.79 | −0.57 | 0.10 |
| | | 4- | | | | |
| | | phosphate 3- | | | | |
| | | kinase, | | | | |
| | | catalytic | | | | |
| | | subunit type 2 | | | | |
| | | alpha | | | | |
| 11226 | GALNT6 | UDP-N- | 2.06 | 0.02 | 0.84 | −0.35 |
| | | acetyl-alpha- | | | | |
| | | D- | | | | |
| | | galactosamine: | | | | |
| | | polypeptide | | | | |
| | | N- | | | | |
| | | acetylgalactosaminyltransferase 6 | | | | |
| | | (GalNAc-T6) | | | | |
| 2762 | GMDS | GDP- | 2.06 | 0.22 | 0.98 | −0.90 |
| | | mannose 4,6- | | | | |
| | | dehydratase | | | | |
| 8908 | GYG2 | glycogenin 2 | 2.06 | −0.24 | −0.32 | −0.68 |
| 10693 | CCT6B | chaperonin | 2.06 | 0.21 | 0.55 | 0.00 |
| | | containing | | | | |
| | | TCP1, subunit | | | | |
| | | 6B (zeta 2) | | | | |
| 199953 | TMEM201 | transmembrane | 2.06 | 0.09 | 0.13 | 0.12 |
| | | protein 201 | | | | |
| 2760 | GM2A | GM2 | 2.06 | 2.00 | 0.33 | 0.39 |
| | | ganglioside | | | | |
| | | activator | | | | |
| 8295 | TRRAP | transformation/ | 2.06 | 0.95 | 0.39 | −1.52 |
| | | transcription | | | | |
| | | domain- | | | | |
| | | associated | | | | |
| | | protein | | | | |
| 1822 | ATN1 | atrophin 1 | 2.06 | 0.48 | −0.10 | −0.77 |
| 55328 | RNLS | renalase, | 2.06 | −0.14 | 0.34 | 1.00 |
| | | FAD- | | | | |
| | | dependent | | | | |
| | | amine oxidase | | | | |
| 2069 | EREG | epiregulin | 2.05 | −0.72 | −0.16 | −0.15 |
| 4329 | ALDH6A1 | aldehyde | 2.05 | −0.31 | 0.26 | −1.79 |
| | | dehydrogenase | | | | |
| | | 6 family, | | | | |
| | | member A1 | | | | |
| 537 | ATP6AP1 | ATPase, H+ | 2.05 | 0.66 | −0.23 | −0.66 |
| | | transporting, | | | | |
| | | lysosomal | | | | |
| | | accessory | | | | |
| | | protein 1 | | | | |
| 64764 | CREB3L2 | cAMP | 2.05 | −0.55 | 0.39 | −0.70 |
| | | responsive | | | | |
| | | element | | | | |
| | | binding | | | | |
| | | protein 3-like 2 | | | | |
| 811 | CALR | calreticulin | 2.05 | −0.06 | 0.62 | −0.98 |
| 9779 | TBC1D5 | TBC1 domain | 2.05 | 0.62 | 1.10 | 0.35 |
| | | family, | | | | |
| | | member 5 | | | | |
| 114815 | SORCS1 | sortilin- | 2.05 | −1.22 | −0.43 | −0.11 |
| | | related VPS10 | | | | |
| | | domain | | | | |
| | | containing | | | | |
| | | receptor 1 | | | | |
| 29927 | SEC61A1 | Sec61 alpha 1 | 2.05 | 0.72 | 0.17 | −0.26 |
| | | subunit (S. cerevisiae) | | | | |
| 6007 | RHD | Rh blood | 2.05 | 0.25 | −0.06 | 0.38 |
| | | group, D | | | | |
| | | antigen | | | | |
| 5192 | PEX10 | peroxisomal | 2.05 | 0.52 | 0.47 | −0.95 |
| | | biogenesis | | | | |
| | | factor 10 | | | | |
| 125875 | CLDND2 | claudin | 2.04 | −0.13 | 0.77 | −0.29 |
| | | domain | | | | |
| | | containing 2 | | | | |
| 10882 | C1QL1 | complement | 2.04 | 0.92 | 0.57 | 0.80 |
| | | component 1, q | | | | |
| | | subcomponent- | | | | |
| | | like 1 | | | | |
| 192683 | SCAMP5 | secretory | 2.04 | −0.03 | −0.60 | −0.44 |
| | | carrier | | | | |
| | | membrane | | | | |
| | | protein 5 | | | | |
| 8028 | MLLT10 | myeloid/lymphoid | 2.04 | 0.60 | 0.73 | 0.59 |
| | | or | | | | |
| | | mixed-lineage | | | | |
| | | leukemia | | | | |
| | | (trithorax | | | | |
| | | homolog, | | | | |
| | | Drosophila); | | | | |
| | | translocated | | | | |
| | | to, 10 | | | | |
| 150368 | FAM109B | family with | 2.04 | −0.82 | 0.11 | 0.24 |
| | | sequence | | | | |
| | | similarity 109, | | | | |
| | | member B | | | | |
| 84206 | MEX3B | mex-3 RNA | 2.04 | −0.09 | 0.73 | 0.38 |
| | | binding | | | | |
| | | family | | | | |
| | | member B | | | | |
| 101929288 | LOC101929288 | uncharacterized | 2.04 | −0.50 | −0.12 | −0.51 |
| | | LOC101929288 | | | | |
| 826 | CAPNS1 | calpain, small | 2.04 | 0.93 | 0.42 | 0.36 |
| | | subunit 1 | | | | |
| 83440 | ADPGK | ADP- | 2.04 | 0.83 | 0.18 | −0.39 |
| | | dependent | | | | |
| | | glucokinase | | | | |
| 1128 | CHRM1 | cholinergic | 2.03 | 0.85 | 0.38 | −0.13 |
| | | receptor, | | | | |
| | | muscarinic 1 | | | | |
| 160287 | LDHAL6A | lactate | 2.03 | −0.04 | −1.64 | 0.41 |
| | | dehydrogenase | | | | |
| | | A-like 6A | | | | |
| 412 | STS | steroid | 2.03 | 1.18 | 0.39 | 0.83 |
| | | sulfatase | | | | |
| | | (microsomal), | | | | |
| | | isozyme S | | | | |
| 64215 | DNAJC1 | DnaJ (Hsp40) | 2.03 | −0.78 | −0.10 | 0.34 |
| | | homolog, | | | | |
| | | subfamily C, | | | | |
| | | member 1 | | | | |
| 7343 | UBTF | upstream | 2.03 | 0.61 | 0.51 | −0.33 |
| | | binding | | | | |
| | | transcription | | | | |
| | | factor, RNA | | | | |
| | | polymerase I | | | | |
| 25840 | METTL7A | methyltransferase | 2.03 | −0.80 | −0.40 | 0.64 |
| | | like 7A | | | | |
| 55315 | SLC29A3 | solute carrier | 2.03 | −0.65 | −1.04 | 0.67 |
| | | family 29 | | | | |
| | | (equilibrative | | | | |
| | | nucleoside | | | | |
| | | transporter), | | | | |
| | | member 3 | | | | |
| 100507513 | LOC100507513 | uncharacterized | 2.03 | −1.15 | 0.05 | 0.97 |
| | | LOC100507513 | | | | |
| 55704 | CCDC88A | coiled-coil | 2.03 | 1.88 | 0.22 | 0.49 |
| | | domain | | | | |
| | | containing | | | | |
| | | 88A | | | | |
| 57604 | KIAA1456 | KIAA1456 | 2.03 | −0.16 | −0.62 | 0.49 |
| 10449 | ACAA2 | acetyl-CoA | 2.03 | 1.55 | 0.62 | 1.05 |
| | | acyltransferase 2 | | | | |
| 1727 | CYB5R3 | cytochrome | 2.03 | 0.58 | 0.39 | 0.45 |
| | | b5 reductase 3 | | | | |
| 9569 | GTF2IRD1 | GTF2I repeat | 2.03 | 0.08 | 0.45 | −0.51 |
| | | domain | | | | |
| | | containing 1 | | | | |
| 10009 | ZBTB33 | zinc finger | 2.02 | 0.68 | 0.25 | −0.42 |
| | | and BTB | | | | |
| | | domain | | | | |
| | | containing 33 | | | | |
| 5188 | PET112 | PET112 | 2.02 | 0.76 | −0.91 | −0.05 |
| | | homolog | | | | |
| | | (yeast) | | | | |
| 2132 | EXT2 | exostosin | 2.02 | −1.04 | −0.77 | 0.03 |
| | | glycosyltransferase 2 | | | | |
| 2239 | GPC4 | glypican 4 | 2.02 | 0.42 | 0.29 | −0.66 |
| 56927 | GPR108 | G protein- | 2.02 | 1.42 | −0.90 | 0.43 |
| | | coupled | | | | |
| | | receptor 108 | | | | |
| 2950 | GSTP1 | glutathione S- | 2.02 | 0.83 | 0.88 | 0.20 |
| | | transferase pi 1 | | | | |
| 342538 | NACA2 | nascent | 2.02 | −0.55 | 0.84 | −0.30 |
| | | polypeptide- | | | | |
| | | associated | | | | |
| | | complex alpha | | | | |
| | | subunit 2 | | | | |
| 55218 | EXD2 | exonuclease | 2.02 | −1.15 | 0.39 | −0.59 |
| | | 3′-5′ domain | | | | |
| | | containing 2 | | | | |
| 151827 | LRRC34 | leucine rich | 2.01 | 0.27 | −0.27 | −0.19 |
| | | repeat | | | | |
| | | containing 34 | | | | |
| 64689 | GORASP1 | golgi | 2.01 | 0.93 | −0.18 | 0.50 |
| | | reassembly | | | | |
| | | stacking | | | | |
| | | protein 1, | | | | |
| | | 65 kDa | | | | |
| 10324 | KLHL41 | kelch-like | 2.01 | 0.59 | −0.19 | −0.04 |
| | | family | | | | |
| | | member 41 | | | | |
| 1314 | COPA | coatomer | 2.01 | 0.99 | 0.14 | 0.28 |
| | | protein | | | | |
| | | complex, | | | | |
| | | subunit alpha | | | | |
| 54928 | IMPAD1 | inositol | 2.01 | 0.18 | −0.91 | 0.38 |
| | | monophosphatase | | | | |
| | | domain | | | | |
| | | containing 1 | | | | |
| 8971 | H1FX | H1 histone | 2.01 | −0.07 | 0.18 | 0.12 |
| | | family, | | | | |
| | | member X | | | | |
| 79832 | QSER1 | glutamine and | 2.01 | −0.02 | −0.81 | 0.24 |
| | | serine rich 1 | | | | |
| 10150 | MBNL2 | muscleblind- | 2.00 | 0.03 | −0.91 | 0.81 |
| | | like splicing | | | | |
| | | regulator 2 | | | | |
| 8744 | TNFSF9 | tumor | 2.00 | 1.07 | 1.23 | −0.89 |
| | | necrosis factor | | | | |
| | | (ligand) | | | | |
| | | superfamily, | | | | |
| | | member 9 | | | | |
| 23287 | AGTPBP1 | ATP/GTP | −2.00 | 0.35 | −0.29 | −0.37 |
| | | binding | | | | |
| | | protein 1 | | | | |
| 606553 | C8orf49 | chromosome | −2.00 | 0.29 | 0.31 | −1.93 |
| | | 8 open | | | | |
| | | reading frame | | | | |
| | | 49 | | | | |
| 103 | ADAR | adenosine | −2.01 | 0.26 | 0.29 | 0.17 |
| | | deaminase, | | | | |
| | | RNA-specific | | | | |
| 55509 | BATF3 | basic leucine | −2.01 | 0.59 | 0.32 | 0.49 |
| | | zipper | | | | |
| | | transcription | | | | |
| | | factor, ATF- | | | | |
| | | like 3 | | | | |
| 729614 | FLJ37453 | uncharacterized | −2.01 | 0.28 | −0.34 | 0.59 |
| | | LOC729614 | | | | |
| 9241 | NOG | noggin | −2.01 | −0.80 | 0.41 | −1.06 |
| 26118 | WSB1 | WD repeat | −2.01 | 1.16 | −0.20 | 0.02 |
| | | and SOCS | | | | |
| | | box | | | | |
| | | containing 1 | | | | |
| 64400 | AKTIP | AKT | −2.01 | 0.09 | −0.33 | −0.94 |
| | | interacting | | | | |
| | | protein | | | | |
| 283237 | TTC9C | tetratricopeptide | −2.01 | 0.08 | 0.60 | −0.31 |
| | | repeat | | | | |
| | | domain 9C | | | | |
| 51155 | HN1 | hematological | −2.01 | 0.74 | 1.29 | 0.20 |
| | | and | | | | |
| | | neurological | | | | |
| | | expressed 1 | | | | |
| 18 | ABAT | 4- | −2.01 | 0.33 | 0.20 | 0.86 |
| | | aminobutyrate | | | | |
| | | aminotransferase | | | | |
| 22990 | PCNX | pecanex | −2.01 | 0.31 | −0.50 | −0.92 |
| | | homolog | | | | |
| | | (Drosophila) | | | | |
| 23347 | SMCHD1 | structural | −2.01 | 0.42 | 0.17 | −0.12 |
| | | maintenance | | | | |
| | | of | | | | |
| | | chromosomes | | | | |
| | | flexible hinge | | | | |
| | | domain | | | | |
| | | containing 1 | | | | |
| 83988 | NCALD | neurocalcin | −2.01 | 0.18 | 1.54 | −1.04 |
| | | delta | | | | |
| 3428 | IFI16 | interferon, | −2.02 | 0.40 | 0.91 | −0.30 |
| | | gamma- | | | | |
| | | inducible | | | | |
| | | protein 16 | | | | |
| 3700 | ITIH4 | inter-alpha- | −2.02 | 0.45 | −0.47 | 1.50 |
| | | trypsin | | | | |
| | | inhibitor | | | | |
| | | heavy chain | | | | |
| | | family, | | | | |
| | | member 4 | | | | |
| 64859 | NABP1 | nucleic acid | −2.02 | 0.54 | −0.50 | −0.09 |
| | | binding | | | | |
| | | protein 1 | | | | |
| 55192 | DNAJC17 | DnaJ (Hsp40) | −2.02 | 0.39 | −0.28 | 0.68 |
| | | homolog, | | | | |
| | | subfamily C, | | | | |
| | | member 17 | | | | |
| 101928869 | LOC101928869 | uncharacterized | −2.02 | 0.74 | 1.59 | −0.82 |
| | | LOC101928869 | | | | |
| 10513 | APPBP2 | amyloid beta | −2.02 | −0.07 | −0.32 | −0.95 |
| | | precursor | | | | |
| | | protein | | | | |
| | | (cytoplasmic | | | | |
| | | tail) binding | | | | |
| | | protein 2 | | | | |
| 11009 | IL24 | interleukin 24 | −2.02 | −0.42 | 1.84 | −1.37 |
| 169355 | IDO2 | indoleamine | −2.02 | −1.28 | 0.08 | 0.42 |
| | | 2,3- | | | | |
| | | dioxygenase 2 | | | | |
| 10109 | ARPC2 | actin related | −2.02 | 0.60 | 0.53 | −0.23 |
| | | protein 2/3 | | | | |
| | | complex, | | | | |
| | | subunit 2, | | | | |
| | | 34 kDa | | | | |
| 9903 | KLHL21 | kelch-like | −2.02 | 0.11 | 0.37 | −0.40 |
| | | family | | | | |
| | | member 21 | | | | |
| 23150 | FRMD4B | FERM | −2.03 | 0.52 | −1.03 | −0.23 |
| | | domain | | | | |
| | | containing 4B | | | | |
| 10865 | ARID5A | AT rich | −2.03 | 1.11 | −0.22 | −0.06 |
| | | interactive | | | | |
| | | domain 5A | | | | |
| | | (MRF1-like) | | | | |
| 5165 | PDK3 | pyruvate | −2.03 | 0.23 | −0.04 | 0.48 |
| | | dehydrogenase | | | | |
| | | kinase, | | | | |
| | | isozyme 3 | | | | |
| 57403 | RAB22A | RAB22A, | −2.03 | 0.07 | −0.46 | −0.43 |
| | | member RAS | | | | |
| | | oncogene | | | | |
| | | family | | | | |
| 22900 | CARD8 | caspase | −2.03 | 0.12 | −0.63 | 0.28 |
| | | recruitment | | | | |
| | | domain | | | | |
| | | family, | | | | |
| | | member 8 | | | | |
| 80345 | ZSCAN16 | zinc finger | −2.03 | 0.25 | 0.38 | −0.29 |
| | | and SCAN | | | | |
| | | domain | | | | |
| | | containing 16 | | | | |
| 10964 | IFI44L | interferon- | −2.03 | 0.51 | 0.65 | 1.14 |
| | | induced | | | | |
| | | protein 44- | | | | |
| | | like | | | | |
| 27040 | LAT | linker for | −2.03 | −1.26 | 0.22 | 0.00 |
| | | activation of | | | | |
| | | T cells | | | | |
| 91010 | FMNL3 | formin-like 3 | −2.03 | −0.46 | 0.53 | −1.06 |
| 29065 | ASAP1- | ASAP1 | −2.04 | −0.05 | −0.37 | 0.50 |
| | IT1 | intronic | | | | |
| | | transcript 1 | | | | |
| | | (non-protein | | | | |
| | | coding) | | | | |
| 84078 | KBTBD7 | kelch repeat | −2.04 | 0.25 | 0.37 | −0.64 |
| | | and BTB | | | | |
| | | (POZ) domain | | | | |
| | | containing 7 | | | | |
| 101927910 | LOC101927910 | keratin- | −2.04 | −0.79 | 0.91 | 0.09 |
| | | associated | | | | |
| | | protein 5-5- | | | | |
| | | like | | | | |
| 51380 | CSAD | cysteine | −2.04 | 0.77 | −0.68 | 0.23 |
| | | sulfinic acid | | | | |
| | | decarboxylase | | | | |
| 10289 | EIF1B | eukaryotic | −2.04 | −0.15 | −0.05 | 0.65 |
| | | translation | | | | |
| | | initiation | | | | |
| | | factor 1B | | | | |
| 10296 | MAEA | macrophage | −2.04 | 0.53 | 0.45 | −0.22 |
| | | erythroblast | | | | |
| | | attacher | | | | |
| 114224 | PRO2852 | uncharacterized | −2.04 | −0.57 | −0.16 | 0.15 |
| | | protein | | | | |
| | | PRO2852 | | | | |
| 2153 | F5 | coagulation | −2.04 | −0.02 | −0.21 | 0.56 |
| | | factor V | | | | |
| | | (proaccelerin, | | | | |
| | | labile factor) | | | | |
| 55825 | PECR | peroxisomal | −2.04 | 0.44 | −0.03 | 0.40 |
| | | trans-2-enoyl- | | | | |
| | | CoA | | | | |
| | | reductase | | | | |
| 6503 | SLA | Src-like- | −2.04 | −1.37 | −0.53 | −0.03 |
| | | adaptor | | | | |
| 7913 | DEK | DEK | −2.04 | 0.66 | 0.32 | −0.64 |
| | | oncogene | | | | |
| 30834 | ZNRD1 | zinc ribbon | −2.04 | 0.24 | 2.03 | −1.06 |
| | | domain | | | | |
| | | containing 1 | | | | |
| 26999 | CYFIP2 | cytoplasmic | −2.05 | −0.51 | 0.45 | −0.46 |
| | | FMR1 | | | | |
| | | interacting | | | | |
| | | protein 2 | | | | |
| 286437 | LOC286437 | uncharacterized | −2.05 | −0.30 | −0.30 | 0.06 |
| | | LOC286437 | | | | |
| 4277 | MICB | MHC class I | −2.05 | −0.08 | 0.21 | −0.19 |
| | | polypeptide- | | | | |
| | | related | | | | |
| | | sequence B | | | | |
| 5698 | PSMB9 | proteasome | −2.05 | −0.12 | 1.18 | 0.14 |
| | | (prosome, | | | | |
| | | macropain) | | | | |
| | | subunit, beta | | | | |
| | | type, 9 | | | | |
| 4318 | MMP9 | matrix | −2.05 | 0.33 | −1.02 | −0.43 |
| | | metallopeptidase 9 | | | | |
| | | (gelatinase B, | | | | |
| | | 92 kDa | | | | |
| | | gelatinase, | | | | |
| | | 92 kDa type | | | | |
| | | IV | | | | |
| | | collagenase) | | | | |
| 10616 | RBCK1 | RanBP-type | −2.06 | 1.08 | 0.35 | 0.94 |
| | | and C3HC4- | | | | |
| | | type zinc | | | | |
| | | finger | | | | |
| | | containing 1 | | | | |
| 23012 | STK38L | serine/threonine | −2.06 | 0.24 | −0.56 | 0.06 |
| | | kinase 38 | | | | |
| | | like | | | | |
| 100129361 | LOC100129361 | chromosome | −2.06 | −0.72 | −1.53 | 1.08 |
| | | X open | | | | |
| | | reading frame | | | | |
| | | 69-like | | | | |
| 10742 | RAI2 | retinoic acid | −2.06 | −0.16 | 0.32 | −1.10 |
| | | induced 2 | | | | |
| 196264 | MPZL3 | myelin protein | −2.06 | 0.06 | −0.05 | −0.10 |
| | | zero-like 3 | | | | |
| 27197 | GPR82 | G protein- | −2.06 | −0.33 | 0.39 | 0.98 |
| | | coupled | | | | |
| | | receptor 82 | | | | |
| 7529 | YWHAB | tyrosine 3- | −2.06 | −0.33 | 0.35 | −0.87 |
| | | monooxygenase/ | | | | |
| | | tryptophan | | | | |
| | | 5- | | | | |
| | | monooxygenase | | | | |
| | | activation | | | | |
| | | protein, beta | | | | |
| | | polypeptide | | | | |
| 94235 | GNG8 | guanine | −2.06 | 0.07 | 0.57 | 1.14 |
| | | nucleotide | | | | |
| | | binding | | | | |
| | | protein (G | | | | |
| | | protein), | | | | |
| | | gamma 8 | | | | |
| 352961 | HCG26 | HLA complex | −2.06 | −0.34 | 0.92 | −0.34 |
| | | group 26 | | | | |
| | | (non-protein | | | | |
| | | coding) | | | | |
| 49856 | WRAP73 | WD repeat | −2.06 | 0.36 | 0.65 | −0.16 |
| | | containing, | | | | |
| | | antisense to | | | | |
| | | TP73 | | | | |
| 9448 | MAP4K4 | mitogen- | −2.06 | −0.11 | −0.96 | −0.07 |
| | | activated | | | | |
| | | protein kinase | | | | |
| | | kinase kinase | | | | |
| | | kinase 4 | | | | |
| 9525 | VPS4B | vacuolar | −2.06 | −0.15 | −0.27 | −1.47 |
| | | protein sorting | | | | |
| | | 4 homolog B | | | | |
| | | (S. cerevisiae) | | | | |
| 100505576 | LINC00672 | long | −2.06 | −0.65 | −1.38 | 0.24 |
| | | intergenic | | | | |
| | | non-protein | | | | |
| | | coding RNA | | | | |
| | | 672 | | | | |
| 151556 | GPR155 | G protein- | −2.06 | 0.09 | 0.08 | −0.13 |
| | | coupled | | | | |
| | | receptor 155 | | | | |
| 284613 | CYB561D1 | cytochrome | −2.06 | −0.63 | 0.38 | −0.70 |
| | | b561 family, | | | | |
| | | member D1 | | | | |
| 286223 | C9orf47 | chromosome | −2.06 | −0.58 | 1.33 | 0.81 |
| | | 9 open | | | | |
| | | reading frame | | | | |
| | | 47 | | | | |
| 57061 | HYMAI | hydatidiform | −2.06 | 0.13 | 1.06 | −0.57 |
| | | mole | | | | |
| | | associated and | | | | |
| | | imprinted | | | | |
| | | (non-protein | | | | |
| | | coding) | | | | |
| 9520 | NPEPPS | aminopeptidase | −2.07 | 0.62 | 0.08 | 0.22 |
| | | puromycin | | | | |
| | | sensitive | | | | |
| 202299 | C5orf27 | chromosome | −2.07 | −0.25 | 0.66 | 1.19 |
| | | 5 open | | | | |
| | | reading frame | | | | |
| | | 27 | | | | |
| 2015 | EMR1 | egf-like | −2.07 | 0.55 | 0.65 | 0.29 |
| | | module | | | | |
| | | containing, | | | | |
| | | mucin-like, | | | | |
| | | hormone | | | | |
| | | receptor-like 1 | | | | |
| 57763 | ANKRA2 | ankyrin | −2.07 | −0.20 | −1.67 | −0.42 |
| | | repeat, family | | | | |
| | | A (RFXANK- | | | | |
| | | like), 2 | | | | |
| 54778 | RNF111 | ring finger | −2.07 | 0.23 | −0.89 | −1.05 |
| | | protein 111 | | | | |
| 762 | CA4 | carbonic | −2.07 | 0.62 | −0.41 | 0.08 |
| | | anhydrase IV | | | | |
| 192668 | CYS1 | cystin 1 | −2.08 | −0.24 | 0.82 | 0.06 |
| 24146 | CLDN15 | claudin 15 | −2.08 | −1.46 | 0.60 | 0.44 |
| 54843 | SYTL2 | synaptotagmin- | −2.08 | −2.09 | −0.12 | −0.38 |
| | | like 2 | | | | |
| 55374 | TMCO6 | transmembrane | −2.08 | −0.03 | 0.41 | −0.36 |
| | | and coiled- | | | | |
| | | coil domains 6 | | | | |
| 79603 | CERS4 | ceramide | −2.08 | −0.47 | 1.17 | 0.72 |
| | | synthase 4 | | | | |
| 3577 | CXCR1 | chemokine | −2.08 | 0.24 | 0.01 | −0.45 |
| | | (C—X—C motif) | | | | |
| | | receptor 1 | | | | |
| 10385 | BTN2A2 | butyrophilin, | −2.08 | −0.42 | 0.63 | −0.30 |
| | | subfamily 2, | | | | |
| | | member A2 | | | | |
| 79792 | GSDMD | gasdermin D | −2.08 | 0.07 | 0.24 | 0.78 |
| 84674 | CARD6 | caspase | −2.09 | −0.13 | −0.05 | 0.28 |
| | | recruitment | | | | |
| | | domain | | | | |
| | | family, | | | | |
| | | member 6 | | | | |
| 51131 | PHF11 | PHD finger | −2.09 | −0.45 | 0.25 | −2.32 |
| | | protein 11 | | | | |
| 5966 | REL | v-rel avian | −2.09 | 0.51 | −0.20 | 0.66 |
| | | reticuloendotheliosis | | | | |
| | | viral | | | | |
| | | oncogene | | | | |
| | | homolog | | | | |
| 9733 | SART3 | squamous cell | −2.09 | −0.26 | 0.49 | 0.20 |
| | | carcinoma | | | | |
| | | antigen | | | | |
| | | recognized by | | | | |
| | | T cells 3 | | | | |
| 10328 | EMC8 | ER membrane | −2.09 | 0.24 | 0.80 | 0.87 |
| | | protein | | | | |
| | | complex | | | | |
| | | subunit 8 | | | | |
| 255231 | MCOLN2 | mucolipin 2 | −2.09 | 0.64 | 1.81 | −0.22 |
| 25801 | GCA | grancalcin, | −2.09 | −0.11 | 0.32 | −0.21 |
| | | EF-hand | | | | |
| | | calcium | | | | |
| | | binding | | | | |
| | | protein | | | | |
| 6352 | CCL5 | chemokine | −2.09 | −1.08 | 0.65 | 0.03 |
| | | (C-C motif) | | | | |
| | | ligand 5 | | | | |
| 54811 | ZNF562 | zinc finger | −2.09 | −1.12 | 0.98 | −0.04 |
| | | protein 562 | | | | |
| 59348 | ZNF350 | zinc finger | −2.09 | 0.46 | −0.12 | −0.32 |
| | | protein 350 | | | | |
| 9967 | THRAP3 | thyroid | −2.09 | 0.95 | 0.42 | 0.34 |
| | | hormone | | | | |
| | | receptor | | | | |
| | | associated | | | | |
| | | protein 3 | | | | |
| 2533 | FYB | FYN binding | −2.10 | −0.23 | −0.56 | 0.49 |
| | | protein | | | | |
| 28985 | MCTS1 | malignant T | −2.10 | −0.66 | −0.50 | −1.27 |
| | | cell amplified | | | | |
| | | sequence 1 | | | | |
| 55234 | SMU1 | smu-1 | −2.10 | 0.13 | −0.04 | −0.20 |
| | | suppressor of | | | | |
| | | mec-8 and | | | | |
| | | unc-52 | | | | |
| | | homolog (C. elegans) | | | | |
| 1301 | COL11A1 | collagen, type | −2.10 | 0.31 | 0.13 | 1.14 |
| | | XI, alpha 1 | | | | |
| 84803 | AGPAT9 | 1- | −2.10 | 0.07 | −0.17 | 0.02 |
| | | acylglycerol- | | | | |
| | | 3-phosphate | | | | |
| | | O- | | | | |
| | | acyltransferase 9 | | | | |
| 100506779 | BZRAP1- | BZRAP1 | −2.10 | −0.43 | 0.34 | 0.18 |
| | AS1 | antisense | | | | |
| | | RNA 1 | | | | |
| 197135 | PATL2 | protein | −2.10 | −0.08 | 0.66 | 0.09 |
| | | associated | | | | |
| | | with | | | | |
| | | topoisomerase | | | | |
| | | II homolog 2 | | | | |
| | | (yeast) | | | | |
| 3614 | IMPDH1 | IMP (inosine | −2.10 | 0.10 | 1.24 | 0.26 |
| | | 5′- | | | | |
| | | monophosphate) | | | | |
| | | dehydrogenase 1 | | | | |
| 80781 | COL18A1 | collagen, type | −2.10 | −1.15 | −0.88 | −0.81 |
| | | XVIII, alpha 1 | | | | |
| 5586 | PKN2 | protein kinase | −2.11 | −0.01 | −1.33 | −1.12 |
| | | N2 | | | | |
| 79415 | C17orf62 | chromosome | −2.11 | 0.36 | 0.69 | −0.55 |
| | | 17 open | | | | |
| | | reading frame | | | | |
| | | 62 | | | | |
| 8893 | EIF2B5 | eukaryotic | −2.11 | −0.42 | 0.14 | 0.02 |
| | | translation | | | | |
| | | initiation | | | | |
| | | factor 2B, | | | | |
| | | subunit 5 | | | | |
| | | epsilon, | | | | |
| | | 82 kDa | | | | |
| 79736 | TEFM | transcription | −2.11 | 0.10 | −0.38 | 0.03 |
| | | elongation | | | | |
| | | factor, | | | | |
| | | mitochondrial | | | | |
| 5770 | PTPN1 | protein | −2.11 | −0.21 | −0.09 | 0.43 |
| | | tyrosine | | | | |
| | | phosphatase, | | | | |
| | | non-receptor | | | | |
| | | type 1 | | | | |
| 9360 | PPIG | peptidylprolyl | −2.11 | 0.14 | −1.09 | −0.02 |
| | | isomerase G | | | | |
| | | (cyclophilin G) | | | | |
| 29940 | DSE | dermatan | −2.11 | 0.22 | 0.11 | 0.18 |
| | | sulfate | | | | |
| | | epimerase | | | | |
| 7150 | TOP1 | topoisomerase | −2.11 | 0.15 | 0.58 | 1.17 |
| | | (DNA) I | | | | |
| 84964 | ALKBH6 | alkB, | −2.11 | 0.05 | 0.45 | 0.79 |
| | | alkylation | | | | |
| | | repair | | | | |
| | | homolog 6 (E. coli) | | | | |
| 10614 | HEXIM1 | hexamethylene | −2.11 | −0.43 | −0.78 | 1.49 |
| | | bis- | | | | |
| | | acetamide | | | | |
| | | inducible 1 | | | | |
| 147923 | ZNF420 | zinc finger | −2.12 | −1.30 | −0.74 | −0.71 |
| | | protein 420 | | | | |
| 284029 | LINC00324 | long | −2.12 | −0.70 | −0.55 | 0.21 |
| | | intergenic | | | | |
| | | non-protein | | | | |
| | | coding RNA | | | | |
| | | 324 | | | | |
| 54952 | TRNAU1AP | tRNA | −2.12 | 0.49 | 0.10 | 0.73 |
| | | selenocysteine | | | | |
| | | 1 associated | | | | |
| | | protein 1 | | | | |
| 57476 | GRAMD1B | GRAM | −2.12 | −0.35 | 1.18 | 0.80 |
| | | domain | | | | |
| | | containing 1B | | | | |
| 8745 | ADAM23 | ADAM | −2.12 | 0.14 | −0.79 | 0.23 |
| | | metallopeptidase | | | | |
| | | domain 23 | | | | |
| 22928 | SEPHS2 | selenophosphate | −2.12 | 0.48 | −0.36 | −0.41 |
| | | synthetase 2 | | | | |
| 25880 | TMEM186 | transmembrane | −2.12 | −0.84 | 0.69 | −0.26 |
| | | protein 186 | | | | |
| 28638 | TRBC2 | T cell receptor | −2.12 | −0.74 | 0.65 | 0.15 |
| | | beta constant 2 | | | | |
| 29933 | GPR132 | G protein- | −2.12 | 1.64 | −0.07 | −0.04 |
| | | coupled | | | | |
| | | receptor 132 | | | | |
| 30009 | TBX21 | T-box 21 | −2.12 | −1.00 | 1.25 | 0.12 |
| 3554 | IL1R1 | interleukin 1 | −2.12 | 0.17 | −0.48 | −0.46 |
| | | receptor, type I | | | | |
| 8821 | INPP4B | inositol | −2.12 | −1.19 | −0.83 | 0.02 |
| | | polyphosphate- | | | | |
| | | 4- | | | | |
| | | phosphatase, | | | | |
| | | type II, | | | | |
| | | 105 kDa | | | | |
| 10943 | MSL3 | male-specific | −2.12 | 0.46 | 0.07 | 0.11 |
| | | lethal 3 | | | | |
| | | homolog | | | | |
| | | (Drosophila) | | | | |
| 57724 | EPG5 | ectopic P- | −2.12 | −0.33 | −0.06 | 0.11 |
| | | granules | | | | |
| | | autophagy | | | | |
| | | protein 5 | | | | |
| | | homolog (C. elegans) | | | | |
| 100506282 | LOC100506282 | uncharacterized | −2.13 | 0.16 | 0.83 | 1.50 |
| | | LOC100506282 | | | | |
| 147699 | PPM1N | protein | −2.13 | −1.19 | −0.54 | 0.79 |
| | | phosphatase, | | | | |
| | | Mg2+/Mn2+ | | | | |
| | | dependent, 1N | | | | |
| | | (putative) | | | | |
| 3002 | GZMB | granzyme B | −2.13 | −1.41 | 0.38 | 0.70 |
| | | (granzyme 2, | | | | |
| | | cytotoxic T- | | | | |
| | | lymphocyte- | | | | |
| | | associated | | | | |
| | | serine esterase | | | | |
| | | 1) | | | | |
| 389320 | C5orf48 | chromosome | −2.13 | 0.36 | 0.46 | −0.93 |
| | | 5 open | | | | |
| | | reading frame | | | | |
| | | 48 | | | | |
| 9914 | ATP2C2 | ATPase, | −2.13 | −0.05 | −0.48 | −0.11 |
| | | Ca++ | | | | |
| | | transporting, | | | | |
| | | type 2C, | | | | |
| | | member 2 | | | | |
| 10804 | GJB6 | gap junction | −2.13 | 0.05 | −0.09 | −0.70 |
| | | protein, beta | | | | |
| | | 6, 30 kDa | | | | |
| 51439 | FAM8A1 | family with | −2.13 | −0.42 | −1.15 | −0.36 |
| | | sequence | | | | |
| | | similarity 8, | | | | |
| | | member A1 | | | | |
| 814 | CAMK4 | calcium/calmodulin- | −2.13 | 0.28 | 0.24 | 0.04 |
| | | dependent | | | | |
| | | protein kinase | | | | |
| | | IV | | | | |
| 5272 | SERPINB9 | serpin | −2.13 | 0.11 | 1.70 | 0.00 |
| | | peptidase | | | | |
| | | inhibitor, | | | | |
| | | clade B | | | | |
| | | (ovalbumin), | | | | |
| | | member 9 | | | | |
| 8428 | STK24 | serine/threonine | −2.13 | −0.94 | 0.54 | 0.28 |
| | | kinase 24 | | | | |
| 23048 | FNBP1 | formin | −2.14 | −0.45 | 1.48 | 0.04 |
| | | binding | | | | |
| | | protein 1 | | | | |
| 344558 | SH3RF3 | SH3 domain | −2.14 | 0.78 | −0.14 | −0.92 |
| | | containing | | | | |
| | | ring finger 3 | | | | |
| 79078 | C1orf50 | chromosome | −2.14 | −0.55 | 1.96 | 1.42 |
| | | 1 open | | | | |
| | | reading frame | | | | |
| | | 50 | | | | |
| 81606 | LBH | limb bud and | −2.14 | −1.45 | 0.75 | −0.43 |
| | | heart | | | | |
| | | development | | | | |
| 64377 | CHST8 | carbohydrate | −2.14 | −0.27 | 0.39 | −0.34 |
| | | (N- | | | | |
| | | acetylgalactosamine | | | | |
| | | 4-0) | | | | |
| | | sulfotransferase 8 | | | | |
| 57501 | KIAA1257 | KIAA1257 | −2.14 | −0.35 | −0.74 | 0.39 |
| 5991 | RFX3 | regulatory | −2.14 | 0.67 | −0.98 | 0.29 |
| | | factor X, 3 | | | | |
| | | (influences | | | | |
| | | HLA class II | | | | |
| | | expression) | | | | |
| 90075 | ZNF30 | zinc finger | −2.14 | −0.45 | 0.73 | −0.98 |
| | | protein 30 | | | | |
| 375341 | C3orf62 | chromosome | −2.14 | 0.54 | 0.27 | 0.16 |
| | | 3 open | | | | |
| | | reading frame | | | | |
| | | 62 | | | | |
| 100130548 | LOC100130548 | uncharacterized | −2.15 | −0.60 | 0.80 | 0.14 |
| | | LOC100130548 | | | | |
| 114786 | XKR4 | XK, Kell | −2.15 | −0.80 | −0.51 | −0.43 |
| | | blood group | | | | |
| | | complex | | | | |
| | | subunit- | | | | |
| | | related family, | | | | |
| | | member 4 | | | | |
| 150084 | IGSF5 | immunoglobulin | −2.15 | 0.53 | 0.83 | −0.22 |
| | | superfamily, | | | | |
| | | member 5 | | | | |
| 55012 | PPP2R3C | protein | −2.15 | 0.50 | −0.18 | 0.28 |
| | | phosphatase | | | | |
| | | 2, regulatory | | | | |
| | | subunit B”, | | | | |
| | | gamma | | | | |
| 147525 | LINC00526 | long | −2.15 | 0.01 | −0.55 | 0.90 |
| | | intergenic | | | | |
| | | non-protein | | | | |
| | | coding RNA | | | | |
| | | 526 | | | | |
| 54557 | SGTB | small | −2.15 | 0.23 | −0.78 | 0.10 |
| | | glutamine- | | | | |
| | | rich | | | | |
| | | tetratricopeptide | | | | |
| | | repeat | | | | |
| | | (TPR)- | | | | |
| | | containing, | | | | |
| | | beta | | | | |
| 56267 | CCBL2 | cysteine | −2.15 | 0.68 | 0.94 | −1.30 |
| | | conjugate- | | | | |
| | | beta lyase 2 | | | | |
| 51176 | LEF1 | lymphoid | −2.15 | −0.06 | −0.04 | 0.50 |
| | | enhancer- | | | | |
| | | binding factor 1 | | | | |
| 10468 | FST | follistatin | −2.15 | −0.20 | 0.87 | 0.11 |
| 23258 | DENND5A | DENN/MADD | −2.15 | 0.12 | 0.07 | −0.02 |
| | | domain | | | | |
| | | containing 5A | | | | |
| 90768 | MGC45800 | uncharacterized | −2.15 | −1.02 | 1.59 | 0.00 |
| | | LOC90768 | | | | |
| 134957 | STXBP5 | syntaxin | −2.16 | 0.01 | −0.56 | −0.01 |
| | | binding | | | | |
| | | protein 5 | | | | |
| | | (tomosyn) | | | | |
| 80342 | TRAF3IP3 | TRAF3 | −2.16 | −0.22 | 1.35 | −0.63 |
| | | interacting | | | | |
| | | protein 3 | | | | |
| 91351 | DDX60L | DEAD (Asp- | −2.16 | 0.33 | −0.81 | −0.18 |
| | | Glu-Ala-Asp) | | | | |
| | | box | | | | |
| | | polypeptide | | | | |
| | | 60-like | | | | |
| 29916 | SNX11 | sorting nexin | −2.16 | 0.44 | 0.38 | 0.62 |
| | | 11 | | | | |
| 8519 | IFITM1 | interferon | −2.16 | 0.04 | 0.54 | 0.00 |
| | | induced | | | | |
| | | transmembrane | | | | |
| | | protein 1 | | | | |
| 2113 | ETS1 | v-ets avian | −2.16 | −0.22 | 1.20 | −0.30 |
| | | erythroblastosis | | | | |
| | | virus E26 | | | | |
| | | oncogene | | | | |
| | | homolog 1 | | | | |
| 55770 | EXOC2 | exocyst | −2.16 | 0.23 | −0.28 | −2.07 |
| | | complex | | | | |
| | | component 2 | | | | |
| 91833 | WDR20 | WD repeat | −2.16 | 0.26 | −0.47 | −0.62 |
| | | domain 20 | | | | |
| 117177 | RAB3IP | RAB3A | −2.16 | −0.84 | −0.71 | 0.52 |
| | | interacting | | | | |
| | | protein | | | | |
| 546 | ATRX | alpha | −2.16 | 0.61 | 0.17 | 0.22 |
| | | thalassemia/mental | | | | |
| | | retardation | | | | |
| | | syndrome X- | | | | |
| | | linked | | | | |
| 4253 | CTAGE5 | CTAGE | −2.17 | −0.29 | −0.89 | −0.52 |
| | | family, | | | | |
| | | member 5 | | | | |
| 23214 | XPO6 | exportin 6 | −2.17 | 0.22 | 0.51 | −0.08 |
| 53347 | UBASH3A | ubiquitin | −2.17 | −0.77 | 0.40 | 0.23 |
| | | associated and | | | | |
| | | SH3 domain | | | | |
| | | containing A | | | | |
| 56888 | KCMF1 | potassium | −2.17 | −1.33 | −0.12 | −0.16 |
| | | channel | | | | |
| | | modulatory | | | | |
| | | factor 1 | | | | |
| 777 | CACNA1E | calcium | −2.17 | 0.75 | −0.59 | 0.85 |
| | | channel, | | | | |
| | | voltage- | | | | |
| | | dependent, R | | | | |
| | | type, alpha 1E | | | | |
| | | subunit | | | | |
| 100216545 | KMT2E- | KMT2E | −2.17 | −0.69 | −0.17 | 0.88 |
| | AS1 | antisense | | | | |
| | | RNA 1 (head | | | | |
| | | to head) | | | | |
| 8879 | SGPL1 | sphingosine- | −2.17 | 0.23 | −0.11 | −1.18 |
| | | 1-phosphate | | | | |
| | | lyase 1 | | | | |
| 9759 | HDAC4 | histone | −2.17 | −0.04 | 0.01 | −0.28 |
| | | deacetylase 4 | | | | |
| 26578 | OSTF1 | osteoclast | −2.18 | 0.31 | −0.12 | 0.48 |
| | | stimulating | | | | |
| | | factor 1 | | | | |
| 4050 | LTB | lymphotoxin | −2.18 | −0.37 | 0.26 | 0.38 |
| | | beta (TNF | | | | |
| | | superfamily, | | | | |
| | | member 3) | | | | |
| 4236 | MFAP1 | microfibrillar- | −2.18 | 0.04 | 2.06 | −1.26 |
| | | associated | | | | |
| | | protein 1 | | | | |
| 54491 | FAM105A | family with | −2.18 | −0.90 | −0.58 | −0.19 |
| | | sequence | | | | |
| | | similarity 105, | | | | |
| | | member A | | | | |
| 55847 | CISD1 | CDGSH iron | −2.18 | −0.27 | −0.72 | −0.14 |
| | | sulfur domain 1 | | | | |
| 100132707 | PAXIP1- | PAXIP1 | −2.19 | 0.30 | 0.46 | −0.02 |
| | AS2 | antisense | | | | |
| | | RNA 2 | | | | |
| 55884 | WSB2 | WD repeat | −2.19 | 0.26 | 0.47 | −0.19 |
| | | and SOCS | | | | |
| | | box | | | | |
| | | containing 2 | | | | |
| 83607 | AMMECR1L | AMMECR1- | −2.19 | −1.27 | −0.33 | −0.57 |
| | | like | | | | |
| 9252 | RPS6KA5 | ribosomal | −2.19 | −0.65 | −0.33 | −0.72 |
| | | protein S6 | | | | |
| | | kinase, | | | | |
| | | 90 kDa, | | | | |
| | | polypeptide 5 | | | | |
| 55119 | PRPF38B | pre-mRNA | −2.19 | 0.01 | −0.47 | −2.06 |
| | | processing | | | | |
| | | factor 38B | | | | |
| 57520 | HECW2 | HECT, C2 | −2.19 | 0.49 | 0.23 | 0.37 |
| | | and WW | | | | |
| | | domain | | | | |
| | | containing E3 | | | | |
| | | ubiquitin | | | | |
| | | protein ligase 2 | | | | |
| 79818 | ZNF552 | zinc finger | −2.19 | 0.12 | −0.32 | −0.44 |
| | | protein 552 | | | | |
| 8320 | EOMES | eomesodermin | −2.19 | −1.37 | 0.09 | 0.09 |
| 2792 | GNGT1 | guanine | −2.19 | −1.06 | 1.52 | −0.42 |
| | | nucleotide | | | | |
| | | binding | | | | |
| | | protein (G | | | | |
| | | protein), | | | | |
| | | gamma | | | | |
| | | transducing | | | | |
| | | activity | | | | |
| | | polypeptide 1 | | | | |
| 54910 | SEMA4C | sema domain, | −2.19 | 0.12 | 1.29 | −0.78 |
| | | immunoglobulin | | | | |
| | | domain | | | | |
| | | (Ig), | | | | |
| | | transmembrane | | | | |
| | | domain | | | | |
| | | (TM) and | | | | |
| | | short | | | | |
| | | cytoplasmic | | | | |
| | | domain, | | | | |
| | | (semaphorin) | | | | |
| | | 4C | | | | |
| 55114 | ARHGAP17 | Rho GTPase | −2.19 | 1.40 | 1.57 | 0.35 |
| | | activating | | | | |
| | | protein 17 | | | | |
| 56253 | CRTAM | cytotoxic and | −2.19 | −1.83 | −1.11 | −0.04 |
| | | regulatory T | | | | |
| | | cell molecule | | | | |
| 65082 | VPS33A | vacuolar | −2.19 | −0.53 | 0.02 | −0.62 |
| | | protein sorting | | | | |
| | | 33 homolog A | | | | |
| | | (S. cerevisiae) | | | | |
| 84719 | LINC00260 | long | −2.20 | −0.34 | −0.71 | −0.06 |
| | | intergenic | | | | |
| | | non-protein | | | | |
| | | coding RNA | | | | |
| | | 260 | | | | |
| 253558 | LCLAT1 | lysocardiolipin | −2.20 | −0.69 | −1.32 | −1.73 |
| | | acyltransferase 1 | | | | |
| 6777 | STAT5B | signal | −2.20 | 0.44 | 0.37 | 0.15 |
| | | transducer and | | | | |
| | | activator of | | | | |
| | | transcription | | | | |
| | | 5B | | | | |
| 80025 | PANK2 | pantothenate | −2.20 | −0.25 | 1.01 | 0.64 |
| | | kinase 2 | | | | |
| 84662 | GLIS2 | GLIS family | −2.20 | 0.50 | 0.17 | 0.54 |
| | | zinc finger 2 | | | | |
| 89890 | KBTBD6 | kelch repeat | −2.20 | 0.15 | −0.79 | −1.41 |
| | | and BTB | | | | |
| | | (POZ) domain | | | | |
| | | containing 6 | | | | |
| 10390 | CEPT1 | choline/ethanolamine | −2.20 | 0.37 | −0.08 | −0.71 |
| | | phosphotransferase 1 | | | | |
| 2841 | GPR18 | G protein- | −2.20 | −0.65 | 0.95 | −0.34 |
| | | coupled | | | | |
| | | receptor 18 | | | | |
| 3904 | LAIR2 | leukocyte- | −2.20 | −1.10 | 1.23 | 0.37 |
| | | associated | | | | |
| | | immunoglobulin- | | | | |
| | | like | | | | |
| | | receptor 2 | | | | |
| 140460 | ASB7 | ankyrin repeat | −2.20 | −0.05 | −0.77 | 0.23 |
| | | and SOCS | | | | |
| | | box | | | | |
| | | containing 7 | | | | |
| 9605 | VPS9D1 | VPS9 domain | −2.20 | 0.25 | −0.40 | 0.11 |
| | | containing 1 | | | | |
| 283349 | RASSF3 | Ras | −2.21 | 0.55 | −0.19 | −0.38 |
| | | association | | | | |
| | | (RalGDS/AF- | | | | |
| | | 6) domain | | | | |
| | | family | | | | |
| | | member 3 | | | | |
| 51246 | SHISA5 | shisa family | −2.21 | −0.38 | −0.02 | −0.61 |
| | | member 5 | | | | |
| 148268 | ZNF570 | zinc finger | −2.21 | 0.02 | −1.49 | −0.35 |
| | | protein 570 | | | | |
| 26589 | MRPL46 | mitochondrial | −2.21 | −0.39 | 0.36 | 0.93 |
| | | ribosomal | | | | |
| | | protein L46 | | | | |
| 7049 | TGFBR3 | transforming | −2.21 | −0.97 | −0.32 | 0.21 |
| | | growth factor, | | | | |
| | | beta receptor | | | | |
| | | III | | | | |
| 2791 | GNG11 | guanine | −2.21 | −0.32 | −1.05 | 0.18 |
| | | nucleotide | | | | |
| | | binding | | | | |
| | | protein (G | | | | |
| | | protein), | | | | |
| | | gamma 11 | | | | |
| 51455 | REV1 | REV1, | −2.21 | −0.30 | −1.28 | −1.13 |
| | | polymerase | | | | |
| | | (DNA | | | | |
| | | directed) | | | | |
| 1178 | CLC | Charcot- | −2.22 | −0.06 | −0.39 | −0.16 |
| | | Leyden | | | | |
| | | crystal | | | | |
| | | galectin | | | | |
| 79153 | GDPD3 | glycerophosphodiester | −2.22 | 0.49 | −0.68 | −0.16 |
| | | phosphodiesterase | | | | |
| | | domain | | | | |
| | | containing 3 | | | | |
| 101928524 | LOC101928524 | uncharacterized | −2.22 | 0.14 | −0.26 | −0.07 |
| | | LOC101928524 | | | | |
| 11064 | CNTRL | centriolin | −2.22 | 0.51 | 0.62 | −0.38 |
| 23429 | RYBP | RING1 and | −2.22 | −1.24 | 0.30 | 1.60 |
| | | YY1 binding | | | | |
| | | protein | | | | |
| 5170 | PDPK1 | 3- | −2.22 | 0.00 | −0.60 | 0.45 |
| | | phosphoinositide | | | | |
| | | dependent | | | | |
| | | protein | | | | |
| | | kinase-1 | | | | |
| 10677 | AVIL | advillin | −2.23 | 0.30 | −0.08 | −0.01 |
| 3772 | KCNJ15 | potassium | −2.23 | 0.23 | 0.26 | −0.02 |
| | | inwardly- | | | | |
| | | rectifying | | | | |
| | | channel, | | | | |
| | | subfamily J, | | | | |
| | | member 15 | | | | |
| 128061 | C1orf131 | chromosome | −2.23 | −0.47 | −0.90 | −0.33 |
| | | 1 open | | | | |
| | | reading frame | | | | |
| | | 131 | | | | |
| 50856 | CLEC4A | C-type lectin | −2.23 | 0.15 | 0.14 | 0.43 |
| | | domain family | | | | |
| | | 4, member A | | | | |
| 6198 | RPS6KB1 | ribosomal | −2.23 | −0.21 | −0.73 | −0.35 |
| | | protein S6 | | | | |
| | | kinase, | | | | |
| | | 70 kDa, | | | | |
| | | polypeptide 1 | | | | |
| 101927588 | LOC101927588 | uncharacterized | −2.24 | 0.09 | 0.17 | −0.36 |
| | | LOC101927588 | | | | |
| 2000 | ELF4 | E74-like | −2.24 | 0.39 | 0.72 | −0.14 |
| | | factor 4 (ets | | | | |
| | | domain | | | | |
| | | transcription | | | | |
| | | factor) | | | | |
| 493753 | COA5 | cytochrome c | −2.24 | −0.80 | −0.63 | −1.24 |
| | | oxidase | | | | |
| | | assembly | | | | |
| | | factor 5 | | | | |
| 23081 | KDM4C | lysine (K)- | −2.24 | −0.52 | −0.13 | −1.00 |
| | | specific | | | | |
| | | demethylase | | | | |
| | | 4C | | | | |
| 4773 | NFATC2 | nuclear factor | −2.24 | −1.02 | 0.77 | −0.22 |
| | | of activated | | | | |
| | | T-cells, | | | | |
| | | cytoplasmic, | | | | |
| | | calcineurin- | | | | |
| | | dependent 2 | | | | |
| 10178 | TENM1 | teneurin | −2.25 | −0.12 | −0.96 | 0.43 |
| | | transmembrane | | | | |
| | | protein 1 | | | | |
| 4974 | OMG | oligodendrocyte | −2.25 | 0.29 | −0.19 | 0.31 |
| | | myelin | | | | |
| | | glycoprotein | | | | |
| 83860 | TAF3 | TAF3 RNA | −2.25 | 0.12 | 0.93 | 0.61 |
| | | polymerase II, | | | | |
| | | TATA box | | | | |
| | | binding | | | | |
| | | protein | | | | |
| | | (TBP)- | | | | |
| | | associated | | | | |
| | | factor, | | | | |
| | | 140 kDa | | | | |
| 91612 | CHURC1 | churchill | −2.25 | −0.24 | 0.27 | −0.30 |
| | | domain | | | | |
| | | containing 1 | | | | |
| 2178 | FANCE | Fanconi | −2.26 | −0.56 | 0.71 | −0.52 |
| | | anemia, | | | | |
| | | complementation | | | | |
| | | group E | | | | |
| 55303 | GIMAP4 | GTPase, | −2.26 | −0.37 | 0.14 | −0.44 |
| | | IMAP family | | | | |
| | | member 4 | | | | |
| 221002 | RASGEF1A | RasGEF | −2.26 | −0.30 | −0.94 | −0.21 |
| | | domain | | | | |
| | | family, | | | | |
| | | member 1A | | | | |
| 3930 | LBR | lamin B | −2.26 | 0.09 | 0.14 | −0.49 |
| | | receptor | | | | |
| 55471 | NDUFAF7 | NADH | −2.26 | 0.97 | −0.55 | −0.62 |
| | | dehydrogenase | | | | |
| | | (ubiquinone) | | | | |
| | | complex I, | | | | |
| | | assembly | | | | |
| | | factor 7 | | | | |
| 9354 | UBE4A | ubiquitination | −2.26 | −0.27 | −0.43 | −2.87 |
| | | factor E4A | | | | |
| 83700 | JAM3 | junctional | −2.26 | 0.00 | −0.65 | 0.67 |
| | | adhesion | | | | |
| | | molecule 3 | | | | |
| 9140 | ATG12 | autophagy | −2.26 | −0.12 | −1.78 | 1.02 |
| | | related 12 | | | | |
| 23526 | HMHA1 | histocompatibility | −2.27 | −0.29 | 0.34 | 0.54 |
| | | (minor) | | | | |
| | | HA-1 | | | | |
| 23590 | PDSS1 | prenyl | −2.27 | 1.23 | 0.14 | −1.07 |
| | | (decaprenyl) | | | | |
| | | diphosphate | | | | |
| | | synthase, | | | | |
| | | subunit 1 | | | | |
| 9270 | ITGB1BP1 | integrin beta 1 | −2.27 | −0.53 | 0.25 | −0.72 |
| | | binding | | | | |
| | | protein 1 | | | | |
| 10627 | MYL12A | myosin, light | −2.27 | 0.89 | 0.00 | 0.29 |
| | | chain 12A, | | | | |
| | | regulatory, | | | | |
| | | non- | | | | |
| | | sarcomeric | | | | |
| 26048 | ZNF500 | zinc finger | −2.27 | −1.18 | 0.18 | 0.15 |
| | | protein 500 | | | | |
| 917 | CD3G | CD3g | −2.27 | −1.58 | 0.01 | −0.12 |
| | | molecule, | | | | |
| | | gamma (CD3- | | | | |
| | | TCR | | | | |
| | | complex) | | | | |
| 94039 | ZNF101 | zinc finger | −2.27 | 0.06 | −0.61 | −0.07 |
| | | protein 101 | | | | |
| 4215 | MAP3K3 | mitogen- | −2.28 | 0.26 | −0.02 | −0.10 |
| | | activated | | | | |
| | | protein kinase | | | | |
| | | kinase kinase 3 | | | | |
| 10663 | CXCR6 | chemokine | −2.28 | −1.52 | 0.47 | −0.52 |
| | | (C—X—C motif) | | | | |
| | | receptor 6 | | | | |
| 10308 | ZNF267 | zinc finger | −2.28 | 0.84 | −0.65 | 0.10 |
| | | protein 267 | | | | |
| 8527 | DGKD | diacylglycerol | −2.28 | 0.47 | 0.01 | 0.03 |
| | | kinase, delta | | | | |
| | | 130 kDa | | | | |
| 926 | CD8B | CD8b | −2.28 | −0.59 | −0.11 | −0.36 |
| | | molecule | | | | |
| 9619 | ABCG1 | ATP-binding | −2.28 | −0.04 | −0.78 | −0.92 |
| | | cassette, sub- | | | | |
| | | family G | | | | |
| | | (WHITE), | | | | |
| | | member 1 | | | | |
| 5998 | RGS3 | regulator of | −2.29 | 0.44 | 0.97 | 1.13 |
| | | G-protein | | | | |
| | | signaling 3 | | | | |
| 132625 | ZFP42 | ZFP42 zinc | −2.29 | 0.20 | −1.36 | 0.61 |
| | | finger protein | | | | |
| 88455 | ANKRD13A | ankyrin repeat | −2.30 | −0.11 | −0.08 | −0.02 |
| | | domain 13A | | | | |
| 3604 | TNFRSF9 | tumor | −2.30 | 0.91 | 0.58 | −0.73 |
| | | necrosis factor | | | | |
| | | receptor | | | | |
| | | superfamily, | | | | |
| | | member 9 | | | | |
| 100507398 | INTS6- | INTS6 | −2.30 | −0.45 | −0.55 | 0.49 |
| | AS1 | antisense | | | | |
| | | RNA1 | | | | |
| 58517 | RBM25 | RNA binding | −2.30 | −0.20 | 0.40 | −0.90 |
| | | motif protein | | | | |
| | | 25 | | | | |
| 51192 | CKLF | chemokine- | −2.31 | −0.14 | −0.30 | −0.10 |
| | | like factor | | | | |
| 2177 | FANCD2 | Fanconi | −2.31 | 0.35 | −0.30 | −0.42 |
| | | anemia, | | | | |
| | | complementation | | | | |
| | | group D2 | | | | |
| 57097 | PARP11 | poly (ADP- | −2.31 | −0.33 | 0.54 | −0.09 |
| | | ribose) | | | | |
| | | polymerase | | | | |
| | | family, | | | | |
| | | member 11 | | | | |
| 11214 | AKAP13 | A kinase | −2.31 | 0.03 | −0.10 | 0.38 |
| | | (PRKA) | | | | |
| | | anchor protein | | | | |
| | | 13 | | | | |
| 4600 | MX2 | myxovirus | −2.31 | 0.55 | 0.95 | 0.27 |
| | | (influenza | | | | |
| | | virus) | | | | |
| | | resistance 2 | | | | |
| | | (mouse) | | | | |
| 84255 | SLC37A3 | solute carrier | −2.31 | 0.14 | −0.49 | 0.05 |
| | | family 37, | | | | |
| | | member 3 | | | | |
| 11216 | AKAP10 | A kinase | −2.32 | 0.60 | 0.72 | −0.32 |
| | | (PRKA) | | | | |
| | | anchor protein | | | | |
| | | 10 | | | | |
| 162417 | NAGS | N- | −2.32 | 0.51 | 0.99 | −0.07 |
| | | acetylglutamate | | | | |
| | | synthase | | | | |
| 101928617 | LOC101928617 | uncharacterized | −2.32 | −0.16 | −0.52 | 0.77 |
| | | LOC101928617 | | | | |
| 10617 | STAMBP | STAM | −2.32 | −0.46 | 1.22 | −0.28 |
| | | binding | | | | |
| | | protein | | | | |
| 1326 | MAP3K8 | mitogen- | −2.32 | 0.35 | −0.48 | −0.27 |
| | | activated | | | | |
| | | protein kinase | | | | |
| | | kinase kinase 8 | | | | |
| 23315 | SLC9A8 | solute carrier | −2.32 | 0.18 | 0.29 | 0.24 |
| | | family 9, | | | | |
| | | subfamily A | | | | |
| | | (NHE8, cation | | | | |
| | | proton | | | | |
| | | antiporter 8), | | | | |
| | | member 8 | | | | |
| 10791 | VAMP5 | vesicle- | −2.32 | 0.11 | 0.52 | 0.99 |
| | | associated | | | | |
| | | membrane | | | | |
| | | protein 5 | | | | |
| 8718 | TNFRSF25 | tumor | −2.32 | −0.41 | −0.63 | −0.05 |
| | | necrosis factor | | | | |
| | | receptor | | | | |
| | | superfamily, | | | | |
| | | member 25 | | | | |
| 51571 | FAM49B | family with | −2.33 | −0.21 | −0.09 | −0.32 |
| | | sequence | | | | |
| | | similarity 49, | | | | |
| | | member B | | | | |
| 283521 | LINC00282 | long | −2.33 | 0.15 | −0.37 | −0.21 |
| | | intergenic | | | | |
| | | non-protein | | | | |
| | | coding RNA | | | | |
| | | 282 | | | | |
| 51122 | COMMD2 | COMM | −2.33 | 0.10 | −0.79 | −0.17 |
| | | domain | | | | |
| | | containing 2 | | | | |
| 5372 | PMM1 | phosphomannomutase 1 | −2.33 | 0.90 | −0.91 | −0.37 |
| 9830 | TRIM14 | tripartite motif | −2.33 | −1.08 | 0.02 | −0.05 |
| | | containing 14 | | | | |
| 314 | AOC2 | amine | −2.33 | −0.51 | −0.06 | −1.03 |
| | | oxidase, | | | | |
| | | copper | | | | |
| | | containing 2 | | | | |
| | | (retina- | | | | |
| | | specific) | | | | |
| 51761 | ATP8A2 | ATPase, | −2.33 | −0.45 | 0.23 | −1.08 |
| | | aminophospholipid | | | | |
| | | transporter, | | | | |
| | | class I, type | | | | |
| | | 8A, member 2 | | | | |
| 493861 | EID3 | EP300 | −2.34 | −0.28 | −0.02 | 0.74 |
| | | interacting | | | | |
| | | inhibitor of | | | | |
| | | differentiation 3 | | | | |
| 6672 | SP100 | SP100 nuclear | −2.34 | 0.20 | 0.18 | 0.29 |
| | | antigen | | | | |
| 55578 | SUPT20H | suppressor of | −2.34 | 0.36 | 0.25 | −1.31 |
| | | Ty 20 | | | | |
| | | homolog (S. cerevisiae) | | | | |
| 159 | ADSS | adenylosuccinate | −2.34 | −0.25 | 0.57 | −1.73 |
| | | synthase | | | | |
| 9683 | N4BP1 | NEDD4 | −2.34 | −0.14 | −0.22 | −0.13 |
| | | binding | | | | |
| | | protein 1 | | | | |
| 26287 | ANKRD2 | ankyrin repeat | −2.35 | 0.50 | 1.19 | −0.57 |
| | | domain 2 | | | | |
| | | (stretch | | | | |
| | | responsive | | | | |
| | | muscle) | | | | |
| 57147 | SCYL3 | SCY1-like 3 | −2.35 | 0.42 | −0.45 | −1.01 |
| | | (S. cerevisiae) | | | | |
| 1808 | DPYSL2 | dihydropyrimidinase- | −2.35 | 1.25 | 0.58 | 0.09 |
| | | like 2 | | | | |
| 23355 | VPS8 | vacuolar | −2.35 | 0.05 | −0.51 | 0.89 |
| | | protein sorting | | | | |
| | | 8 homolog (S. cerevisiae) | | | | |
| 8115 | TCL1A | T-cell | −2.35 | 0.74 | 1.27 | 0.86 |
| | | leukemia/lymphoma | | | | |
| | | 1A | | | | |
| 94120 | SYTL3 | synaptotagmin- | −2.35 | −0.12 | 0.30 | −0.73 |
| | | like 3 | | | | |
| 22950 | SLC4A1AP | solute carrier | −2.35 | 0.00 | 0.35 | 0.02 |
| | | family 4 | | | | |
| | | (anion | | | | |
| | | exchanger), | | | | |
| | | member 1, | | | | |
| | | adaptor | | | | |
| | | protein | | | | |
| 27342 | RABGEF1 | RAB guanine | −2.35 | 0.57 | −0.34 | −0.05 |
| | | nucleotide | | | | |
| | | exchange | | | | |
| | | factor (GEF) 1 | | | | |
| 64926 | RASAL3 | RAS protein | −2.35 | −0.38 | 1.18 | −0.59 |
| | | activator like 3 | | | | |
| 100507331 | ZSWIM8- | ZSWIM8 | −2.36 | 0.19 | 0.03 | −0.24 |
| | AS1 | antisense | | | | |
| | | RNA 1 | | | | |
| 4068 | SH2D1A | SH2 domain | −2.36 | −0.89 | −0.83 | 0.04 |
| | | containing 1A | | | | |
| 60481 | ELOVL5 | ELOVL fatty | −2.36 | 0.40 | 0.75 | −0.39 |
| | | acid elongase 5 | | | | |
| 2634 | GBP2 | guanylate | −2.36 | −0.33 | −0.07 | −0.24 |
| | | binding | | | | |
| | | protein 2, | | | | |
| | | interferon- | | | | |
| | | inducible | | | | |
| 55824 | PAG1 | phosphoprotein | −2.36 | −0.30 | −0.42 | −0.20 |
| | | associated | | | | |
| | | with | | | | |
| | | glycosphingolipid | | | | |
| | | microdomains 1 | | | | |
| 79074 | C2orf49 | chromosome | −2.36 | 0.81 | 0.03 | 0.36 |
| | | 2 open | | | | |
| | | reading frame | | | | |
| | | 49 | | | | |
| 220930 | ZEB1- | ZEB1 | −2.36 | 0.53 | −1.05 | −1.07 |
| | AS1 | antisense | | | | |
| | | RNA 1 | | | | |
| 64421 | DCLRE1C | DNA cross- | −2.36 | 0.02 | −1.42 | 0.49 |
| | | link repair 1C | | | | |
| 64121 | RRAGC | Ras-related | −2.37 | −0.29 | 0.24 | 1.00 |
| | | GTP binding C | | | | |
| 137835 | TMEM71 | transmembrane | −2.37 | −0.30 | −0.26 | −0.04 |
| | | protein 71 | | | | |
| 57674 | RNF213 | ring finger | −2.37 | 0.30 | 0.22 | 0.34 |
| | | protein 213 | | | | |
| 728392 | LOC728392 | uncharacterized | −2.37 | 0.26 | −0.69 | −0.19 |
| | | LOC728392 | | | | |
| 329 | BIRC2 | baculoviral | −2.38 | −0.70 | −2.00 | 0.48 |
| | | IAP repeat | | | | |
| | | containing 2 | | | | |
| 55279 | ZNF654 | zinc finger | −2.38 | 0.07 | −1.37 | −0.19 |
| | | protein 654 | | | | |
| 7107 | GPR137B | G protein- | −2.38 | 0.31 | −0.09 | 0.76 |
| | | coupled | | | | |
| | | receptor 137B | | | | |
| 9416 | DDX23 | DEAD (Asp- | −2.38 | 0.38 | 0.91 | −0.29 |
| | | Glu-Ala-Asp) | | | | |
| | | box | | | | |
| | | polypeptide | | | | |
| | | 23 | | | | |
| 137964 | AGPAT6 | 1- | −2.38 | −0.25 | 0.41 | 0.24 |
| | | acylglycerol- | | | | |
| | | 3-phosphate | | | | |
| | | O- | | | | |
| | | acyltransferase 6 | | | | |
| 3431 | SP110 | SP110 nuclear | −2.38 | 0.04 | 0.94 | 0.17 |
| | | body protein | | | | |
| 4140 | MARK3 | MAP/microtubule | −2.38 | −0.45 | −0.24 | 0.14 |
| | | affinity- | | | | |
| | | regulating | | | | |
| | | kinase 3 | | | | |
| 59269 | HIVEP3 | human | −2.38 | 0.03 | −0.99 | −0.81 |
| | | immunodeficiency | | | | |
| | | virus | | | | |
| | | type I | | | | |
| | | enhancer | | | | |
| | | binding | | | | |
| | | protein 3 | | | | |
| 7846 | TUBA1A | tubulin, alpha | −2.38 | 0.29 | −0.02 | 0.07 |
| | | 1a | | | | |
| 100652740 | C16orf98 | chromosome | −2.38 | 0.39 | −1.72 | 0.89 |
| | | 16 open | | | | |
| | | reading frame | | | | |
| | | 98 | | | | |
| 23398 | PPWD1 | peptidylprolyl | −2.38 | 0.12 | −0.31 | −0.95 |
| | | isomerase | | | | |
| | | domain and | | | | |
| | | WD repeat | | | | |
| | | containing 1 | | | | |
| 158747 | MOSPD2 | motile sperm | −2.39 | 0.20 | −0.33 | −0.87 |
| | | domain | | | | |
| | | containing 2 | | | | |
| 285512 | FAM13A- | FAM13A | −2.39 | −0.02 | −0.56 | 0.34 |
| | AS1 | antisense | | | | |
| | | RNA1 | | | | |
| 3937 | LCP2 | lymphocyte | −2.39 | −0.40 | −0.18 | −0.43 |
| | | cytosolic | | | | |
| | | protein 2 | | | | |
| | | (SH2 domain | | | | |
| | | containing | | | | |
| | | leukocyte | | | | |
| | | protein of | | | | |
| | | 76 kDa) | | | | |
| 730051 | ZNF814 | zinc finger | −2.39 | 0.06 | 0.80 | 1.10 |
| | | protein 814 | | | | |
| 9320 | TRIP12 | thyroid | −2.39 | 0.32 | 0.77 | −0.44 |
| | | hormone | | | | |
| | | receptor | | | | |
| | | interactor 12 | | | | |
| 6932 | TCF7 | transcription | −2.39 | −0.20 | 0.72 | −0.19 |
| | | factor 7 (T- | | | | |
| | | cell specific, | | | | |
| | | HMG-box) | | | | |
| 79722 | ANKRD55 | ankyrin repeat | −2.39 | −0.17 | −0.26 | 0.25 |
| | | domain 55 | | | | |
| 115362 | GBP5 | guanylate | −2.39 | −0.84 | −0.21 | 0.15 |
| | | binding | | | | |
| | | protein 5 | | | | |
| 29909 | GPR171 | G protein- | −2.39 | −0.45 | −0.41 | −0.68 |
| | | coupled | | | | |
| | | receptor 171 | | | | |
| 440503 | PLIN5 | perilipin 5 | −2.40 | 0.00 | −0.42 | −0.55 |
| 80183 | KIAA0226L | KIAA0226- | −2.40 | 1.05 | 0.47 | 0.24 |
| | | like | | | | |
| 80709 | AKNA | AT-hook | −2.40 | 0.01 | 0.62 | −0.58 |
| | | transcription | | | | |
| | | factor | | | | |
| 10562 | OLFM4 | olfactomedin 4 | −2.40 | −0.05 | −0.92 | −0.43 |
| 64780 | MICAL1 | microtubule | −2.41 | 0.42 | −1.67 | −0.46 |
| | | associated | | | | |
| | | monooxygenase, | | | | |
| | | calponin | | | | |
| | | and LIM | | | | |
| | | domain | | | | |
| | | containing 1 | | | | |
| 155038 | GIMAP8 | GTPase, | −2.41 | −0.10 | 0.79 | −0.31 |
| | | IMAP family | | | | |
| | | member 8 | | | | |
| 1783 | DYNC1LI2 | dynein, | −2.41 | 0.19 | −0.77 | −0.07 |
| | | cytoplasmic 1, | | | | |
| | | light | | | | |
| | | intermediate | | | | |
| | | chain 2 | | | | |
| 100287569 | LINC00173 | long | −2.42 | 0.45 | 0.86 | 0.65 |
| | | intergenic | | | | |
| | | non-protein | | | | |
| | | coding RNA | | | | |
| | | 173 | | | | |
| 5696 | PSMB8 | proteasome | −2.42 | 0.36 | 0.79 | 0.06 |
| | | (prosome, | | | | |
| | | macropain) | | | | |
| | | subunit, beta | | | | |
| | | type, 8 | | | | |
| 11066 | SNRNP35 | small nuclear | −2.42 | 0.50 | 0.48 | 0.36 |
| | | ribonucleoprotein | | | | |
| | | 35 kDa | | | | |
| | | (U11/U12) | | | | |
| 122553 | TRAPPC6B | trafficking | −2.42 | 0.43 | −0.02 | −0.27 |
| | | protein | | | | |
| | | particle | | | | |
| | | complex 6B | | | | |
| 50484 | RRM2B | ribonucleotide | −2.43 | 0.65 | −0.60 | −0.74 |
| | | reductase M2B | | | | |
| | | (TP53 | | | | |
| | | inducible) | | | | |
| 9847 | C2CD5 | C2 calcium- | −2.43 | −0.11 | −0.34 | −0.35 |
| | | dependent | | | | |
| | | domain | | | | |
| | | containing 5 | | | | |
| 129285 | PPP1R21 | protein | −2.43 | 0.74 | 0.78 | −0.73 |
| | | phosphatase | | | | |
| | | 1, regulatory | | | | |
| | | subunit 21 | | | | |
| 9794 | MAML1 | mastermind- | −2.43 | −0.38 | 0.25 | −1.74 |
| | | like 1 | | | | |
| | | (Drosophila) | | | | |
| 84309 | NUDT16L1 | nudix | −2.43 | −0.63 | 0.85 | 0.02 |
| | | (nucleoside | | | | |
| | | diphosphate | | | | |
| | | linked moiety | | | | |
| | | X)-type motif | | | | |
| | | 16-like 1 | | | | |
| 65258 | MPPE1 | metallophosphoesterase 1 | −2.43 | 0.16 | 0.09 | −0.45 |
| 5562 | PRKAA1 | protein | −2.44 | −0.34 | −0.85 | 0.48 |
| | | kinase, AMP- | | | | |
| | | activated, | | | | |
| | | alpha 1 | | | | |
| | | catalytic | | | | |
| | | subunit | | | | |
| 123036 | TC2N | tandem C2 | −2.44 | 0.01 | −0.08 | −0.30 |
| | | domains, | | | | |
| | | nuclear | | | | |
| 84166 | NLRC5 | NLR family, | −2.45 | −0.28 | −0.25 | −0.33 |
| | | CARD | | | | |
| | | domain | | | | |
| | | containing 5 | | | | |
| 253143 | PRR14L | proline rich | −2.45 | −0.36 | −0.38 | −0.61 |
| | | 14-like | | | | |
| 1236 | CCR7 | chemokine | −2.45 | 0.05 | 0.49 | −0.12 |
| | | (C-C motif) | | | | |
| | | receptor 7 | | | | |
| 266747 | RGL4 | ral guanine | −2.46 | −0.19 | 0.00 | −0.25 |
| | | nucleotide | | | | |
| | | dissociation | | | | |
| | | stimulator- | | | | |
| | | like 4 | | | | |
| 25988 | HINFP | histone H4 | −2.46 | −0.11 | 2.05 | −0.48 |
| | | transcription | | | | |
| | | factor | | | | |
| 6515 | SLC2A3 | solute carrier | −2.46 | 0.39 | 0.08 | 0.20 |
| | | family 2 | | | | |
| | | (facilitated | | | | |
| | | glucose | | | | |
| | | transporter), | | | | |
| | | member 3 | | | | |
| 90592 | ZNF700 | zinc finger | −2.46 | 0.71 | −1.32 | −0.79 |
| | | protein 700 | | | | |
| 6867 | TACC1 | transforming, | −2.47 | 1.05 | −0.07 | 0.50 |
| | | acidic coiled- | | | | |
| | | coil | | | | |
| | | containing | | | | |
| | | protein 1 | | | | |
| 23041 | MON2 | MON2 | −2.47 | 0.02 | −1.18 | −0.17 |
| | | homolog (S. cerevisiae) | | | | |
| 100289230 | LOC100289230 | uncharacterized | −2.47 | −0.65 | 0.54 | −0.02 |
| | | LOC100289230 | | | | |
| 57169 | ZNFX1 | zinc finger, | −2.48 | 0.01 | −0.28 | 0.54 |
| | | NFX1-type | | | | |
| | | containing 1 | | | | |
| 1105 | CHD1 | chromodomain | −2.48 | 0.32 | −1.07 | 0.09 |
| | | helicase | | | | |
| | | DNA binding | | | | |
| | | protein 1 | | | | |
| 943 | TNFRSF8 | tumor | −2.48 | 1.03 | 1.09 | 0.96 |
| | | necrosis factor | | | | |
| | | receptor | | | | |
| | | superfamily, | | | | |
| | | member 8 | | | | |
| 3275 | PRMT2 | protein | −2.48 | 0.64 | −0.31 | 0.34 |
| | | arginine | | | | |
| | | methyltransferase 2 | | | | |
| 8851 | CDK5R1 | cyclin- | −2.49 | 0.52 | 0.25 | −0.10 |
| | | dependent | | | | |
| | | kinase 5, | | | | |
| | | regulatory | | | | |
| | | subunit 1 | | | | |
| | | (p35) | | | | |
| 168537 | GIMAP7 | GTPase, | −2.49 | −0.53 | −0.08 | −0.09 |
| | | IMAP family | | | | |
| | | member 7 | | | | |
| 256236 | NAPSB | napsin B | −2.49 | 0.84 | 0.02 | 0.41 |
| | | aspartic | | | | |
| | | peptidase, | | | | |
| | | pseudogene | | | | |
| 26235 | FBXL4 | F-box and | −2.50 | −0.08 | −0.17 | 0.44 |
| | | leucine-rich | | | | |
| | | repeat protein 4 | | | | |
| 9363 | RAB33A | RAB33A, | −2.50 | −0.30 | 0.49 | 0.73 |
| | | member RAS | | | | |
| | | oncogene | | | | |
| | | family | | | | |
| 729683 | LOC729683 | uncharacterized | −2.50 | 0.17 | 0.43 | 0.05 |
| | | LOC729683 | | | | |
| 388969 | C2orf68 | chromosome | −2.51 | −0.79 | 0.31 | 0.01 |
| | | 2 open | | | | |
| | | reading frame | | | | |
| | | 68 | | | | |
| 4818 | NKG7 | natural killer | −2.51 | −0.88 | −0.05 | 0.13 |
| | | cell group 7 | | | | |
| | | sequence | | | | |
| 93953 | ACRC | acidic repeat | −2.51 | 0.21 | −0.99 | 0.29 |
| | | containing | | | | |
| 28982 | FLVCR1 | feline | −2.51 | 0.14 | 0.27 | −2.04 |
| | | leukemia | | | | |
| | | virus | | | | |
| | | subgroup C | | | | |
| | | cellular | | | | |
| | | receptor 1 | | | | |
| 84911 | ZNF382 | zinc finger | −2.52 | −0.59 | 0.61 | −0.52 |
| | | protein 382 | | | | |
| 66036 | MTMR9 | myotubularin | −2.52 | −0.62 | −1.12 | 0.10 |
| | | related protein 9 | | | | |
| 9750 | FAM65B | family with | −2.52 | −0.02 | 0.69 | −0.35 |
| | | sequence | | | | |
| | | similarity 65, | | | | |
| | | member B | | | | |
| 9938 | ARHGAP25 | Rho GTPase | −2.52 | −0.43 | −0.14 | −0.46 |
| | | activating | | | | |
| | | protein 25 | | | | |
| 120425 | AMICA1 | adhesion | −2.52 | 0.03 | −0.08 | 0.09 |
| | | molecule, | | | | |
| | | interacts with | | | | |
| | | CXADR | | | | |
| | | antigen 1 | | | | |
| 6840 | SVIL | supervillin | −2.53 | −0.28 | −0.45 | 0.12 |
| 26268 | FBXO9 | F-box protein 9 | −2.53 | −1.93 | −1.19 | 0.51 |
| 55206 | SBNO1 | strawberry | −2.53 | −0.52 | −1.01 | −0.74 |
| | | notch | | | | |
| | | homolog 1 | | | | |
| | | (Drosophila) | | | | |
| 355 | FAS | Fas cell | −2.54 | 0.71 | 0.41 | 0.30 |
| | | surface death | | | | |
| | | receptor | | | | |
| 50615 | IL21R | interleukin 21 | −2.54 | −0.27 | 0.71 | 0.03 |
| | | receptor | | | | |
| 221178 | SPATA13 | spermatogenesis | −2.54 | −0.05 | 0.34 | −0.29 |
| | | associated | | | | |
| | | 13 | | | | |
| 11104 | KATNA1 | katanin p60 | −2.54 | 0.66 | −0.03 | −1.98 |
| | | (ATPase | | | | |
| | | containing) | | | | |
| | | subunit A1 | | | | |
| 1117 | CHI3L2 | chitinase 3- | −2.54 | 0.43 | 1.36 | −0.47 |
| | | like2 | | | | |
| 374969 | CCDC23 | coiled-coil | −2.54 | 0.15 | −0.06 | 0.88 |
| | | domain | | | | |
| | | containing 23 | | | | |
| 6285 | S100B | S100 calcium | −2.55 | −0.38 | −1.44 | −0.44 |
| | | binding | | | | |
| | | protein B | | | | |
| 387357 | THEMIS | thymocyte | −2.55 | −0.69 | −0.77 | −0.13 |
| | | selection | | | | |
| | | associated | | | | |
| 2960 | GTF2E1 | general | −2.56 | −0.21 | 0.05 | −1.50 |
| | | transcription | | | | |
| | | factor IIE, | | | | |
| | | polypeptide 1, | | | | |
| | | alpha 56 kDa | | | | |
| 100129550 | LOC100129550 | uncharacterized | −2.56 | −0.35 | −0.66 | −0.15 |
| | | LOC100129550 | | | | |
| 54520 | CCDC93 | coiled-coil | −2.56 | 0.13 | −0.47 | −0.50 |
| | | domain | | | | |
| | | containing 93 | | | | |
| 10302 | SNAPC5 | small nuclear | −2.56 | −0.81 | 0.12 | 0.51 |
| | | RNA | | | | |
| | | activating | | | | |
| | | complex, | | | | |
| | | polypeptide 5, | | | | |
| | | 19 kDa | | | | |
| 50939 | IMPG2 | interphotoreceptor | −2.56 | 0.15 | 0.32 | −1.16 |
| | | matrix | | | | |
| | | proteoglycan 2 | | | | |
| 8802 | SUCLG1 | succinate- | −2.56 | −0.31 | 0.44 | −0.77 |
| | | CoA ligase, | | | | |
| | | alpha subunit | | | | |
| 23332 | CLASP1 | cytoplasmic | −2.57 | 0.16 | −1.50 | −0.09 |
| | | linker | | | | |
| | | associated | | | | |
| | | protein 1 | | | | |
| 203328 | SUSD3 | sushi domain | −2.57 | 0.78 | −0.01 | 0.01 |
| | | containing 3 | | | | |
| 126231 | ZNF573 | zinc linger | −2.58 | −0.54 | 0.54 | −1.59 |
| | | protein 573 | | | | |
| 6993 | DYNLT1 | dynein, light | −2.58 | −0.22 | 0.12 | 0.22 |
| | | chain, Tctex- | | | | |
| | | type 1 | | | | |
| 58500 | ZNF250 | zinc finger | −2.59 | 0.73 | 0.63 | −0.05 |
| | | protein 250 | | | | |
| 10507 | SEMA4D | sema domain, | −2.59 | 0.09 | 0.59 | 0.54 |
| | | immunoglobulin | | | | |
| | | domain | | | | |
| | | (Ig), | | | | |
| | | transmembrane | | | | |
| | | domain | | | | |
| | | (TM) and | | | | |
| | | short | | | | |
| | | cytoplasmic | | | | |
| | | domain, | | | | |
| | | (semaphorin) | | | | |
| | | 4D | | | | |
| 170482 | CLEC4C | C-type lectin | −2.59 | 0.33 | 1.01 | 0.08 |
| | | domain family | | | | |
| | | 4, member C | | | | |
| 3004 | GZMM | granzyme M | −2.59 | −0.20 | −0.15 | 0.23 |
| | | (lymphocyte | | | | |
| | | met-ase 1) | | | | |
| 64895 | PAPOLG | poly(A) | −2.59 | −0.46 | −0.57 | −0.15 |
| | | polymerase | | | | |
| | | gamma | | | | |
| 7850 | IL1R2 | interleukin 1 | −2.59 | 0.24 | −0.03 | 0.07 |
| | | receptor, type | | | | |
| | | II | | | | |
| 93594 | TBC1D31 | TBC1 domain | −2.59 | −0.35 | 0.31 | −1.26 |
| | | family, | | | | |
| | | member 31 | | | | |
| 23215 | PRRC2C | proline-rich | −2.60 | 0.23 | 0.47 | 0.90 |
| | | coiled-coil 2C | | | | |
| 80196 | RNF34 | ring finger | −2.60 | −0.06 | 0.49 | −0.53 |
| | | protein 34, E3 | | | | |
| | | ubiquitin | | | | |
| | | protein ligase | | | | |
| 284415 | VSTM1 | V-set and | −2.60 | −0.60 | 0.30 | 0.84 |
| | | transmembrane | | | | |
| | | domain | | | | |
| | | containing 1 | | | | |
| 56882 | CDC42SE1 | CDC42 small | −2.60 | −0.19 | 0.12 | −0.34 |
| | | effector 1 | | | | |
| 6508 | SLC4A3 | solute carrier | −2.60 | −0.21 | −0.74 | 0.20 |
| | | family 4 | | | | |
| | | (anion | | | | |
| | | exchanger), | | | | |
| | | member 3 | | | | |
| 79663 | HSPBAP1 | HSPB (heat | −2.61 | −0.19 | 0.15 | −0.38 |
| | | shock 27 kDa) | | | | |
| | | associated | | | | |
| | | protein 1 | | | | |
| 474344 | GIMAP6 | GTPase, | −2.61 | −0.31 | 0.86 | −0.14 |
| | | IMAP family | | | | |
| | | member 6 | | | | |
| 100505746 | ITGB2- | ITGB2 | −2.62 | 0.34 | −0.47 | 0.12 |
| | AS1 | antisense | | | | |
| | | RNA 1 | | | | |
| 1293 | COL6A3 | collagen, type | −2.62 | −0.52 | −1.29 | −0.64 |
| | | VI, alpha 3 | | | | |
| 10294 | DNAJA2 | DnaJ (Hsp40) | −2.62 | 0.13 | 0.40 | −0.18 |
| | | homolog, | | | | |
| | | subfamily A, | | | | |
| | | member 2 | | | | |
| 3091 | HIF1A | hypoxia | −2.62 | 0.83 | −0.75 | −0.17 |
| | | inducible | | | | |
| | | factor 1, alpha | | | | |
| | | subunit (basic | | | | |
| | | helix-loop- | | | | |
| | | helix | | | | |
| | | transcription | | | | |
| | | factor) | | | | |
| 79573 | TTC13 | tetratricopeptide | −2.63 | −0.32 | −0.92 | −0.75 |
| | | repeat | | | | |
| | | domain 13 | | | | |
| 9208 | LRRFIP1 | leucine rich | −2.63 | 0.81 | 0.12 | −0.23 |
| | | repeat (in | | | | |
| | | FLII) | | | | |
| | | interacting | | | | |
| | | protein 1 | | | | |
| 29116 | MYLIP | myosin | −2.63 | −0.28 | −0.62 | 0.05 |
| | | regulatory | | | | |
| | | light chain | | | | |
| | | interacting | | | | |
| | | protein | | | | |
| 92370 | ACPL2 | acid | −2.63 | −1.54 | −0.45 | −0.71 |
| | | phosphatase- | | | | |
| | | like 2 | | | | |
| 9546 | APBA3 | amyloid beta | −2.64 | 0.21 | 1.01 | −0.23 |
| | | (A4) | | | | |
| | | precursor | | | | |
| | | protein- | | | | |
| | | binding, | | | | |
| | | family A, | | | | |
| | | member 3 | | | | |
| 253018 | HCG27 | HLA complex | −2.64 | −0.05 | 0.09 | 0.27 |
| | | group 27 | | | | |
| | | (non-protein | | | | |
| | | coding) | | | | |
| 10096 | ACTR3 | ARP3 actin- | −2.65 | 0.49 | −0.28 | 0.34 |
| | | related protein | | | | |
| | | 3 homolog | | | | |
| | | (yeast) | | | | |
| 84138 | SLC7A6OS | solute carrier | −2.65 | −0.42 | 1.70 | −0.20 |
| | | family 7, | | | | |
| | | member 6 | | | | |
| | | opposite | | | | |
| | | strand | | | | |
| 8440 | NCK2 | NCK adaptor | −2.65 | −1.07 | 0.79 | 0.13 |
| | | protein 2 | | | | |
| 7277 | TUBA4A | tubulin, alpha | −2.65 | 0.48 | 0.19 | 0.19 |
| | | 4a | | | | |
| 8269 | TMEM187 | transmembrane | −2.66 | −0.27 | 0.56 | −1.62 |
| | | protein 187 | | | | |
| 8807 | IL18RAP | interleukin 18 | −2.66 | −0.04 | −0.09 | 0.08 |
| | | receptor | | | | |
| | | accessory | | | | |
| | | protein | | | | |
| 1235 | CCR6 | chemokine | −2.67 | −0.28 | 1.66 | −0.57 |
| | | (C-C motif) | | | | |
| | | receptor 6 | | | | |
| 7059 | THBS3 | thrombospondin 3 | −2.67 | −0.20 | −0.07 | −0.91 |
| 54971 | BANP | BTG3 | −2.67 | −0.34 | 0.29 | −0.19 |
| | | associated | | | | |
| | | nuclear | | | | |
| | | protein | | | | |
| 57677 | ZFP14 | ZFP14 zinc | −2.68 | 0.38 | −1.14 | −0.27 |
| | | finger protein | | | | |
| 6890 | TAP1 | transporter 1, | −2.68 | 0.05 | 0.37 | 0.20 |
| | | ATP-binding | | | | |
| | | cassette, sub- | | | | |
| | | family B | | | | |
| | | (MDR/TAP) | | | | |
| 22944 | KIN | KIN, | −2.69 | 0.36 | −1.20 | −0.67 |
| | | antigenic | | | | |
| | | determinant of | | | | |
| | | recA protein | | | | |
| | | homolog | | | | |
| | | (mouse) | | | | |
| 26512 | INTS6 | integrator | −2.69 | −0.14 | 0.22 | −0.75 |
| | | complex | | | | |
| | | subunit 6 | | | | |
| 92797 | HELB | helicase | −2.69 | 0.17 | −0.33 | 0.01 |
| | | (DNA) B | | | | |
| 5481 | PPID | peptidylprolyl | −2.70 | 0.38 | 1.67 | −0.63 |
| | | isomerase D | | | | |
| 22897 | CEP164 | centrosomal | −2.71 | −0.53 | 1.08 | −0.97 |
| | | protein | | | | |
| | | 164 kDa | | | | |
| 3620 | IDO1 | indoleamine | −2.71 | −0.70 | 0.25 | 0.59 |
| | | 2,3- | | | | |
| | | dioxygenase 1 | | | | |
| 64766 | S100PBP | S100P | −2.71 | −0.27 | 0.58 | −1.38 |
| | | binding | | | | |
| | | protein | | | | |
| 8809 | IL18R1 | interleukin 18 | −2.71 | 0.21 | 0.39 | −0.32 |
| | | receptor 1 | | | | |
| 116984 | ARAP2 | ArfGAP with | −2.71 | −0.28 | −0.80 | −0.35 |
| | | RhoGAP | | | | |
| | | domain, | | | | |
| | | ankyrin repeat | | | | |
| | | and PH | | | | |
| | | domain 2 | | | | |
| 440823 | MIAT | myocardial | −2.72 | −0.95 | −0.55 | −0.22 |
| | | infarction | | | | |
| | | associated | | | | |
| | | transcript | | | | |
| | | (non-protein | | | | |
| | | coding) | | | | |
| 59340 | HRH4 | histamine | −2.72 | 0.21 | −0.90 | −0.79 |
| | | receptor H4 | | | | |
| 197259 | MLKL | mixed lineage | −2.73 | 0.03 | 0.50 | −0.43 |
| | | kinase | | | | |
| | | domain-like | | | | |
| 23670 | TMEM2 | transmembrane | −2.73 | 0.16 | 0.22 | −0.71 |
| | | protein 2 | | | | |
| 643314 | KIAA0754 | KIAA0754 | −2.73 | 0.87 | 0.06 | 0.11 |
| 28526 | TRDC | T cell receptor | −2.73 | −1.25 | 0.37 | 0.10 |
| | | delta constant | | | | |
| 55096 | EBLN2 | endogenous | −2.74 | −0.05 | −0.03 | 0.57 |
| | | Bornavirus- | | | | |
| | | like | | | | |
| | | nucleoprotein 2 | | | | |
| 2796 | GNRH1 | gonadotropin- | −2.74 | −0.88 | 0.11 | −0.38 |
| | | releasing | | | | |
| | | hormone 1 | | | | |
| | | (luteinizing- | | | | |
| | | releasing | | | | |
| | | hormone) | | | | |
| 54509 | RHOF | ras homolog | −2.74 | 0.31 | 0.83 | 1.43 |
| | | family | | | | |
| | | member F (in | | | | |
| | | filopodia) | | | | |
| 11120 | BTN2A1 | butyrophilin, | −2.75 | 0.52 | −0.22 | −1.12 |
| | | subfamily 2, | | | | |
| | | member A1 | | | | |
| 84869 | CBR4 | carbonyl | −2.75 | 0.31 | −0.35 | −0.28 |
| | | reductase 4 | | | | |
| 28991 | COMMD5 | COMM | −2.76 | 0.41 | 0.45 | −0.17 |
| | | domain | | | | |
| | | containing 5 | | | | |
| 154141 | MBOAT1 | membrane | −2.79 | 0.06 | 0.08 | 0.22 |
| | | bound O- | | | | |
| | | acyltransferase | | | | |
| | | domain | | | | |
| | | containing 1 | | | | |
| 3659 | IRF1 | interferon | −2.79 | −0.54 | −0.36 | −0.28 |
| | | regulatory | | | | |
| | | factor 1 | | | | |
| 154007 | SNRNP48 | small nuclear | −2.80 | 0.44 | −0.26 | −0.32 |
| | | ribonucleoprotein | | | | |
| | | 48 kDa | | | | |
| | | (U11/U12) | | | | |
| 116835 | HSPA12B | heat shock | −2.81 | 0.14 | −0.16 | 0.30 |
| | | 70 kD protein | | | | |
| | | 12B | | | | |
| 9488 | PIGB | phosphatidylinositol | −2.82 | 0.68 | 0.08 | −0.07 |
| | | glycan | | | | |
| | | anchor | | | | |
| | | biosynthesis, | | | | |
| | | class B | | | | |
| 100293516 | ZNF587B | zinc finger | −2.83 | −0.09 | 0.21 | −1.06 |
| | | protein 587B | | | | |
| 9217 | VAPB | VAMP | −2.84 | −1.12 | −1.05 | −0.01 |
| | | (vesicle- | | | | |
| | | associated | | | | |
| | | membrane | | | | |
| | | protein)- | | | | |
| | | associated | | | | |
| | | protein B and C | | | | |
| 10538 | BATF | basic leucine | −2.84 | 0.51 | 0.53 | 0.52 |
| | | zipper | | | | |
| | | transcription | | | | |
| | | factor, ATF- | | | | |
| | | like | | | | |
| 6935 | ZEB1 | zinc finger E- | −2.87 | 0.56 | −0.27 | −0.22 |
| | | box binding | | | | |
| | | homeobox 1 | | | | |
| 10443 | N4BP2L2 | NEDD4 | −2.88 | −0.84 | −1.29 | −0.96 |
| | | binding | | | | |
| | | protein 2-like 2 | | | | |
| 3837 | KPNB1 | karyopherin | −2.88 | −1.28 | 0.25 | −0.60 |
| | | (importin) | | | | |
| | | beta 1 | | | | |
| 81698 | LINC00597 | long | −2.88 | −0.07 | 0.72 | −0.14 |
| | | intergenic | | | | |
| | | non-protein | | | | |
| | | coding RNA | | | | |
| | | 597 | | | | |
| 2643 | GCH1 | GTP | −2.89 | 0.40 | −0.06 | 0.56 |
| | | cyclohydrolase 1 | | | | |
| 57559 | STAMBPL1 | STAM | −2.89 | 0.00 | 0.60 | −0.53 |
| | | binding | | | | |
| | | protein-like 1 | | | | |
| 116842 | LEAP2 | liver | −2.89 | −0.25 | −1.30 | −0.48 |
| | | expressed | | | | |
| | | antimicrobial | | | | |
| | | peptide 2 | | | | |
| 202 | AIM1 | absent in | −2.90 | 0.49 | 0.63 | −1.01 |
| | | melanoma 1 | | | | |
| 9934 | P2RY14 | purinergic | −2.90 | 0.11 | 0.00 | 0.08 |
| | | receptor P2Y, | | | | |
| | | G-protein | | | | |
| | | coupled, 14 | | | | |
| 100216546 | LINC01004 | long | −2.90 | 0.05 | −1.36 | −0.34 |
| | | intergenic | | | | |
| | | non-protein | | | | |
| | | coding RNA | | | | |
| | | 1004 | | | | |
| 9692 | KIAA0391 | KIAA0391 | −2.90 | 0.17 | 0.26 | −1.79 |
| 89845 | ABCC10 | ATP-binding | −2.92 | −0.33 | 0.85 | −1.06 |
| | | cassette, sub- | | | | |
| | | family C | | | | |
| | | (CFTR/MRP), | | | | |
| | | member 10 | | | | |
| 26034 | IPCEF1 | interaction | −2.92 | −0.27 | 0.37 | −0.66 |
| | | protein for | | | | |
| | | cytohesin | | | | |
| | | exchange | | | | |
| | | factors 1 | | | | |
| 10906 | TRAFD1 | TRAF-type | −2.93 | 0.40 | 0.36 | 0.88 |
| | | zinc finger | | | | |
| | | domain | | | | |
| | | containing 1 | | | | |
| 84811 | BUD13 | BUD13 | −2.94 | −0.56 | −0.52 | −0.18 |
| | | homolog (S. cerevisiae) | | | | |
| 100527964 | LOC100527964 | uncharacterized | −2.96 | −0.38 | −0.49 | −1.17 |
| | | LOC100527964 | | | | |
| 118426 | LOH12CR1 | loss of | −2.97 | 0.02 | −0.17 | −0.21 |
| | | heterozygosity, | | | | |
| | | 12, | | | | |
| | | chromosomal | | | | |
| | | region 1 | | | | |
| 54331 | GNG2 | guanine | −2.98 | 0.37 | 0.07 | −0.18 |
| | | nucleotide | | | | |
| | | binding | | | | |
| | | protein (G | | | | |
| | | protein), | | | | |
| | | gamma 2 | | | | |
| 94081 | SFXN1 | sideroflexin 1 | −2.99 | 0.15 | −0.22 | −0.48 |
| 145474 | LOC145474 | uncharacterized | −2.99 | 0.22 | −0.36 | 0.36 |
| | | LOC145474 | | | | |
| 84969 | TOX2 | TOX high | −3.00 | 0.02 | −0.66 | −0.27 |
| | | mobility | | | | |
| | | group box | | | | |
| | | family | | | | |
| | | member 2 | | | | |
| 4907 | NT5E | 5′- | −3.02 | 0.12 | 1.19 | −0.48 |
| | | nucleotidase, | | | | |
| | | ecto (CD73) | | | | |
| 51735 | RAPGEF6 | Rap guanine | −3.03 | −0.09 | −0.81 | −0.46 |
| | | nucleotide | | | | |
| | | exchange | | | | |
| | | factor (GEF) 6 | | | | |
| 9953 | HS3ST3B1 | heparan | −3.03 | −0.12 | −1.70 | −1.64 |
| | | sulfate | | | | |
| | | (glucosamine) | | | | |
| | | 3-O- | | | | |
| | | sulfotransferase | | | | |
| | | 3B1 | | | | |
| 23112 | TNRC6B | trinucleotide | −3.04 | −0.93 | 0.39 | −0.92 |
| | | repeat | | | | |
| | | containing 6B | | | | |
| 91526 | ANKRD44 | ankyrin repeat | −3.05 | 0.24 | 0.25 | −0.51 |
| | | domain 44 | | | | |
| 101928017 | LOC101928017 | uncharacterized | −3.08 | −0.04 | −0.52 | −0.33 |
| | | LOC101928017 | | | | |
| 84859 | LRCH3 | leucine-rich | −3.08 | −0.37 | −0.52 | −0.57 |
| | | repeats and | | | | |
| | | calponin | | | | |
| | | homology | | | | |
| | | (CH) domain | | | | |
| | | containing 3 | | | | |
| 159013 | CXorf38 | chromosome | −3.11 | 0.44 | 0.69 | −0.68 |
| | | X open | | | | |
| | | reading frame | | | | |
| | | 38 | | | | |
| 23208 | SYT11 | synaptotagmin | −3.19 | 0.00 | 0.62 | −0.55 |
| | | XI | | | | |
| 101928649 | LOC101928649 | uncharacterized | −3.23 | −0.33 | −0.10 | 1.08 |
| | | LOC101928649 | | | | |
| 85459 | KIAA1731 | KIAA1731 | −3.28 | 0.57 | 0.67 | 0.61 |
| 9617 | MTRF1 | mitochondrial | −3.31 | 0.69 | 0.22 | −0.22 |
| | | translational | | | | |
| | | release factor 1 | | | | |
| 56898 | BDH2 | 3- | −3.50 | 0.36 | 0.59 | −0.70 |
| | | hydroxybutyrate | | | | |
| | | dehydrogenase, | | | | |
| | | type 2 | | | | |
| 387882 | C12orf75 | chromosome | −3.61 | −0.75 | 0.39 | 0.39 |
| | | 12 open | | | | |
| | | reading frame | | | | |
| | | 75 | | | | |
| 677769 | SCARNA17 | small Cajal | −3.64 | −1.27 | −0.31 | 0.25 |
| | | body-specific | | | | |
| | | RNA17 | | | | |

| gene | 37 | 38 | 39 | 40 | 41 | 42 | 43 | 44 | 45 | 46 | 47 | 48 | 49 |
|---|---|---|---|---|---|---|---|---|---|---|---|---|---|
| 56904 | 0.71 | 0.24 | −0.28 | −0.23 | −0.13 | 0.98 | 1.54 | 1.43 | 0.73 | 1.10 | 1.44 | 0.98 | 0.90 |
| 6464 | −0.27 | 0.99 | 1.19 | −0.30 | 0.90 | 1.11 | 0.57 | 2.34 | 1.40 | 0.45 | 1.58 | 0.72 | 0.74 |
| 79087 | 0.10 | −1.45 | 0.11 | 1.19 | 0.28 | −0.15 | 1.43 | 1.19 | 0.41 | 0.95 | 1.63 | 0.72 | 1.15 |
| 101928190 | 0.31 | 0.91 | −1.41 | 0.23 | 0.13 | 1.68 | 0.42 | 0.86 | 0.70 | 0.39 | 1.90 | −0.10 | 1.52 |
| 3416 | −0.57 | −0.36 | 1.61 | 0.57 | −0.39 | 1.73 | 0.21 | 1.68 | −0.09 | 1.19 | 1.50 | 0.54 | 1.52 |
| 8001 | 0.98 | 1.09 | 0.47 | 0.48 | 1.52 | −0.17 | 1.03 | −0.17 | 0.38 | 0.08 | 0.91 | 2.51 | −0.03 |
| 3727 | 1.09 | −0.01 | 1.40 | −0.11 | 0.06 | 1.11 | 1.34 | 1.26 | 0.72 | −0.14 | 2.08 | 0.75 | 0.33 |
| 10945 | 0.81 | −0.57 | 0.88 | 0.08 | 0.06 | 1.39 | 1.53 | 1.53 | 0.60 | 1.11 | 1.64 | −0.08 | 1.05 |
| 5025 | 0.62 | −1.94 | 0.26 | −0.16 | 0.05 | 1.94 | 0.87 | 1.26 | 1.11 | 1.26 | 1.57 | 0.26 | 1.03 |
| 29920 | −0.15 | −2.15 | 0.68 | 0.75 | 0.48 | 1.18 | 1.28 | 1.23 | 0.34 | 1.12 | 0.99 | 0.24 | 0.75 |
| 101929248 | 0.01 | 1.61 | 0.18 | 0.53 | −0.20 | 2.59 | 0.99 | 1.20 | 0.97 | −0.79 | 1.22 | 1.06 | 0.49 |
| 23646 | 0.24 | −1.13 | 0.96 | 0.34 | 0.11 | 1.35 | 1.31 | 1.68 | 0.05 | 1.50 | 1.28 | 0.51 | 1.07 |
| 10280 | −0.50 | 0.10 | 0.93 | 0.34 | −0.69 | 1.01 | 0.31 | 2.78 | 0.98 | 0.78 | 1.56 | −0.44 | 1.14 |
| 140699 | −0.07 | 0.94 | 0.59 | −0.16 | −0.42 | 1.76 | −0.96 | 0.53 | 2.62 | 0.53 | 0.98 | 0.67 | 0.42 |
| 4669 | 0.46 | −0.07 | 0.27 | −0.03 | −0.46 | 1.51 | 0.78 | 2.04 | 1.01 | 1.38 | 1.43 | 0.27 | 1.47 |
| 8341 | −0.67 | 0.97 | 0.77 | 0.30 | −0.01 | 2.01 | 1.24 | 0.65 | 1.12 | 0.86 | 1.20 | 0.48 | 0.38 |
| 2519 | −0.27 | −1.09 | 0.71 | 0.23 | 0.25 | 1.18 | 1.07 | 1.39 | −0.27 | 2.25 | 0.60 | 0.35 | 1.70 |
| 8985 | 1.02 | −1.43 | 0.95 | −0.02 | −0.03 | 1.07 | 0.69 | 1.71 | 0.83 | 0.14 | 1.52 | 0.71 | 1.59 |
| 1690 | 0.15 | −0.68 | 1.61 | 0.98 | 0.17 | −0.16 | 0.42 | 1.49 | 0.75 | −0.39 | 0.43 | 1.26 | 1.76 |
| 51114 | 0.73 | −1.38 | 0.85 | −0.58 | 0.03 | 1.59 | 0.00 | 1.31 | 1.14 | 0.51 | 1.71 | 0.63 | 1.78 |
| 55907 | 0.81 | −2.74 | 0.89 | 0.74 | 0.54 | 0.58 | 0.73 | −0.47 | 0.55 | 1.49 | 1.08 | 1.05 | 1.41 |
| 144811 | 0.37 | −0.37 | −0.14 | 0.49 | 0.06 | 1.38 | 0.64 | 0.73 | 1.51 | 1.22 | 0.69 | 0.76 | 1.00 |
| 92421 | 0.22 | 1.10 | −0.83 | 1.09 | 0.01 | 0.80 | −0.05 | 1.13 | 0.48 | 1.25 | 1.21 | 0.87 | 0.92 |
| 23475 | 1.13 | 0.03 | 1.45 | 0.18 | −1.05 | 2.00 | 0.68 | 0.81 | 0.87 | 0.86 | 0.95 | 0.15 | 1.62 |
| 54913 | −0.37 | −0.10 | 0.09 | 0.93 | −0.40 | 0.59 | 1.07 | 0.84 | 0.03 | 2.18 | 1.76 | 0.04 | 1.03 |
| 4645 | 0.10 | 1.08 | 0.83 | 1.07 | −0.70 | 2.24 | −0.22 | 0.09 | 0.28 | 1.06 | 1.74 | 1.21 | 1.03 |
| 9917 | 0.28 | −2.09 | 1.80 | 0.57 | −0.51 | 0.78 | 1.31 | 0.41 | 1.53 | 1.01 | 1.80 | 0.01 | 0.21 |
| 57213 | −0.17 | −0.47 | 1.72 | 0.94 | −0.10 | 0.64 | 0.21 | 0.64 | 1.98 | 1.98 | 0.86 | 0.23 | 1.56 |
| 113829 | −0.15 | −1.18 | 0.18 | 0.72 | −0.67 | 1.02 | 0.55 | 1.40 | 0.75 | 1.65 | 1.90 | −0.25 | 1.33 |
| 100128164 | 0.00 | 0.66 | 0.24 | 0.79 | −0.33 | 1.45 | 0.81 | −0.54 | 2.29 | 0.34 | −0.05 | 1.56 | −0.04 |
| 26284 | 0.49 | −0.61 | 1.23 | 0.18 | −1.13 | 0.39 | 1.23 | 1.88 | 0.52 | 1.45 | 1.48 | 0.12 | 1.12 |
| 64847 | 0.25 | 0.15 | 1.21 | 0.86 | −0.40 | 1.35 | 0.50 | 1.60 | 0.61 | 0.38 | 0.91 | 0.46 | 1.37 |
| 55974 | 0.44 | −1.19 | 0.22 | −0.25 | −0.17 | 1.56 | 1.31 | 1.64 | 0.74 | 0.76 | 1.89 | −0.13 | 1.52 |
| 64405 | 0.79 | 2.12 | 1.83 | −0.24 | 0.12 | 0.77 | 0.44 | −0.42 | 0.71 | 0.71 | 1.44 | 1.18 | −0.31 |
| 6510 | 0.83 | −0.44 | 0.26 | −0.15 | −0.18 | 1.32 | 1.39 | 1.65 | 0.26 | 0.81 | 1.40 | −0.12 | 1.32 |
| 196463 | −0.66 | 0.81 | 1.69 | 0.38 | −0.60 | 0.20 | 0.29 | 2.42 | 1.83 | −0.31 | 1.22 | 0.44 | 1.67 |
| 741 | 1.43 | 0.01 | 1.55 | 0.14 | 0.43 | −0.19 | 0.73 | 0.61 | 0.99 | 0.83 | 0.88 | 0.39 | 0.75 |
| 23568 | 1.22 | −0.32 | 0.30 | −0.88 | −0.15 | 1.16 | 0.99 | 1.12 | 0.79 | 0.98 | 1.69 | 1.16 | 0.98 |
| 79144 | 0.46 | 0.47 | 0.81 | −0.24 | 0.16 | 0.77 | 1.88 | 1.68 | 1.18 | 1.54 | 1.28 | −0.38 | −0.60 |
| 91319 | 0.15 | 1.29 | 0.46 | −0.42 | −0.44 | 0.92 | 0.20 | 2.64 | −0.29 | 1.45 | 1.91 | 0.19 | 0.85 |
| 79654 | 0.36 | −1.15 | 1.05 | 0.58 | 0.20 | 1.06 | 1.44 | 1.00 | 0.38 | 0.57 | 2.48 | −0.53 | 0.64 |
| 115950 | 0.98 | −2.01 | 0.67 | 0.83 | −0.26 | 0.33 | 1.07 | 0.48 | 1.16 | 1.11 | 0.89 | 0.15 | 1.50 |
| 1374 | 1.44 | −1.60 | 1.16 | 0.66 | 0.87 | 0.43 | 1.77 | 0.21 | −0.73 | 0.37 | 2.11 | 1.09 | 1.00 |
| 81533 | 0.46 | −0.86 | 0.92 | 0.52 | 0.43 | 1.00 | −0.07 | 0.63 | 0.37 | 1.91 | 0.63 | 0.03 | 1.80 |
| 201931 | −0.82 | −2.06 | −0.53 | 0.31 | −0.18 | 2.23 | 1.04 | 1.20 | 0.53 | 1.56 | 1.34 | 0.23 | 0.69 |
| 7979 | 0.73 | −1.07 | 0.61 | 0.16 | −0.93 | 0.91 | 0.91 | 1.92 | 0.99 | 1.14 | 0.74 | 0.36 | 0.72 |
| 159296 | −0.20 | 0.57 | 1.82 | −0.28 | −0.20 | 0.96 | 1.31 | −0.11 | 1.55 | 0.42 | −0.02 | 0.07 | 1.47 |
| 51102 | 0.04 | −0.44 | 0.71 | −0.09 | −0.58 | 1.10 | 0.34 | 1.23 | 1.17 | 0.60 | 2.12 | 0.26 | 1.67 |
| 8729 | −1.28 | 0.35 | 0.14 | 0.64 | 0.31 | 1.47 | 0.10 | 1.60 | 0.65 | 0.89 | 1.14 | 0.80 | 0.37 |
| 118460 | −0.58 | −0.27 | −0.35 | 0.30 | −0.12 | 1.27 | 1.31 | 0.60 | 1.80 | 0.06 | 2.06 | 0.33 | 0.56 |
| 79586 | 0.24 | −0.60 | −1.04 | 0.71 | −0.82 | 0.39 | 0.20 | 2.85 | 1.40 | 1.12 | 0.23 | 0.71 | 1.54 |
| 2052 | 1.11 | −0.86 | 2.09 | 0.72 | 0.28 | 1.09 | 0.47 | 1.12 | 0.36 | 1.57 | 0.82 | 0.19 | 0.90 |
| 84893 | −0.01 | −0.98 | 2.23 | 0.80 | −0.29 | 0.24 | 0.32 | 1.65 | 0.78 | 0.83 | 0.44 | 0.61 | 1.43 |
| 54726 | 0.36 | −2.67 | 1.55 | 0.71 | 1.09 | 1.10 | 0.96 | 1.20 | 0.32 | −0.03 | 0.73 | 0.08 | 1.29 |
| 813 | 0.26 | −1.77 | 0.48 | −0.37 | −0.09 | 2.63 | 0.58 | 1.67 | 0.30 | 1.18 | 1.13 | 0.50 | 1.34 |
| 54431 | −0.41 | −1.83 | 1.24 | 0.87 | −0.15 | 1.57 | −0.13 | 1.61 | 0.61 | 0.78 | 1.91 | −0.13 | 1.95 |
| 9612 | −0.10 | 0.09 | 0.14 | −0.93 | 0.39 | 1.90 | 0.95 | 1.41 | −0.26 | −0.32 | 1.42 | 0.35 | 0.75 |
| 9829 | −1.21 | 0.40 | 0.88 | 0.92 | 0.09 | 1.51 | 0.12 | 0.63 | 1.34 | 0.66 | 1.03 | 1.20 | 1.07 |
| 79036 | 0.68 | −1.54 | −1.19 | −0.11 | −0.10 | 1.36 | 1.66 | 0.70 | 0.74 | 1.82 | 0.10 | 0.48 | 0.54 |
| 284361 | 1.36 | −1.21 | 0.58 | −0.70 | −0.61 | 1.09 | 0.91 | 1.93 | 0.32 | 1.33 | 1.47 | 0.50 | 1.25 |
| 131118 | −0.13 | 0.65 | 0.03 | −0.07 | −1.13 | 0.97 | −0.11 | 2.78 | 1.05 | 0.56 | 1.43 | 0.50 | 1.46 |
| 644809 | 1.00 | 0.30 | −0.28 | 0.83 | 1.34 | 0.41 | 0.93 | 1.19 | −0.58 | 0.70 | 0.54 | 0.97 | 0.36 |
| 2119 | 1.06 | 0.15 | 0.03 | 0.09 | −0.07 | 1.55 | 1.30 | 1.41 | 1.18 | 0.92 | −0.35 | 0.12 | 1.33 |
| 5833 | 0.58 | 1.23 | 0.12 | 0.06 | −1.29 | 1.50 | 1.58 | 0.65 | 0.81 | −0.02 | 1.44 | 0.61 | 0.56 |
| 922 | 0.77 | −0.08 | 0.71 | 0.85 | −0.09 | −0.20 | 0.71 | 2.07 | 0.84 | 1.11 | 1.13 | 0.30 | 1.17 |
| 9261 | 0.75 | −0.75 | 0.99 | −0.36 | 0.30 | 0.92 | 1.06 | 1.74 | 0.14 | 0.06 | 1.46 | 0.69 | 0.45 |
| 57190 | 0.02 | −1.13 | 0.88 | 0.47 | 0.45 | 0.55 | 1.53 | 0.97 | 0.91 | 1.00 | 1.45 | 0.83 | 0.39 |
| 79644 | 0.87 | 0.35 | 0.34 | 0.38 | −0.24 | 2.00 | −0.02 | 0.97 | 0.20 | 1.39 | 1.28 | −0.14 | 1.40 |
| 79058 | 1.45 | 0.52 | 0.80 | 0.92 | −0.07 | 0.73 | 0.52 | −0.13 | 1.04 | 0.44 | 1.18 | 0.39 | 0.97 |
| 7832 | −0.31 | −0.16 | −0.29 | 0.98 | 1.51 | 1.17 | 0.00 | 0.95 | 0.05 | 0.18 | 0.88 | 2.64 | 0.53 |
| 11070 | 0.98 | −2.10 | 0.09 | 0.54 | 1.10 | 0.79 | 1.15 | 0.75 | 0.01 | −0.40 | 1.70 | 1.24 | 0.96 |
| 10847 | 0.38 | −1.04 | 0.23 | 1.10 | −0.37 | 1.36 | 1.79 | −0.19 | 0.04 | 1.17 | 0.48 | 1.85 | −0.04 |
| 4597 | 0.62 | 1.96 | 0.27 | −0.81 | −0.64 | 1.37 | 0.62 | 0.46 | 0.04 | 1.90 | −0.04 | 0.65 | 1.32 |
| 100506696 | 0.92 | −0.60 | 1.38 | −0.08 | −1.22 | 1.34 | −0.28 | 1.60 | 0.90 | 0.86 | 1.72 | −0.26 | 1.49 |
| 100507459 | 0.11 | 0.49 | 0.31 | 0.51 | −1.15 | 1.83 | 0.59 | 0.69 | 0.69 | 1.37 | 0.93 | −1.09 | 1.81 |
| 100130613 | 0.89 | −0.92 | 0.94 | −0.43 | 0.70 | 2.62 | 0.62 | 1.00 | −0.14 | 0.72 | 1.62 | 0.67 | −0.06 |
| 5081 | −0.11 | −0.28 | 0.03 | 0.14 | −0.03 | 1.12 | 1.76 | 1.27 | 0.76 | −0.18 | 0.73 | 0.94 | 0.62 |
| 100240728 | −0.08 | 1.21 | 0.24 | 0.14 | −0.85 | 1.34 | −0.18 | 0.72 | 0.70 | 0.95 | 2.04 | 0.91 | 0.79 |
| 26229 | 0.60 | 0.07 | −0.02 | −0.13 | −0.99 | 0.96 | 0.01 | 1.74 | 1.11 | 0.29 | 2.12 | 0.36 | 1.40 |
| 950 | 0.56 | −1.75 | 0.16 | −0.31 | −0.33 | 2.17 | 0.29 | 0.82 | 0.32 | 1.94 | 1.07 | 0.30 | 1.53 |
| 161882 | 1.03 | −1.82 | 0.50 | 0.42 | 0.44 | 0.06 | 1.41 | 1.20 | 1.53 | 1.01 | 1.28 | 0.50 | 0.03 |
| 5034 | 1.07 | −0.87 | 1.03 | −0.19 | −0.65 | 1.45 | 1.15 | 1.79 | 0.03 | 1.05 | 1.54 | −0.09 | 0.67 |
| 123355 | −0.04 | −1.58 | 1.12 | 0.24 | −0.15 | −0.31 | 0.69 | 0.79 | 0.81 | −0.05 | 1.64 | 0.68 | 1.25 |
| 6734 | −0.77 | −1.71 | 0.60 | 0.75 | 0.84 | 0.69 | −0.07 | 2.54 | −0.27 | −0.67 | 2.25 | 1.01 | 0.82 |
| 9911 | 2.21 | 0.29 | 0.89 | 0.19 | 0.45 | 0.69 | 1.42 | 1.32 | −0.55 | 1.64 | 1.70 | −0.42 | 0.14 |
| 222642 | 0.63 | −0.26 | 1.40 | 0.44 | 0.78 | 0.73 | 1.00 | 1.86 | 0.59 | 1.95 | 0.76 | −0.74 | 1.21 |
| 3643 | 0.88 | −0.54 | 0.97 | 0.74 | 0.48 | 1.94 | 1.22 | 0.41 | −0.68 | 1.40 | 0.37 | 0.39 | 0.70 |
| 85462 | 1.72 | 0.44 | 1.64 | 1.34 | 0.98 | 0.71 | −0.13 | 0.84 | 1.24 | 0.87 | −0.77 | 0.78 | 0.63 |
| 149175 | 0.94 | −0.11 | 0.62 | 0.36 | −0.87 | 1.31 | −0.24 | 1.27 | 0.71 | 1.31 | 1.65 | 0.25 | 1.16 |
| 1819 | 1.15 | −1.28 | 1.82 | 0.23 | −1.07 | 1.27 | 0.83 | 0.77 | 1.38 | 0.79 | 1.44 | 0.17 | 0.66 |
| 10370 | −0.11 | −1.87 | 1.52 | 1.11 | 0.32 | 1.35 | 1.00 | 2.19 | −0.28 | 0.37 | 1.78 | −0.04 | 0.50 |
| 25792 | 1.23 | −3.21 | 1.91 | 0.78 | 0.20 | 0.41 | 1.30 | 0.87 | 0.30 | 0.40 | 1.17 | −0.14 | 0.71 |
| 4072 | 1.47 | 1.86 | 1.30 | −0.31 | −0.08 | 1.58 | 0.17 | −0.06 | 1.18 | 1.61 | 1.63 | −0.46 | 0.14 |
| 84447 | 0.17 | −0.81 | 0.81 | 0.33 | 0.20 | 0.83 | 0.15 | 3.04 | −0.74 | −0.15 | 1.60 | 1.15 | 1.11 |
| 527 | 1.67 | −1.27 | 0.53 | −0.19 | 0.22 | 0.99 | 2.28 | 1.05 | 0.52 | 1.37 | 0.07 | −0.15 | 0.44 |
| 10629 | 0.72 | −0.79 | 0.18 | −0.37 | −0.96 | 0.72 | 1.25 | 1.36 | 0.81 | 0.49 | 1.62 | 0.83 | 1.07 |
| 51150 | 1.14 | −1.29 | 0.94 | −0.01 | −0.16 | 0.84 | 1.00 | 2.12 | −0.61 | 0.65 | 1.83 | 0.48 | 1.32 |
| 57143 | 1.06 | −1.36 | 0.60 | 0.80 | 0.12 | 1.28 | 0.45 | 1.08 | 0.00 | 0.96 | 1.52 | 0.37 | 1.31 |
| 682 | 1.17 | −1.40 | 1.79 | 0.29 | 0.58 | 0.24 | 1.35 | 1.35 | 0.01 | 0.28 | 0.94 | 0.86 | 0.64 |
| 10139 | 0.67 | 0.22 | 0.55 | −1.51 | −0.77 | 1.82 | 1.50 | 1.33 | 1.42 | 0.31 | 0.73 | 0.40 | 0.56 |
| 203238 | 0.81 | 0.36 | 0.98 | 0.27 | 0.00 | 2.16 | −0.98 | 0.49 | 1.18 | 0.10 | 1.11 | 0.59 | 1.07 |
| 54805 | 0.69 | −1.02 | 1.36 | 0.29 | −0.21 | 1.05 | −0.04 | 1.84 | 0.68 | 0.60 | 2.24 | −0.39 | 1.30 |
| 1200 | −0.89 | −0.66 | 1.35 | 0.23 | 0.58 | 0.63 | 1.99 | 0.41 | 0.33 | 0.30 | 1.89 | 0.03 | 0.51 |
| 2271 | 0.05 | −1.23 | 0.68 | −0.07 | −0.06 | 1.25 | 1.51 | 0.16 | 0.33 | 1.52 | 0.89 | 0.46 | 0.12 |
| 376412 | 0.11 | −0.29 | 0.56 | 0.07 | −0.79 | 2.48 | 0.85 | −0.14 | 1.86 | 0.35 | 1.91 | 0.17 | 0.12 |
| 4924 | 0.29 | −1.49 | 0.63 | −0.05 | 1.13 | −0.32 | 1.41 | 1.06 | 0.50 | 0.08 | 1.40 | 0.89 | 0.68 |
| 55132 | −0.41 | −0.06 | 1.04 | 0.62 | −0.36 | 2.03 | −0.55 | 2.66 | 0.91 | 0.20 | 1.79 | 0.48 | 0.96 |
| 9650 | −0.59 | 0.91 | 0.94 | 0.64 | −0.88 | 1.53 | 0.05 | 2.22 | 0.00 | 1.53 | 0.97 | 0.24 | 1.52 |
| 29803 | 0.72 | 0.31 | 1.07 | 0.30 | −0.59 | 0.61 | 0.78 | 0.74 | 0.33 | 0.58 | 2.47 | 0.04 | 0.49 |
| 4507 | 0.50 | −2.09 | −0.40 | −0.55 | −0.25 | 1.46 | 0.89 | 0.40 | 0.50 | 1.74 | 2.40 | 0.24 | 0.89 |
| 816 | 1.51 | 0.03 | 0.88 | −0.66 | 0.50 | 1.32 | 0.83 | 1.59 | −0.29 | −0.16 | 0.82 | 1.01 | 0.12 |
| 120379 | −0.51 | 1.52 | 0.81 | 0.62 | −0.08 | 1.13 | 0.31 | 0.51 | 0.91 | 1.86 | 0.68 | −0.05 | 0.60 |
| 91289 | 1.31 | −0.30 | −0.03 | 0.09 | −0.47 | 1.31 | 1.83 | 2.32 | 0.17 | 0.26 | 1.18 | 0.08 | 0.76 |
| 10905 | 0.26 | −2.42 | 0.90 | 1.97 | −0.62 | 1.19 | −0.05 | 1.84 | 0.33 | −0.35 | 1.88 | 0.67 | 0.92 |
| 25930 | −0.12 | 1.67 | 0.28 | 0.92 | 0.50 | 0.02 | 0.77 | 0.84 | 1.54 | −0.81 | 1.10 | 0.40 | 0.38 |
| 57648 | 1.23 | −3.21 | 1.11 | 0.50 | −0.47 | 2.10 | 1.08 | 0.71 | 0.23 | 1.36 | 0.17 | 0.16 | 0.50 |
| 7249 | 0.67 | −1.88 | 1.60 | 1.13 | 0.49 | 0.06 | 0.98 | 0.12 | 1.11 | −0.24 | 1.74 | 0.08 | 0.95 |
| 55151 | −1.50 | 0.61 | −0.02 | −0.12 | 0.00 | 2.10 | 0.27 | 2.35 | 1.09 | 0.85 | 1.35 | 0.51 | 1.28 |
| 160728 | 0.37 | 0.89 | 0.94 | 1.04 | −0.98 | 0.68 | 0.87 | 0.89 | −0.03 | 1.18 | 0.88 | −0.30 | 0.24 |
| 148418 | 0.56 | 0.26 | −0.01 | 0.66 | 0.27 | 0.01 | 0.99 | 0.48 | 1.10 | 0.63 | 2.51 | −0.04 | 1.03 |
| 646762 | 1.62 | −1.26 | 1.30 | 0.75 | −0.35 | 0.56 | 0.69 | 0.75 | 0.65 | 0.29 | 1.65 | −0.33 | 1.30 |
| 79109 | 0.88 | 0.18 | −0.23 | −0.30 | −0.65 | 0.49 | 0.44 | 1.00 | −0.21 | 0.50 | 1.70 | −0.01 | 1.53 |
| 81037 | 0.69 | −0.33 | 0.40 | −0.03 | −0.82 | 1.54 | 0.31 | 2.15 | 0.68 | 1.36 | 1.40 | −0.36 | 1.46 |
| 51523 | 0.55 | −1.23 | 1.07 | 0.16 | −0.55 | 1.11 | 0.59 | 1.55 | 0.71 | 1.32 | 1.90 | −0.56 | 1.92 |
| 55322 | −1.06 | 0.01 | −0.25 | 0.45 | 0.07 | 0.44 | 1.40 | 1.68 | −1.07 | 0.53 | 2.29 | 0.47 | 0.65 |
| 100996671 | 0.08 | 1.80 | 0.16 | 1.43 | 0.04 | 0.39 | 0.99 | 1.48 | 0.91 | −0.57 | 0.38 | 0.90 | 0.25 |
| 55222 | 0.24 | 1.55 | 1.03 | 0.51 | −1.06 | 0.66 | 0.18 | 0.59 | 2.49 | 0.37 | 0.82 | −0.19 | 1.05 |
| 55624 | 0.71 | −0.89 | 0.73 | 0.36 | −0.90 | 1.34 | 0.54 | 2.05 | 0.93 | 0.16 | 1.95 | −0.11 | 0.91 |
| 100505983 | 1.41 | 1.83 | 1.16 | 0.13 | −0.75 | 1.06 | −0.02 | 0.56 | 0.93 | 0.48 | 1.69 | 1.73 | 0.10 |
| 3069 | 0.76 | −1.02 | 0.55 | 0.09 | −0.32 | 1.25 | 0.11 | 2.33 | 0.43 | 0.56 | 1.76 | 0.03 | 1.39 |
| 10959 | 0.52 | −1.89 | 0.52 | 0.09 | −0.10 | 1.62 | 1.12 | 1.53 | −0.30 | 0.48 | 1.62 | 0.76 | 0.43 |
| 6598 | 0.70 | 0.16 | 1.80 | 0.11 | −0.46 | 1.24 | 1.23 | 1.83 | 1.00 | −0.41 | 0.86 | −0.20 | 0.45 |
| 60412 | 0.01 | −2.40 | 0.19 | −0.05 | 0.04 | 1.19 | 0.19 | 0.03 | 0.92 | 0.66 | 1.98 | 0.47 | 1.10 |
| 54982 | 0.73 | −0.65 | 0.76 | 0.09 | −1.20 | 1.12 | 0.57 | 1.61 | 0.39 | 1.37 | 1.49 | −0.31 | 1.79 |
| 91752 | 0.13 | −0.40 | 2.06 | 0.44 | 0.71 | 2.19 | 0.00 | −0.85 | 0.36 | 0.48 | −0.20 | 0.59 | 2.17 |
| 113178 | −0.20 | −2.04 | 1.61 | 0.32 | 0.51 | 0.52 | 1.13 | 0.55 | 1.03 | 0.38 | 1.46 | 1.48 | 0.38 |
| 10 | −0.91 | 1.22 | −0.17 | 0.67 | 0.24 | 1.69 | 0.65 | −0.79 | 1.15 | 0.49 | 1.03 | 1.20 | −0.08 |
| 375 | 0.74 | −2.81 | 0.22 | −0.31 | 0.50 | 1.12 | 1.33 | 0.52 | −0.06 | 0.47 | 1.27 | 1.07 | 0.74 |
| 5589 | 0.65 | −2.02 | 0.91 | 0.53 | 0.31 | 0.81 | 1.04 | 1.69 | 0.50 | −0.72 | 1.94 | 0.34 | 0.73 |
| 9136 | −0.65 | −1.29 | 0.83 | 0.24 | −0.27 | 0.33 | 1.05 | 0.96 | 0.76 | 1.39 | 1.46 | −0.50 | 2.04 |
| 23193 | −0.26 | −1.54 | 0.58 | 0.22 | −0.34 | 1.11 | 1.13 | 2.19 | 0.18 | 1.02 | 2.08 | 0.17 | 0.50 |
| 1798 | 0.51 | −0.74 | 0.31 | −0.10 | −0.74 | 1.69 | 0.40 | 1.59 | 0.99 | 1.79 | 1.26 | −0.13 | 1.36 |
| 85440 | 1.02 | −2.22 | 1.47 | 0.95 | −0.35 | 1.14 | −0.12 | 1.20 | 0.71 | −0.01 | 2.02 | 0.22 | 1.28 |
| 100505794 | 0.14 | 1.13 | −1.40 | 0.80 | −0.18 | 1.52 | 0.28 | 1.05 | 1.35 | 0.82 | 1.63 | 0.98 | 0.10 |
| 1173 | 0.65 | −3.44 | 1.20 | 0.32 | 0.34 | 0.23 | 1.42 | 0.43 | 0.23 | 0.47 | 1.69 | −0.21 | 0.91 |
| 124540 | 1.53 | −1.37 | 1.31 | 0.75 | −1.35 | 0.55 | 0.43 | 0.72 | 0.45 | −0.09 | 2.03 | −0.18 | 1.83 |
| 8372 | 0.57 | 0.63 | 0.87 | −0.29 | 0.03 | 1.00 | 2.11 | 1.17 | 0.30 | −0.05 | 1.43 | 0.16 | −0.04 |
| 64755 | 0.24 | −0.44 | 1.02 | 0.33 | −0.45 | 1.37 | 0.83 | 1.34 | 0.29 | 0.65 | 2.50 | 0.19 | 1.08 |
| 79102 | 1.17 | −0.35 | 1.28 | 0.15 | −0.79 | 0.77 | 1.30 | 0.89 | 0.00 | 0.46 | 1.97 | 0.33 | 0.63 |
| 10963 | 0.39 | −2.44 | 0.54 | 0.24 | 0.18 | 1.18 | 1.18 | 1.06 | −0.52 | 0.50 | 1.12 | 0.88 | 0.39 |
| 375387 | −1.49 | −2.04 | 1.82 | 1.02 | 0.80 | 0.78 | 2.50 | 0.31 | 0.28 | 0.63 | 1.24 | 0.04 | 0.99 |
| 972 | 0.45 | −1.10 | 0.36 | 0.22 | −0.06 | 0.94 | 1.05 | 0.53 | 1.14 | 0.80 | 1.59 | 0.02 | 0.34 |
| 54676 | 0.41 | 1.10 | 1.61 | 0.46 | 0.82 | 0.86 | 0.85 | 0.48 | −0.30 | 0.08 | 1.32 | 0.31 | −0.23 |
| 192286 | −0.93 | −1.19 | 1.07 | −0.25 | −0.86 | 1.32 | 1.09 | 1.57 | 1.38 | 1.15 | 1.75 | −0.10 | 1.02 |
| 3295 | 0.87 | −2.80 | 1.49 | 0.77 | −0.06 | 1.32 | 1.16 | −0.03 | 0.15 | 1.18 | 0.17 | 0.00 | 1.16 |
| 80852 | 0.71 | 0.57 | 0.30 | 0.05 | 0.54 | 1.02 | −0.38 | 2.14 | −0.44 | 1.91 | 0.67 | 0.34 | 0.32 |
| 1298 | 0.64 | 1.33 | 0.56 | 0.37 | 0.00 | 2.13 | 0.83 | 0.67 | −0.08 | 0.10 | 0.21 | 0.35 | 0.61 |
| 2004 | 0.61 | 0.13 | 0.42 | 0.09 | −0.66 | 1.55 | 0.98 | 1.45 | 0.65 | 1.35 | 1.54 | 0.13 | 0.37 |
| 10279 | 0.31 | 0.82 | −0.14 | 0.21 | −0.26 | 1.11 | −1.71 | 2.05 | 1.03 | 1.08 | 1.35 | 1.07 | 0.80 |
| 151790 | 0.87 | −1.62 | 0.76 | 0.78 | 0.00 | 2.14 | 1.03 | −0.14 | 1.45 | 2.44 | 0.44 | −0.90 | 0.42 |
| 399664 | 0.08 | −0.89 | 0.24 | 0.58 | −0.83 | 2.15 | 0.82 | 0.42 | 0.57 | 1.84 | 1.06 | 0.51 | 0.60 |
| 5664 | −0.14 | 0.28 | 0.06 | 0.64 | −0.30 | 2.65 | −0.12 | 0.78 | −0.07 | 1.74 | 0.55 | −0.24 | 2.05 |
| 51614 | 0.90 | −0.92 | 0.50 | 0.13 | −0.86 | 1.60 | 0.13 | 2.12 | 0.34 | 0.84 | 2.19 | 0.03 | 1.23 |
| 79178 | 0.68 | −1.53 | 0.69 | −0.05 | −0.69 | 0.93 | 0.76 | 1.46 | −0.25 | 2.19 | 1.12 | −0.18 | 1.61 |
| 51726 | 1.02 | −0.53 | 0.36 | 0.10 | −0.70 | 1.88 | 0.46 | 1.61 | 0.24 | 1.27 | 1.21 | −0.02 | 1.16 |
| 3425 | 0.23 | −0.33 | 1.39 | 0.21 | −0.83 | 1.37 | 0.55 | 2.21 | 0.42 | 0.93 | 1.79 | −0.31 | 1.22 |
| 1642 | 0.82 | 0.26 | 0.71 | −0.23 | −1.49 | 1.42 | 0.80 | 1.27 | 1.21 | 0.97 | 1.91 | −0.64 | 0.89 |
| 101928770 | −0.05 | −0.75 | 1.30 | −0.42 | 0.22 | 0.39 | 0.06 | −0.18 | 0.88 | 1.13 | 1.91 | 0.96 | 0.40 |
| 11015 | 0.96 | 0.95 | −0.77 | 0.24 | −0.94 | 1.34 | −0.28 | 1.49 | −0.18 | 1.11 | 1.12 | 1.25 | 1.41 |
| 35 | 1.25 | 0.88 | 0.45 | 0.33 | −1.07 | −0.63 | 0.70 | 2.04 | 0.58 | −0.21 | 0.90 | 0.85 | 0.93 |
| 64798 | 1.14 | −0.50 | 0.90 | 0.64 | −0.81 | 0.91 | 0.34 | 1.30 | 1.17 | 0.54 | 2.26 | −0.13 | 1.32 |
| 23457 | −0.22 | 0.74 | 0.32 | 0.07 | 0.08 | 0.78 | 0.09 | 2.03 | 1.05 | 0.73 | 0.22 | 0.46 | 0.65 |
| 56132 | −0.10 | 0.17 | 0.49 | 1.08 | 0.93 | 2.27 | 0.23 | −0.14 | 1.28 | −0.57 | 0.24 | 1.04 | 0.39 |
| 23400 | −0.57 | 0.06 | 0.84 | 0.17 | −1.01 | 0.01 | 1.55 | 1.71 | 1.58 | 0.81 | 1.67 | 0.46 | 1.14 |
| 3980 | 0.36 | −0.54 | 0.48 | 0.86 | −0.21 | 1.56 | 0.80 | 0.39 | 0.02 | 1.11 | 1.28 | 0.03 | 1.27 |
| 5690 | 0.57 | −0.70 | 1.14 | 0.01 | −0.55 | 0.18 | 0.83 | 1.46 | 0.33 | −0.16 | 2.18 | 0.18 | 1.27 |
| 7917 | 1.38 | −1.40 | 1.08 | 0.01 | −0.80 | 0.97 | 1.34 | 1.73 | 0.60 | 1.00 | 1.81 | −0.75 | 0.71 |
| 1739 | −0.75 | −0.96 | 1.19 | 0.48 | −0.38 | 1.57 | −0.07 | 1.94 | 0.87 | −0.62 | 2.35 | 0.56 | 0.51 |
| 2837 | 0.22 | 0.05 | 0.23 | −0.10 | 0.19 | 0.95 | 0.95 | 0.99 | 1.56 | −0.42 | 1.90 | 0.81 | 0.66 |
| 83746 | 0.89 | −0.51 | 0.42 | 0.04 | −0.22 | 1.29 | 1.21 | 1.06 | −0.15 | 1.10 | 1.67 | 0.02 | 0.65 |
| 5962 | −0.63 | 0.90 | 0.24 | 0.52 | −0.06 | 2.04 | 0.60 | 1.77 | 0.50 | 0.72 | 1.01 | 1.07 | 0.56 |
| 9956 | 0.27 | 0.74 | 0.52 | −0.45 | −0.25 | 3.02 | 0.28 | −0.01 | 1.34 | 0.80 | 0.38 | 0.50 | 0.39 |
| 162968 | 1.56 | 2.47 | −1.23 | 0.27 | −0.36 | 0.45 | 0.25 | 0.24 | 1.17 | 1.14 | −0.12 | 0.59 | 0.97 |
| 4192 | 0.81 | 0.61 | 0.59 | −0.39 | −0.31 | 0.92 | 0.47 | 2.08 | 0.74 | 1.48 | 0.74 | −0.09 | 1.14 |
| 54919 | 0.26 | 0.87 | 1.46 | 0.50 | −0.57 | 1.29 | 0.54 | 0.96 | −0.12 | 1.96 | 1.12 | −0.28 | 1.02 |
| 10972 | 0.23 | −2.06 | −0.13 | 0.02 | −0.74 | 1.77 | 0.23 | 2.46 | 0.42 | 1.56 | 1.44 | 0.21 | 1.12 |
| 23061 | 1.47 | −0.17 | 0.78 | −0.15 | −0.54 | 1.50 | 0.81 | 0.61 | 0.50 | −0.02 | 1.88 | −0.13 | 0.96 |
| 3792 | 0.27 | 0.74 | 1.85 | 0.31 | −0.05 | 0.68 | 0.56 | 1.10 | 0.83 | 1.13 | 0.58 | −0.81 | 1.84 |
| 2063 | 0.43 | 2.13 | 0.83 | 0.19 | −0.08 | 0.90 | 0.72 | 0.27 | 0.73 | 2.32 | −0.26 | −0.53 | 0.72 |
| 55177 | 0.44 | 0.38 | 0.90 | −0.31 | −0.97 | 1.51 | −0.17 | 1.73 | 0.72 | 1.80 | 1.84 | −0.38 | 1.52 |
| 11147 | 0.13 | −2.30 | 0.11 | 0.71 | −0.24 | 1.31 | 0.62 | 1.61 | 1.44 | 1.52 | 0.99 | −0.64 | 1.50 |
| 57486 | 0.19 | −1.92 | 0.21 | 0.51 | 0.33 | 1.15 | 1.70 | −0.82 | 0.09 | 1.67 | 0.27 | 0.48 | 1.34 |
| 30814 | 0.91 | 0.07 | 0.49 | −0.23 | −1.35 | 0.01 | 0.00 | 2.32 | 1.05 | 0.30 | 1.56 | 0.28 | 0.57 |
| 57414 | 0.76 | −0.84 | −0.49 | −0.74 | 0.32 | 2.32 | −0.19 | 1.24 | 0.89 | 0.83 | 0.18 | 1.65 | 1.21 |
| 84836 | 0.50 | −1.38 | 1.10 | −0.20 | −0.91 | 1.01 | 0.94 | 1.68 | 1.17 | 1.06 | 2.18 | −0.34 | 0.87 |
| 8516 | −0.09 | −0.90 | −0.03 | 1.49 | 0.25 | 1.21 | 0.02 | 2.41 | 1.07 | 0.22 | 0.17 | 1.02 | 2.16 |
| 89866 | 0.30 | 0.07 | 0.92 | 1.22 | −0.67 | 1.20 | −0.94 | 0.19 | 1.74 | 0.58 | 1.43 | −0.32 | 0.67 |
| 10491 | 0.31 | −2.19 | 0.24 | −0.11 | −0.70 | 0.36 | 1.16 | 1.65 | 0.39 | 1.08 | 1.97 | −0.10 | 1.15 |
| 11047 | 1.78 | 0.53 | 1.42 | −0.82 | −0.35 | 1.12 | 1.04 | 0.91 | 0.66 | 1.05 | 0.97 | −0.49 | 0.27 |
| 28971 | 2.32 | 0.38 | 0.56 | −0.35 | −0.63 | 0.91 | −0.04 | 0.74 | 0.41 | 0.55 | 0.75 | 0.52 | 1.27 |
| 8078 | 1.26 | −1.97 | 1.65 | −0.60 | −0.79 | 0.83 | 1.03 | 1.70 | 1.40 | −0.20 | 1.71 | −0.58 | 0.82 |
| 37 | 1.26 | −0.92 | 1.19 | 0.62 | −0.34 | 1.34 | 0.92 | 0.66 | 0.12 | 0.99 | 1.25 | −0.18 | 1.30 |
| 145508 | −0.20 | 0.67 | 0.60 | 0.28 | −0.19 | 1.59 | −0.82 | 1.68 | 1.14 | 1.20 | 1.82 | −0.36 | 0.73 |
| 49861 | 1.58 | −1.01 | 0.53 | 0.41 | −0.64 | 1.75 | 0.12 | 1.45 | 0.94 | −1.19 | 0.64 | 0.71 | 0.43 |
| 5905 | 0.78 | −0.96 | 0.63 | −0.14 | −1.29 | 0.65 | 1.09 | 1.62 | 0.41 | 0.84 | 1.96 | 0.02 | 0.88 |
| 7542 | 0.51 | −1.65 | 1.04 | −0.56 | 0.27 | 0.76 | 1.26 | 1.06 | 0.37 | 0.64 | 0.81 | 0.68 | 0.85 |
| 9562 | −0.65 | −1.76 | 0.37 | 1.29 | 0.08 | 0.78 | 0.44 | 1.99 | 0.48 | 1.37 | 1.34 | 0.26 | 1.74 |
| 79095 | 0.89 | 0.96 | −0.21 | −0.58 | −0.46 | −0.13 | 1.85 | 1.41 | 1.20 | −0.57 | 1.83 | 0.41 | −0.24 |
| 8815 | 0.39 | −3.22 | 0.98 | 0.62 | 0.38 | 0.81 | 0.91 | 0.85 | 0.22 | 0.02 | 1.60 | −0.32 | 0.80 |
| 54867 | −0.14 | −1.99 | 0.06 | 0.49 | 0.55 | 0.73 | 0.90 | 1.61 | 0.99 | −1.05 | 1.10 | 0.44 | 0.55 |
| 6721 | −0.36 | −0.39 | 1.66 | −0.57 | −0.09 | 0.41 | 2.01 | 1.07 | 0.36 | −0.13 | 0.81 | 0.28 | 0.77 |
| 23133 | 0.16 | 0.98 | 0.12 | 0.56 | 1.06 | 1.95 | 0.14 | 1.21 | 0.50 | −0.54 | 0.82 | 0.11 | 0.49 |
| 113675 | 0.65 | −0.53 | 1.95 | 0.34 | −1.22 | 1.71 | 0.59 | 0.29 | 1.64 | 1.52 | −0.21 | −0.65 | 1.96 |
| 129303 | 0.71 | −0.48 | −0.19 | −0.01 | 0.70 | −0.43 | 1.43 | 0.59 | 1.14 | 0.72 | 1.91 | 0.34 | 0.60 |
| 57658 | 1.18 | −1.65 | 0.80 | 1.39 | 0.87 | 0.49 | 1.24 | 0.11 | 0.05 | −0.45 | 1.13 | 0.89 | −0.26 |
| 162 | 1.27 | −0.68 | 1.09 | −0.54 | −0.32 | 0.67 | 1.06 | 0.49 | 0.79 | 0.53 | 1.48 | 0.46 | 0.87 |
| 2873 | 0.31 | −1.03 | 0.65 | 0.32 | −0.93 | 0.91 | 1.18 | 1.35 | 0.91 | 0.61 | 1.78 | 0.24 | 1.14 |
| 64978 | 0.07 | −1.17 | 0.11 | 0.22 | −0.02 | 0.45 | 1.55 | 1.48 | 0.58 | 0.56 | 2.00 | 0.13 | 0.90 |
| 220323 | 0.73 | −0.83 | 1.76 | 0.61 | −0.26 | 0.80 | 1.35 | 1.56 | −0.15 | 0.53 | 1.59 | −0.16 | 1.20 |
| 6006 | 0.71 | 0.38 | 1.81 | −0.52 | 0.25 | 0.25 | 0.61 | 0.83 | 1.04 | 1.81 | 1.10 | 0.07 | 0.74 |
| 389812 | 0.45 | 1.73 | 0.80 | −0.48 | −0.46 | 1.02 | 0.52 | 2.10 | −1.40 | 1.21 | 0.82 | −0.27 | 0.91 |
| 2628 | −0.16 | 0.93 | 0.13 | 0.50 | −0.81 | 1.86 | −0.24 | 0.43 | 0.20 | 0.65 | 2.88 | −0.31 | 0.94 |
| 6509 | 0.07 | 0.08 | 0.64 | 0.35 | −0.34 | 1.69 | −0.05 | 1.52 | 0.04 | 2.06 | 0.78 | 0.89 | 1.80 |
| 3888 | 1.04 | 0.07 | 0.05 | −0.43 | −1.20 | 1.84 | −0.10 | 1.27 | 1.39 | −0.20 | 1.34 | 0.54 | 0.02 |
| 5211 | 1.47 | −0.49 | 0.49 | −0.55 | −0.40 | 0.72 | 1.58 | 0.63 | 0.70 | 0.30 | 1.64 | −0.31 | 0.77 |
| 55080 | 0.26 | −3.02 | −0.36 | −0.02 | 0.35 | −0.03 | 0.62 | 1.57 | −1.06 | 1.71 | 1.57 | 0.45 | 1.03 |
| 2139 | −0.28 | 0.42 | 0.05 | 0.27 | −0.62 | 1.63 | −0.03 | 1.74 | 0.11 | 2.34 | 1.54 | 0.63 | 0.55 |
| 2030 | 1.33 | −0.19 | 1.48 | 0.26 | −0.95 | 0.67 | 1.88 | 0.44 | 0.37 | 1.31 | 1.52 | −0.80 | 0.96 |
| 56005 | 0.99 | −1.11 | 0.16 | −0.30 | −0.75 | 1.32 | 0.38 | 2.37 | 0.01 | 0.96 | 1.24 | −0.03 | 2.13 |
| 6414 | 0.31 | 0.01 | 1.70 | 0.16 | −0.48 | 0.45 | 0.27 | 1.23 | 1.28 | 1.49 | 0.67 | −0.01 | 1.71 |
| 5184 | 1.00 | −3.74 | 0.88 | −0.36 | −0.47 | 0.26 | 0.95 | 0.76 | 0.83 | 1.24 | 0.61 | −0.30 | 1.24 |
| 643783 | −0.25 | 0.81 | −0.06 | 1.37 | −1.02 | 2.72 | 0.30 | 0.24 | 0.11 | 2.25 | −0.47 | 0.80 | 1.42 |
| 101928185 | 0.04 | 0.22 | 0.57 | −0.44 | 0.27 | 1.62 | −0.09 | 0.39 | 0.27 | 1.04 | 1.36 | −0.27 | 0.32 |
| 22883 | 0.78 | −0.52 | 0.11 | 0.20 | −0.78 | 0.83 | 0.11 | 1.34 | 1.41 | 0.80 | 2.32 | −0.15 | 0.83 |
| 25800 | −0.17 | −1.15 | 0.57 | 0.92 | 0.61 | 1.45 | 0.29 | 1.08 | 0.34 | −0.45 | 1.40 | 0.76 | 1.11 |
| 54681 | 0.75 | −0.35 | 0.64 | −0.03 | −0.56 | 1.97 | 0.06 | 1.79 | 0.92 | 0.52 | 1.43 | 0.09 | 0.95 |
| 644150 | 0.67 | 1.88 | −0.20 | −0.63 | −0.96 | 1.19 | −0.90 | 1.97 | 1.37 | 0.78 | 1.04 | 0.38 | 0.75 |
| 9048 | 0.42 | −0.31 | 1.27 | 0.26 | −0.87 | 2.97 | 0.99 | 0.80 | 0.44 | 0.17 | 0.56 | −0.49 | 0.30 |
| 3054 | 0.88 | 0.82 | 0.36 | −0.16 | −0.94 | 1.52 | 0.39 | 1.81 | 0.82 | 0.29 | 1.87 | −0.23 | 0.53 |
| 115098 | 1.66 | −1.30 | 1.67 | 0.58 | −0.42 | 0.22 | 1.05 | 0.24 | 0.41 | −0.38 | 1.85 | 0.01 | 0.78 |
| 55558 | 0.83 | −0.91 | 0.78 | 0.31 | −0.98 | 1.18 | 0.35 | 1.76 | 1.14 | 0.35 | 2.29 | 0.12 | 0.93 |
| 675 | −0.24 | −0.02 | 3.09 | 0.09 | −0.64 | 0.94 | 0.65 | 0.36 | 1.01 | 0.61 | 1.52 | 0.45 | 0.48 |
| 9777 | −0.38 | −1.21 | 0.51 | −0.17 | 0.05 | 1.45 | 1.76 | 0.99 | −0.82 | 1.31 | 1.67 | 0.50 | 1.49 |
| 83707 | 0.43 | −2.69 | 0.74 | 0.16 | −0.10 | 1.04 | 0.33 | 1.49 | 0.78 | 1.06 | 0.48 | 0.29 | 0.96 |
| 55334 | 0.12 | 0.94 | −0.80 | 0.19 | 0.14 | 1.20 | 0.80 | −0.06 | 1.19 | 1.37 | −0.87 | 1.39 | 0.28 |
| 9793 | 0.54 | 0.26 | 1.41 | 0.58 | −0.83 | 1.16 | 0.98 | 0.68 | 0.72 | 0.41 | 2.14 | −0.53 | 0.27 |
| 56834 | −0.34 | 0.81 | 0.36 | −0.05 | 0.56 | 1.20 | 1.44 | 0.83 | −0.19 | 1.06 | −0.02 | 1.44 | 0.42 |
| 25921 | 0.57 | −0.90 | 0.11 | 0.66 | 0.16 | 0.73 | 1.29 | 1.18 | −0.32 | 1.05 | 0.46 | 0.36 | 1.11 |
| 6520 | 0.66 | −1.50 | 1.41 | 0.09 | −0.74 | 1.85 | 0.84 | 1.55 | −0.55 | 0.56 | 1.54 | −0.14 | 1.60 |
| 728591 | 0.41 | 1.32 | 1.49 | −0.12 | 0.40 | 0.33 | 0.33 | 0.74 | 0.72 | −0.82 | 1.65 | 0.69 | 0.17 |
| 7415 | 1.27 | −0.80 | 1.30 | 0.34 | −0.40 | 1.57 | 0.03 | 1.03 | 0.73 | 0.36 | 1.30 | −0.08 | 1.33 |
| 130827 | 0.17 | −0.95 | 0.49 | 0.94 | −0.69 | 0.85 | −0.21 | 1.07 | 1.40 | 0.88 | 2.10 | 0.50 | 1.22 |
| 9620 | −1.74 | 1.90 | 0.13 | 0.81 | −0.25 | 1.91 | 0.05 | −0.65 | 1.54 | 1.44 | 0.76 | −0.01 | 0.47 |
| 55335 | 0.75 | −3.17 | 0.61 | 0.07 | 0.04 | 1.59 | 0.27 | 0.23 | 0.88 | 0.22 | 2.57 | 0.02 | 1.21 |
| 84988 | 0.53 | −1.39 | 0.79 | 0.62 | −0.42 | 1.14 | 1.45 | 0.97 | 0.35 | 1.71 | 0.57 | 0.14 | 0.69 |
| 100192378 | −0.51 | 3.12 | −0.66 | 0.35 | 0.57 | 0.93 | 0.54 | −0.86 | 1.05 | 0.27 | −0.40 | 0.80 | 0.75 |
| 442524 | −0.37 | 0.85 | 1.79 | 0.02 | −0.39 | 1.24 | 1.42 | −0.45 | −0.65 | 0.75 | 1.42 | −0.31 | 2.31 |
| 51661 | 0.37 | −0.06 | 0.46 | 0.66 | −1.13 | 0.65 | 0.33 | 1.51 | 0.87 | −0.21 | 2.34 | −0.48 | 1.88 |
| 84922 | −0.69 | −1.35 | 0.98 | −0.19 | −0.25 | 0.91 | 0.94 | 1.99 | 1.15 | −0.01 | 1.58 | 1.00 | 0.23 |
| 84954 | 1.93 | −1.93 | 0.97 | −0.04 | −0.08 | 0.28 | 0.53 | 1.07 | −0.52 | 1.06 | 1.48 | −0.35 | 1.49 |
| 245972 | 0.73 | 0.96 | −0.51 | −1.18 | −0.41 | 1.48 | −0.24 | 1.44 | −0.04 | 1.00 | 0.54 | 0.87 | 0.85 |
| 4316 | 0.50 | −0.74 | 0.00 | −0.69 | 0.28 | 2.65 | 0.49 | 0.76 | 1.87 | 0.90 | 1.03 | −0.57 | 0.71 |
| 55157 | −1.34 | 2.38 | 2.03 | 0.58 | −0.35 | 0.16 | 0.51 | 0.50 | 0.78 | 0.68 | 0.81 | 0.00 | −0.10 |
| 7466 | −0.24 | −0.07 | −1.03 | −0.58 | −0.50 | 2.42 | −0.37 | 2.50 | 0.94 | 1.76 | 0.61 | 0.18 | 0.91 |
| 79365 | 0.08 | 0.73 | −1.29 | 0.71 | −0.12 | 2.56 | −1.17 | 1.10 | 0.06 | 1.88 | 0.41 | 0.94 | 1.97 |
| 10066 | 0.20 | −1.15 | −0.05 | −0.17 | −0.69 | 1.39 | 0.88 | 1.43 | −0.21 | 1.61 | 1.91 | 0.43 | 0.98 |
| 203068 | 0.71 | −0.84 | 1.86 | −0.22 | −0.95 | 1.14 | 1.02 | 0.59 | 0.57 | 0.72 | 2.13 | −0.52 | 0.91 |
| 5780 | 0.51 | −1.65 | 0.38 | 0.53 | 0.14 | 0.48 | 1.64 | −0.13 | 0.64 | −0.46 | 0.92 | 0.83 | 1.05 |
| 285627 | −0.58 | 1.13 | −0.65 | 0.35 | 0.49 | 1.01 | 0.69 | 1.52 | 0.44 | 0.72 | −0.07 | 0.39 | −0.01 |
| 2058 | 0.72 | −0.88 | 0.55 | 0.12 | −1.04 | 1.09 | −0.01 | 1.42 | 0.45 | 1.01 | 1.70 | 0.16 | 1.26 |
| 1737 | −0.45 | −1.31 | 1.03 | 0.49 | −0.54 | 1.36 | 0.57 | 1.87 | 0.02 | 1.13 | 1.88 | −0.02 | 1.39 |
| 22924 | 0.07 | 0.88 | 0.45 | −0.25 | 0.11 | 1.38 | 1.16 | −0.52 | 0.97 | 0.99 | 0.18 | 1.74 | 0.64 |
| 6005 | 0.86 | 0.57 | 3.03 | 0.53 | 0.47 | 0.50 | 1.47 | 0.28 | 0.66 | 0.59 | 1.08 | −0.15 | 0.00 |
| 5286 | −1.19 | −1.43 | 0.09 | 0.38 | 0.29 | 1.07 | 0.06 | 2.18 | 0.35 | −0.03 | 2.31 | 1.09 | −0.01 |
| 11226 | 0.53 | −1.82 | 0.65 | −0.03 | −1.12 | 1.89 | 0.71 | 0.39 | 0.59 | 3.15 | 0.17 | 0.41 | 0.42 |
| 2762 | 0.90 | 0.10 | 1.55 | 0.55 | −0.41 | 0.02 | 0.53 | 0.41 | −0.39 | 0.63 | 1.39 | 0.18 | 2.47 |
| 8908 | 1.05 | −1.41 | 0.20 | 0.81 | −0.82 | 0.61 | 0.23 | 1.55 | 1.12 | 0.69 | 2.25 | 0.15 | 0.33 |
| 10693 | 0.92 | −0.10 | 1.02 | −0.68 | −1.48 | 1.36 | −0.08 | 0.87 | 0.36 | 0.52 | 2.67 | 0.11 | 1.01 |
| 199953 | 0.77 | −0.08 | 1.18 | 0.08 | −0.88 | 0.76 | 1.19 | 1.04 | 1.03 | 1.33 | 1.95 | −1.28 | 1.17 |
| 2760 | −0.75 | 0.96 | 0.03 | −0.31 | 1.15 | 0.97 | 1.25 | −0.21 | −0.02 | 0.89 | −0.31 | 1.09 | 1.20 |
| 8295 | −0.66 | −1.04 | 0.80 | 0.51 | −0.24 | 0.95 | 0.18 | 1.77 | 1.14 | 0.54 | 2.43 | −0.75 | 0.82 |
| 1822 | 1.14 | −0.25 | 0.58 | −0.28 | 0.19 | 1.99 | 0.05 | 0.06 | 0.65 | 1.87 | 0.66 | 0.22 | 0.19 |
| 55328 | 0.33 | −0.12 | 0.29 | 0.14 | −0.25 | 0.92 | 0.14 | 1.12 | 0.14 | 1.28 | 0.84 | −0.08 | 1.21 |
| 2069 | −0.18 | −2.29 | 2.31 | 1.04 | 0.24 | 1.36 | 0.69 | −0.88 | 1.00 | 0.78 | 1.25 | 1.02 | 0.40 |
| 4329 | 1.04 | −0.62 | 1.18 | 1.06 | −0.76 | 0.97 | 0.40 | 1.85 | −0.13 | 0.26 | 1.84 | −0.13 | 0.76 |
| 537 | 1.00 | −0.94 | 0.77 | 0.13 | 0.12 | 1.97 | 1.12 | 0.18 | −0.33 | 1.86 | 0.64 | 0.06 | 1.40 |
| 64764 | 0.62 | −1.03 | 0.46 | −0.15 | −0.75 | 1.59 | 0.40 | 1.99 | 0.36 | 1.11 | 2.16 | −0.35 | 1.33 |
| 811 | 1.94 | −0.71 | 2.17 | 0.49 | −0.11 | 0.57 | 0.99 | 1.10 | 0.03 | 0.56 | 0.67 | 0.12 | 0.04 |
| 9779 | 0.80 | −1.77 | 0.91 | 0.05 | 0.19 | 1.03 | 0.86 | 0.13 | 1.84 | 0.24 | 0.47 | 0.56 | 0.13 |
| 114815 | 0.82 | 0.65 | −0.25 | −0.06 | 0.04 | 0.99 | 0.57 | 0.76 | 1.29 | −0.08 | 1.90 | 0.36 | 1.41 |
| 29927 | 0.80 | −1.90 | 0.82 | −0.28 | −0.38 | 1.10 | 0.65 | 2.00 | −0.15 | 1.09 | 1.95 | 0.27 | 0.98 |
| 6007 | −0.30 | −2.21 | 3.60 | −0.25 | 0.13 | 0.60 | 0.99 | −0.08 | 0.48 | 0.37 | 0.65 | 0.37 | 0.58 |
| 5192 | 0.35 | −0.98 | 0.89 | 0.02 | −0.94 | 1.06 | 1.04 | 1.71 | 0.04 | 1.83 | 2.25 | −0.46 | 1.06 |
| 125875 | 0.93 | −0.02 | 0.38 | 0.44 | −1.04 | 1.04 | 0.88 | 2.06 | 0.43 | −0.30 | 1.44 | −0.09 | 0.34 |
| 10882 | −0.87 | −0.34 | −1.38 | 0.32 | −0.02 | 1.27 | 1.32 | 0.79 | 1.36 | 0.30 | 0.22 | 1.82 | 0.33 |
| 192683 | 1.06 | 0.02 | −1.29 | 0.08 | −0.28 | 1.01 | −0.25 | 1.66 | −0.27 | 0.06 | 1.83 | −0.39 | 1.72 |
| 8028 | 1.30 | −2.16 | 2.07 | 0.31 | −0.49 | 0.97 | 1.24 | 0.85 | −0.16 | 0.70 | 1.64 | −0.36 | 0.04 |
| 150368 | 1.27 | 1.03 | −0.45 | −0.18 | 0.17 | 0.44 | 1.40 | 0.28 | 0.88 | 1.54 | 0.22 | −0.17 | 0.13 |
| 84206 | 0.97 | −0.59 | 1.57 | −0.11 | −0.57 | 1.38 | 0.68 | 0.54 | 0.48 | −0.44 | 2.61 | −0.37 | 1.23 |
| 101929288 | −0.28 | 2.96 | −0.22 | −0.54 | −0.33 | 2.22 | 0.77 | 0.83 | −0.08 | −0.07 | 0.05 | 0.09 | 1.00 |
| 826 | 0.76 | −3.26 | 0.77 | 0.52 | −0.19 | 0.63 | 1.61 | 0.43 | 0.34 | 0.84 | 1.83 | −0.77 | 0.68 |
| 83440 | 0.42 | −2.56 | 1.02 | −0.08 | 0.21 | 1.32 | 1.35 | 0.92 | −0.34 | 0.12 | 1.56 | 0.15 | 0.35 |
| 1128 | −0.37 | 2.25 | −0.48 | 0.10 | −0.18 | 0.73 | 0.05 | 1.25 | 0.26 | −0.43 | 0.61 | 1.71 | 0.51 |
| 160287 | −0.28 | 0.07 | 0.91 | 0.85 | 0.11 | 0.18 | 0.66 | 0.28 | 0.68 | 0.11 | 2.53 | −0.18 | 0.41 |
| 412 | −1.11 | −1.51 | 0.72 | 0.07 | 0.57 | 1.03 | 0.93 | −0.82 | 1.08 | 1.31 | −0.81 | 1.12 | 1.14 |
| 64215 | 0.20 | −1.07 | 0.99 | 0.80 | −0.64 | 0.86 | −0.78 | 2.20 | 0.77 | 0.66 | 1.91 | 0.24 | 0.70 |
| 7343 | 0.98 | −1.66 | 0.77 | 0.02 | −0.03 | 0.29 | 0.99 | 0.42 | 0.51 | 0.18 | 1.34 | 0.59 | 0.16 |
| 25840 | 0.14 | −1.69 | 1.19 | 1.40 | 0.47 | 0.39 | 1.15 | −0.10 | −0.54 | 1.51 | 1.92 | −0.12 | 0.55 |
| 55315 | −0.05 | −2.89 | 0.22 | 0.73 | 0.18 | 1.30 | 0.53 | −0.16 | 0.98 | 1.67 | 0.74 | 0.33 | 1.12 |
| 100507513 | −0.41 | 1.70 | 1.86 | −0.44 | 1.18 | 0.40 | 0.37 | 0.99 | 0.10 | −0.43 | 1.39 | 0.32 | 0.33 |
| 55704 | 0.02 | −1.39 | 1.97 | 0.78 | 0.13 | 0.51 | 1.04 | 0.49 | 0.85 | −0.34 | 0.43 | 0.52 | 0.76 |
| 57604 | 0.26 | 2.66 | 0.32 | 0.10 | 0.14 | 1.47 | 0.03 | 1.45 | 0.27 | −0.63 | 0.66 | −0.09 | 0.52 |
| 10449 | −0.83 | −0.87 | 1.95 | 0.82 | 0.20 | 0.95 | 0.10 | −0.05 | −0.18 | 0.42 | 0.62 | 1.18 | 1.64 |
| 1727 | 1.79 | −0.87 | 0.66 | −0.72 | 0.46 | 1.17 | 1.60 | 0.04 | −0.02 | 1.71 | 0.91 | −0.16 | −0.05 |
| 9569 | 0.73 | −0.35 | −0.45 | 0.62 | 0.33 | 2.30 | −0.20 | 1.25 | 0.37 | −0.25 | 1.63 | 0.86 | 0.20 |
| 10009 | 0.91 | −2.59 | 0.73 | 0.21 | −0.67 | 1.84 | 0.66 | 1.07 | 0.79 | 0.11 | 2.29 | −0.96 | 0.99 |
| 5188 | 0.70 | −0.48 | 0.86 | −0.21 | −1.05 | 0.26 | 0.91 | 0.62 | 0.23 | 1.35 | 1.55 | 0.61 | 1.43 |
| 2132 | 0.95 | −2.00 | 0.62 | 0.74 | −0.48 | 0.94 | −0.04 | 0.48 | 0.27 | 0.65 | 1.17 | 0.66 | 1.71 |
| 2239 | −0.15 | −0.28 | 0.39 | 0.79 | −0.19 | 1.41 | −0.42 | 0.93 | 1.42 | 2.37 | −1.21 | 0.29 | 0.45 |
| 56927 | −1.02 | −1.68 | −0.23 | −0.22 | 0.77 | 2.08 | 0.82 | 0.82 | 0.14 | 0.79 | 0.49 | 0.45 | 1.53 |
| 2950 | 0.98 | −1.26 | 0.89 | −0.03 | −0.99 | 1.37 | 1.32 | 1.22 | −0.36 | 1.90 | 0.92 | −0.83 | 0.91 |
| 342538 | 1.23 | −1.03 | 0.64 | 0.13 | −0.73 | 1.23 | 0.43 | 1.25 | 1.38 | −0.27 | 1.43 | 0.73 | −0.15 |
| 55218 | 0.53 | −0.23 | −0.13 | −0.15 | −1.17 | 1.10 | 0.06 | 1.99 | 0.33 | 1.73 | 1.83 | 0.10 | 0.96 |
| 151827 | 1.39 | −0.77 | 1.21 | 0.52 | −1.65 | 1.81 | 0.37 | 0.06 | 0.74 | 1.01 | 1.65 | −0.42 | 1.26 |
| 64689 | 0.08 | −2.07 | 0.91 | 0.29 | 0.13 | 2.07 | 0.49 | −0.23 | 0.57 | 2.58 | 0.79 | 0.00 | 0.58 |
| 10324 | −0.29 | −1.73 | 0.06 | 0.91 | 0.17 | 0.97 | −0.64 | 0.64 | 0.86 | 0.92 | 2.07 | 1.62 | 0.33 |
| 1314 | −0.31 | −1.53 | 0.17 | −0.25 | 0.03 | 1.81 | 0.95 | 0.85 | −0.14 | 1.37 | 2.06 | 0.17 | 0.19 |
| 54928 | −0.21 | −0.95 | −0.60 | 0.54 | −0.73 | 1.67 | −0.06 | 2.31 | −0.34 | 1.27 | 1.44 | 0.61 | 2.35 |
| 8971 | 1.22 | −1.08 | 1.32 | 0.14 | −0.62 | 1.00 | 0.80 | 1.63 | −0.05 | 0.09 | 2.19 | −0.36 | 1.27 |
| 79832 | −2.49 | −1.52 | 0.70 | 1.10 | 0.11 | 1.21 | 0.61 | 0.89 | 0.57 | 0.98 | 1.65 | 0.73 | 0.10 |
| 10150 | −0.96 | −2.54 | 0.40 | 0.41 | 0.14 | 1.33 | 0.56 | 1.87 | 0.64 | 0.77 | 1.81 | 0.32 | 0.70 |
| 8744 | 0.62 | −0.81 | 2.05 | −0.42 | −0.12 | 0.96 | 1.22 | 0.80 | 1.53 | −0.82 | 1.58 | 0.02 | 0.57 |
| 23287 | −0.35 | −0.87 | 0.12 | 0.37 | 1.18 | −1.12 | −0.39 | −1.56 | −1.62 | −1.70 | −0.40 | 0.63 | −1.34 |
| 606553 | 0.80 | 0.43 | −1.17 | 0.19 | −0.21 | 0.00 | −0.54 | −2.01 | −0.21 | −0.94 | −0.65 | 0.62 | −0.49 |
| 103 | −1.46 | −0.07 | −1.40 | 0.49 | 1.34 | −2.23 | −0.75 | −2.19 | −0.03 | −0.75 | −0.40 | −0.28 | −1.14 |
| 55509 | 0.04 | 1.55 | −1.39 | −0.53 | 0.07 | −0.14 | −0.66 | −1.83 | −0.27 | 0.13 | −2.02 | −0.56 | −0.41 |
| 729614 | 0.07 | −1.57 | 0.43 | 0.27 | 0.77 | −1.70 | −0.66 | −1.90 | 0.16 | −2.60 | −0.54 | 0.29 | −0.58 |
| 9241 | 0.88 | −1.73 | 0.06 | 0.16 | −0.73 | −0.95 | −0.22 | −0.28 | −0.44 | −1.29 | 0.18 | −1.27 | −1.07 |
| 26118 | −0.15 | −0.19 | −0.31 | 0.00 | 1.41 | −0.86 | 0.27 | −2.11 | −1.09 | −1.61 | −2.21 | 0.74 | −1.83 |
| 64400 | −0.99 | −0.87 | −1.59 | 0.31 | 0.97 | −1.54 | −1.03 | −0.14 | −0.57 | −1.48 | −1.36 | 0.78 | −0.56 |
| 283237 | −0.96 | 1.03 | −1.94 | −1.20 | −0.43 | −1.28 | −0.77 | 1.16 | −2.11 | 0.05 | −0.53 | −0.09 | −0.63 |
| 51155 | 0.79 | 0.88 | 0.23 | −0.33 | 0.52 | −2.59 | −0.80 | −2.20 | −0.80 | −0.61 | −1.84 | −0.24 | −0.84 |
| 18 | −0.07 | 0.23 | −1.00 | 0.09 | 1.20 | −0.87 | −0.67 | −1.89 | −1.07 | −0.72 | −2.52 | 0.26 | −1.09 |
| 22990 | −0.70 | −0.04 | −0.10 | 0.09 | 1.21 | −1.80 | −0.41 | −1.79 | −0.56 | −1.40 | −0.91 | 0.81 | −1.51 |
| 23347 | −0.57 | −1.02 | −0.30 | 0.96 | 1.36 | −1.86 | 0.02 | −1.45 | −1.07 | −1.65 | −1.25 | 0.48 | −1.67 |
| 83988 | −1.32 | 0.80 | −0.67 | −0.18 | −1.06 | −1.26 | −1.77 | −0.36 | −0.25 | 0.63 | 0.14 | −2.05 | −0.35 |
| 3428 | −0.71 | −0.35 | −2.27 | 0.36 | 1.03 | −1.98 | −0.26 | −1.64 | −0.84 | −0.24 | −0.74 | 0.07 | −1.35 |
| 3700 | −1.37 | −0.94 | −0.27 | 0.57 | 0.90 | −0.48 | −1.42 | −1.48 | 0.17 | −1.47 | −0.58 | −0.73 | −0.61 |
| 64859 | −0.71 | 0.95 | −0.52 | 0.24 | 1.35 | −1.50 | −0.53 | −0.83 | −1.35 | −1.05 | −1.81 | 0.25 | −0.76 |
| 55192 | −0.36 | −1.27 | −1.02 | 0.30 | 0.26 | −0.04 | 0.26 | −1.02 | −1.41 | −0.09 | −1.61 | 0.07 | −0.88 |
| 101928869 | −0.04 | −0.18 | −0.34 | −0.03 | −0.20 | −1.62 | 0.28 | −0.38 | −1.14 | −2.14 | −1.96 | 0.32 | −0.51 |
| 10513 | −0.91 | −2.38 | −1.45 | 1.01 | 0.73 | −1.27 | −0.41 | −0.50 | −1.08 | −0.79 | −0.63 | 0.36 | −0.28 |
| 11009 | −0.17 | −0.02 | −1.06 | −0.11 | −0.40 | −0.24 | −0.31 | −0.49 | 1.07 | −1.28 | −0.76 | −0.82 | −1.43 |
| 169355 | −0.74 | 0.89 | −1.74 | −0.24 | −0.11 | −1.68 | −0.53 | −1.14 | 0.17 | −0.75 | −0.36 | 0.76 | −0.22 |
| 10109 | −0.15 | −0.55 | −0.35 | −0.38 | 1.11 | −2.14 | 0.28 | −1.34 | 0.37 | −1.69 | −0.56 | −1.07 | −1.15 |
| 9903 | 0.16 | −1.06 | −1.62 | 0.12 | 0.82 | −2.22 | 0.19 | −0.73 | −1.04 | −0.25 | −1.14 | −0.14 | −0.84 |
| 23150 | −0.48 | −1.24 | −0.60 | 0.19 | 1.33 | −1.92 | 0.26 | −1.59 | −0.43 | −1.37 | −1.04 | 0.84 | −1.00 |
| 10865 | −0.30 | 0.80 | −1.15 | −0.75 | 0.53 | −2.27 | 0.05 | −1.26 | −0.97 | −2.05 | −0.69 | 0.88 | −0.42 |
| 5165 | −1.00 | 0.12 | −0.47 | −0.12 | 1.13 | −1.66 | −0.37 | −2.01 | −0.97 | −0.88 | −1.78 | 0.70 | −0.63 |
| 57403 | −1.54 | −1.06 | −1.47 | 0.28 | 0.75 | −1.03 | −0.80 | −0.40 | −1.70 | −0.82 | −0.52 | 1.01 | −0.26 |
| 22900 | −1.94 | −1.12 | −0.87 | 0.59 | 0.84 | −2.04 | −0.17 | −0.92 | −0.63 | −1.41 | −0.57 | 0.67 | −1.07 |
| 80345 | −0.21 | −2.60 | −0.38 | 0.41 | −0.12 | −1.55 | −0.83 | −0.44 | −1.71 | −1.62 | 0.69 | 0.30 | 0.35 |
| 10964 | −0.66 | 0.15 | −2.49 | −1.00 | −0.09 | −1.41 | −0.56 | −0.82 | −0.56 | 0.68 | −0.79 | −1.42 | −0.16 |
| 27040 | −0.32 | −1.34 | −1.39 | 0.27 | −0.40 | −1.06 | −0.92 | −0.17 | 0.50 | −1.30 | 0.11 | −0.74 | −0.80 |
| 91010 | −0.53 | 0.41 | −1.24 | 0.65 | −0.86 | 0.52 | −1.00 | −0.68 | 1.01 | −0.58 | −0.89 | −0.51 | −0.66 |
| 29065 | −0.60 | 0.41 | −0.52 | 0.23 | 1.63 | −1.15 | −0.81 | −1.37 | −0.47 | −0.63 | −2.39 | 0.67 | −1.85 |
| 84078 | −0.88 | −0.25 | −1.55 | 0.01 | 1.20 | −1.58 | −0.27 | −0.77 | −0.63 | −0.74 | −1.72 | 0.06 | −0.83 |
| 101927910 | −2.83 | −0.49 | −0.84 | 0.04 | 0.19 | 0.00 | −0.97 | 0.65 | −1.69 | −0.02 | −0.74 | 0.07 | −0.89 |
| 51380 | −0.27 | −0.09 | −0.14 | 0.06 | 1.55 | −1.54 | −0.38 | −1.37 | −1.24 | −1.69 | −1.78 | 0.90 | −1.71 |
| 10289 | 0.70 | 0.21 | 0.46 | 0.08 | 0.41 | −0.51 | −0.02 | −0.29 | −1.10 | −1.16 | −0.63 | −0.81 | −0.43 |
| 10296 | 0.57 | −2.49 | −0.23 | 0.10 | 1.41 | −1.69 | 0.09 | −0.72 | −1.92 | −1.38 | −0.70 | −0.29 | −1.12 |
| 114224 | 1.01 | −2.13 | 0.84 | −0.01 | 1.21 | −0.48 | −1.25 | −1.88 | −1.10 | −0.21 | −1.29 | 0.11 | −2.10 |
| 2153 | −1.29 | 0.22 | −0.50 | −0.01 | 1.46 | −1.68 | −0.34 | −1.54 | −0.84 | −0.80 | −1.89 | 0.71 | −1.23 |
| 55825 | −1.39 | 1.16 | −1.50 | 0.14 | 0.50 | −1.38 | −0.96 | −1.34 | −0.69 | −0.12 | −1.71 | −0.01 | −0.95 |
| 6503 | −0.44 | −0.09 | −0.92 | 0.16 | 1.22 | −2.09 | 0.47 | −1.85 | 0.18 | −0.46 | −1.63 | 0.31 | −1.76 |
| 7913 | 0.15 | −1.37 | 1.58 | 0.40 | 0.13 | −1.63 | 0.91 | −0.68 | −0.60 | −1.29 | −0.28 | −1.34 | −1.48 |
| 30834 | 1.04 | −1.30 | −0.83 | −0.48 | 0.06 | −1.36 | −0.55 | 0.30 | −2.11 | −0.36 | −0.68 | −1.02 | −0.18 |
| 26999 | −1.11 | 0.10 | −0.79 | −0.07 | −0.13 | −2.15 | −0.90 | −0.81 | −0.06 | −1.87 | 0.74 | −0.38 | −0.59 |
| 286437 | −1.27 | −0.01 | −1.52 | 0.19 | 0.46 | −1.54 | −1.44 | −0.75 | −0.03 | −0.47 | −1.27 | 0.36 | −0.61 |
| 4277 | −1.44 | 0.62 | −1.13 | 0.56 | 0.54 | −1.42 | −0.74 | −0.78 | −1.71 | −0.92 | −0.51 | −0.43 | 0.06 |
| 5698 | −0.74 | −0.24 | −2.61 | −0.38 | −0.29 | −1.17 | −0.56 | −0.36 | −0.77 | 0.95 | −0.86 | −0.57 | −0.11 |
| 4318 | −0.23 | 1.00 | 0.27 | −0.31 | 1.28 | −1.31 | −0.21 | −1.42 | −0.85 | −0.67 | −2.52 | 0.23 | −0.94 |
| 10616 | 0.00 | −0.64 | −0.41 | 0.26 | 1.27 | −2.22 | 0.34 | −1.99 | −1.68 | −1.40 | −1.02 | −0.42 | −0.76 |
| 23012 | −1.49 | 0.02 | −1.09 | 0.19 | 0.86 | −1.70 | −0.05 | −1.23 | −1.13 | −1.30 | −0.69 | 0.13 | −0.95 |
| 100129361 | −0.01 | 0.92 | 0.17 | −0.23 | −0.04 | −1.34 | 0.07 | −2.27 | −0.84 | −1.20 | −1.04 | −0.25 | −0.24 |
| 10742 | −0.44 | 1.06 | −0.25 | −0.59 | 0.49 | −1.56 | −1.22 | −1.14 | −0.12 | −1.02 | −1.37 | −0.42 | −0.51 |
| 196264 | −0.69 | −0.50 | −1.07 | 0.00 | 1.02 | −1.32 | −0.48 | −1.80 | −0.41 | −0.64 | −1.82 | 0.63 | −0.78 |
| 27197 | −1.16 | −0.23 | −1.77 | 0.46 | 0.82 | −1.73 | 0.57 | −1.82 | −0.67 | −0.18 | −1.45 | −0.35 | −0.68 |
| 7529 | −0.63 | −0.57 | −0.75 | −0.06 | 0.72 | −0.99 | 0.33 | −1.52 | −0.06 | 0.35 | −1.67 | −0.38 | −1.73 |
| 94235 | −0.82 | −0.23 | −0.73 | 0.29 | 0.22 | −0.97 | −0.62 | −0.56 | −0.99 | −0.15 | −1.35 | 0.41 | −1.24 |
| 352961 | −1.67 | 0.70 | −1.42 | −0.25 | −0.11 | −1.35 | −2.01 | −0.19 | −0.28 | 0.23 | −0.23 | −0.65 | −1.23 |
| 49856 | −0.78 | −0.95 | −1.29 | 0.12 | 1.13 | −1.50 | 0.37 | −0.93 | −0.90 | −1.08 | −2.01 | 0.19 | −0.77 |
| 9448 | −0.59 | −0.35 | 0.04 | 0.68 | 1.15 | −2.04 | −0.12 | −1.91 | −0.40 | −1.10 | −1.66 | 0.12 | −1.12 |
| 9525 | −1.02 | −0.95 | −1.20 | 0.70 | 0.48 | −1.00 | −0.15 | −0.43 | −0.62 | −1.54 | −0.17 | −1.44 | −0.08 |
| 100505576 | −0.20 | 1.06 | −0.59 | 0.47 | 1.10 | −1.72 | −0.75 | −0.39 | −0.39 | −2.04 | −1.16 | −0.01 | −1.34 |
| 151556 | −1.59 | −0.55 | −1.77 | 0.02 | 0.50 | −1.78 | −0.89 | −0.59 | −0.56 | −0.09 | −1.22 | 0.53 | −0.98 |
| 284613 | 0.91 | 0.39 | −0.85 | −0.12 | 0.97 | −2.62 | −0.18 | −0.51 | −0.35 | −0.17 | −2.08 | −0.40 | −1.31 |
| 286223 | −0.48 | 0.97 | −0.62 | 0.21 | 0.71 | −0.77 | −0.45 | −1.48 | −1.30 | 0.34 | −2.48 | −0.60 | −0.58 |
| 57061 | 1.15 | 0.29 | 0.43 | −0.06 | 0.34 | −1.58 | −1.46 | −0.71 | −0.12 | −0.65 | −1.03 | −0.97 | −1.63 |
| 9520 | −0.82 | −1.10 | −0.44 | 0.24 | 1.34 | −0.81 | 0.12 | −2.17 | −1.52 | −0.54 | −1.61 | 0.38 | −0.92 |
| 202299 | 0.02 | 1.74 | −0.68 | −0.85 | −0.39 | −0.23 | −1.28 | −0.44 | −0.75 | 0.44 | −1.28 | −1.92 | 0.19 |
| 2015 | −0.20 | −0.94 | 0.30 | −0.01 | 1.19 | −2.03 | 0.02 | −2.16 | −0.53 | −1.57 | −1.09 | 0.30 | −0.51 |
| 57763 | −1.70 | −1.43 | −0.82 | 0.78 | −0.05 | −0.21 | −0.99 | 0.70 | −1.42 | −0.84 | 0.16 | −0.67 | −0.40 |
| 54778 | −1.92 | −0.72 | −0.97 | 0.80 | 1.12 | −0.70 | 0.23 | −0.85 | −1.81 | −0.24 | −1.06 | −0.22 | −0.91 |
| 762 | 0.07 | 0.13 | −0.68 | −0.21 | 1.50 | −0.79 | −0.07 | −1.15 | −1.51 | −0.94 | −2.12 | 0.33 | −1.40 |
| 192668 | 0.07 | 0.31 | −0.07 | 0.20 | −0.21 | −1.54 | −0.90 | −1.06 | 0.13 | −0.71 | −2.03 | 0.03 | −0.05 |
| 24146 | 0.57 | −0.18 | 1.72 | 0.41 | 0.85 | −1.60 | −0.24 | −1.45 | −0.07 | −0.51 | −2.01 | −1.50 | −1.37 |
| 54843 | −1.38 | −1.01 | −1.25 | 0.18 | −0.75 | −0.04 | −1.66 | −0.28 | −0.58 | 1.24 | 0.13 | −0.37 | −0.93 |
| 55374 | −0.83 | 0.19 | −1.37 | 0.42 | 0.91 | −1.48 | −0.88 | −0.97 | −0.16 | 0.36 | −1.93 | −0.18 | −1.23 |
| 79603 | −0.73 | −0.21 | −0.90 | 0.12 | −0.61 | −1.48 | −0.26 | −0.66 | −0.63 | −0.38 | −1.90 | −0.01 | −0.32 |
| 3577 | −0.04 | 0.43 | −0.27 | 0.43 | 1.29 | −1.93 | −0.32 | −1.75 | −1.66 | −0.23 | −1.89 | −0.08 | −1.31 |
| 10385 | −1.49 | 0.56 | −1.86 | −0.62 | −0.12 | −0.69 | −1.36 | −1.25 | 0.44 | 0.16 | −0.50 | −0.48 | −0.10 |
| 79792 | 1.85 | 0.17 | 1.00 | −0.35 | −0.52 | −2.17 | 0.45 | −0.87 | −1.13 | −0.69 | −1.75 | −0.79 | −0.88 |
| 84674 | −0.39 | 0.05 | −0.19 | 0.34 | 1.37 | −1.70 | 0.42 | −2.50 | −1.07 | −0.34 | −2.10 | −0.21 | −1.12 |
| 51131 | −0.38 | 0.81 | −0.74 | −0.55 | −0.13 | −0.73 | −0.79 | −0.86 | −0.73 | 0.52 | 0.05 | −1.42 | −0.95 |
| 5966 | −1.03 | −0.68 | 0.41 | −0.67 | 0.93 | −0.94 | 0.69 | −1.60 | −0.40 | −0.69 | 0.27 | −0.70 | −1.64 |
| 9733 | −1.84 | −2.27 | −1.93 | 0.44 | −0.36 | −0.14 | 0.08 | −0.14 | −0.95 | 0.00 | −0.16 | −0.46 | −0.12 |
| 10328 | −0.84 | −0.29 | 0.70 | −1.49 | −0.78 | −0.56 | −0.38 | −0.75 | −2.76 | 0.90 | −1.61 | −0.76 | 0.54 |
| 255231 | −1.14 | 1.95 | −1.46 | −0.26 | −0.20 | −0.85 | −1.39 | −0.57 | 0.18 | −0.52 | −1.59 | −0.56 | −1.02 |
| 25801 | −0.06 | 0.10 | −0.23 | 0.59 | 1.47 | −1.92 | 0.28 | −1.92 | −1.40 | −0.67 | −1.50 | −0.35 | −1.36 |
| 6352 | −1.13 | −0.16 | −1.00 | 0.05 | −0.07 | −1.25 | −0.09 | −1.39 | −0.26 | 0.33 | 0.52 | −0.51 | −1.62 |
| 54811 | 1.20 | 2.25 | −1.15 | −0.23 | −0.50 | −0.68 | −0.11 | −1.60 | −1.46 | −0.18 | −1.10 | −0.54 | −0.62 |
| 59348 | 0.55 | −0.83 | −0.28 | −0.22 | 1.63 | −1.49 | −0.35 | −0.93 | −0.55 | −2.61 | −1.32 | 0.26 | −0.61 |
| 9967 | −0.31 | −0.87 | −0.25 | 0.01 | 0.70 | −0.70 | 0.62 | −2.98 | −1.66 | −0.15 | −1.91 | 0.73 | −1.22 |
| 2533 | −0.40 | −0.87 | −0.40 | 0.06 | 1.06 | −2.02 | 0.17 | −1.55 | −0.84 | −0.88 | −1.34 | 0.27 | −1.79 |
| 28985 | −1.57 | −0.57 | −0.96 | −0.69 | 0.21 | −0.17 | −0.96 | 0.87 | −1.37 | 0.61 | −0.33 | −1.51 | −0.31 |
| 55234 | −1.39 | −2.16 | −1.95 | 0.25 | 0.29 | −0.42 | −0.78 | −0.59 | −0.55 | −1.61 | −0.87 | 0.97 | −0.28 |
| 1301 | −0.13 | 1.76 | −1.45 | −0.27 | −0.05 | −1.79 | −1.32 | −0.94 | 0.75 | −0.96 | −1.31 | −0.01 | −0.94 |
| 84803 | −0.92 | −0.99 | −1.14 | −0.01 | 1.86 | −0.86 | 0.07 | −1.55 | −0.99 | −0.15 | −1.58 | 0.12 | −1.23 |
| 100506779 | 0.11 | −0.93 | 0.03 | −0.08 | −0.09 | −1.76 | −0.13 | −0.93 | −0.30 | −2.04 | 0.68 | −1.88 | −1.36 |
| 197135 | −0.71 | −1.13 | −0.76 | 0.19 | 0.09 | −0.76 | −1.29 | −1.80 | 0.64 | −0.31 | −0.37 | −0.89 | −1.55 |
| 3614 | 0.41 | −0.03 | −0.14 | 0.07 | 1.18 | −2.13 | 0.07 | −1.98 | −0.72 | −0.93 | −1.80 | −0.74 | −1.13 |
| 80781 | −0.36 | 1.09 | 0.29 | 0.23 | 0.48 | −2.93 | −0.38 | −1.04 | −0.37 | −0.56 | −1.58 | 0.24 | −0.97 |
| 5586 | −0.31 | −0.29 | 0.73 | 1.12 | 0.96 | −0.76 | −0.58 | −0.99 | −1.76 | −0.22 | −1.49 | −0.10 | −1.25 |
| 79415 | −0.20 | 1.48 | −1.49 | 0.25 | 0.88 | −1.73 | −0.49 | −1.76 | −0.33 | −0.83 | −2.30 | 0.17 | −1.22 |
| 8893 | −0.65 | −0.39 | 0.08 | 0.31 | 0.39 | −1.42 | −0.96 | −0.56 | 0.04 | −1.19 | −0.43 | −1.52 | 0.22 |
| 79736 | −1.14 | 3.19 | −1.06 | 0.01 | −0.12 | −0.70 | −0.69 | 0.46 | −1.94 | 0.18 | −1.26 | −0.34 | −0.43 |
| 5770 | −1.06 | 2.10 | −1.28 | −1.53 | 0.49 | 0.56 | −1.06 | −0.82 | −1.28 | −0.47 | −2.00 | 0.18 | −0.38 |
| 9360 | −0.68 | −2.03 | −0.70 | 0.86 | 0.70 | −0.97 | −0.23 | −0.08 | −0.90 | −2.21 | −1.17 | 1.25 | −1.04 |
| 29940 | −0.62 | −0.15 | −1.11 | −0.67 | 1.04 | −1.20 | 0.40 | −2.00 | −0.83 | −0.01 | −1.81 | −0.31 | −0.37 |
| 7150 | 1.30 | −0.27 | 1.04 | −0.87 | 0.47 | −0.47 | −1.72 | −0.75 | −0.91 | 0.28 | −1.96 | −0.92 | −1.33 |
| 84964 | −1.20 | −1.09 | −0.79 | 0.00 | −0.12 | −1.18 | −1.16 | −0.27 | −0.20 | −1.63 | −0.65 | −0.37 | −0.76 |
| 10614 | 0.54 | −0.15 | −0.65 | 0.38 | 0.47 | −0.81 | 0.27 | −1.58 | −1.76 | −0.81 | −1.05 | −0.18 | −0.73 |
| 147923 | −1.74 | −0.94 | −1.51 | 0.60 | −0.09 | −1.63 | −0.55 | 0.36 | −0.63 | −0.38 | 0.39 | −1.12 | 0.25 |
| 284029 | −1.11 | −0.39 | −1.60 | 0.16 | 0.48 | −1.30 | −0.09 | −0.50 | −0.76 | −1.67 | −1.15 | 0.35 | −0.48 |
| 54952 | −1.46 | 1.17 | −0.06 | −0.13 | 0.17 | −1.51 | −0.47 | −1.76 | −0.16 | −1.05 | −1.15 | −0.21 | −0.62 |
| 57476 | 0.02 | −0.31 | 0.34 | −0.79 | 0.02 | −0.67 | 0.11 | −1.52 | 0.28 | −1.39 | −3.16 | −0.48 | −0.86 |
| 8745 | −1.00 | 2.21 | −1.70 | 0.15 | −0.53 | −1.38 | −0.67 | 0.52 | −0.98 | −0.15 | −0.31 | −1.72 | −0.57 |
| 22928 | −1.01 | 1.46 | −1.28 | 0.09 | 1.20 | −1.64 | 0.24 | −1.43 | −0.05 | −1.22 | −1.37 | −0.81 | −0.45 |
| 25880 | −1.50 | −0.80 | −2.08 | −0.40 | −1.10 | 0.01 | 0.03 | −0.25 | −1.55 | −0.01 | −0.60 | −0.71 | 0.09 |
| 28638 | −0.81 | −0.39 | −0.41 | 0.46 | −0.02 | −2.19 | −0.98 | −0.89 | −0.47 | −0.37 | −0.06 | −1.20 | −1.32 |
| 29933 | −2.06 | −0.25 | −0.38 | −0.49 | 0.42 | −0.91 | 0.62 | −0.86 | −0.39 | −1.87 | −1.08 | 1.13 | −1.16 |
| 30009 | −0.97 | −1.71 | −0.78 | 0.60 | −0.43 | −1.69 | 0.15 | −0.49 | −1.41 | −0.50 | −0.50 | 0.12 | −0.86 |
| 3554 | −0.65 | 0.83 | −0.21 | −0.33 | 1.37 | −0.71 | −0.41 | −1.50 | −0.30 | −0.57 | −2.20 | 0.41 | −0.67 |
| 8821 | −0.97 | −1.09 | −1.49 | 0.05 | −0.07 | −1.31 | −1.14 | −0.75 | 0.07 | −0.20 | 0.56 | −0.75 | −0.39 |
| 10943 | −1.31 | −1.19 | −1.00 | −0.44 | 1.30 | −1.21 | −0.83 | −0.87 | −0.80 | −1.68 | −1.18 | 0.84 | −1.16 |
| 57724 | −0.68 | −1.69 | −0.69 | 0.63 | 1.42 | −1.81 | −0.82 | −1.44 | −0.38 | −1.68 | −0.92 | 0.62 | −0.92 |
| 100506282 | −0.29 | 0.27 | −0.26 | −0.12 | 0.68 | −0.35 | −0.98 | −0.98 | −1.08 | −0.06 | −2.92 | −0.12 | −0.82 |
| 147699 | −0.62 | 1.75 | −1.43 | −0.70 | 0.66 | −0.47 | −0.55 | −0.25 | 0.21 | −1.24 | −2.57 | −0.15 | −1.07 |
| 3002 | −1.15 | 0.41 | −0.41 | 0.43 | −0.49 | −2.73 | −0.62 | −0.77 | 0.01 | −0.74 | −0.32 | −0.57 | −0.47 |
| 389320 | −0.31 | −0.38 | 0.35 | 0.10 | −0.39 | −0.84 | −0.47 | −1.76 | 0.86 | −0.73 | −1.12 | 0.34 | −1.18 |
| 9914 | −0.06 | −0.08 | −0.22 | −0.27 | 0.80 | 0.22 | −0.95 | −2.55 | −0.89 | −0.10 | −2.35 | 0.50 | −0.73 |
| 10804 | −1.01 | 0.54 | −0.19 | −0.43 | 0.58 | −1.29 | −0.76 | −1.39 | −0.73 | −1.88 | −1.23 | 0.69 | −0.43 |
| 51439 | −0.48 | −0.61 | −0.63 | 0.86 | 1.25 | −1.58 | −0.76 | −1.01 | −1.56 | −0.15 | −1.57 | −0.26 | −0.82 |
| 814 | −1.73 | −0.04 | −0.95 | −0.33 | 0.13 | −1.58 | −1.12 | −0.34 | 0.13 | −1.29 | −0.65 | −0.19 | −1.21 |
| 5272 | −1.10 | −0.25 | −2.20 | −0.80 | −0.23 | −1.26 | −0.35 | −0.72 | −0.86 | −0.60 | −0.26 | −0.12 | −0.92 |
| 8428 | −1.13 | 0.33 | −1.44 | −0.51 | 0.40 | −1.37 | −1.13 | −1.10 | −1.13 | 0.08 | −0.07 | −1.59 | 0.17 |
| 23048 | 0.03 | −1.09 | −0.69 | −0.25 | −0.71 | −1.04 | 0.16 | −2.43 | 0.32 | −1.89 | 0.24 | −0.36 | −0.47 |
| 344558 | 0.38 | −1.43 | −0.23 | 0.37 | 0.84 | −0.85 | −1.23 | −0.98 | −0.77 | −0.98 | −2.71 | 0.02 | −0.55 |
| 79078 | 0.84 | 1.26 | −0.45 | −0.84 | −0.66 | −1.15 | −1.62 | −0.53 | −1.13 | −0.96 | −0.37 | −1.31 | −0.53 |
| 81606 | −0.77 | −0.26 | −0.68 | 0.22 | −0.84 | −0.89 | −1.14 | −0.64 | −0.51 | 0.12 | −0.38 | −1.14 | −0.68 |
| 64377 | −0.28 | −0.62 | 0.40 | −1.62 | 0.74 | 0.06 | 0.41 | −0.88 | −1.46 | 0.18 | −2.43 | −1.63 | −0.24 |
| 57501 | −0.26 | −1.75 | −0.53 | 0.75 | 1.97 | 0.31 | −0.22 | −1.43 | −0.32 | −0.91 | −2.19 | 0.08 | −1.54 |
| 5991 | −0.25 | −0.83 | −0.59 | −0.03 | 0.64 | −0.01 | −1.33 | −1.75 | −0.60 | −1.37 | −1.44 | 0.78 | −0.46 |
| 90075 | −0.52 | −3.01 | −0.88 | 0.01 | −0.66 | −0.83 | −0.40 | 1.16 | −0.24 | −1.24 | 0.28 | −0.67 | −0.14 |
| 375341 | 0.05 | −0.22 | −0.54 | 0.14 | 1.34 | −1.74 | 0.35 | −1.40 | −0.79 | −0.99 | −2.42 | 0.03 | −1.27 |
| 100130548 | 0.03 | 0.85 | −0.60 | −1.07 | 0.34 | −1.10 | −0.90 | −1.79 | −0.35 | −1.83 | −0.97 | 0.72 | −1.00 |
| 114786 | −0.34 | 2.61 | −0.79 | −0.89 | 0.13 | −0.43 | −0.67 | −0.14 | −0.52 | −0.96 | −1.69 | −0.07 | −0.36 |
| 150084 | 0.26 | 2.32 | −0.73 | −0.69 | −0.08 | −1.24 | −0.37 | −1.74 | −0.43 | −1.38 | −1.92 | 0.06 | −0.44 |
| 55012 | −0.01 | −1.68 | −0.07 | 0.12 | 1.40 | −1.31 | 0.07 | −0.91 | −1.72 | −0.95 | −1.60 | −0.18 | −0.83 |
| 147525 | −0.70 | −1.00 | −0.83 | −0.17 | −1.52 | −0.38 | −0.91 | 0.19 | −0.71 | −0.84 | −0.58 | −0.82 | −0.39 |
| 54557 | −1.25 | −0.02 | −1.12 | −0.10 | 1.46 | −0.88 | −0.39 | −0.96 | −0.64 | −1.86 | −1.56 | 0.65 | −1.02 |
| 56267 | −0.02 | −0.14 | −0.75 | −0.28 | −1.12 | −0.42 | −1.14 | −0.82 | −0.48 | −0.27 | 1.02 | −2.34 | −0.40 |
| 51176 | −1.53 | −0.45 | −1.54 | −0.32 | −0.05 | −1.34 | −0.85 | −0.51 | −0.22 | −1.20 | −0.16 | −0.27 | −1.17 |
| 10468 | −0.41 | 0.93 | −0.11 | −1.12 | −0.03 | −1.05 | −1.27 | 0.56 | −0.16 | −0.64 | −2.11 | −0.52 | −0.69 |
| 23258 | 0.32 | −0.76 | 0.02 | −0.59 | 0.55 | −1.68 | 0.62 | −2.38 | −0.05 | −0.91 | −1.51 | 0.21 | −2.12 |
| 90768 | −0.58 | 1.12 | 0.26 | 0.23 | −0.48 | −1.60 | −0.91 | −1.54 | 0.08 | −0.85 | −0.77 | −0.32 | −0.98 |
| 134957 | −0.54 | −0.10 | −0.13 | 0.71 | 1.17 | −1.58 | −0.11 | −2.33 | −0.72 | −1.75 | −0.99 | 0.26 | −1.15 |
| 80342 | −0.25 | −0.55 | −0.84 | 0.10 | 0.11 | −1.93 | −0.35 | −1.02 | 0.37 | −1.99 | 0.27 | −0.76 | −1.84 |
| 91351 | −0.74 | 0.01 | −0.59 | 0.60 | 1.32 | −1.63 | −0.05 | −2.06 | −0.68 | −0.68 | −1.66 | −0.08 | −1.48 |
| 29916 | −0.46 | 1.95 | −0.44 | −0.31 | 0.77 | −1.33 | 0.26 | −1.32 | −1.11 | −0.88 | −2.44 | −0.26 | −0.66 |
| 8519 | −0.76 | −0.30 | −1.74 | 0.29 | 0.34 | −2.52 | −0.89 | −0.22 | −0.60 | −1.53 | 0.06 | −0.06 | −1.10 |
| 2113 | −1.60 | −0.14 | −1.05 | 0.30 | −0.03 | −1.65 | −0.90 | −1.05 | −0.60 | −1.34 | 0.39 | −0.66 | −1.13 |
| 55770 | −2.56 | 0.21 | −1.23 | 0.15 | −0.30 | −0.49 | −0.58 | −1.55 | 0.49 | −0.51 | 0.16 | −0.60 | −0.17 |
| 91833 | −1.23 | −1.50 | −0.67 | 0.59 | 1.04 | −1.43 | −0.63 | −0.27 | −1.31 | −1.88 | −0.88 | 0.38 | −0.19 |
| 117177 | −1.59 | 1.31 | −0.65 | −0.08 | 0.89 | −0.71 | −0.88 | −1.16 | −0.75 | −1.01 | −1.50 | 0.26 | −0.90 |
| 546 | −0.93 | −1.67 | −0.52 | 1.13 | 1.20 | −0.88 | −0.27 | −2.36 | −1.28 | −0.88 | −1.67 | 0.53 | −1.32 |
| 4253 | −1.90 | −0.69 | −1.65 | 0.19 | 0.88 | −1.89 | −0.62 | 0.12 | −0.65 | 0.22 | −0.93 | 0.02 | −1.04 |
| 23214 | −0.12 | 0.65 | −0.96 | −0.26 | 1.31 | −2.20 | −0.32 | −1.58 | −0.25 | −1.06 | −1.95 | 0.27 | −1.48 |
| 53347 | −1.17 | 0.06 | −1.49 | 0.38 | 0.10 | −2.00 | −1.10 | −1.07 | −0.09 | −0.73 | −0.10 | −0.42 | −0.77 |
| 56888 | −1.40 | 0.87 | −1.07 | −0.32 | 0.72 | −1.51 | −0.61 | −0.42 | −0.43 | 0.28 | −1.98 | −0.08 | −1.65 |
| 777 | −0.79 | 1.01 | −0.83 | −0.03 | 0.84 | −1.28 | −0.47 | −1.27 | −0.72 | −1.42 | −1.81 | 0.15 | −0.79 |
| 100216545 | −0.54 | 0.46 | −0.48 | −0.56 | −0.03 | −0.90 | −0.53 | −1.13 | −0.29 | 0.82 | −0.67 | −1.17 | −1.23 |
| 8879 | −1.11 | 0.24 | −1.42 | 0.65 | 0.59 | −0.06 | −0.38 | −1.86 | −1.11 | −0.18 | −2.25 | 0.61 | −0.61 |
| 9759 | 0.23 | −0.08 | 0.09 | 0.27 | 1.34 | −1.51 | −0.13 | −1.25 | −1.48 | −0.76 | −2.24 | 0.33 | −1.13 |
| 26578 | −0.67 | 0.07 | −1.40 | −0.27 | 1.20 | −0.79 | −0.03 | −2.32 | −1.19 | 0.08 | −2.20 | 0.24 | −1.08 |
| 4050 | −0.82 | 0.62 | −1.48 | −0.55 | 0.43 | −2.57 | −0.56 | −0.84 | 0.10 | −1.01 | −0.82 | 0.03 | −1.25 |
| 4236 | −0.18 | −1.23 | −2.07 | 0.31 | 0.18 | −0.97 | 0.20 | −0.26 | −0.47 | 0.19 | −1.47 | −0.87 | −0.39 |
| 54491 | −0.18 | −0.19 | −0.82 | −0.18 | 1.20 | −2.00 | −0.23 | −1.14 | −0.81 | −0.98 | −1.99 | 0.94 | −1.25 |
| 55847 | −1.02 | 0.61 | −1.21 | 0.24 | 0.40 | −0.68 | −0.97 | −0.65 | −0.96 | 0.74 | −2.26 | −0.27 | −0.24 |
| 100132707 | −0.76 | 0.74 | −1.35 | 0.02 | 0.17 | −1.37 | −0.59 | −1.98 | 0.34 | −0.03 | −1.43 | −0.28 | −1.92 |
| 55884 | 0.05 | −0.61 | −0.29 | −0.28 | 0.99 | −0.99 | 0.25 | −1.75 | −1.46 | 0.14 | −2.04 | 0.02 | −0.57 |
| 83607 | −0.59 | −0.06 | −1.35 | 0.21 | −0.43 | −0.91 | −1.12 | −0.48 | −1.57 | −1.31 | −0.27 | −0.09 | 0.72 |
| 9252 | −1.32 | −0.44 | −1.20 | 0.91 | 0.26 | −1.21 | −0.52 | −0.80 | 0.20 | −0.28 | −1.31 | −0.59 | −1.33 |
| 55119 | −1.09 | 0.27 | −0.06 | 0.39 | 0.22 | −0.87 | −0.57 | 0.05 | −1.42 | −2.27 | 0.10 | 0.15 | −1.08 |
| 57520 | −0.74 | 0.28 | −0.53 | 0.15 | 0.91 | −1.68 | −0.49 | −1.38 | −0.61 | −0.37 | −2.56 | 0.35 | −0.97 |
| 79818 | −0.47 | 0.26 | −0.51 | 0.22 | 0.99 | −0.88 | −0.62 | −1.53 | −1.27 | −0.62 | −1.71 | 0.09 | −0.82 |
| 8320 | −1.77 | 0.29 | −1.43 | 0.48 | −0.02 | −1.81 | −0.84 | −0.37 | 0.13 | −0.95 | −0.36 | 0.13 | −1.27 |
| 2792 | 0.02 | 2.78 | −0.16 | −0.25 | −1.65 | 0.61 | −0.71 | −0.92 | 0.08 | −1.85 | −1.34 | −0.51 | −1.02 |
| 54910 | 0.05 | −0.12 | −1.15 | 1.12 | −0.47 | −1.37 | −0.55 | 0.69 | −1.74 | 0.02 | −1.01 | −1.07 | −1.56 |
| 55114 | −0.95 | −1.23 | −0.04 | −0.11 | −1.05 | −0.74 | 0.80 | −0.73 | −0.56 | −1.20 | −0.93 | −0.46 | −1.20 |
| 56253 | −1.84 | −1.29 | −0.99 | 0.87 | 0.45 | −0.64 | −0.53 | 0.08 | 0.29 | −0.75 | −1.28 | −0.12 | −1.25 |
| 65082 | −0.83 | 1.05 | −1.49 | 0.36 | 0.74 | −1.11 | −0.28 | −0.95 | 0.35 | −0.90 | −0.98 | −1.28 | −1.16 |
| 84719 | −0.86 | −0.17 | −0.67 | −0.08 | 0.75 | −2.13 | −0.68 | −1.17 | −0.22 | 0.45 | −1.53 | 0.12 | −1.57 |
| 253558 | −1.11 | −1.35 | −0.66 | 1.02 | −0.05 | −1.53 | −0.55 | −0.37 | 0.62 | −0.35 | −1.38 | −0.65 | −0.24 |
| 6777 | 0.02 | −0.23 | −0.53 | −0.12 | 0.89 | −2.10 | 0.25 | −2.31 | −0.90 | −1.35 | −1.31 | 0.73 | −1.74 |
| 80025 | −0.20 | 0.07 | −0.74 | −0.02 | 0.85 | −1.77 | 0.02 | −2.35 | −0.53 | 0.04 | −1.74 | −0.74 | −0.84 |
| 84662 | 0.43 | 0.14 | −1.59 | −0.74 | 0.25 | −0.94 | −0.71 | −1.16 | 0.07 | −0.24 | −2.45 | −0.66 | −0.34 |
| 89890 | −1.72 | −0.04 | 0.42 | 0.18 | 0.43 | −1.23 | −0.10 | −0.07 | −0.81 | −0.85 | −1.57 | −0.42 | −0.54 |
| 10390 | −0.80 | −0.63 | −1.10 | 0.83 | 0.88 | −0.61 | 0.05 | −1.78 | −1.48 | 0.33 | −2.30 | −0.44 | −0.26 |
| 2841 | −1.00 | 0.35 | −0.03 | −0.24 | −1.02 | −1.34 | −0.62 | −0.20 | −0.38 | −0.87 | −0.60 | −0.68 | −0.92 |
| 3904 | −1.27 | −0.05 | −0.10 | 0.25 | −0.57 | −1.85 | −0.92 | −0.17 | −1.43 | −1.08 | −0.08 | −0.63 | −0.98 |
| 140460 | −0.82 | −0.71 | −0.46 | 0.20 | 1.10 | −1.47 | −1.03 | −0.58 | −0.39 | −2.59 | −0.74 | 0.08 | −0.34 |
| 9605 | −0.29 | 0.40 | −0.60 | −0.59 | 1.34 | −1.24 | 0.07 | −2.10 | −0.90 | −0.63 | −1.97 | −0.03 | −1.15 |
| 283349 | −0.92 | −0.34 | −0.79 | 0.51 | 1.04 | −1.69 | 0.23 | −1.49 | −0.90 | −0.82 | −1.37 | 0.11 | −1.75 |
| 51246 | 0.34 | 0.24 | −1.03 | 0.03 | 0.78 | −2.49 | 0.37 | −1.40 | −1.50 | −0.04 | −1.70 | −0.38 | 0.05 |
| 148268 | −0.99 | −1.73 | −1.60 | −0.07 | −0.57 | 0.77 | −0.95 | 1.75 | −0.12 | −0.87 | −0.64 | −1.25 | 0.08 |
| 26589 | −1.74 | 0.63 | −1.78 | −0.28 | −0.38 | −1.26 | −0.14 | −0.30 | −1.52 | −0.79 | −0.24 | −0.62 | 0.24 |
| 7049 | −1.90 | −1.61 | −1.36 | 0.04 | −0.01 | −1.15 | −0.85 | −0.57 | −0.04 | 0.33 | −0.21 | −0.29 | −1.06 |
| 2791 | −1.21 | 0.83 | −1.14 | 0.12 | 1.08 | −1.49 | −0.33 | −1.29 | −0.73 | −0.55 | −1.54 | −0.67 | −0.20 |
| 51455 | −1.21 | −1.82 | 0.29 | 0.51 | 0.49 | −0.63 | −0.75 | −1.54 | 0.65 | −1.07 | −0.93 | 0.16 | −1.02 |
| 1178 | −0.98 | −1.21 | −0.20 | 0.76 | 1.10 | −1.44 | 0.64 | −1.52 | −1.51 | −0.40 | −1.14 | −0.19 | −1.44 |
| 79153 | −0.96 | 0.09 | −0.61 | 0.39 | 1.34 | −2.08 | −0.64 | −1.63 | −0.33 | −1.50 | −1.42 | 0.37 | −0.93 |
| 101928524 | −0.96 | −0.25 | 0.18 | 0.69 | 1.46 | −1.91 | −0.79 | −1.20 | −0.18 | −0.88 | −1.38 | −0.31 | −2.25 |
| 11064 | −0.04 | −1.09 | 0.45 | 0.85 | 0.69 | −0.39 | −0.56 | −2.19 | −1.16 | −1.33 | −0.95 | −0.48 | −2.11 |
| 23429 | −0.59 | −0.34 | −1.62 | 0.38 | 0.67 | −0.86 | −0.27 | −0.37 | −1.80 | −1.65 | −0.85 | 0.28 | −0.75 |
| 5170 | −0.25 | 0.04 | −1.85 | 0.36 | 1.42 | −1.14 | −0.82 | −1.01 | −0.75 | −1.27 | −2.15 | 0.19 | −0.62 |
| 10677 | −0.59 | −1.56 | −1.14 | 0.60 | 1.13 | −0.90 | −0.31 | −1.31 | −1.48 | −0.50 | −1.58 | 0.66 | −1.78 |
| 3772 | −0.26 | 0.26 | −0.83 | 0.31 | 1.35 | −1.79 | −0.07 | −1.86 | −1.14 | −0.39 | −1.96 | 0.17 | −1.31 |
| 128061 | −1.37 | −1.22 | −1.10 | 0.05 | 0.13 | −1.44 | −0.10 | 0.09 | −1.60 | −1.12 | −0.13 | −0.55 | −0.13 |
| 50856 | −0.80 | −0.14 | −0.73 | −0.26 | 1.51 | −1.39 | 0.13 | −1.57 | −0.86 | −0.45 | −1.89 | −0.14 | −1.09 |
| 6198 | −0.80 | −0.72 | 0.28 | 0.63 | −0.06 | −0.70 | −0.59 | −0.05 | −0.17 | −1.42 | −0.33 | 0.07 | −0.55 |
| 101927588 | −0.41 | −1.22 | 0.16 | 0.27 | 0.13 | −2.50 | −0.30 | −1.65 | −0.53 | −0.11 | 0.85 | −1.05 | −0.52 |
| 2000 | −0.44 | −0.50 | 0.34 | 0.29 | 1.34 | −1.71 | 0.34 | −2.31 | −0.96 | −1.05 | −1.26 | −0.58 | −1.61 |
| 493753 | −1.03 | −0.96 | −0.07 | 0.34 | 0.31 | −0.40 | −0.77 | 0.51 | −1.30 | −0.67 | −0.03 | −1.50 | −0.01 |
| 23081 | −0.29 | 0.76 | −1.48 | −0.18 | −0.42 | −0.09 | −1.54 | −0.30 | 0.17 | 0.03 | −0.59 | −1.80 | −1.60 |
| 4773 | −1.07 | −0.29 | −1.21 | 0.64 | −0.31 | −2.29 | −1.08 | −1.41 | −0.48 | −0.99 | 0.59 | −0.23 | −0.44 |
| 10178 | −0.60 | −1.74 | −0.17 | −0.15 | 0.71 | −1.79 | −0.36 | −0.90 | −0.05 | −1.71 | −1.43 | 0.64 | −1.03 |
| 4974 | −0.02 | −0.46 | 0.21 | −0.48 | 2.07 | −0.83 | 0.00 | −1.24 | −1.35 | −1.53 | −1.45 | 0.10 | −1.83 |
| 83860 | −1.02 | −0.93 | −0.32 | 0.41 | 0.59 | −1.52 | −1.30 | −0.48 | −0.02 | −1.12 | −1.50 | −0.25 | −0.28 |
| 91612 | −1.06 | −0.04 | −0.28 | −0.52 | 1.40 | −2.64 | −0.26 | −1.08 | −0.26 | −0.72 | −0.86 | −0.69 | −1.00 |
| 2178 | −1.03 | −0.35 | 0.26 | −0.13 | −0.49 | −1.83 | −0.98 | 0.03 | 0.31 | −1.52 | −1.42 | −0.93 | 0.00 |
| 55303 | −0.87 | −1.15 | −1.59 | 0.33 | 0.66 | −1.98 | −0.02 | −1.10 | −0.42 | −0.09 | −1.03 | −0.12 | −1.45 |
| 221002 | −0.36 | −0.68 | 0.34 | −0.10 | 0.88 | −2.51 | −0.46 | −1.53 | −0.07 | −0.60 | −1.68 | 0.48 | −1.14 |
| 3930 | −0.30 | −0.94 | 0.64 | 1.15 | 0.88 | −2.36 | 0.28 | −1.48 | −0.55 | −1.62 | −0.79 | −0.69 | −1.40 |
| 55471 | −1.46 | −0.11 | −0.98 | 0.29 | 0.52 | −1.80 | −0.54 | −0.64 | −1.01 | −1.67 | −0.87 | 0.30 | −0.50 |
| 9354 | −0.96 | 0.06 | −0.87 | 1.19 | 0.75 | −1.29 | −0.70 | 0.18 | −2.14 | −0.21 | −0.90 | 0.03 | −0.44 |
| 83700 | −0.42 | 0.58 | −0.84 | −0.28 | 0.75 | −1.51 | 0.07 | −1.05 | −0.33 | −3.25 | −0.20 | −0.58 | −0.77 |
| 9140 | −1.95 | −1.03 | −1.15 | 0.04 | 0.41 | −0.50 | −0.97 | 0.50 | −1.80 | −1.54 | −0.29 | 0.65 | −0.46 |
| 23526 | −0.41 | −0.11 | −0.57 | 0.13 | 0.31 | −2.31 | −0.19 | −1.01 | 0.47 | −2.56 | −0.43 | −0.27 | −1.12 |
| 23590 | −0.31 | 1.31 | 0.51 | −0.76 | 0.14 | −0.39 | −1.10 | −1.56 | −1.76 | −1.89 | −1.66 | 0.54 | −0.07 |
| 9270 | 0.76 | 1.53 | 0.81 | −0.86 | −1.50 | −1.05 | 0.26 | −0.42 | −0.21 | −0.32 | −1.49 | −2.35 | −1.03 |
| 10627 | −0.64 | 0.67 | −1.59 | 0.02 | 0.56 | −1.76 | −0.04 | −1.38 | −1.28 | −0.41 | −1.13 | −1.24 | −0.71 |
| 26048 | −0.86 | −0.82 | −1.17 | −0.58 | −1.27 | −1.06 | −0.65 | 0.24 | −0.03 | 0.13 | −0.37 | −0.83 | −0.30 |
| 917 | −0.61 | −0.23 | −0.59 | −0.63 | −0.25 | −1.30 | −0.55 | −0.61 | −0.94 | −0.64 | −0.88 | 0.01 | −1.05 |
| 94039 | −1.58 | −0.20 | −0.55 | −0.21 | 0.60 | −1.98 | −1.00 | −0.46 | −0.66 | −2.00 | −0.56 | 0.33 | −0.69 |
| 4215 | −0.33 | 0.94 | −0.99 | 0.16 | 1.29 | −1.86 | −0.07 | −2.04 | −0.76 | −0.96 | −1.93 | 0.09 | −0.84 |
| 10663 | −0.61 | 0.00 | −0.11 | −0.52 | −0.32 | −1.86 | −1.03 | −0.32 | −0.78 | −0.29 | −1.10 | −0.34 | −0.28 |
| 10308 | −1.27 | 0.56 | −0.76 | 0.39 | 1.09 | −1.16 | −0.14 | −0.73 | −1.57 | −1.56 | −1.40 | 0.17 | −1.00 |
| 8527 | 0.72 | −1.78 | 0.40 | −0.07 | 0.08 | −1.31 | 0.56 | −0.50 | −0.08 | −2.37 | −0.25 | −1.31 | −1.08 |
| 926 | −1.18 | −0.55 | −0.81 | 0.55 | −0.10 | −1.69 | −0.98 | −1.18 | −0.39 | −0.43 | −1.18 | −0.37 | −0.57 |
| 9619 | −0.95 | −1.71 | −1.39 | 0.19 | 0.25 | −1.65 | −0.97 | −0.44 | 0.85 | −0.19 | −1.13 | 0.71 | −0.82 |
| 5998 | 0.12 | −0.09 | −0.71 | −0.41 | 0.61 | −2.74 | −1.13 | −1.33 | −1.18 | −1.52 | −1.56 | 0.59 | −0.17 |
| 132625 | −0.55 | 0.75 | −1.95 | 0.44 | 0.70 | −1.62 | −0.62 | −0.68 | −1.55 | −0.90 | −1.92 | 0.44 | −0.61 |
| 88455 | −0.84 | −2.13 | −0.37 | 0.72 | 0.85 | −1.99 | −0.03 | −1.34 | −0.09 | −0.81 | −1.18 | −0.69 | −0.76 |
| 3604 | −1.34 | 1.26 | −1.09 | −0.57 | 0.06 | −1.35 | −0.53 | −1.48 | −0.11 | 0.01 | −1.98 | −0.13 | −0.90 |
| 100507398 | −0.85 | 0.37 | −0.46 | 0.34 | 0.77 | −1.77 | −0.71 | −1.14 | −0.32 | −2.18 | 0.16 | 0.06 | −1.13 |
| 58517 | 0.10 | −1.85 | 0.94 | 0.79 | −0.21 | 0.21 | −0.47 | −0.65 | −0.35 | −1.86 | −0.87 | −0.62 | −1.17 |
| 51192 | −0.27 | −0.11 | −0.68 | 0.28 | 1.55 | −1.90 | −0.08 | −1.66 | −1.17 | −1.09 | −1.56 | −0.01 | −1.36 |
| 2177 | −0.66 | 1.24 | 1.48 | 0.09 | −0.26 | −1.98 | −0.30 | −1.33 | 0.22 | −2.11 | −0.83 | −0.27 | −0.81 |
| 57097 | −0.32 | 0.82 | 0.52 | 0.03 | −0.01 | −1.43 | −0.35 | −3.05 | −1.39 | −0.09 | −0.76 | −0.41 | −0.66 |
| 11214 | −1.00 | −1.64 | −1.56 | 0.34 | 1.77 | −1.50 | 0.10 | −1.01 | −1.23 | −1.10 | −1.81 | 0.71 | −1.36 |
| 4600 | −0.80 | 0.35 | −1.01 | 0.08 | 0.25 | −1.65 | −0.19 | −2.24 | −0.30 | −0.11 | −2.28 | −0.48 | −0.62 |
| 84255 | −1.38 | 0.56 | −1.07 | 0.15 | 1.24 | −1.24 | −0.65 | −1.50 | −0.74 | −0.80 | −1.81 | 0.14 | −0.97 |
| 11216 | 0.00 | −1.12 | 0.04 | −0.06 | 0.95 | −2.21 | −0.23 | −2.27 | −0.62 | −0.37 | −0.24 | −0.09 | −2.24 |
| 162417 | −0.45 | 0.84 | −1.89 | −0.91 | 0.31 | −1.88 | −0.31 | −0.40 | −0.18 | −0.15 | −2.05 | 0.12 | −0.51 |
| 101928617 | −0.33 | −0.13 | −1.42 | −0.77 | 0.14 | −0.75 | −1.36 | 0.18 | 0.28 | −1.93 | −0.63 | −0.22 | −1.64 |
| 10617 | 0.00 | −1.32 | 0.00 | 0.79 | 0.29 | −1.28 | 0.10 | −0.42 | −1.20 | −2.04 | −1.04 | −1.88 | −0.16 |
| 1326 | −0.16 | 0.20 | 0.31 | 0.65 | 0.85 | −1.70 | −0.27 | −1.87 | −1.05 | −0.40 | −1.26 | −0.31 | −1.05 |
| 23315 | 0.29 | −1.41 | −0.05 | 0.21 | 1.18 | −1.91 | 0.16 | −1.48 | −1.08 | −1.48 | −1.43 | 0.32 | −1.70 |
| 10791 | 0.51 | 0.67 | −0.26 | −0.75 | −0.17 | −1.95 | −0.11 | −1.46 | −1.66 | −0.85 | −1.80 | 0.15 | −1.38 |
| 8718 | −0.19 | −0.08 | −0.68 | −0.95 | −0.16 | −1.46 | −0.49 | −0.53 | 0.53 | −1.09 | −0.42 | −0.44 | −1.31 |
| 51571 | −0.36 | 0.28 | −0.19 | −0.30 | 1.04 | −2.19 | 0.09 | −1.88 | −0.54 | −1.55 | −1.73 | 0.20 | −1.32 |
| 283521 | −0.31 | 0.14 | −0.78 | −0.12 | 0.31 | −2.02 | −0.58 | −0.39 | 0.07 | −1.90 | −0.61 | 0.01 | −1.31 |
| 51122 | −1.84 | −0.88 | −0.11 | 0.65 | −0.23 | −1.70 | −0.71 | −0.94 | −0.83 | −0.72 | −0.30 | −0.81 | 0.68 |
| 5372 | −2.17 | −1.48 | −0.74 | −0.21 | 0.48 | −0.93 | −1.09 | −0.48 | −0.66 | −0.38 | −1.91 | 0.83 | −0.14 |
| 9830 | 0.03 | 0.09 | −0.20 | −0.73 | 0.40 | −1.10 | −0.73 | −3.08 | −1.12 | 0.74 | −1.12 | −0.29 | −0.62 |
| 314 | −0.02 | −0.33 | −2.07 | 1.07 | 1.57 | −1.23 | −0.80 | −1.22 | −0.38 | −0.59 | −1.39 | −0.19 | −1.00 |
| 51761 | −0.93 | −0.14 | −0.03 | −0.38 | −1.36 | −0.65 | −0.54 | 1.55 | −0.02 | −0.98 | −0.61 | −1.49 | −0.85 |
| 493861 | −1.01 | −1.02 | −0.25 | −0.37 | −0.05 | −1.53 | −2.15 | 0.38 | −0.49 | −2.59 | −0.08 | 0.16 | 0.02 |
| 6672 | −0.41 | 0.50 | −0.77 | −0.06 | 1.15 | −1.70 | −0.90 | −1.74 | −1.19 | −0.95 | −2.04 | 0.43 | −1.38 |
| 55578 | −0.42 | −0.89 | −0.07 | 0.42 | 0.77 | −1.89 | −0.01 | −1.18 | −0.27 | −0.89 | −1.29 | −0.89 | −1.58 |
| 159 | −0.38 | −2.06 | −0.30 | 1.09 | 0.51 | −0.30 | −0.27 | −1.80 | −1.71 | −0.41 | −0.20 | −0.33 | −0.77 |
| 9683 | −1.00 | 0.16 | −1.58 | −0.06 | 1.46 | −1.10 | −0.25 | −1.54 | −0.89 | −0.87 | −1.95 | 0.43 | −1.02 |
| 26287 | −0.28 | 0.14 | −2.07 | −0.55 | −0.55 | 0.17 | −0.35 | −1.33 | −0.93 | −2.17 | −1.12 | 0.32 | −0.24 |
| 57147 | −1.47 | 0.05 | −1.41 | 0.71 | 0.69 | −2.01 | −1.00 | −1.14 | −0.81 | −1.05 | −0.23 | 0.39 | −0.94 |
| 1808 | −0.22 | −0.47 | −1.14 | −0.65 | 0.75 | −0.78 | 0.37 | −2.35 | −0.52 | 0.18 | −1.30 | −2.15 | −1.19 |
| 23355 | −0.48 | −0.24 | −0.28 | −0.07 | 1.12 | −1.94 | −0.06 | −2.23 | 0.37 | −0.82 | −1.73 | −0.97 | −1.56 |
| 8115 | −0.57 | 0.60 | −0.25 | 0.24 | −0.46 | −2.10 | −0.35 | −1.33 | −0.75 | −0.66 | −1.09 | −1.39 | −0.80 |
| 94120 | −0.86 | 0.19 | −1.81 | −0.17 | 0.71 | −1.58 | −0.70 | −1.19 | −0.14 | −0.54 | −2.10 | 0.21 | −0.36 |
| 22950 | −0.66 | −0.53 | −0.79 | 0.36 | 0.13 | −1.50 | −0.47 | −1.02 | −1.13 | −0.41 | −1.89 | −0.61 | 0.07 |
| 27342 | −1.20 | −0.21 | −0.90 | −0.53 | 0.85 | −1.17 | −0.26 | −1.49 | −1.08 | −1.28 | −1.85 | 0.56 | −0.35 |
| 64926 | 0.67 | −0.44 | −0.48 | −0.27 | −0.27 | −2.56 | −0.64 | −0.64 | 0.56 | −2.27 | −0.17 | −0.48 | −1.15 |
| 100507331 | 1.34 | −0.27 | 0.43 | −0.89 | 0.40 | −2.05 | −1.05 | −0.39 | −0.93 | −2.45 | −0.39 | 0.96 | −1.35 |
| 4068 | −1.01 | −0.22 | 0.40 | 0.02 | −0.21 | −2.34 | −0.74 | −0.80 | 0.12 | −1.55 | 0.21 | −0.10 | −1.40 |
| 60481 | −0.39 | −0.20 | −0.29 | 0.00 | 1.18 | −1.30 | −0.65 | −1.44 | −1.94 | −1.89 | 0.69 | −0.40 | −1.84 |
| 2634 | −0.85 | 0.18 | −1.26 | 0.37 | 0.70 | −2.05 | −0.70 | −1.66 | −0.31 | −0.49 | −1.33 | 0.63 | −1.57 |
| 55824 | −1.00 | 0.72 | −1.17 | −0.25 | 0.35 | −1.53 | −0.45 | −1.67 | −0.32 | −0.81 | −1.38 | −0.13 | −1.04 |
| 79074 | −0.95 | −1.85 | 0.70 | 0.02 | −0.21 | −1.12 | −1.22 | 0.15 | −3.02 | −1.03 | 0.18 | −0.03 | −0.02 |
| 220930 | −0.97 | −0.59 | −0.10 | 0.55 | 0.82 | −1.48 | −0.65 | −1.51 | −0.43 | −2.00 | −0.83 | 0.09 | −0.45 |
| 64421 | −1.38 | −0.41 | 1.35 | −0.05 | 0.33 | −2.20 | −1.18 | 0.35 | 0.33 | −1.79 | −0.72 | −1.04 | −1.03 |
| 64121 | −0.12 | −1.83 | −1.21 | −1.34 | 0.81 | −0.98 | −0.65 | −0.70 | −1.84 | −0.51 | −0.63 | −0.09 | 0.21 |
| 137835 | 0.07 | −0.14 | 0.09 | 0.36 | 1.00 | −1.85 | 0.42 | −2.21 | −1.08 | −1.10 | −0.98 | −0.35 | −1.63 |
| 57674 | −0.63 | −0.06 | −1.12 | −0.34 | 0.91 | −1.72 | −0.17 | −2.68 | −0.41 | −0.08 | −1.31 | 0.01 | −1.34 |
| 728392 | −0.47 | −1.67 | −1.25 | 0.51 | 0.90 | −1.31 | −0.73 | −1.15 | −0.31 | −0.39 | −1.57 | 0.52 | −1.60 |
| 329 | −1.04 | −0.08 | −0.52 | 0.06 | 0.31 | −0.83 | −1.06 | −0.26 | −1.27 | −1.30 | −0.16 | 0.34 | −0.37 |
| 55279 | −1.93 | −0.33 | −0.99 | 0.23 | 1.02 | −0.61 | −0.75 | −0.51 | −0.88 | −1.07 | −1.48 | 0.68 | −1.01 |
| 7107 | −1.59 | 0.04 | −1.07 | −0.16 | 0.53 | 0.03 | −0.78 | −1.82 | −0.40 | 0.21 | −1.56 | −0.30 | −0.94 |
| 9416 | 0.12 | −0.24 | −0.34 | 1.24 | 0.90 | −2.37 | −0.67 | −1.75 | −1.61 | −0.95 | −0.70 | −1.50 | −0.29 |
| 137964 | 0.48 | 0.30 | −1.74 | −1.21 | −0.72 | −0.46 | −0.21 | −0.05 | 0.01 | −1.19 | −0.90 | −0.49 | −0.57 |
| 3431 | −0.81 | 0.06 | −1.66 | 0.16 | 0.61 | −2.33 | −0.79 | −1.41 | −0.68 | −0.59 | −1.17 | 0.04 | −1.18 |
| 4140 | 0.33 | −2.26 | 1.20 | 0.34 | 1.36 | −1.48 | −0.08 | −1.08 | −1.61 | −0.20 | −1.79 | −0.41 | −1.11 |
| 59269 | −1.56 | 0.96 | −0.87 | 0.22 | 0.42 | −2.34 | −0.13 | −1.08 | −0.23 | −0.51 | −1.23 | 0.23 | −0.72 |
| 7846 | 1.15 | 0.38 | 0.81 | −0.15 | 0.60 | −1.60 | 0.50 | −3.59 | −0.28 | −0.39 | −1.42 | −1.04 | −1.45 |
| 100652740 | −1.07 | 0.95 | 0.20 | 1.02 | 0.50 | −1.37 | −0.22 | −1.35 | 0.04 | −2.28 | −1.01 | −0.74 | −1.02 |
| 23398 | −0.18 | −2.18 | −0.09 | 0.31 | 0.17 | −1.09 | −1.11 | 0.12 | −0.20 | −2.03 | 0.44 | −0.38 | −1.37 |
| 158747 | −0.72 | −0.10 | −0.85 | 0.40 | 1.42 | −1.32 | −0.33 | −1.63 | −1.05 | −0.12 | −2.05 | 0.13 | −1.29 |
| 285512 | −0.14 | −1.10 | 0.10 | 0.28 | 1.52 | −1.51 | −0.57 | −1.58 | −1.01 | −1.46 | −1.35 | 0.30 | −1.66 |
| 3937 | −0.89 | −1.73 | −0.40 | 0.43 | 1.05 | −1.78 | 0.46 | −1.89 | −0.05 | −1.12 | 0.40 | −0.53 | −2.09 |
| 730051 | −1.31 | −1.84 | 0.31 | 0.38 | −0.96 | −0.51 | −0.83 | −0.49 | −1.42 | −1.81 | 0.15 | −0.75 | −0.79 |
| 9320 | 0.26 | −1.55 | −0.22 | 0.30 | 0.50 | −0.17 | −0.29 | −1.37 | −2.81 | 0.55 | −0.58 | −0.84 | −1.13 |
| 6932 | −0.92 | −0.43 | −1.09 | −0.03 | −0.53 | −1.59 | −1.02 | 0.11 | 0.18 | −1.29 | −0.63 | −0.51 | −1.80 |
| 79722 | −0.99 | 0.95 | −0.94 | −0.09 | 0.87 | −1.27 | −0.47 | −1.36 | −0.59 | −1.49 | −1.80 | 0.39 | −1.29 |
| 115362 | −0.30 | −1.02 | −1.53 | 0.78 | −0.34 | −2.54 | −0.70 | −0.59 | −0.85 | −0.17 | 0.08 | −0.48 | −1.08 |
| 29909 | −1.22 | −0.44 | −1.16 | 0.03 | 0.30 | −1.52 | −0.96 | −1.01 | −0.31 | −0.55 | −0.79 | −0.09 | −0.92 |
| 440503 | 0.25 | −0.90 | −0.19 | 0.33 | 1.56 | −1.19 | −0.22 | −0.90 | −0.78 | 0.32 | −2.39 | −0.32 | −2.34 |
| 80183 | −0.83 | 0.89 | −0.52 | 0.15 | 0.73 | −2.17 | −0.34 | −1.21 | −0.48 | −0.74 | −2.22 | −0.53 | −0.99 |
| 80709 | 0.09 | −2.39 | −0.62 | 0.31 | 0.80 | −2.55 | −0.14 | −1.07 | −0.94 | −0.94 | −0.22 | 0.00 | −1.57 |
| 10562 | −0.75 | 0.44 | 0.17 | −0.56 | 1.51 | −1.13 | −0.46 | −1.88 | −0.91 | −0.68 | −0.89 | −0.47 | −1.15 |
| 64780 | −0.90 | 1.05 | 0.18 | 0.23 | 0.51 | −1.46 | 0.12 | −1.72 | −0.22 | −1.71 | −1.15 | −0.35 | −1.39 |
| 155038 | −0.94 | −0.77 | −1.95 | 0.23 | 0.80 | −1.81 | −0.05 | −1.44 | −0.35 | −0.69 | −1.34 | 0.14 | −1.36 |
| 1783 | −0.57 | 0.21 | −0.31 | 0.22 | 0.85 | −0.48 | −0.89 | −2.29 | −1.25 | −0.18 | −1.93 | 0.60 | −1.22 |
| 100287569 | −0.35 | −0.49 | 0.06 | 0.02 | 0.75 | −2.53 | −0.06 | −2.53 | −0.08 | −1.06 | −1.68 | 0.15 | −1.27 |
| 5696 | −0.81 | 0.01 | −1.36 | −1.04 | −0.42 | −1.07 | −0.25 | −0.81 | −1.51 | 0.41 | 0.08 | −1.32 | 0.22 |
| 11066 | 0.13 | −0.68 | −0.85 | 0.13 | 0.95 | −2.53 | −0.69 | −0.99 | −1.24 | −1.33 | −1.92 | 0.26 | −0.60 |
| 122553 | −0.16 | −2.71 | 0.00 | 0.32 | 0.98 | −1.18 | 0.01 | −0.87 | −1.43 | −1.56 | −0.59 | −0.44 | −0.71 |
| 50484 | −1.12 | −1.43 | −1.00 | 0.67 | 0.90 | −1.30 | 0.32 | −1.62 | −0.86 | −0.36 | −1.86 | 0.18 | −0.46 |
| 9847 | −1.83 | −2.61 | −0.61 | 1.06 | −0.25 | −0.77 | −0.92 | −0.46 | −0.99 | 0.45 | −0.49 | −0.83 | −0.10 |
| 129285 | −1.01 | −0.36 | −1.61 | 0.01 | 0.43 | −1.50 | −0.58 | −1.33 | −0.80 | −0.92 | −0.14 | −0.04 | −2.08 |
| 9794 | −0.52 | 0.35 | −1.25 | 0.17 | 0.85 | −1.59 | −0.01 | −1.42 | −0.69 | −1.64 | −0.49 | −0.21 | −1.03 |
| 84309 | 1.49 | 0.08 | 0.02 | −0.09 | 0.08 | −1.18 | −0.84 | −0.74 | −1.04 | −2.16 | −1.98 | 0.01 | 0.06 |
| 65258 | −0.21 | −0.75 | 0.15 | 0.66 | 0.67 | −2.29 | −0.16 | −1.27 | −1.19 | −0.31 | −1.91 | −0.08 | −0.93 |
| 5562 | −0.29 | −0.88 | −0.50 | −0.09 | 1.12 | −1.01 | −0.88 | −1.07 | −1.58 | −2.04 | −0.86 | 0.59 | −1.07 |
| 123036 | −1.17 | −1.17 | −1.30 | 0.09 | 0.14 | −0.60 | −1.59 | −0.52 | −0.41 | −0.32 | −0.38 | −0.26 | −1.35 |
| 84166 | −0.79 | 0.79 | −0.88 | −0.40 | 0.14 | −2.19 | −1.22 | −1.14 | 0.27 | −1.78 | −0.60 | 0.19 | −1.49 |
| 253143 | −0.68 | −0.07 | −0.86 | 0.70 | 1.64 | −0.89 | 0.03 | −2.02 | −0.93 | −0.57 | −2.32 | 0.25 | −0.87 |
| 1236 | −1.52 | 0.66 | −1.45 | −0.86 | −0.73 | −0.87 | −1.44 | 0.22 | −0.11 | −1.92 | 0.11 | −0.20 | −1.57 |
| 266747 | −0.28 | −0.18 | −0.28 | −0.09 | 0.92 | −2.31 | −0.37 | −1.04 | −1.25 | −1.62 | −1.49 | 0.51 | −0.93 |
| 25988 | −0.34 | −1.43 | −2.67 | 0.53 | −0.80 | −0.42 | −0.62 | −0.04 | −2.16 | 0.17 | −0.14 | −0.49 | −0.67 |
| 6515 | −0.25 | 0.29 | −0.33 | −0.24 | 1.00 | −1.44 | 0.08 | −2.10 | −1.18 | −1.10 | −1.80 | 0.39 | −1.73 |
| 90592 | −0.72 | 0.55 | 0.31 | 0.50 | 0.89 | −1.42 | −1.32 | −0.56 | −1.19 | −1.39 | −1.18 | −0.12 | −1.47 |
| 6867 | −0.74 | −0.18 | −0.34 | 0.25 | 1.10 | −1.44 | 0.24 | −1.55 | −0.49 | −1.01 | −1.79 | −0.35 | −0.95 |
| 23041 | −2.09 | 0.71 | −0.91 | 0.18 | 0.34 | −0.58 | −1.37 | −0.78 | −0.13 | −0.60 | −1.10 | −0.26 | −1.54 |
| 100289230 | −0.74 | −0.15 | −0.65 | 0.44 | −0.37 | −0.81 | −1.55 | −0.52 | 0.23 | −0.16 | −1.57 | −0.03 | −0.99 |
| 57169 | −0.37 | −0.23 | −1.52 | −0.22 | 1.44 | −1.50 | −0.82 | −1.07 | −1.29 | −1.01 | −1.68 | 0.38 | −1.13 |
| 1105 | −1.16 | −1.37 | 0.21 | 0.35 | 0.63 | −0.87 | 0.00 | −0.31 | −1.67 | −2.36 | −0.12 | 0.20 | −1.18 |
| 943 | −1.09 | 0.72 | −1.48 | −0.68 | −0.11 | −1.16 | −0.49 | −0.72 | −0.28 | −0.76 | −1.78 | −0.22 | −1.05 |
| 3275 | −0.11 | −1.52 | −2.05 | −0.60 | 1.07 | −1.13 | −0.52 | −1.13 | −0.80 | −1.50 | −0.76 | 0.33 | −1.53 |
| 8851 | −1.27 | 0.09 | −0.75 | 0.10 | 0.45 | −1.69 | −0.87 | −0.37 | −0.74 | −1.32 | −2.10 | 0.18 | −1.17 |
| 168537 | −1.13 | −1.10 | −1.52 | 0.37 | 0.42 | −1.80 | −0.20 | −0.77 | −0.64 | −1.19 | −0.60 | −0.16 | −1.21 |
| 256236 | −1.01 | 1.15 | −0.08 | −0.58 | −0.57 | −1.84 | 0.10 | −1.95 | 0.05 | −0.45 | −0.98 | −1.51 | −0.67 |
| 26235 | −0.25 | −0.66 | −0.27 | 0.77 | 0.54 | −1.25 | −1.17 | −1.40 | −1.58 | −0.75 | −1.63 | 0.03 | −0.55 |
| 9363 | −0.84 | 1.47 | −1.22 | −1.17 | −0.10 | −0.94 | −1.70 | −0.30 | −1.62 | −0.65 | −0.77 | −0.17 | −0.65 |
| 729683 | −1.51 | −1.17 | −1.00 | 0.20 | 0.39 | −2.26 | −1.09 | −1.07 | −1.69 | −0.11 | −0.15 | 0.29 | −0.48 |
| 388969 | −0.04 | −0.67 | −0.04 | 0.22 | 0.21 | −2.41 | 0.05 | −0.82 | −0.96 | −0.78 | −0.39 | −0.91 | −1.23 |
| 4818 | −0.26 | −0.75 | 0.53 | 0.48 | −0.50 | −2.79 | 0.79 | −1.43 | −0.75 | −1.10 | −0.10 | −0.96 | −1.22 |
| 93953 | −1.27 | −0.44 | −0.68 | 0.34 | 0.61 | −1.30 | −0.72 | −0.35 | −1.00 | −2.04 | −1.08 | 0.47 | −1.21 |
| 28982 | −1.05 | 0.19 | −0.75 | 1.20 | 0.49 | −1.00 | −0.62 | −0.80 | −0.40 | −0.43 | −0.71 | −1.68 | −0.66 |
| 84911 | −0.32 | −1.23 | −0.59 | −0.19 | −0.09 | −0.52 | 0.34 | −1.03 | −0.06 | −0.71 | 0.23 | −2.34 | −1.33 |
| 66036 | −1.26 | −0.33 | −1.11 | −0.15 | −0.23 | −0.73 | −1.47 | −0.41 | −0.08 | −1.49 | −1.18 | −0.11 | −0.14 |
| 9750 | −0.24 | −0.58 | −0.72 | 0.43 | 0.77 | −2.15 | −0.04 | −2.13 | −0.75 | −1.28 | −1.12 | 0.23 | −1.78 |
| 9938 | −0.08 | 0.04 | −0.70 | 0.37 | 1.18 | −2.93 | −0.02 | −1.38 | −0.37 | −1.88 | −1.30 | 0.26 | −1.17 |
| 120425 | −0.83 | 0.04 | −1.15 | −0.35 | 1.21 | −2.05 | −0.06 | −2.25 | 0.07 | −0.63 | −1.63 | −0.35 | −1.23 |
| 6840 | 0.18 | 0.30 | −0.33 | −0.12 | 1.48 | −1.28 | −0.22 | −2.26 | −0.72 | −0.77 | −2.56 | 0.14 | −1.39 |
| 26268 | −0.65 | 0.06 | −0.29 | −0.20 | 0.81 | −1.06 | −0.47 | −0.45 | −1.31 | 0.04 | −1.88 | 0.01 | −0.93 |
| 55206 | −0.15 | −0.54 | −0.19 | 0.59 | 0.92 | −1.47 | −0.44 | −0.58 | −0.84 | −1.87 | −1.48 | 0.18 | −0.88 |
| 355 | −0.75 | −0.30 | −0.75 | 0.02 | 0.98 | −1.06 | −0.27 | −1.99 | −1.22 | −0.61 | −1.13 | 0.19 | −1.96 |
| 50615 | −1.20 | 1.61 | −1.16 | −0.50 | −0.32 | −1.31 | −0.43 | −0.83 | 0.10 | −0.72 | −1.50 | −0.50 | −0.95 |
| 221178 | −0.17 | −0.98 | −1.13 | −0.24 | 0.81 | −1.02 | −0.12 | −1.30 | −0.70 | −1.05 | −0.99 | −0.18 | −1.39 |
| 11104 | −1.00 | −1.29 | 0.07 | −0.20 | 0.39 | −0.45 | −0.69 | −0.95 | −1.39 | −0.74 | −0.44 | −0.90 | 0.07 |
| 1117 | −0.37 | −0.35 | −0.97 | 0.81 | −0.79 | −0.82 | −1.19 | −0.97 | −0.89 | −1.52 | −1.55 | −0.62 | −0.26 |
| 374969 | 0.47 | −1.69 | −0.62 | −0.62 | 0.37 | −1.67 | 0.07 | −1.58 | −0.60 | −0.27 | −1.79 | −0.36 | −0.59 |
| 6285 | −0.37 | −0.57 | −0.75 | −1.27 | −0.47 | −0.98 | −0.60 | −1.02 | −0.51 | 0.51 | 0.26 | −1.69 | −0.19 |
| 387357 | −1.81 | −0.37 | −1.07 | 0.36 | 0.17 | −1.84 | −0.85 | −0.59 | 0.09 | −0.85 | −0.32 | −0.57 | −0.95 |
| 2960 | −2.33 | −0.14 | −1.93 | −0.30 | 0.30 | −0.34 | −0.32 | 0.37 | −1.63 | 0.30 | −1.06 | −0.21 | −0.49 |
| 100129550 | −0.56 | −0.87 | −0.55 | 0.51 | 1.52 | −1.52 | −0.52 | −1.34 | −0.69 | −0.99 | −1.72 | 0.73 | −1.33 |
| 54520 | −0.61 | −0.60 | −1.14 | −0.17 | 0.93 | 0.13 | −1.33 | −0.77 | −0.86 | −1.40 | −2.10 | 0.55 | −0.69 |
| 10302 | −2.51 | 0.24 | −1.32 | −0.11 | −0.67 | −1.51 | −0.84 | −0.30 | −1.00 | 0.64 | −1.00 | −0.62 | −0.01 |
| 50939 | 0.55 | 1.26 | 0.53 | 0.01 | −0.91 | −2.42 | −1.58 | −1.16 | −0.05 | −0.93 | −0.40 | 0.13 | −0.90 |
| 8802 | −0.26 | −0.40 | 0.15 | −0.86 | 0.56 | −0.18 | −0.38 | −0.80 | −0.66 | −0.41 | −1.01 | −1.39 | 0.16 |
| 23332 | −0.73 | −0.60 | 0.25 | 0.40 | 0.05 | −1.67 | 0.15 | −0.75 | −1.95 | −0.31 | −0.74 | −0.40 | −0.16 |
| 203328 | −1.37 | 0.50 | −1.02 | −0.79 | 0.35 | −1.87 | 0.00 | −0.97 | −0.06 | −0.86 | −1.48 | −0.06 | −1.23 |
| 126231 | 0.39 | −2.26 | −1.23 | 0.00 | −0.56 | −0.68 | −1.32 | −1.10 | −0.61 | 0.39 | −0.15 | −0.50 | −0.74 |
| 6993 | −0.51 | 0.41 | −1.39 | −0.72 | 1.05 | −1.49 | −0.91 | −1.51 | −1.17 | −0.20 | −1.94 | 0.11 | −0.78 |
| 58500 | −0.54 | −0.79 | −1.35 | 0.42 | 0.53 | −1.82 | −0.65 | −0.18 | −1.35 | −0.15 | −2.17 | 0.21 | −1.24 |
| 10507 | −0.38 | −1.18 | −1.00 | 0.33 | 0.36 | −2.64 | −0.26 | −0.57 | −0.68 | −1.26 | −1.14 | −0.30 | −1.26 |
| 170482 | −0.98 | 0.02 | −0.07 | −0.63 | −0.22 | −1.15 | −0.35 | −1.45 | −0.35 | −0.72 | −0.99 | −1.52 | −0.54 |
| 3004 | −0.95 | −0.97 | −0.91 | −0.77 | −0.18 | −1.88 | −0.71 | −1.09 | −0.30 | −1.04 | −0.53 | −0.22 | −1.04 |
| 64895 | −1.67 | −0.42 | −1.03 | 0.44 | 0.98 | −1.73 | −0.15 | −0.71 | −0.87 | −1.84 | −0.75 | 0.61 | −1.30 |
| 7850 | −0.67 | 1.29 | −0.64 | 0.00 | 1.32 | −1.10 | −0.32 | −1.90 | −1.20 | −0.47 | −2.35 | 0.09 | −1.24 |
| 93594 | −0.52 | −0.18 | −1.15 | 1.02 | −0.26 | −1.63 | −0.65 | −0.63 | −0.60 | −0.77 | −1.58 | −0.53 | −0.92 |
| 23215 | −0.02 | −0.02 | −1.17 | −0.27 | 0.75 | −0.74 | −0.41 | −1.26 | −0.24 | −1.50 | −1.71 | 0.38 | −2.86 |
| 80196 | −1.40 | −1.60 | −1.67 | 0.19 | 0.54 | −1.47 | −0.38 | −0.43 | −1.78 | −0.28 | −1.10 | −0.15 | −0.36 |
| 284415 | −0.02 | −0.90 | 0.30 | −0.09 | 0.39 | −1.80 | −0.45 | −1.49 | −1.23 | −0.58 | −1.27 | −0.30 | −0.93 |
| 56882 | −0.02 | −0.23 | −0.45 | 0.56 | 1.08 | −1.26 | 0.00 | −1.70 | −1.27 | −0.51 | −2.48 | −0.24 | −1.13 |
| 6508 | −0.25 | −1.12 | −0.36 | −0.53 | 0.56 | −2.53 | −0.17 | −0.62 | −0.30 | −1.95 | −1.06 | 1.04 | −1.00 |
| 79663 | −0.27 | −1.40 | −0.24 | 0.30 | 1.15 | −1.04 | −0.50 | −1.65 | −0.67 | −0.77 | −1.79 | −0.15 | −1.43 |
| 474344 | −0.72 | −1.37 | −2.10 | 0.05 | 0.03 | −1.97 | −0.13 | −1.39 | −0.03 | −0.36 | −0.19 | −0.44 | −1.05 |
| 100505746 | −1.28 | −0.13 | −0.74 | −0.08 | 0.88 | −1.59 | −0.13 | −1.86 | 0.40 | −1.70 | −1.08 | −0.35 | −1.67 |
| 1293 | −1.17 | 1.62 | −0.54 | −0.45 | 0.44 | −1.67 | −0.99 | −1.27 | 0.38 | −1.26 | −1.50 | −0.17 | −0.57 |
| 10294 | −0.59 | −1.32 | −0.43 | 0.01 | 0.45 | −0.06 | −0.97 | −0.98 | −1.82 | −0.63 | −0.48 | −0.63 | −1.05 |
| 3091 | −0.70 | 0.99 | 0.89 | −0.76 | 0.21 | −1.25 | 0.00 | −2.32 | −0.13 | −0.97 | −1.08 | −0.59 | −0.88 |
| 79573 | −1.41 | −0.10 | −0.94 | 0.62 | 0.43 | −0.44 | −1.60 | −0.96 | −0.73 | −1.13 | −0.97 | −0.08 | −0.50 |
| 9208 | 0.56 | −0.76 | −0.82 | 0.46 | 0.85 | −1.06 | 0.13 | −1.92 | −1.27 | −1.78 | −0.81 | 0.08 | −1.97 |
| 29116 | −0.27 | −2.58 | 1.19 | 0.46 | 0.32 | −0.74 | −0.61 | −0.90 | −0.53 | −0.89 | −1.47 | −0.42 | −1.07 |
| 92370 | −1.62 | −0.80 | −0.74 | 0.67 | 0.91 | −1.12 | −0.78 | −0.24 | −0.84 | −1.53 | −1.46 | 0.25 | −0.82 |
| 9546 | 0.00 | 0.07 | −0.75 | −0.16 | 0.97 | −0.85 | −1.10 | −2.63 | −1.00 | −0.08 | −2.03 | −0.36 | −1.09 |
| 253018 | −0.64 | −0.72 | −0.47 | −0.28 | 1.22 | −1.80 | −0.80 | −1.19 | −0.64 | −0.53 | −2.19 | 0.31 | −1.22 |
| 10096 | −0.79 | −0.13 | 0.15 | 0.42 | 1.60 | −1.46 | −0.39 | −1.67 | −0.94 | −2.34 | −1.00 | −0.52 | −1.31 |
| 84138 | 0.84 | −1.16 | −0.64 | 0.40 | 0.22 | −2.19 | −0.53 | −0.75 | −1.75 | 0.11 | −1.27 | −1.19 | −0.40 |
| 8440 | −0.60 | −0.88 | −1.96 | −0.14 | −0.16 | −0.66 | −0.67 | −0.59 | −0.68 | −0.08 | −0.17 | −1.75 | −1.26 |
| 7277 | −1.39 | 0.66 | −1.25 | −0.13 | 0.96 | −1.88 | −0.51 | −0.40 | −0.36 | −1.78 | −2.08 | −0.03 | −1.07 |
| 8269 | 0.26 | −2.71 | −2.09 | 0.21 | −0.25 | −0.22 | −0.46 | 0.48 | −0.52 | −0.13 | −1.15 | −0.51 | 0.06 |
| 8807 | −0.75 | 0.15 | −1.18 | 0.04 | 0.86 | −1.98 | −0.68 | −1.33 | −0.87 | −1.01 | −1.47 | −0.05 | −1.30 |
| 1235 | −1.09 | −0.52 | −1.19 | −0.47 | −0.71 | −0.95 | −0.92 | −1.19 | −0.36 | −0.29 | −0.45 | −0.48 | −0.84 |
| 7059 | 1.44 | 0.21 | 0.44 | −0.70 | 0.17 | −0.78 | 0.31 | −2.87 | −0.05 | −0.46 | −0.82 | −0.56 | −2.14 |
| 54971 | −0.04 | 0.35 | −1.94 | 0.12 | 0.56 | −0.90 | −0.97 | −1.54 | −0.70 | −0.60 | −2.09 | 0.10 | −0.72 |
| 57677 | −0.82 | 1.21 | −1.02 | 0.25 | 1.22 | −1.04 | −1.24 | −0.91 | −1.59 | −1.39 | −1.38 | 0.08 | −1.02 |
| 6890 | −1.12 | 1.35 | −2.35 | −0.28 | 0.60 | −1.63 | −1.24 | −1.25 | −0.70 | −0.27 | −1.54 | −0.02 | −0.33 |
| 22944 | −0.59 | −0.67 | −0.86 | 0.25 | 1.10 | −1.47 | −0.52 | −0.53 | −1.37 | −2.18 | −0.74 | −0.02 | −0.84 |
| 26512 | −0.86 | −0.50 | −0.82 | 0.25 | 0.70 | −1.38 | −0.56 | −0.29 | −0.82 | −1.28 | −1.37 | −0.09 | −1.73 |
| 92797 | −1.34 | −0.36 | −0.87 | 0.98 | 1.14 | −1.92 | 0.04 | −1.62 | −1.14 | −1.06 | −1.18 | −0.46 | −1.27 |
| 5481 | 0.41 | −0.37 | −0.66 | −0.08 | 0.36 | −0.38 | −0.04 | −1.15 | −3.10 | −0.34 | −0.76 | −1.56 | −0.92 |
| 22897 | −0.12 | −0.91 | −0.53 | −0.46 | −0.49 | −0.66 | −0.50 | −1.20 | −0.15 | −0.12 | −0.78 | −0.17 | −1.82 |
| 3620 | −0.02 | 0.84 | −1.89 | −0.16 | 0.34 | −0.54 | −0.76 | −1.61 | −0.41 | 0.02 | −2.38 | −0.88 | −0.33 |
| 64766 | −0.78 | 0.05 | −0.24 | 1.04 | 0.31 | −1.50 | −0.70 | −1.26 | −1.77 | −1.72 | 0.08 | −0.33 | −1.45 |
| 8809 | −1.30 | −0.28 | −1.05 | 0.20 | 1.05 | −1.61 | −0.70 | −1.51 | −0.97 | −1.04 | −1.44 | 0.04 | −1.06 |
| 116984 | −1.14 | −0.26 | −1.85 | −0.25 | 0.54 | −1.26 | −0.93 | −0.94 | −0.72 | −0.79 | −0.48 | −0.15 | −0.90 |
| 440823 | −1.34 | 1.31 | −1.31 | −0.75 | 0.29 | −1.84 | −1.26 | −0.78 | −0.53 | −0.71 | −0.46 | 0.38 | −1.74 |
| 59340 | −0.83 | −0.38 | −2.29 | 0.82 | 0.85 | −0.82 | −0.18 | −0.27 | −0.82 | −1.15 | −0.41 | −1.10 | −1.82 |
| 197259 | −0.21 | 0.32 | −0.72 | 0.01 | 1.28 | −1.69 | −0.61 | −2.04 | −0.74 | −0.70 | −1.19 | −0.54 | −1.66 |
| 23670 | −0.67 | −1.12 | −0.96 | 0.44 | 0.99 | −1.40 | −0.20 | −1.98 | −0.94 | −1.09 | −1.23 | 0.23 | −1.20 |
| 643314 | −0.32 | 0.34 | −0.53 | 0.57 | 0.62 | −2.15 | −1.32 | −1.91 | −0.16 | −1.25 | −1.16 | −0.01 | −2.19 |
| 28526 | −0.33 | 0.10 | −1.60 | 0.78 | −0.68 | −2.09 | −0.85 | −0.73 | −0.60 | −1.17 | −0.48 | −0.75 | −0.92 |
| 55096 | 0.28 | −0.35 | 0.33 | −0.78 | 0.63 | −2.07 | −1.96 | −1.31 | −0.66 | −0.55 | −1.70 | 0.36 | −1.25 |
| 2796 | −0.66 | −2.90 | 0.38 | −0.08 | −0.04 | −2.13 | 0.07 | 0.42 | −0.22 | −0.28 | −0.14 | −0.85 | −1.54 |
| 54509 | −0.80 | 0.32 | −1.43 | −1.08 | −0.54 | −0.76 | 0.25 | −0.90 | −1.00 | −0.42 | −0.88 | −0.38 | −1.12 |
| 11120 | 0.07 | −1.63 | −0.62 | −0.13 | 0.65 | −0.96 | −0.54 | −1.58 | −1.21 | −1.08 | −0.76 | 0.42 | −1.52 |
| 84869 | −1.53 | −0.83 | −1.22 | 0.27 | 0.12 | −0.85 | −0.74 | −1.13 | −1.13 | 0.70 | −1.53 | −1.57 | −0.51 |
| 28991 | −0.58 | −0.24 | −1.90 | −0.12 | 1.08 | −0.75 | −0.13 | −0.51 | −2.03 | −0.07 | −2.53 | −0.10 | −0.26 |
| 154141 | 0.00 | −2.65 | −0.96 | 0.23 | 0.95 | −0.25 | 0.11 | −2.56 | −0.86 | −0.20 | −1.10 | −0.21 | −1.12 |
| 3659 | −0.18 | −0.21 | −1.33 | −0.05 | 1.12 | −2.24 | −0.61 | −1.14 | −1.09 | −0.73 | −0.41 | 0.38 | −1.25 |
| 154007 | −0.20 | −0.06 | −0.13 | 0.34 | 0.70 | −2.02 | −0.47 | −1.60 | −0.35 | −1.79 | −1.87 | 0.29 | −1.08 |
| 116835 | −1.12 | 0.68 | −1.54 | 0.24 | 0.80 | −1.18 | −0.33 | −2.58 | −1.03 | −0.89 | −1.03 | 0.09 | −1.15 |
| 9488 | −1.72 | −0.07 | −0.55 | 0.53 | 0.32 | −1.55 | 0.30 | −2.07 | −1.93 | −0.62 | −1.05 | −0.04 | −1.24 |
| 100293516 | −1.76 | −1.15 | −0.55 | 0.34 | −0.74 | −1.19 | −0.96 | −0.71 | −0.49 | −1.11 | 0.00 | −0.92 | −0.87 |
| 9217 | −1.39 | 0.45 | −1.31 | 0.22 | 0.31 | −1.69 | −0.91 | −0.14 | −0.47 | 0.21 | −1.60 | −0.55 | −1.38 |
| 10538 | −0.20 | 1.16 | −0.96 | −1.16 | 0.40 | −2.02 | −0.61 | −1.84 | −0.45 | −1.32 | −1.77 | 0.27 | −1.54 |
| 6935 | −1.18 | −0.24 | −0.24 | −0.04 | 0.41 | −2.43 | −0.58 | −0.53 | −0.58 | −3.17 | −0.48 | 0.80 | −0.66 |
| 10443 | −0.53 | −0.16 | −0.41 | 0.56 | 0.54 | −2.02 | −0.84 | −1.19 | 0.30 | −1.54 | −0.75 | 0.00 | −1.76 |
| 3837 | −0.67 | −3.06 | −0.44 | 0.00 | 0.76 | −0.97 | 0.33 | −1.43 | −0.63 | 0.10 | −1.19 | −0.47 | −0.87 |
| 81698 | 0.15 | 0.90 | −0.22 | −0.31 | 0.27 | −1.29 | −2.39 | −2.45 | 0.03 | 0.43 | −1.90 | −0.50 | −1.09 |
| 2643 | −1.53 | −1.49 | −0.47 | −0.24 | −0.10 | −0.92 | −0.26 | −0.91 | 0.08 | −0.70 | −1.58 | −0.30 | −0.27 |
| 57559 | −1.08 | 0.72 | −0.73 | 0.38 | −0.75 | −1.66 | −1.63 | −1.12 | −1.00 | −1.40 | −0.22 | −0.50 | −0.78 |
| 116842 | −0.83 | −0.27 | −0.92 | −0.42 | 0.50 | −0.31 | −1.47 | −0.82 | −0.86 | −1.33 | −1.20 | 0.53 | −0.94 |
| 202 | −0.59 | 0.69 | −0.56 | −0.53 | 0.42 | −1.52 | 0.10 | −2.24 | −0.36 | −0.70 | −1.94 | −0.25 | −1.80 |
| 9934 | −0.93 | −0.16 | −0.92 | 0.22 | 0.90 | −2.26 | 0.01 | −1.89 | −0.72 | −0.65 | −1.42 | −0.25 | −1.14 |
| 100216546 | −0.56 | −0.82 | −0.80 | 0.04 | 0.65 | −0.82 | −2.30 | −0.93 | −0.17 | −0.24 | −1.64 | −0.02 | −0.84 |
| 9692 | 0.38 | −1.90 | −1.18 | −0.97 | −0.22 | −0.70 | 0.15 | 0.14 | −2.01 | −0.92 | 0.28 | −0.30 | −0.59 |
| 89845 | 0.54 | −0.16 | −2.42 | 0.41 | 0.04 | −0.12 | −1.78 | −0.04 | −0.80 | −0.84 | −1.59 | −0.56 | −0.79 |
| 26034 | −0.98 | 0.48 | −1.28 | 0.09 | 0.38 | −1.26 | −0.81 | −1.65 | −0.45 | −1.23 | −1.57 | −0.25 | −1.32 |
| 10906 | −0.61 | −0.68 | −1.48 | 0.02 | 0.72 | −1.60 | −0.28 | −2.06 | −1.44 | −0.57 | −1.66 | −0.44 | −0.41 |
| 84811 | −1.59 | −1.10 | −0.68 | −0.44 | −0.21 | −0.77 | −1.39 | −1.63 | −1.12 | −0.57 | −0.80 | 0.75 | −0.03 |
| 100527964 | 0.05 | 1.04 | −0.40 | 0.00 | −0.34 | −1.39 | −1.20 | −0.92 | 0.41 | −1.88 | −0.90 | −0.84 | −1.32 |
| 118426 | −1.60 | −0.77 | −1.09 | −0.21 | 0.73 | −1.44 | −1.03 | −1.56 | −0.64 | −0.62 | −1.07 | 0.54 | −1.33 |
| 54331 | −0.48 | 0.46 | −0.38 | 0.16 | 0.78 | −2.96 | −0.45 | −1.09 | −1.12 | −1.74 | −0.94 | −0.35 | −1.45 |
| 94081 | −2.28 | 1.02 | −0.14 | −0.39 | 0.15 | −2.23 | −0.54 | −0.65 | −0.66 | −1.46 | −1.12 | 0.10 | −0.57 |
| 145474 | −0.03 | −0.28 | −0.80 | 0.23 | 0.89 | −1.38 | −1.25 | −1.71 | −0.39 | −1.06 | −1.50 | −0.67 | −1.24 |
| 84969 | −1.53 | 1.55 | −0.78 | −0.37 | −0.02 | −1.42 | −1.61 | 0.04 | −1.08 | −1.09 | −1.23 | −0.27 | −0.35 |
| 4907 | 0.06 | −1.19 | −0.32 | −0.13 | −0.29 | −0.78 | −1.18 | −0.57 | −1.72 | −0.69 | −1.63 | −0.32 | −1.49 |
| 51735 | −1.21 | −0.10 | −0.94 | 0.31 | 0.41 | −1.39 | −0.94 | −0.52 | −1.20 | −1.41 | −1.43 | −0.01 | −0.69 |
| 9953 | −0.53 | −0.67 | −0.82 | 0.27 | 0.65 | −1.31 | −0.71 | −0.50 | −0.78 | −1.37 | −1.29 | −0.06 | −1.19 |
| 23112 | 0.28 | −0.77 | −0.81 | 0.84 | 0.47 | −1.45 | −1.08 | −1.48 | −0.92 | −0.27 | −1.72 | −0.35 | −1.58 |
| 91526 | −1.20 | −0.61 | −1.21 | −0.03 | 0.86 | −1.42 | −0.82 | −0.96 | −0.97 | −2.04 | −0.91 | 0.44 | −1.56 |
| 101928017 | −0.42 | 1.09 | −0.81 | −0.38 | −0.52 | −1.45 | −0.60 | −1.46 | −0.57 | −0.22 | −1.75 | −0.35 | −0.22 |
| 84859 | −1.38 | −0.85 | −2.06 | 0.23 | 0.48 | −0.68 | −0.84 | −0.74 | −1.35 | −0.29 | −0.76 | 0.04 | −1.66 |
| 159013 | −0.49 | −1.10 | −0.70 | −0.08 | 0.46 | −1.66 | 0.47 | −1.78 | −1.91 | −0.81 | −0.82 | −0.56 | −1.14 |
| 23208 | −0.27 | −1.12 | −0.69 | −0.33 | −0.57 | −1.61 | −0.26 | −2.00 | −0.48 | −1.64 | −0.96 | 0.46 | −0.34 |
| 101928649 | −0.60 | −0.31 | −0.38 | 0.03 | −0.07 | −1.16 | −0.43 | −1.38 | −1.48 | −1.13 | −0.90 | −0.62 | −0.67 |
| 85459 | −0.61 | 0.37 | −0.22 | 0.15 | 0.36 | −2.16 | −1.14 | −1.03 | −0.96 | −1.76 | −1.59 | 0.22 | −1.32 |
| 9617 | −0.97 | −1.58 | −0.41 | −0.30 | −0.06 | −1.36 | −1.09 | −0.40 | −0.87 | −1.44 | −0.19 | −1.02 | −1.03 |
| 56898 | −0.03 | −0.59 | 0.11 | −0.28 | −1.73 | −2.44 | −0.08 | −0.40 | 0.31 | −2.06 | −0.63 | −1.07 | −0.16 |
| 387882 | −0.76 | −0.21 | −0.74 | 0.20 | −0.52 | −1.77 | −1.07 | −0.65 | −1.69 | 0.18 | 0.42 | −1.73 | −1.48 |
| 677769 | 0.69 | 0.61 | −0.80 | −0.90 | −0.17 | −1.40 | −1.81 | −0.17 | −0.69 | −0.97 | −1.39 | −0.39 | −1.39 |

High level gene sets for deleterious genes (increase with age) and beneficial genes (decrease with age) were identified, as shown in FIG. 2, and as summarized here:

For high level gene sets, the following sets of genes were found to be deleterious (that is, they increase with age): UNFOLDED_PROTEIN-RESPONSE, OXIDATIVE_PHOSPHORYLATION, MYC_TARGETS_V1, ADIPOGENESIS, GLYCOLYSIS, UV_RESPONSE_UP, DNA_REPAIR, FATTY_ACID_METABOLISM, SPERMATOGENESIS, E2F_TARGETS, BILE_ACID_METABOLISM, MTORC1_SIGNALING, ESTROGEN_RESPONSE_LATE.

The following genes were found to be beneficial (that is, they decrease with age): APOPTOSIS, APICAL_SURFACE, UV_RESPONSE_DN, EPITHELIAL_MESENCHYMAL_TRANSITION, ANGIOGENESIS, KRAS_SIGNALING_UP, TNFA_SIGNALING_VIA_NFKB, IL2_STAT5_SIGNALING COMPLEMENT, INFLAMMATORY_RESPONSE, INTERFERON_ALPHA_RESPONSE, ALLOGRAFT_REJECTION AND INTERFERENCE_GAMMA_RESPONSE.

Table 4 summarizes the data from the high level genesets:

TABLE 4
High Level Genesets

| gs.id | gs.desc | gs.ngenes | fisherz |
|---|---|---|---|
| UNFOLDED_PROTEIN_RESPONSE | Genes up-regulated during unfolded | 109 | 6.005 |
| | protein response, a cellular stress | | |
| | response related to the endoplasmic | | |
| | reticulum. | | |
| OXIDATIVE_PHOSPHORYLATION | Genes encoding proteins involved in | 188 | 5.683 |
| | oxidative phosphorylation. | | |
| MYC_TARGETS_V1 | A subgroup of genes regulated by | 179 | 5.562 |
| | MYC - version 1 (v1). | | |
| HEME_METABOLISM | Genes involved in metabolism of | 197 | 4.56 |
| | heme (a cofactor consisting of iron | | |
| | and porphyrin) and erythroblast | | |
| | differentiation. | | |
| MYC_TARGETS_V2 | A subgroup of genes regulated by | 55 | 3.473 |
| | MYC - version 2 (v2). | | |
| ADIPOGENESIS | Genes up-regulated during adipocyte | 196 | 3.441 |
| | differentiation (adipogenesis). | | |
| GLYCOLYSIS | Genes encoding proteins involved in | 198 | 3.428 |
| | glycolysis and gluconeogenesis. | | |
| UV_RESPONSE_UP | Genes up-regulated in response to | 157 | 3.197 |
| | ultraviolet (UV) radiation. | | |
| DNA_REPAIR | Genes involved in DNA repair. | 146 | 2.991 |
| FATTY_ACID_METABOLISM | Genes encoding proteins involved in | 151 | 2.425 |
| | metabolism of fatty acids. | | |
| SPERMATOGENESIS | Genes up-regulated during production | 132 | 2.34 |
| | of male gametes (sperm), as in | | |
| | spermatogenesis. | | |
| E2F_TARGETS | Genes encoding cell cycle related | 192 | 2.266 |
| | targets of E2F transcription factors. | | |
| BILE_ACID_METABOLISM | Genes involve in metabolism of bile | 111 | 2.149 |
| | acids and salts. | | |
| MTORC1_SIGNALING | Genes up-regulated through | 191 | 2.144 |
| | activation of mTORC1 complex. | | |
| ESTROGEN_RESPONSE_LATE | Genes defining late response to | 195 | 2.135 |
| | estrogen. | | |
| APOPTOSIS | Genes mediating programmed cell | 155 | −2.002 |
| | death (apoptosis) by activation of | | |
| | caspases. | | |
| APICAL_SURFACE | Genes encoding proteins over- | 43 | −2.272 |
| | represented on the apical surface of | | |
| | epithelial cells, e.g., important for cell | | |
| | polarity (apical area). | | |
| UV_RESPONSE_DN | Genes down-regulated in response to | 142 | −2.494 |
| | ultraviolet (UV) radiation. | | |
| EPITHELIAL_MESEN- | Genes defining epithelial- | 197 | −2.866 |
| CHYMAL_TRANSITION | mesenchymal transition, as in wound | | |
| | healing, fibrosis and metastasis. | | |
| ANGIOGENESIS | Genes up-regulated during formation | 36 | −3.103 |
| | of blood vessels (angiogenesis). | | |
| KRAS_SIGNALING_UP | Genes up-regulated by KRAS | 198 | −3.697 |
| | activation. | | |
| TNFA_SIGNALING_VIA_NFKB | Genes regulated by NF-kB in | 199 | −3.726 |
| | response to TNF [GeneID = 7124]. | | |
| IL2_STAT5_SIGNALING | Genes up-regulated by STAT5 in | 196 | −4.764 |
| | response to IL2 stimulation. | | |
| IL6_JAK_STAT3_SIGNALING | Genes up-regulated by IL6 | 84 | −5.798 |
| | [GeneID = 3569] via STAT3 | | |
| | [GeneID = 6774], e.g., during acute | | |
| | phase response. | | |
| COMPLEMENT | Genes encoding components of the | 199 | −6.829 |
| | complement system, which is part of | | |
| | the innate immune system. | | |
| INFLAMMATORY_RESPONSE | Genes defining inflammatory | 200 | −6.963 |
| | response. | | |
| INTERFERON_ALPHA_RESPONSE | Genes up-regulated in response to | 94 | −7.819 |
| | alpha interferon proteins. | | |
| ALLOGRAFT_REJECTION | Genes up-regulated during transplant | 196 | −8.52 |
| | rejection. | | |
| INTERFERON_GAMMA_RESPONSE | Genes up-regulated in response to | 194 | −10.885 |
| | IFNG [GeneID = 3458]. | | |

Table 5 shows GO Biological Processes.

TABLE 5
GO Biological Processes

| gs.id | gs.desc | gs.ngenes | fisherz |
|---|---|---|---|
| GO:0070085 | glycosylation | 166 | 5.578 |
| GO:0006487 | protein N-linked glycosylation | 90 | 5.533 |
| GO:0006486 | protein glycosylation | 164 | 5.486 |
| GO:0043413 | macromolecule glycosylation | 164 | 5.486 |
| GO:0018279 | protein N-linked glycosylation via | 84 | 4.97 |
| | asparagine | | |
| GO:0018196 | peptidyl-asparagine modification | 84 | 4.97 |
| GO:0043687 | post-translational protein modification | 126 | 4.513 |
| GO:0009101 | glycoprotein biosynthetic process | 193 | 4.488 |
| GO:0030433 | ER-associated protein catabolic process | 29 | 4.424 |
| GO:0016051 | carbohydrate biosynthetic process | 148 | 4.249 |
| GO:0008652 | cellular amino acid biosynthetic process | 87 | 4.198 |
| GO:0045333 | cellular respiration | 120 | 4.099 |
| GO:0033014 | tetrapyrrole biosynthetic process | 24 | 4.026 |
| GO:0006779 | porphyrin biosynthetic process | 24 | 4.026 |
| GO:0006094 | gluconeogenesis | 39 | 4.011 |
| GO:0006783 | heme biosynthetic process | 19 | 3.979 |
| GO:0009311 | oligosaccharide metabolic process | 61 | 3.866 |
| GO:0006457 | protein folding | 195 | 3.86 |
| GO:0051289 | protein homotetramerization | 40 | 3.789 |
| GO:0019319 | hexose biosynthetic process | 47 | 3.712 |
| GO:0009086 | methionine biosynthetic process | 11 | 3.628 |
| GO:0009060 | aerobic respiration | 33 | 3.628 |
| GO:0000097 | sulfur amino acid biosynthetic process | 14 | 3.626 |
| GO:0043039 | tRNA aminoacylation | 49 | 3.592 |
| GO:0043038 | amino acid activation | 49 | 3.592 |
| GO:0006418 | tRNA aminoacylation for protein translation | 49 | 3.592 |
| GO:0009067 | aspartate family amino acid biosynthetic | 17 | 3.582 |
| | process | | |
| GO:0006084 | acetyl-CoA metabolic process | 35 | 3.544 |
| GO:0046148 | pigment biosynthetic process | 43 | 3.475 |
| GO:0009312 | oligosaccharide biosynthetic process | 42 | 3.465 |
| GO:0022900 | electron transport chain | 122 | 3.425 |
| GO:0046395 | carboxylic acid catabolic process | 148 | 3.408 |
| GO:0016054 | organic acid catabolic process | 148 | 3.408 |
| GO:0046364 | monosaccharide biosynthetic process | 59 | 3.363 |
| GO:0048194 | Golgi vesicle budding | 11 | 3.306 |
| GO:0009309 | amine biosynthetic process | 122 | 3.303 |
| GO:0070972 | protein localization in endoplasmic | 18 | 3.29 |
| | reticulum | | |
| GO:0042254 | ribosome biogenesis | 117 | 3.267 |
| GO:0022613 | ribonucleoprotein complex biogenesis | 190 | 3.229 |
| GO:0061136 | regulation of proteasomal protein catabolic | 50 | 3.181 |
| | process | | |
| GO:0006399 | tRNA metabolic process | 124 | 3.157 |
| GO:0006839 | mitochondrial transport | 77 | 3.144 |
| GO:0006555 | methionine metabolic process | 13 | 3.107 |
| GO:0046165 | alcohol biosynthetic process | 80 | 3.079 |
| GO:0048200 | Golgi transport vesicle coating | 10 | 3.059 |
| GO:0048205 | COPI coating of Golgi vesicle | 10 | 3.059 |
| GO:0048146 | positive regulation of fibroblast proliferation | 32 | 3.048 |
| GO:0006488 | dolichol-linked oligosaccharide biosynthetic | 31 | 3.029 |
| | process | | |
| GO:0009066 | aspartate family amino acid metabolic | 34 | 3.028 |
| | process | | |
| GO:0006829 | zinc ion transport | 20 | 3.018 |
| GO:0051262 | protein tetramerization | 63 | 3.008 |
| GO:0022904 | respiratory electron transport chain | 87 | 3.007 |
| GO:0006664 | glycolipid metabolic process | 35 | 2.997 |
| GO:0006099 | tricarboxylic acid cycle | 22 | 2.993 |
| GO:0048199 | vesicle targeting, to, from or within Golgi | 21 | 2.972 |
| GO:0060425 | lung morphogenesis | 27 | 2.963 |
| GO:0009566 | fertilization | 79 | 2.939 |
| GO:0016042 | lipid catabolic process | 188 | 2.931 |
| GO:0007040 | lysosome organization | 27 | 2.926 |
| GO:0046356 | acetyl-CoA catabolic process | 23 | 2.917 |
| GO:0034637 | cellular carbohydrate biosynthetic process | 93 | 2.914 |
| GO:0042559 | pteridine-containing compound biosynthetic | 19 | 2.897 |
| | process | | |
| GO:0045454 | cell redox homeostasis | 51 | 2.89 |
| GO:0006900 | membrane budding | 26 | 2.887 |
| GO:0009064 | glutamine family amino acid metabolic | 53 | 2.884 |
| | process | | |
| GO:0042558 | pteridine-containing compound metabolic | 31 | 2.87 |
| | process | | |
| GO:0007005 | mitochondrion organization | 151 | 2.799 |
| GO:0019438 | aromatic compound biosynthetic process | 29 | 2.797 |
| GO:0050885 | neuromuscular process controlling balance | 35 | 2.744 |
| GO:0034976 | response to endoplasmic reticulum stress | 38 | 2.736 |
| GO:0046685 | response to arsenic-containing substance | 13 | 2.714 |
| GO:0006901 | vesicle coating | 25 | 2.685 |
| GO:0044243 | multicellular organismal catabolic process | 25 | 2.68 |
| GO:0009310 | amine catabolic process | 101 | 2.665 |
| GO:0006364 | rRNA processing | 87 | 2.656 |
| GO:0034470 | ncRNA processing | 188 | 2.656 |
| GO:0046034 | ATP metabolic process | 125 | 2.655 |
| GO:0006081 | cellular aldehyde metabolic process | 34 | 2.619 |
| GO:0042440 | pigment metabolic process | 51 | 2.579 |
| GO:0032320 | positive regulation of Ras GTPase activity | 79 | −2.624 |
| GO:0032675 | regulation of interleukin-6 production | 48 | −2.634 |
| GO:0030041 | actin filament polymerization | 12 | −2.643 |
| GO:0032651 | regulation of interleukin-1 beta production | 26 | −2.65 |
| GO:0042098 | T cell proliferation | 20 | −2.652 |
| GO:0042742 | defense response to bacterium | 101 | −2.653 |
| GO:0060491 | regulation of cell projection assembly | 36 | −2.665 |
| GO:0016064 | immunoglobulin mediated immune response | 59 | −2.671 |
| GO:0007062 | sister chromatid cohesion | 19 | −2.678 |
| GO:0001837 | epithelial to mesenchymal transition | 37 | −2.68 |
| GO:0032735 | positive regulation of interleukin-12 | 19 | −2.682 |
| | production | | |
| GO:0032814 | regulation of natural killer cell activation | 14 | −2.684 |
| GO:0046634 | regulation of alpha-beta T cell activation | 45 | −2.69 |
| GO:0045580 | regulation of T cell differentiation | 68 | −2.695 |
| GO:0045622 | regulation of T-helper cell differentiation | 19 | −2.701 |
| GO:0045637 | regulation of myeloid cell differentiation | 97 | −2.706 |
| GO:0040017 | positive regulation of locomotion | 172 | −2.712 |
| GO:0032733 | positive regulation of interleukin-10 | 15 | −2.721 |
| | production | | |
| GO:0051480 | cytosolic calcium ion homeostasis | 132 | −2.725 |
| GO:0070665 | positive regulation of leukocyte proliferation | 84 | −2.726 |
| GO:0032946 | positive regulation of mononuclear cell | 83 | −2.731 |
| | proliferation | | |
| GO:0040001 | establishment of mitotic spindle localization | 11 | −2.737 |
| GO:0043903 | regulation of symbiosis, encompassing | 17 | −2.751 |
| | mutualism through parasitism | | |
| GO:0045648 | positive regulation of erythrocyte | 15 | −2.755 |
| | differentiation | | |
| GO:0045070 | positive regulation of viral genome | 10 | −2.756 |
| | replication | | |
| GO:0050718 | positive regulation of interleukin-1 beta | 13 | −2.766 |
| | secretion | | |
| GO:0050716 | positive regulation of interleukin-1 secretion | 13 | −2.766 |
| GO:0002275 | myeloid cell activation involved in immune | 19 | −2.766 |
| | response | | |
| GO:0002714 | positive regulation of B cell mediated | 10 | −2.78 |
| | immunity | | |
| GO:0002891 | positive regulation of immunoglobulin | 10 | −2.78 |
| | mediated immune response | | |
| GO:0050671 | positive regulation of lymphocyte | 82 | −2.793 |
| | proliferation | | |
| GO:0019724 | B cell mediated immunity | 60 | −2.796 |
| GO:0045954 | positive regulation of natural killer cell | 13 | −2.819 |
| | mediated cytotoxicity | | |
| GO:0002717 | positive regulation of natural killer cell | 13 | −2.819 |
| | mediated immunity | | |
| GO:0044126 | regulation of growth of symbiont in host | 14 | −2.829 |
| GO:0044146 | negative regulation of growth of symbiont | 14 | −2.829 |
| | involved in interaction with host | | |
| GO:0044130 | negative regulation of growth of symbiont in | 14 | −2.829 |
| | host | | |
| GO:0044144 | modulation of growth of symbiont involved | 14 | −2.829 |
| | in interaction with host | | |
| GO:0044060 | regulation of endocrine process | 14 | −2.834 |
| GO:0032319 | regulation of Rho GTPase activity | 65 | −2.852 |
| GO:0046636 | negative regulation of alpha-beta T cell | 13 | −2.852 |
| | activation | | |
| GO:0043901 | negative regulation of multi-organism | 21 | −2.856 |
| | process | | |
| GO:0002218 | activation of innate immune response | 113 | −2.865 |
| GO:0051653 | spindle localization | 13 | −2.871 |
| GO.0051293 | establishment of spindle localization | 13 | −2.871 |
| GO:0016197 | endosome transport | 108 | −2.881 |
| GO:0009612 | response to mechanical stimulus | 107 | −2.911 |
| GO:0032677 | regulation of interleukin-8 production | 31 | −2.925 |
| GO:0070661 | leukocyte proliferation | 39 | −2.935 |
| GO:0060389 | pathway-restricted SMAD protein | 10 | −2.941 |
| | phosphorylation | | |
| GO:0050706 | regulation of interleukin-1 beta secretion | 15 | −2.942 |
| GO:0002274 | myeloid leukocyte activation | 48 | −2.942 |
| GO:0050704 | regulation of interleukin-1 secretion | 15 | −2.942 |
| GO:0045582 | positive regulation of T cell differentiation | 44 | −2.966 |
| GO:0043547 | positive regulation of GTPase activity | 132 | −2.978 |
| GO:0045646 | regulation of erythrocyte differentiation | 23 | −2.985 |
| GO:0050715 | positive regulation of cytokine secretion | 34 | −2.999 |
| GO:0002286 | T cell activation involved in immune | 15 | −3.021 |
| | response | | |
| GO:0060323 | head morphogenesis | 19 | −3.03 |
| GO:0002822 | regulation of adaptive immune response | 63 | −3.033 |
| | based on somatic recombination of immune | | |
| | receptors built from immunoglobulin | | |
| | superfamily domains | | |
| GO:0002819 | regulation of adaptive immune response | 66 | −3.039 |
| GO:0045785 | positive regulation of cell adhesion | 90 | −3.039 |
| GO:0045619 | regulation of lymphocyte differentiation | 81 | −3.053 |
| GO:0051607 | defense response to virus | 39 | −3.064 |
| GO:0030335 | positive regulation of cell migration | 163 | −3.068 |
| GO:0032760 | positive regulation of tumor necrosis factor | 28 | −3.07 |
| | production | | |
| GO:2000147 | positive regulation of cell motility | 164 | −3.087 |
| GO:0032943 | mononuclear cell proliferation | 38 | −3.098 |
| GO:0032647 | regulation of interferon-alpha production | 11 | −3.108 |
| GO:0001912 | positive regulation of leukocyte mediated | 22 | −3.119 |
| | cytotoxicity | | |
| GO:0002366 | leukocyte activation involved in immune | 53 | −3.122 |
| | response | | |
| GO:0002263 | cell activation involved in immune response | 53 | −3.122 |
| GO:0050707 | regulation of cytokine secretion | 48 | −3.123 |
| GO:0032479 | regulation of type I interferon production | 48 | −3.129 |
| GO:0032680 | regulation of tumor necrosis factor | 54 | −3.135 |
| | production | | |
| GO:0071214 | cellular response to abiotic stimulus | 95 | −3.137 |
| GO:0070663 | regulation of leukocyte proliferation | 122 | −3.169 |
| GO:0042102 | positive regulation of T cell proliferation | 56 | −3.18 |
| GO:0045621 | positive regulation of lymphocyte | 51 | −3.181 |
| | differentiation | | |
| GO:0032480 | negative regulation of type I interferon | 30 | −3.217 |
| | production | | |
| GO:0007259 | JAK-STAT cascade | 36 | −3.221 |
| GO:0045069 | regulation of viral genome replication | 20 | −3.223 |
| GO:0042119 | neutrophil activation | 11 | −3.23 |
| GO:0006569 | tryptophan catabolic process | 11 | −3.232 |
| GO:0046218 | indolalkylamine catabolic process | 11 | −3.232 |
| GO:0042436 | indole-containing compound catabolic | 11 | −3.232 |
| | process | | |
| GO:0007204 | elevation of cytosolic calcium ion | 117 | −3.245 |
| | concentration | | |
| GO:0032312 | regulation of ARF GTPase activity | 24 | −3.246 |
| GO:0006959 | humoral immune response | 78 | −3.258 |
| GO:0051272 | positive regulation of cellular component | 173 | −3.283 |
| | movement | | |
| GO:0071346 | cellular response to interferon-gamma | 73 | −3.295 |
| GO:0032653 | regulation of interleukin-10 production | 22 | −3.307 |
| GO:0033363 | secretory granule organization | 10 | −3.319 |
| GO:0035023 | regulation of Rho protein signal transduction | 132 | −3.344 |
| GO:0050853 | B cell receptor signaling pathway | 19 | −3.349 |
| GO:0019835 | cytolysis | 20 | −3.366 |
| GO:0045022 | early endosome to late endosome transport | 12 | −3.38 |
| GO:0002443 | leukocyte mediated immunity | 94 | −3.383 |
| GO:0042129 | regulation of T cell proliferation | 81 | −3.406 |
| GO:0032944 | regulation of mononuclear cell proliferation | 119 | −3.406 |
| GO:0050856 | regulation of T cell receptor signaling | 15 | −3.407 |
| | pathway | | |
| GO:0008625 | induction of apoptosis via death domain | 14 | −3.411 |
| | receptors | | |
| GO:0050727 | regulation of inflammatory response | 123 | −3.42 |
| GO:0002460 | adaptive immune response based on somatic | 83 | −3.433 |
| | recombination of immune receptors built | | |
| | from immunoglobulin superfamily domains | | |
| GO:0050670 | regulation of lymphocyte proliferation | 118 | −3.458 |
| GO:0033209 | tumor necrosis factor-mediated signaling | 23 | −3.486 |
| | pathway | | |
| GO:0032103 | positive regulation of response to external | 111 | −3.501 |
| | stimulus | | |
| GO:0045089 | positive regulation of innate immune | 138 | −3.502 |
| | response | | |
| GO:0006568 | tryptophan metabolic process | 12 | −3.522 |
| GO:0042108 | positive regulation of cytokine biosynthetic | 55 | −3.527 |
| | process | | |
| GO:0046651 | lymphocyte proliferation | 36 | −3.536 |
| GO:0032318 | regulation of Ras GTPase activity | 119 | −3.562 |
| GO:0070670 | response to interleukin-4 | 12 | −3.598 |
| GO:0002250 | adaptive immune response | 87 | −3.66 |
| GO:0002697 | regulation of immune effector process | 161 | −3.675 |
| GO:0060333 | interferon-gamma-mediated signaling | 62 | −3.711 |
| | pathway | | |
| GO:0002715 | regulation of natural killer cell mediated | 16 | −3.715 |
| | immunity | | |
| GO:0042269 | regulation of natural killer cell mediated | 16 | −3.715 |
| | cytotoxicity | | |
| GO:0002699 | positive regulation of immune effector | 74 | −3.722 |
| | process | | |
| GO:0045071 | negative regulation of viral genome | 11 | −3.738 |
| | replication | | |
| GO:0048525 | negative regulation of viral reproduction | 11 | −3.738 |
| GO:0002708 | positive regulation of lymphocyte mediated | 35 | −3.741 |
| | immunity | | |
| GO:0002705 | positive regulation of leukocyte mediated | 35 | −3.741 |
| | immunity | | |
| GO:0043122 | regulation of I-kappaB kinase/NF-kappaB | 144 | −3.748 |
| | cascade | | |
| GO:0031343 | positive regulation of cell killing | 26 | −3.794 |
| GO:0001910 | regulation of leukocyte mediated | 27 | −3.795 |
| | cytotoxicity | | |
| GO:0034341 | response to interferon-gamma | 90 | −3.815 |
| GO:0032655 | regulation of interleukin-12 production | 32 | −3.816 |
| GO:0030811 | regulation of nucleotide catabolic process | 199 | −3.851 |
| GO:0033121 | regulation of purine nucleotide catabolic | 199 | −3.851 |
| | process | | |
| GO:0002703 | regulation of leukocyte mediated immunity | 70 | −3.862 |
| GO:0050854 | regulation of antigen receptor-mediated | 17 | −3.905 |
| | signaling pathway | | |
| GO:0050900 | leukocyte migration | 169 | −4.01 |
| GO:0002675 | positive regulation of acute inflammatory | 20 | −4.028 |
| | response | | |
| GO:0043123 | positive regulation of I-kappaB kinase/NF- | 132 | −4.059 |
| | kappaB cascade | | |
| GO:0002706 | regulation of lymphocyte mediated | 57 | −4.059 |
| | immunity | | |
| GO:0001818 | negative regulation of cytokine production | 100 | −4.164 |
| GO:0002407 | dendritic cell chemotaxis | 13 | −4.165 |
| GO:0043087 | regulation of GTPase activity | 176 | −4.208 |
| GO:0042035 | regulation of cytokine biosynthetic process | 84 | −4.23 |
| GO:0031341 | regulation of cell killing | 32 | −4.238 |
| GO:0033124 | regulation of GTP catabolic process | 178 | −4.291 |
| GO:0071260 | cellular response to mechanical stimulus | 46 | −4.33 |
| GO:0001816 | cytokine production | 60 | −4.396 |
| GO:0071356 | cellular response to tumor necrosis factor | 41 | −4.406 |
| GO:0034612 | response to tumor necrosis factor | 61 | −4.513 |
| GO:0072676 | lymphocyte migration | 11 | −4.521 |
| GO:0002252 | immune effector process | 161 | −4.563 |
| GO:0046632 | alpha-beta T cell differentiation | 20 | −4.616 |
| GO:0030595 | leukocyte chemotaxis | 56 | −4.649 |
| GO:0060326 | cell chemotaxis | 71 | −4.667 |
| GO:0031349 | positive regulation of defense response | 186 | −4.725 |
| GO:0030217 | T cell differentiation | 71 | −4.741 |
| GO:0006968 | cellular defense response | 53 | −4.787 |
| GO:0050729 | positive regulation of inflammatory response | 57 | −4.817 |
| GO:0045088 | regulation of innate immune response | 185 | −4.824 |
| GO:0031295 | T cell costimulation | 65 | −5.003 |
| GO:0031294 | lymphocyte costimulation | 65 | −5.003 |
| GO:0030098 | lymphocyte differentiation | 106 | −5.066 |
| GO:0046631 | alpha-beta T cell activation | 26 | −5.072 |
| GO:0001819 | positive regulation of cytokine production | 137 | −5.304 |
| GO:0071357 | cellular response to type I interferon | 59 | −5.319 |
| GO:0060337 | type I interferon-mediated signaling | 59 | −5.319 |
| | pathway | | |
| GO:0009615 | response to virus | 162 | −5.352 |
| GO:0002521 | leukocyte differentiation | 141 | −5.391 |
| GO:0034340 | response to type I interferon | 60 | −5.511 |
| GO:0050852 | T cell receptor signaling pathway | 74 | −6.248 |
| GO:0051251 | positive regulation of lymphocyte activation | 180 | −6.311 |
| GO:0019221 | cytokine-mediated signaling pathway | 188 | −6.417 |
| GO:0042110 | T cell activation | 130 | −6.458 |
| GO:0002696 | positive regulation of leukocyte activation | 194 | −6.505 |
| GO:0050867 | positive regulation of cell activation | 200 | −6.557 |
| GO:0050870 | positive regulation of T cell activation | 146 | −6.662 |
| GO:0050851 | antigen receptor-mediated signaling pathway | 92 | −6.764 |
| GO:0050863 | regulation of T cell activation | 193 | −6.787 |
| GO:0002757 | immune response-activating signal | 175 | −6.928 |
| | transduction | | |
| GO:0002764 | immune response-regulating signaling | 178 | −6.938 |
| | pathway | | |
| GO:0002768 | immune response-regulating cell surface | 100 | −7.307 |
| | receptor signaling pathway | | |
| GO:0002429 | immune response-activating cell surface | 98 | −7.32 |
| | receptor signaling pathway | | |

Table 6 shows MeSH Anatomical Contexts.

TABLE 6
MeSH Anatomical Contexts

| gs.id | gs.desc | gs.ngenes | fisherz |
|---|---|---|---|
| A11.284.430.214.190.875.811 | ribosomes | 134 | 3.695 |
| A11.118.290 | erythrocytes | 111 | 3.469 |
| A11.284.430.214.190.875.190.550 | lysosomes | 114 | 3.012 |
| A11.284.430.214.190.875.336 | golgi_apparatus | 183 | 3.011 |
| A11.118.290.270 | erythrocyte_membrane | 34 | 2.932 |
| A11.284.430.106.279.345.175 | cell_nucleolus | 151 | 2.897 |
| A11.443 | erythroid_cells | 157 | 2.822 |
| A06.407.071 | adrenal_glands | 26 | 2.787 |
| A11.284.187.520.300.505.757 | chromosomes_human_y | 22 | 2.778 |
| A11.284.187.865 | sex_chromosomes | 191 | 2.769 |
| A11.284.430.214.190.875.117.570 | micronuclei_chromosome-defective | 13 | 2.675 |
| A11.284.835.626.461 | mitochondria_liver | 29 | 2.641 |
| A10.272.491 | endothelium | 139 | −2.555 |
| A11.284.180.700 | pseudopodia | 40 | −2.672 |
| A15.145.229.637.555.567.650 | lymphocytes_tumor-infiltrating | 11 | −2.723 |
| A02.835.583 | joints | 56 | −2.735 |
| A11.284.187.520.300.370.375 | chromosomes_human_pair_13 | 78 | −2.794 |
| A11.284.187.520.300.370 | chromosomes_human_13-15 | 191 | −2.882 |
| A15.382.520.604.750 | thymus_gland | 28 | −3.166 |
| A11.284.187.520.300.325.330 | chromosomes_human_pair_6 | 161 | −3.337 |
| A15.378.316.340 | granulocytes | 22 | −3.725 |
| A15.145.229.637.415.345 | eosinophils | 42 | −4.039 |
| A15.382.680.397 | macrophages | 135 | −4.173 |
| A11.118.637.555.567.569.200.400.905 | th2_cells | 42 | −4.25 |
| A11.284.149.165.570 | membrane_microdomains | 61 | −4.286 |
| A15.382.490.555.567.569.200.700 | t-lymphocytes_regulatory | 30 | −4.303 |
| A15.145.229.188 | blood_platelets | 100 | −4.352 |
| A11.329.372 | macrophages | 171 | −4.359 |
| A15.382.520 | lymphatic_system | 96 | −4.44 |
| A15.382.032 | antibody-producing_cells | 131 | −4.67 |
| A15.382.520.604 | lymphoid_tissue | 86 | −4.791 |
| A11.118.637.555.567.569.200.400.900 | th1_cells | 35 | −5.106 |
| A15.145.229.637.555.567.569.220.200 | t-lymphocytes_cytotoxic | 38 | −5.175 |
| A11.627.624 | monocytes | 142 | −5.806 |
| A15.382.066.270 | dendritic_cells | 115 | −5.827 |
| A11.118.637.555.567.537 | killer_cells_natural | 95 | −5.966 |
| A15.378.316.580 | monocytes | 117 | −6.038 |
| A15.382.490.555.567.569.200.400 | t-lymphocytes_helper-inducer | 68 | −6.297 |
| A11.066 | antigen-presenting_cells | 127 | −6.61 |
| A15.382.490.315.583 | neutrophils | 86 | −6.829 |
| A11.627.340 | granulocytes | 158 | −7.124 |
| A11.118.637.555.567.569.500 | t-lymphocyte_subsets | 57 | −7.292 |
| A15.382.490.555.567.569.440 | jurkat_cells | 180 | −7.396 |
| A15.145.229.637.555.567.550 | lymphocyte_subsets | 81 | −7.646 |
| A15.145.229.637.415 | granulocytes | 140 | −7.65 |
| A11.118.637.555.567.569.220 | cd8-positive_t-lymphocytes | 90 | −7.728 |
| A11.118.637.555.567.569.200 | cd4-positive_t-lymphocytes | 150 | −8.198 |

Table 7 shows chemical genetic perturbations.

TABLE 7
Chemical Genetic Perturbations

| gs.id | gs.desc | gs.ngenes | fisherz |
|---|---|---|---|
| WELCSH_BRCA1_TARGETS_DN | Down-regulated by induction of exogenous | 135 | 6.537 |
| | BRCA1 in EcR-293 cells | | |
| TARTE_PLASMA_CELL_VS_B_LYMPHOCYTE_UP | Genes up-regulated in plasma cells compared with | 77 | 6.053 |
| | B lymphocytes. | | |
| YAO_TEMPORAL_RESPONSE_TO_PROGESTERONE_CLUSTER_17 | Genes co-regulated in uterus during a time course response to progesterone | 175 | 6.038 |
| | [PubChem = 5994]: SOM cluster 17. | | |
| SCHLOSSER_SERUM_RESPONSE_AUGMENTED_BY_MYC | Cluster 2: genes up-regulated in B493-6 cells (B lymphocytes) by serum | 103 | 5.872 |
| | alone or in combination with MYC [GeneID = 4609] but not by MYC alone. | | |
| GRUETZMANN_PANCREATIC_CANCER_DN | Genes down-regulated in pancreatic ductal adenocarcinoma (PDAC) | 192 | 5.84 |
| | identified in a meta analysis across four independent studies. | | |
| TIEN_INTESTINE_PROBIOTICS_24HR_DN | Genes down-regulated in Caco-2 cells (intestinal epithelium) after coculture | 197 | 5.404 |
| | with the probiotic bacteria L. casei for 24 h. | | |
| SCHLOSSER_MYC_TARGETS_AND_SERUM_RESPONSE_UP | Cluster 5: genes up-regulated in B493-6 cells (B lymphocytes) by MYC | 45 | 5.36 |
| | [GeneID = 4609] alone or in combination with | | |
| | serum but not by serum alone. | | |
| ELVIDGE_HYPDXIA_DN | Genes down-regulated in MCF7 cells (breast cancer) under hypoxia conditions. | 143 | 4.834 |
| YAO_TEMPORAL_RESPONSE_TO_PROGESTERONE_CLUSTER_14 | Genes co-regulated in uterus during a time course | 137 | 4.817 |
| | response to progesterone [PubChem = 5994]: SOM | | |
| | cluster 14. | | |
| YAO_TEMPORAL_RESPONSE_TO_PROGESTERONE_CLUSTER_11 | Genes co-regulated in uterus during a time course | 100 | 4.763 |
| | response to progesterone [PubChem = 5994]: SOM | | |
| | cluster 11. | | |
| JIANG_AGING_HYPOTHALAMUS_DN | Down-regulated in the hypothalamus of aged (22 | 39 | 4.74 |
| | months) BALB/c mice, compared to young (2 | | |
| | months) controls | | |
| LIN_APC_TARGETS | Genes up-regulated by forced expression of APC | 76 | 4.631 |
| | [GeneID = 324] in the APC-deficient SW480 cell | | |
| | line (colon cancer). | | |
| PENG_LEUCINE_DEPRIVATION_DN | Genes down-regulated in BJAB cells (B- | 176 | 4.446 |
| | lymphoma) after leucine [PubChem = 857] | | |
| | deprivation. | | |
| CHIANG_LIVER_CANCER_SUBCLASS_UNANNOTATED_DN | Marker genes down-regulated in the ‘unannotated’ | 186 | 4.441 |
| | subclass of hepatocellular carcinoma (HCC) | | |
| | samples. | | |
| PURBEY_TARGETS_OF_CTBP1_AND_SATB1_DN | Genes down-regulated in HEK-293 cells (fibroblast) upon knockdown | 168 | 4.261 |
| | of both CTBP1 and SATB1 [GeneID = 1487, 6304] by RNAi. | | |
| SHAFFER_IRF4_TARGETS_IN_MYELOMA_VS_MATURE_B_LYMPHOCYTE | IRF4 [GeneID = 3662] target genes up-regulated in | 94 | 4.132 |
| | primary myeloma vs. mature B lymphocytes. | | |
| MENSSEN_MYC_TARGETS | Genes up-regulated by adenoviral expression of c-MYC [GeneID = 4609] | 49 | 4.121 |
| | in HUVEC cells (umbilical vein endothelium). | | |
| HOFMANN_MYELODYSPLASTIC_SYNDROM_RISK_UP | Genes up-regulated in bone marrow hematopoietic stem cells | 21 | 4.077 |
| | (HSC, CD34+[GeneID = 947]) from patients with high risk of | | |
| | myelodysplastic syndrome (MDS) compared tothe low risk patients. | | |
| BARRIER_COLON_CANCER_RECURRENCE_DN | Down-regulated genes from the 70-gene prognosis predictor for | 19 | 4.048 |
| | stage 2 colon cancer, based on non-neoplastic mucosa | | |
| | gene expression profiles. | | |
| CHAUHAN_RESPONSE_TO_METHOXYESTRADIOL_DN | Genes down-regulated by 2-methoxyestradiol | 93 | 3.989 |
| | (2ME2) [PubChem = 1573] in the MM.1S cell line | | |
| | (multiple myeloma) sensitive to dexamethasone | | |
| | [PubChem = 5743]. | | |
| MORI_PLASMA_CELL_UP | Up-regulated genes in the B lymphocyte developmental signature, based | 50 | 3.979 |
| | on expression profiling of lymphomas from the Emu-myc | | |
| | transgenic mice: plasma cell. | | |
| JOHNSTONE_PARVB_TARGE_TS_2_UP | Genes up-regulated upon overexpression of PARVB [GeneID = 29780] | 132 | 3.97 |
| | in MDA-MB-231 cells (breast cancer) cultured in 3D collagen I and 3D | | |
| | Matrigel only. | | |
| PRAMOONJAGO_SOX4_TARGETS_DN | Genes down-regulated in ACC3 cells (adenoid cystic carcinoma) after | 48 | 3.959 |
| | knockdown of SOX4 [GeneID = 6659] by RNAi. | | |
| SCHUHMACHER_MYC_TARGETS_UP | Genes up-regulated in P493-6 cells (Burkitt's | 74 | 3.947 |
| | lymphoma) induced to express MYC | | |
| | [GeneID = 4609]. | | |
| SCHLOSSER_MYC_TARGETS_AND_SERUM_RESPONSE_DN | Cluster 6: genes down-regulated in B493-6 cells | 44 | 3.925 |
| | (B lymphocytes) by MYC [GeneID = 4609] in | | |
| | combination with serum but not affected by serum | | |
| | alone; they are also up-regulated by MYC alone. | | |
| CREIGHTON_AKT1_SIGNALING_VIA_MTOR_UP | Genes in the AKT1 [GeneID = 207] pathway which | 34 | 3.89 |
| | are independent of MTOR [GeneID = 2475], | | |
| | insensitive to RAD001 (everolimus) | | |
| | [PubChem = 6442177]. | | |
| NIKOLSKY_BREAST_CANCER_16P13_AMPLICON | Genes within amplicon 16p13 identified in a study | 110 | 3.832 |
| | of 191 breast tumor samples. | | |
| PELLICCIOTTA_HDAC_IN_ANTIGEN_PRESENTATION_UP | Antigen processing and presentation genes up- | 63 | 3.823 |
| | regulated in JY cells (B lymphocytes) treated with | | |
| | TSA [PubChem = 5562]. | | |
| VALK_AML_CLUSTER_7 | Top 40 genes from cluster 7 of acute myeloid | 27 | 3.775 |
| | leukemia (AML) expression profile; 61% of the | | |
| | samples are FAB M1 or M2 subtype. | | |
| DACOSTA_UV_RESPONSE_VIA_ERCC3_TTD_UP | Genes exclusively up-regulated in fibroblasts | 61 | 3.769 |
| | expressing the TTD mutant form of ERCC3 | | |
| | [GeneID = 2071], after UVC irradiation. | | |
| SANSOM_APC_TARGETS_REQUIRE_MYC | Genes up-regulated after Cre-lox knockout of | 197 | 3.65 |
| | APC [GeneID = 324] in the small intestine that | | |
| | require functional MYC [GeneID = 4609]. | | |
| BURTON_ADIPOGENESIS_5 | Up-regulated at 48-96 h during differentiation of | 120 | 3.641 |
| | 3T3-L1 cells (fibroblast) into adipocytes. | | |
| DAZARD_UV_RESPONSE_CLUSTER_G5 | Cluster G5: genes up-regulated in NHEK cells | 11 | 3.638 |
| | (normal keratinocyte) at 3 h and 24 h time points | | |
| | after UV-B irradiation. | | |
| JIANG_AGING_CEREBRAL_CORTEX_DN | Down-regulated in the cerebral cortex of aged (22 | 52 | 3.618 |
| | months) BALB/c mice, compared to young (2 | | |
| | months) controls | | |
| LI_AMPLIFIED_IN_LUNG_CANCER | Genes with increased copy number that correlates | 151 | 3.578 |
| | with increased expression across six different lung | | |
| | adenocarcinoma cell lines. | | |
| LUI_THYROID_CANCER_PAX8_PPARG_DN | Top down-regulated genes distinguishing between follicular thyroid carcinoma | 36 | 3.554 |
| | (FTC) samples by the presence or absence of the PAX8-PPARG [GeneID = 7849; | | |
| | 5468] fusion protein. | | |
| VALK_AML_WITH_FLT3_ITD | Genes that best predicted acute myeloid leukemia (AML) with internal tandem | 35 | 3.538 |
| | duplications (IDT) in FLT3 [GeneID = 2322]. | | |
| WOO_LIVER_CANCER_RECURRENCE_DN | Genes negatively correlated with recurrence free survival in patients with | 77 | 3.529 |
| | hepatitis B-related (HBV) hepatocellular carcinoma (HCC). | | |
| MORI_LARGE_PRE_BII_LYMPHOCYTE_UP | Up-regulated genes in the B lymphocyte | 80 | 3.485 |
| | developmental signature, based on expression | | |
| | profiling of lymphomas from the Emu-myc | | |
| | transgenic mice: the Large Pre-BII stage. | | |
| JAIN_NFKB_SIGNALING | Genes abnormally regulated in response to CD40L | 73 | 3.481 |
| | and IL4 [GeneID = 959; 3565] stimulation of B | | |
| | lymphocytes from patients with a hypomorphic | | |
| | mutation of IKBKG [GeneID = 8517]. | | |
| GUTIERREZ_MULTIPLE_MYELOMA_DN | Genes exclusively down-regulated in plasma cells | 32 | 3.481 |
| | from MM (multiple myeloma) patients but with a | | |
| | similar expression pattern in the normal cells and | | |
| | in the cells from WM (Waldenstroem's | | |
| | macroblobulinemia) patients. | | |
| FALVELLA_SMOKERS_WITH_LUNG_CANCER | Genes that distinguish normal from cancer (lung | 76 | 3.474 |
| | adenocarcinoma) samples and smokers from non smoking subjects. | - | |
| ZHONG_SECRETOME_OF_LUNG_CANCER_AND_FIBROBLAST | Proteins secreted in co-culture of LKR-13 tumor | 125 | 3.466 |
| | cells (non-small cell lung cancer, NSCLC) and | | |
| | MLg stroma cells (fibroblasts). | | |
| JIANG_VHL_TARGETS | Genes up-regulated in 786-0 cells (renal | 126 | 3.408 |
| | carcinoma, RCC) upon expression of VHL | | |
| | [GeneID = 7428] off a retroviral vector under | | |
| | normoxia (normal oxygen) condition. | | |
| MCCLUNG_DELTA_FOSB_TARGETS_8WK | Genes up-regulated in the nucleus accumbens (a major reward center in | 46 | 3.327 |
| | brain) 8 weeks after induction of deltaFosB, a FOSB [GeneID = 2354] | | |
| | splice variant. | | |
| JIANG_TIP30_TARGETS_DN | Down-regulated genes in HepG2 cells (liver | 22 | 3.262 |
| | cancer) overexpressing an oncogenic variant of | | |
| | tumor suppressor TIP30 [GeneID = 10553] | | |
| | compared to its wild type form. | | |
| LI_DCP2_BOUND_MRNA | Genes encoding mRNA transcripts specifically | 84 | 3.245 |
| | bound by DCP2 [GeneID = 167227]. | | |
| MODY_HIPPOCAMPUS_POSTNATAL | Genes up-regulated in hyppocampus at late | 61 | 3.24 |
| | postnatal stages (clusters 11 and 15). | | |
| ZHAN_LATE_DIFFERENTIATION_GENES_UP | B lymphocyte late differentiation genes (LDG): | 32 | 3.22 |
| | top genes up-regulated in plasma cells from | | |
| | tonsils (TPC) compared to those from bone | | |
| | marrow (BPC). | | |
| WAKABAYASHI_ADIPOGENESIS_PPARG_RXRA_BOUND_WITH_H4K20ME1_MARK | Genes with promoters bound by both PPARG and | 138 | 3.213 |
| | RXRA [GeneID = 5468, 6256] at 8 (but not 0) day | | |
| | time point of adipocyte differentiation of 3T3-L1 | | |
| | cells (preadipocyte) and that were newly modified | | |
| | by H4K20me1. | | |
| SCHLOSSER_MYC_AND_SERUM_RESPONSE_SYNERGY | Cluster 3: genes strongly up-regulated in B493-6 | 30 | 3.178 |
| | cells (B lymphocytes) by a combination of MYC | | |
| | [GeneID = 4609] and serum but not by each of | | |
| | them alone. | | |
| ACOSTA_PROLIFERATION_INDEPENDENT_MYC_TARGETS_UP | Genes up-regulated in K562 cells (lymphoblast) | 82 | 3.167 |
| | by MYC [GeneID = 4609] in the presence of | | |
| | CKN1B [GeneID = 1027]. | | |
| SHIPP_DLBCL_VS_FOLLICULAR_LYMPHOMA_UP | Top 50 up-regulated markers distinguishing | 40 | 3.166 |
| | diffuse large B-cell lymphoma (DLBCL) from | | |
| | follicular lymphoma (FL) samples. | | |
| DAZARD_UV_RESPONSE_CLUSTER_G4 | Cluster G4: genes increasingly up-regulated in | 20 | 3.164 |
| | NHEK cells (normal keratinocyte) after 12 h time | | |
| | point after UV-B irradiation. | | |
| NIKOLSKY_BREAST_CANCER_7P15_AMPLICON | Genes within amplicon 7p15 identified in a copy | 10 | 3.162 |
| | number alterations study of 191 breast tumor | | |
| | samples. | | |
| MCCLUNG_COCAINE_REWARD_5D | Genes up-regulated in the nucleus accumbens (a major reward center in the brain) | 78 | 3.156 |
| | after 5 days of cocaine [PubChem = 5760] treatment. | | |
| MOOTHA_FFA_OXYDATION | Genes involved in free fatty acid oxidation; based | 21 | 3.09 |
| | on literature and sequence annotation resources | | |
| | and coverted to Affymetrix HG-U133A probe sets. | | |
| AMIT_SERUM_RESPONSE_40_MCF10A | Genes whose expression peaked at 40 min after | 32 | 3.088 |
| | stimulation of MCF10A cells with serum. | | |
| WELCH_GATA1_TARGETS | Genes up-regulated after GATA1 [GeneID = 2623] | 20 | 3.072 |
| | activation in G1E-ER4 cells (erythroid precursors | | |
| | engineered to express GATA1 upon addition of | | |
| | estradiol [PubChemID = 5757]). | | |
| SCHLOSSER_MYC_TARGETS_REPRESSED_BY_SERUM | Cluster 7: genes up-regulated in B493-6 cells (B | 156 | 3.069 |
| | lymphocytes) by MYC [GeneID = 4609] and | | |
| | down-regulated by the combination of MYC and | | |
| | serum. | | |
| WALLACE_PROSTATE_CANCER_UP | Genes up-regulated in prostate tumor vs normal | 19 | 3.065 |
| | tissue samples. | | |
| MOOTHA_VOXPHOS | Genes involved in oxidative phosphorylation; | 81 | 3.053 |
| | based on literature and sequence annotation | | |
| | resources and converted to Affymetrix HG-U133A | | |
| | probe sets. | | |
| ZHANG_RESPONSE_TO_CANTHARIDIN_DN | Genes down-regulated in HL-60 cells (promyeloid | 62 | 3.052 |
| | leukemia) by cantharidin [PubChem = 6708701]. | | |
| POMEROY_MEDULLOBLASTOMA_PROGNOSIS_DN | Top marker genes in medulloblastoma associated | 35 | 3.052 |
| | with poor response to treatment (poor outcome). | | |
| KIM_GASTRIC_CANCER_CHEMOSENSITIVITY | Genes up-regulated in gastric cancer patients | 101 | 3.033 |
| | refractory to chemotherapy treatment with 5- | | |
| | fluorouracil and cisplatin [PubChem = 3385; 2767] | | |
| | compared to the sensitive state. | | |
| OXFORD_RALA_OR_RALB_TARGETS_UP | Genes up-regulated after knockdown of RALA or | 46 | 3.013 |
| | RALB [GeneiD = 5898; 5899], which were also | | |
| | differentially expressed in bladder cancer | | |
| | compared to normal bladder urothelium tissue. | | |
| SESTO_RESPONSE_TO_UV_C6 | Cluster 6: genes changed in primary keratinocytes | 33 | 3.006 |
| | by UVB irradiation. | | |
| NAKAMURA_CANCER_MICROENVIRONMENT_DN | Genes down-regulated in pancreatic cancer cells | 45 | 3.005 |
| | grown in orthotopic xenograft tumors compared to | | |
| | those grown in vitro. | | |
| BILANGES_RAPAMYCIN_SENSITIVE_VIA_TSC1_AND_TSC2 | Genes translationally repressed by rapamycin | 63 | 3 |
| | (sirolimus) [PubChemID = 6610346] in MEF cells | | |
| | (embryonic fibroblast) lacking either TSC1 or | | |
| | TSC2 [GeneID = 7248, 7249] but not in the wild | | |
| | type cells. | | |
| KAYO_CALORIE_RESTRICTION_MUSCLE_DN | Downregulated in the vastus lateralis muscle of middle aged rhesus monkeys | 80 | 2.996 |
| | subjected to caloric restriction since young adulthood vs age matched controls | | |
| SHAFFER_IRF4_TARGETS_IN_PLASMA_CELL_VS_MATURE_B_LYMPHOCYTE | IRF4 [GeneID = 3662] target genes up-regulated in | 62 | 2.992 |
| | plasma cells compared to mature B lymphocytes. | | |
| LUI_TARGETS_OF_PAX8_PPARG_FUSION | Genes down-regulated in follicular thyroid | 26 | 2.949 |
| | carcinoma (FTC) samples that bear PAX8-PPARG | | |
| | [GeneID = 7849; 5468] fusion protein. | | |
| YAO_TEMPORAL_RESPONSE_TO_PROGESTERONE_CLUSTER_12 | Genes co-regulated in uterus during a time course | 76 | 2.949 |
| | response to progesterone [PubChem = 5994]: SOM | | |
| | cluster 12. | | |
| RHODES_CANCER_META_SIGNATURE | Genes commonly up-regulated in cancer relative | 58 | 2.948 |
| | to normal tissue, according to the meta-analysis of | | |
| | the OncoMine gene expression database. | | |
| QI_PLASMACYTOMA_DN | Down-regulated genes that best discriminate | 98 | 2.922 |
| | plasmablastic plasmacytoma from plasmacytic | | |
| | plasmacytoma tumors. | | |
| HOFFMANN_LARGE_TO_SMALL_PRE_BII_LYMPHOCYTE_UP | Genes up-regulated during differentiation from | 157 | 2.899 |
| | large pre-BII to small pre-BII lymphocyte. | | |
| FAELT_B_CLL_WITH_VH3_21_UP | Genes up-regulated in samples from B-CLL (B- | 42 | 2.895 |
| | cell chronic lymphocytic leukemia) with the | | |
| | immunoglobulin heavy chain VH3-21 gene. | | |
| ROSS_AML_OF_FAB_M7_TYPE | Top 100 probe sets for pediatric acute myeloid | 67 | 2.875 |
| | leukemia (AML) subtype FAB M7 (also known as | | |
| | acute megakaryoblastic leukemia, AMKL). | | |
| KEEN_RESPONSE_TO_ROSIGLITAZONE_UP | Genes up-regulated in aorta biopsies from mice | 35 | 2.873 |
| | treated with rosiglitazone [PubChem = 77999]. | | |
| QI_HYPOXIA_TARGETS_OF_HIF1A_AND_FOXA2 | Genes up-regulated by hypoxia in TRAMP-C | 36 | 2.872 |
| | cells (prostatic cancer) expressing HIF1A and | | |
| | FOXA2 [GeneID = 3091, 3170] off plasmid | | |
| | vectors. | | |
| GAZDA_DIAMOND_BLACKFAN_ANEMIA_PROGENITOR_DN | Genes down-regulated in common hematopoietic | 62 | 2.851 |
| | progenitor cells isolated from bone marrow of | | |
| | patients with Diamond-Blackfan anemia (DBA) | | |
| | and mutated RPS19 [GeneID = 6223]. | | |
| ELVIDGE_HYPOXIA_BY_DMOG_DN | Genes down-regulated in MCF7 cells (breast | 58 | 2.837 |
| | cancer) treated with hypoxia mimetic DMOG | | |
| | [PubChem = 3080614]. | | |
| GRAHAM_CML_QUIESCENT_VS_NORMAL_QUIESCENT_UP | Genes up-regulated in quiescent (G0) CD34+ | 85 | 2.824 |
| | [GeneID = 8842] cells isolated from peripheral | | |
| | blood of CML (chronic myeloid leukemia) | | |
| | patients compared to the quiescent cells from | | |
| | normal donors. | | |
| GALE_APL_WITH_FLT3_MUTATED_DN | Genes down-regulated in acute promyelocytic | 17 | 2.813 |
| | leukemia (APL) patients with mutated FLT3 | | |
| | [GeneID = 2322]. | | |
| TONKS_TARGETS_OF_RUNX1_RUNX1T1_FUSION_SUSTAINED_IN_MONOCYTE_UP | Genes up-regulated by RUNX1-RUNX1T1 | 20 | 2.78 |
| | [GeneID = 861; 862] fusion protein in normal | | |
| | hematopoietic progenitors; their expression was | | |
| | sustained in subsequently developing monocytes. | | |
| KANG_FLUOROURACIL_RESISTANCE_UP | Genes up-regulated in gastric cancer cell lines | 22 | 2.755 |
| | resistant to 5-fluorouracil [PubChem = 3385]. | | |
| CHAUHAN_RESPONSE_TO_METHOXYESTRADIOL_UP | Genes up-regulated by 2-methoxyestradiol | 46 | 2.755 |
| | (2ME2) [PubChem = 1573] in the MM.1S cell line | | |
| | (multiple myeloma) sensitive to dexamethasone | | |
| | [PubChem = 5743]. | | |
| HEDENFALK_BREAST_CANCER_BRCA1_VS_BRCA2 | Genes differentially expressed in hereditary breast | 154 | 2.731 |
| | cancer tumors with mutated BRCA1 | | |
| | [GeneID = 672] compared to those with mutated | | |
| | BRCA2 [GeneID = 675]. | | |
| MALONEY_RESPONSE_TO_17AAG_DN | Down-regulated genes in A2780 cells (ovarian cancer) treated with 17AAG | 69 | 2.725 |
| | [PubChem = 6440175], a chemical with anticancer properties. | | |
| IRITANI_MAD1_TARGETS_DN | Genes down-regulated by overexpression of | 40 | 2.719 |
| | MAD1 [GeneID = 4084] in primary thymocytes | | |
| | from RAG2 [GeneID = 5897] knockout mice. | | |
| DELPUECH_FOXO3_TARGETS_DN | Genes down-regulated in DL23 cells (colon | 39 | 2.711 |
| | cancer) upon expression of an activated form of | | |
| | FOXO3 [GeneID = 2309]. | | |
| LUI_THYROID_CANCER_CLUSTER_3 | Cluster 3: genes with similar expression profiles | 18 | 2.693 |
| | across follicular thyroid carcinoma (FTC) | | |
| | samples; genes in this cluster correlated well with | | |
| | the presence of PAX8-PPARG | | |
| | [GeneID = 7849; 5468] fusion protein. | | |
| DAVIES_MULTIPLE_MYELOMA_VS_MGUS_UP | Genes up-regulated in multiple myeloma (MM) | 13 | 2.692 |
| | compared to monoclonal gammopathy of | | |
| | uncertain significance (MGUS). | | |
| ELLWOOD_MYC_TARGETS_DN | Genes down-regulated in transgenic mice | 40 | 2.691 |
| | expressing human MYC [GeneID = 4609] in | | |
| | prostate. | | |
| TONKS_TARGETS_OF_RUNX1_RUNX1T1_FUSION_MONOCYTE_UP | Genes up-regulated in monocytes by RUNX1- | 193 | 2.685 |
| | RUNX1T1 [GeneID = 861; 862] fusion. | | |
| WANG_TARGETS_OF_MLL_CBP_FUSION_DN | Top 50 genes down-regulated in granulocyte/macrophage | 43 | 2.666 |
| | progenitors (GMP) upon expression of MLL-CBP fusion | | |
| | [GeneID = 4297; 1387]. | | |
| RODWELL_AGING_KIDNEY_NO_BLOOD_DN | Genes whose expression decreases with age in | 146 | 2.664 |
| | normal kidney, excluding those with higher | | |
| | expression in blood. | | |
| MUELLER_COMMON_TARGETS_OF_AML_FUSIONS_UP | Up-regulated target genes shared by acute | 14 | 2.661 |
| | myeloid leukemia (AML) translocation products | | |
| | PML RARA [GeneID = 5371; 5914], AML1 ETO | | |
| | [GeneID = 861; 862], and PLZF RARA | | |
| | [GeneID = 5914; 7704]. | | |
| SUZUKI_AMPLIFIED_IN_ORAL_CANCER | High-level amplifications detected in oral | 16 | 2.655 |
| | squamous cell carcinoma (OSCC) lines by array- | | |
| | CGH analysis. | | |
| MARCHINI_TRABECTEDIN_RESISTANCE_DN | Genes down-regulated in chondrosarcoma and | 48 | 2.628 |
| | ovarian carcinoma cell lines which developed | | |
| | resistance to trabectedin [PubChem = 3199]. | | |
| NUTT_GBM_VS_AO_GLIOMA_UP | Top 50 marker genes for glioblastoma multiform | 45 | 2.621 |
| | (GBM), a class of high grade glioma. | | |
| ALCALAY_AML_BY_NPM1_LOCALIZATION_UP | Genes up-regulated in acute myeloid leukemia | 133 | 2.619 |
| | (AML) with respect to cellular localization of | | |
| | NPM1 [GeneID = 4869]: cytoplasmic vs. nucleolar. | | |
| AMIT_EGF_RESPONSE_480_HELA | Genes whose expression peaked at 480 min after | 162 | 2.602 |
| | stimulation of HeLa cells with EGF | | |
| | [GeneID = 1950]. | | |
| MOOTHA_TCA | Tricarboxylic acid related genes; based on | 13 | 2.579 |
| | literature and sequence annotation resources and | | |
| | converted to Affymetrix HG-U133A probe sets. | | |
| PUJANA_BREAST_CANCER_LIT_INT_NETWORK | Genes constituting the LIT-Int network of proteins interacting with breast cancer | 99 | 2.576 |
| | reference proteins BRCA1, BRCA2, ATM, and CHEK2 [GeneID = 672; 675; | | |
| | 472; 11200]; the interactions were manually curated from the literature. | | |
| ZHONG_SECRETOME_OF_LUNG_CANCER_AND_ENDOTHELIUM | Proteins secreted in co-culture of LKR-13 tumor | 64 | 2.555 |
| | cells (non-small cell lung cancer, NSCLC) and | | |
| | MEC stroma cells (endothelium). | | |
| LI_WILMS_TUMOR_VS_FETAL_KIDNEY_1_DN | Genes down-regulated in Wilm's tumor samples | 157 | 2.553 |
| | compared to fetal kidney. | | |
| MUNSHI_MULTIPLE_MYELOMA_UP | Genes up-regulated in multiple myeloma (MM) | 79 | 2.551 |
| | compared to normal plasma cells from the | | |
| | patient's identical twin. | | |
| CHEN_HOXA5_TARGETS_9HR_DN | Genes down-regulated 9 h after induction of | 40 | 2.541 |
| | HoxA5 [GeneID = 3205] expression in a breast | | |
| | cancer cell line. | | |
| AZARE_STAT3_TARGETS | Genes up-regulated in RPWE-1 cells by activated | 23 | 2.537 |
| | STAT3 [GeneID = 6774]. | | |
| DANG_MYC_TARGETS_UP | Genes up-regulated by MYC [GeneID = 4609] and | 125 | 2.534 |
| | whose promoters are bound by MYC, according | | |
| | to MYC Target Gene Database. | | |
| MAYBURD_RESPONSE_TO_L663536_UP | Genes up-regulated in H720 cells (lung cancer) | 29 | 2.527 |
| | after treatment with L663536 (MK886) | | |
| | [PubChem = 105049], an inhibitor of leukotriene | | |
| | biosynthesis. | | |
| YAO_TEMPORAL_RESPONSE_TO_PROGESTERONE_CLUSTER_10 | Genes co-regulated in uterus during a time course | 63 | 2.519 |
| | response to progesterone [PubChem = 5994]: SOM | | |
| | cluster 10. | | |
| SHAFFER_IRF4_TARGETS_IN_ACTIVATED_DENDRITIC_CELL | IRF4 [GeneID = 3662] target genes up-regulated in | 63 | 2.509 |
| | plasmacytoid dendritic cells compared to | | |
| | monocytes. | | |
| HUMMERICH_SKIN_CANCER_PROGRESSION_DN | Selected genes down-regulated during progression | 97 | 2.501 |
| | through benign to malignant skin tumors formed | | |
| | by treatment with DMBA and TPA | | |
| | [PubChem = 6001; 4792] chemicals in the two stage | | |
| | skin carcinogenesis model. | | |
| ZHAN_MULTIPLE_MYELOMA_CD1_DN | Top 50 down-regulated genes in cluster CD-1 of | 44 | 2.501 |
| | multiple myeloma samples with the characteristic | | |
| | expression spike of CCND1 [GeneID = 595]. | | |
| LIU_LIVER_CANCER | Low abundance transcripts specific to | 37 | 2.485 |
| | hepatocellular carcinoma (HCC). | | |
| ROVERSI_GLIOMA_COPY_NUMBER_UP | Genes in the most frequently gained loci in a | 94 | 2.479 |
| | panel of glioma cell lines. | | |
| MOOTHA_GLUCONEOGENESIS | Genes involved in gluconeogenesis; based on | 30 | 2.474 |
| | literature and sequence annotation resources and | | |
| | converted to Affymetrix HG-U133A probe sets. | | |
| HONMA_DOCETAXEL_RESISTANCE | Genes up-regulated in MCF7-ADR cell line | 30 | 2.467 |
| | (breast cancer) resistant to docetaxel | | |
| | [PubChem = 148124]. | | |
| LIU_PROSTATE_CANCER_UP | Genes up-regulated in prostate cancer samples. | 91 | 2.463 |
| COLLER_MYC_TARGETS_UP | Genes up-regulated in 293T (transformed fetal | 23 | 2.455 |
| | renal cell) upon expression of MYC | | |
| | [GeneID = 4609]. | | |
| BOYAULT_LIVER_CANCER_SUBCLASS_G123_UP | Up-regulated genes in hepatocellular carcinoma (HCC) subclass G123, | 42 | 2.453 |
| | defined by unsupervised clustering. | | |
| DANG_REGULATED_BY_MYC_UP | Genes up-regulated by MYC [GeneID = 4609], | 66 | 2.45 |
| | according to the MYC Target Gene Database. | | |
| AMIT_SERUM_RESPONSE_480_MCF10A | Genes whose expression peaked at 480 min after | 34 | 2.442 |
| | stimulation of MCF10A cells with serum. | | |
| NADLER_HYPERGLYCEMIA_AT_OBESITY | Genes correlated with the development of | 56 | 2.395 |
| | hyperglycemia in obese mice. | | |
| XU_RESPONSE_TO_TRETINOIN_AND_NSC682994_DN | Genes down-regulated synergistically in NB4 | 14 | 2.363 |
| | cells (acute promyelocytic leukemia, APL) by | | |
| | tretinoin and NSC682994 | | |
| | [PubChem = 444795; 388304]. | | |
| TONKS_TARGETS_OF_RUNX1_RUNX1T1_FUSION_ERYTHROCYTE_UP | Genes up-regulated in erythroid lineage cells by | 153 | −2.338 |
| | RUNX1-RUNX1T1 [GeneID = 861; 862] fusion. | | |
| BOYAULT_LIVER_CANCER_SUBCLASS_G2 | Genes in hepatocellular carcinoma (HCC) | 27 | −2.358 |
| | subclass G2, defined by unsupervised clustering. | | |
| TSAI_DNAJB4_TARGETS_UP | Genes up-regulated in CL1-5 cells (lung cancer) | 13 | −2.389 |
| | overexpressing DNAJB4 [GeneID = 11080] off a | | |
| | plasmid vector. | | |
| WILCOX_RESPONSE_TO_PROGESTERONE_UP | Genes up-regulated in primary cultures of ovarian | 149 | −2.394 |
| | surface epithlium cells exposed to progesterone | | |
| | [PubChem = 5994] for 5 days. | | |
| LEE_AGING_CEREBELLUM_UP | Upregulated in the cerebellum of aged adult mice | 82 | −2.409 |
| | (30-month) vs young adult (5-month) | | |
| LIEN_BREAST_CARCINOMA_METAPLASTIC_VS_DUCTAL_UP | Genes up-regulated between two breast carcinoma | 82 | −2.421 |
| | subtypes: metaplastic (MCB) and ductal (DCB). | | |
| STEARMAN_TUMOR_FIELD_EFFECT_UP | Up-regulated genes in the ‘Field Effect’ signature | 35 | −2.421 |
| | of normal lung tissue adjacent to the tumor. | | |
| MENSE_HYPOXIA_UP | Hypoxia response genes up-regulated in both | 97 | −2.424 |
| | astrocytes and HeLa cell line. | | |
| PENG_GLUTAMINE_DEPRIVATION_UP | Genes up-regulated in BJAB cells (B-lymphoma) | 36 | −2.426 |
| | after glutamine [PubChem = 738] deprivation. | | |
| WU_CELL_MIGRATION | Genes associated with migration rate of 40 human | 177 | −2.433 |
| | bladder cancer cells. | | |
| KYNG_DNA_DAMAGE_BY_UV | UV only responding genes in primary fibroblasts | 60 | −2.441 |
| | from young donors. | | |
| KOBAYASHI_RESPONSE_TO_ROMIDEPSIN | Genes up-regulated in MM-LH cells (malignant | 19 | −2.446 |
| | melanoma) after treatment with the HDAC | | |
| | inhibitor romidepsin (FK228) | | |
| | [PubChem = 5352062]. | | |
| MCMURRAY_TP53_HRAS_COOPERATION_RESPONSE_DN | Down-regulated ‘cooperation response genes’: | 64 | −2.447 |
| | responded synergystically to the combination of | | |
| | mutant TP53 and HRAS [GeneID = 7157; 3265] in | | |
| | YAMC cells (colon). | | |
| OSAWA_TNF_TARGETS | Genes up-regulated in Hc cells (normal | 10 | −2.454 |
| | hepatocyte) by TNF [GeneID = 7124]. | | |
| TAKEDA_TARGETS_OF_NUP98_HOXA9_FUSION_3D_DN | Genes down-regulated in CD34+ [GeneID = 947] | 29 | −2.467 |
| | hematopoetic cells by expression of NUP98- | | |
| | HOXA9 fusion [GeneID = 4928; 3205] off a | | |
| | retroviral vector at 3 days after transduction. | | |
| BROWNE_HCMV_INFECTION_12HR_UP | Genes up-regulated in primary fibroblast cell | 107 | −2.477 |
| | culture after infection with HCMV (AD169 strain) | | |
| | at 12 h time point that were not up-regulated at the | | |
| | previous time point, 10 h. | | |
| KENNY_CTNNB1_TARGETS_UP | Genes up-regulated in HC11 cells (mammary | 50 | −2.489 |
| | epithelium) by expression of constantly active | | |
| | CTNNB1 [GeneID = 1499]. | | |
| HUMMEL_BURKITTS_LYMPHOMA_DN | Down-regulated genes constituting the molecular | 15 | −2.493 |
| | signature of Burkitt's lymphoma. | | |
| GARGALOVIC_RESPONSE_TO_OXIDIZED_PHOSPHOLIPIDS_YELLOW_UP | Genes from the yellow module which are up-regulated in HAEC cells | 31 | −2.509 |
| | (primary aortic endothelium) after exposure to the oxidized 1- | | |
| | palmitoyl-2-arachidonyl-sn-3-glycerophosphorylcholine (oxPAPC). | | |
| YE_METASTATIC_LIVER_CANCER | Genes up-regulated in hepatocellular carcinoma | 25 | −2.517 |
| | (HCC) with intra-hepatic metastasis compared to | | |
| | the non-metastatic tumors. | | |
| NAKAMURA_ADIPOGENESIS_EARLY_DN | Genes down-regulated in mesenchymal stem cells during early phase of adipogenesis, | 38 | −2.518 |
| | defined as days 1 to 5 of culturing with adipogenic hormones. | | |
| DORSAM_HOXA9_TARGETS_DN | HOXA9 [GeneID = 3205] targets down-regulated | 29 | −2.536 |
| | in hematopoietic stem cells. | | |
| PLASARI_TGFB1_TARGETS_1HR_UP | Genes up-regulated in MEF cells (embryonic | 33 | −2.546 |
| | fibroblast) upon stimulation with TGFB1 | | |
| | [GeneID = 7040] for 1 h. | | |
| CHIARETTI_T_ALL_RELAPSE_PROGNOSIS | Genes whose expression predicted relapse in less | 19 | −2.546 |
| | than 2 years after chemotherapy for adult patients | | |
| | with T-ALL (T cell lymphoblastic leukemia). | | |
| LINDSTEDT_DENDRITIC_CELL_MATURATION_D | Genes down-regulated during the course of | 66 | −2.549 |
| | maturation of monocyte-derived dendritic cells | | |
| | (DC) in response to inflammatory stimuli (cluster | | |
| | D). | | |
| VERHAAK_AML_WITH_NPM1_MUTATED_UP | Genes up-regulated in acute myeloid leukemia | 173 | −2.552 |
| | (AML) patients with mutated NPM1 | | |
| | [GeneID = 4869]. | | |
| BROWNE_HCMV_INFECTION_20HR_DN | Genes down-regulated in primary fibroblast cell | 100 | −2.568 |
| | culture after infection with HCMV (AD169 strain) | | |
| | at 20 h time point that were not down-regulated at | | |
| | the previous time point, 18 h. | | |
| PEREZ_TP53_AND_TP63_TARGETS | Genes up-regulated in HMEC cells (primary | 196 | −2.578 |
| | mammary epithelium) upon expression of both of | | |
| | TP53 [GeneID = 7157] and the transcriptionally | | |
| | active isoform of TP63 [GeneID = 8626] off | | |
| | adenoviral vectors. | | |
| HOFMANN_CELL_LYMPHOMA_DN | Genes down-regulated in lymph nodes from | 39 | −2.578 |
| | patients with mantle cell lymphoma (MCL) | | |
| | compared to the non-malignant hyperplastic | | |
| | lymph nodes. | | |
| CASORELLI_ACUTE_PROMYELOCYTIC_LEUKEMIA_UP | Genes up-regulated in APL (acute promyeolocytic | 167 | −2.582 |
| | leukemia) blasts expressing PML-RARA fusion | | |
| | [GeneID = 5371; 5914] compared to normal | | |
| | promyeloblasts. | | |
| XU_CREBBP_TARGETS_DN | Genes down-regulated in pro-B lymphocytes after | 44 | −2.586 |
| | knockout of CREBBP [GeneID = 1387]. | | |
| DAIRKEE_TERT_TARGETS_DN | Genes down-regulated in non-spontaneously | 117 | −2.589 |
| | immortalizing (NSI) primary breast cancer tumor | | |
| | cultures upon expression of TERT | | |
| | [GeneID = 7015] off a retroviral vector. | | |
| DAVIES_MULTIPLE_MYELOMA_VS_MGUS_DN | Genes down-regulated in multiple myeloma | 26 | −2.591 |
| | (MM) compared to monoclonal gammopathy of | | |
| | uncertain significance (MGUS). | | |
| RASHI_RESPONSE_TO_IONIZING_RADIATION_6 | Cluster 6: late responding genes activated in ATM | 83 | −2.593 |
| | [GeneID = 472] deficient but not in the wild type | | |
| | tissues. | | |
| GALE_APL_WITH_FLT3_MUTATED_UP | Genes up-regulated in acute promyelocytic | 55 | −2.593 |
| | leukemia (APL) patients with mutated FLT3 | | |
| | [GeneID = 2322]. | | |
| WAMUNYOKOLI_OVARIAN_CANCER_GRADES_1_2_DN | Genes down-regulated in mucinous ovarian | 64 | −2.597 |
| | carcinoma tumors of grades 1 and 2 compared to | | |
| | the normal ovarian survace epithelium tissue. | | |
| KLEIN_PRIMARY_EFFUSION_LYMPHOMA_DN | Genes down-regulated in AIDS-related primary | 56 | −2.616 |
| | effusion lymphoma (PEL) samples compared to | | |
| | other tumor subtypes and normal B lymphocytes. | | |
| HOFMANN_MYELODYSPLASTIC_SYNDROM_RISK_DN | Genes down-regulated in bone marrow hematopoietic stem cells | 22 | −2.617 |
| | (HSC, CD34+ [GeneID = 947]) from patients with high risk of | | |
| | myelodysplastic syndrome (MDS) compared to the low risk patients. | | |
| TONKS_TARGETS_OF_RUNX1_RUNX1T1_FUSION_HSC_UP | Genes up-regulated in normal hematopoietic | 182 | −2.617 |
| | progenitors by RUNX1-RUNX1T1 | | |
| | [GeneID = 861; 862] fusion. | | |
| NEWMAN_ERCC6_TARGETS_UP | Genes up-regulated in Cockayne syndrome | 24 | −2.631 |
| | fibroblasts rescued by expression of ERCC6 | | |
| | [GeneID = 2074] off a plasmid vector. | | |
| KEEN_RESPONSE_TO_ROSIGLITAZONE_DN | Genes down-regulated in aorta biopsies from mice | 101 | −2.643 |
| | treated with rosiglitazone [PubChem = 77999]. | | |
| MCDOWELL_ACUTE_LUNG_INJURY_UP | Genes up-regulated in the mouse model of acute | 44 | −2.647 |
| | lung injury induced by inhaling nickel sulfate | | |
| | [PubChem = 24586]. | | |
| PARK_TRETINOIN_RESPONSE_AND_RARA_PLZF_FUSION | Genes up-regulated by tretinoin (ATRA) [PubChem = 444795] in | 22 | −2.65 |
| | U937 cells (acute promyelocytic leukemia, APL) made resistant to | | |
| | the drug by expression of the PLZF-RARA fusion [GeneID = 7704, 5914]. | | |
| WILLIAMS_ESR2_TARGETS_DN | Genes uniquely down-regulated in T47D cells | 10 | −2.655 |
| | (breast cancer) by induction of ESR2 | | |
| | [GeneID = 2100] expression in the Tet-Off system. | | |
| LIEN_BREAST_CARCINOMA_METAPLASTIC | Genes up-regulated in metaplastic carcinoma of | 33 | −2.665 |
| | the breast (MCB) subclass 2 compared to the | | |
| | MCB subclass 1. | | |
| NIKOLSKY_BREAST_CANCER_6P24_P22_AMPLICON | Genes within amplicon 6p24-p22 identified in a | 20 | −2.666 |
| | copy number alterations study of 191 breast tumor | | |
| | samples. | | |
| SHETH_LIVER_CANCER_VS_TXNIP_LOSS_PAM2 | Cluster PAM2: genes up-regulated in | 149 | −2.671 |
| | hepatocellular carcinoma (HCC) vs normal liver | | |
| | tissue from mice deficient for TXNIP | | |
| | [GeneID = 10628]. | | |
| LINDSTEDT_DENDRITIC_CELL_MATURATION_A | Maturation of monocyte-derived dendritic cells | 63 | −2.675 |
| | (DC) in response to inflammatory stimuli: genes | | |
| | up-regulated only at 8 hr after the stimulation | | |
| | (cluster A). | | |
| GOLDRATH_IMMUNE_MEMORY | ‘Memory genes’ expressed uniquely in CD8+ | 64 | −2.688 |
| | [GeneID = 925] memory T lymphocytes (compared | | |
| | with effector or naive cells) | | |
| CHIANG_LIVER_CANCER_SUBCLASS_POLYSOMY7_DN | Marker genes down-regulated in the ‘chromosome | 23 | −2.706 |
| | 7 polysomy’ subclass of hepatocellular carcinoma | | |
| | (HCC); characterized by polysomy of | | |
| | chromosome 7 and by a lack of gains of | | |
| | chromosome 8q. | | |
| GILDEA_METASTASIS | Top genes down-regulated in metastatic (T24T) vs | 30 | −2.724 |
| | non-metastatic (T24) bladder cancer cell lines. | | |
| FARMER_BREAST_CANCER_CLUSTER_5 | Cluster 5: selected 17q21_23 amplicon genes | 19 | −2.74 |
| | clustered together across breast cancer samples. | | |
| BOYLAN_MULTIPLE_MYELOMA_C_CLUSTER_DN | Genes from cluster 2: down-regulated in group C | 31 | −2.74 |
| | of tumors arising from overexpression of BCL2L1 | | |
| | and MYC [GeneID = 598; 4609] in plasma cells. | | |
| GRABARCZYK_BCL11B_TARGETS_UP | Genes up-regulated in Jurkat cells (transformed T | 77 | −2.756 |
| | lymphocytes) after knockdown of BCL11B | | |
| | [GeneID = 64919] byRNAi. | | |
| HALMOS_CEBPA_TARGETS_UP | Genes up-regulated in H358 cells (lung cancer) by | 48 | −2.76 |
| | inducible expression of CEBPA [GeneID = 1050] | | |
| | off plasmid vector. | | |
| BROWNE_HCMV_INFECTION_6HR_DN | Genes down-regulated in primary fibroblast cell | 157 | −2.762 |
| | culture point after infection with HCMV (AD169 | | |
| | strain) at 6 h time point that were not down- | | |
| | regulated at the previous time point, 4 h. | | |
| GUTIERREZ_CHRONIC_LYMPHOCYTIC_LEUKEMIA_DN | Genes exclusively down-regulated in B | 56 | −2.787 |
| | lymphocytes from CLL (chronic lymphocytic | | |
| | leukemia) patients but with a similar expression | | |
| | pattern in the normal cells and in the cells from | | |
| | WM (Waldenstroem's macroblobulinemia) | | |
| | patients. | | |
| GENTILE_UV_RESPONSE_CLUSTER_D2 | Cluster d2: genes down-regulated consistently in | 41 | −2.788 |
| | WS1 cells (fibroblast) between 6 h and 24 h after | | |
| | irradiation with high dose UV-C. | | |
| CHIARETTI_ACUTE_LYMPHOBLASTIC_LEUKEMIA_ZAP70 | Differentially expressed genes between high vs low ZAP70 [GeneID = 7535] | 66 | −2.788 |
| | acute lymphoblastic leukemia (ALL) cases with no known molecular aberrations. | | |
| RAMALHO_STEMNESS_DN | Genes depleted in embryonic, neural and | 70 | −2.796 |
| | hematopoietic stem cells. | | |
| HOOI_ST7_TARGETS_DN | Genes down-regulated in PC-3 cells (prostate | 117 | −2.798 |
| | cancer) stably expressing ST7 [GeneID = 7982] off | | |
| | a plasmid vector. | | |
| GARGALOVIC_RESPONSE_TO_OXIDIZED_PHOSPHOLIPIDS_BLUE_UP | Genes from the blue module which are up- | 132 | −2.806 |
| | regulated in HAEC cells (primary aortic | | |
| | endothelium) after exposure to the oxidized 1- | | |
| | palmitoyl-2-arachidonyl-sn-3- | | |
| | glycerophosphorylcholine (oxPAPC). | | |
| GROSS_HYPOXIA_VIA_ELK3_ONLY_UP | Genes specifically up-regulated in SEND cells | 32 | −2.815 |
| | (skin endothelium) at hypoxia after knockdown of | | |
| | ELK3 [GeneID = 2004] by RNAi. | | |
| BOYAULT_LIVER_CANCER_SUBCLASS_G5_DN | Down-regulated genes in hepatocellular | 27 | −2.816 |
| | carcinoma (HCC) subclass G5, defined by | | |
| | unsupervised clustering. | | |
| PARK_TRETINOIN_RESPONSE_AND_PML_RARA_FUSION | Genes up-regulated by tretinoin (ATRA) | 30 | −2.832 |
| | [PubChem = 444795] in U937 cells (acute | | |
| | promyelocytic leukemia, APL) made sensitive to | | |
| | the drug by expression of the PML-RARA fusion | | |
| | [GeneID = 5371; 5914]. | | |
| TIAN_TNF_SIGNALING_VIA_NFKB | Genes modulated in HeLa cells (cervical | 28 | −2.834 |
| | carcinoma) by TNF [GeneID = 7124] via NFKB | | |
| | pathway. | | |
| GROSS_HYPOXIA_VIA_ELK3_DN | Genes down-regulated in SEND cells (skin | 153 | −2.87 |
| | endothelium) at hypoxia with ELK3 | | |
| | [GeneID = 2004] knockdown by RNAi. | | |
| OKUMURA_INFLAMMATORY_RESPONSE_LPS | Genes up-regulated in mast cells (MC) after | 177 | −2.885 |
| | stimulation with a bacterial lipopolysaccharide | | |
| | (LPS). | | |
| HAHTOLA_MYCOSIS_FUNGOIDES_CD4_DN | Genes down-regulated in T helper cells (defines as CD4+) isolated from patients | 114 | −2.885 |
| | with mucosis fungoides compared to those from normal control donors. | | |
| SCHAEFFER_PROSTATE_DEVELOPMENT_6HR_UP | Genes up-regulated in the urogenital sinus (UGS) | 168 | −2.891 |
| | of day E16 females exposed to the androgen | | |
| | dihydrotestosterone [PubChem = 10635] for 6 h. | | |
| ZHAN_MULTIPLE_MYELOMA_PR_DN | Top 50 down-regulated genes in cluster PR of | 44 | −2.897 |
| | multiple myeloma samples characterized by | | |
| | increased expression of proliferation and cell | | |
| | cycle genes. | | |
| LU_TUMOR_VASCULATURE_UP | Genes up-regulated in endothelial cells derived | 29 | −2.899 |
| | from invasive ovarian cancer tissue. | | |
| LEE_DOUBLE_POLAR_THYMOCYTE | Genes enriched in the double polar (DP) | 26 | −2.906 |
| | thymocyte compared to all other T lymphocyte | | |
| | differentiation stages. | | |
| MAHADEVAN_RESPONSE_TO_MP470_UP | Top genes up-regulated in the GIST | 19 | −2.906 |
| | (gastrointestinal stromal tumor) cell line resistant | | |
| | to imatinib [PubChem = 5291] after treatment with | | |
| | MP470, a protein kinase inhibitor. | | |
| MORI_IMMATURE_B_LYMPHOCYTE_UP | Up-regulated genes in the B lymphocyte | 52 | −2.907 |
| | developmental signature based on expression | | |
| | profiling of lymphomas from the Emu-myc | | |
| | transgenic mice: the immature B stage. | | |
| SAKAI_CHRONIC_HEPATITIS_VS_LIVER_CANCER_UP | Selected genes up-regulated in peripheral blood | 82 | −2.907 |
| | monocytes (PBMC) of patients with | | |
| | hepatocellular carcinoma (HCC) compared to | | |
| | those with chronic hepatitis. | | |
| ROETH_TERT_TARGETS_UP | Genes up-regulated in T lymphocytes | 14 | −2.909 |
| | overexpressing TERT [GeneID = 7015] off a | | |
| | retrovirus vector. | | |
| STARK_PREFRONTAL_CORTEX_22Q11_DELETION_UP | Genes up-regulated in prefrontal cortex (PFC) of | 190 | −2.911 |
| | mice carrying a hemizygotic microdeletion in the | | |
| | 22q11.2 region. | | |
| CHIANG_LIVER_CANCER_SUBCLASS_UNANNOTATED_UP | Marker genes up-regulated in the ‘unannotated’ | 83 | −2.918 |
| | subclass of hepatocellular carcinoma (HCC) | | |
| | samples. | | |
| YOSHIOKA_LIVER_CANCER_EARLY_RECURRENCE_UP | Genes up-regulated in hepatocellular carcinoma | 38 | −2.919 |
| | (HCC) samples from patients with early | | |
| | recurrence (within 2 years after surgery) after | | |
| | resection. | | |
| VANASSE_BCL2_TARGETS_DN | Genes down-regulated in primary B lymphocytes | 72 | −2.924 |
| | engineered to overexpress BCL2 | | |
| | [GeneID = 12043]. | | |
| BROCKE_APOPTOSIS_REVERSED_BY_IL6 | Genes changed in INA-6 cells (multiple myeloma, | 141 | −2.925 |
| | MM) by re-addition of IL6 [GeneID = 3569] after | | |
| | its initial withdrawal for 12 h. | | |
| NIKOLSKY_BREAST_CANCER_8P12_P11_AMPLICON | Genes within amplicon 8p12-p11 identified in a | 52 | −2.928 |
| | copy number alterations study of 191 breast tumor | | |
| | samples. | | |
| JOHNSTONE_PARVB_TARGETS_1_DN | Genes down-regulated in MDA-MB-231 cells | 61 | −2.937 |
| | (breast cancer) upon overexpression of PARVB | | |
| | [GeneID = 29780] under all three culture | | |
| | conditions. | | |
| CLASPER_LYMPHATIC_VESSELS_DURING_METASTASIS_DN | Selected genes down-regulated during invasion of | 36 | −2.937 |
| | lymphatic vessels during metastasis. | | |
| WORSCHECH_TUMOR_EVASION_AND_TOLEROGENICITY_UP | Selected genes with immunologic function which | 30 | −2.95 |
| | were reciprocally changed in evasion and | | |
| | tolerogenic tumor models. | | |
| TURASHVILI_BREAST_DUCTAL_CARCINOMA_VS_LOBULAR_NORMAL_UP | Genes up-regulated in ductal carcinoma vs normal | 72 | −2.956 |
| | lobular breast cells. | | |
| ZHANG_RESPONSE_TO_CANTHARIDIN_UP | Genes up-regulated in HL-60 cells (promyeloid | 19 | −2.961 |
| | leukemia) by cantharidin [PubChem = 6708701]. | | |
| KANG_GIST_WITH_PDGFRA_UP | Genes up-regulated in gastrointestinal stromal | 48 | −2.965 |
| | tumors (GIST) with PDGFRA [GeneID = 5156] | | |
| | mutations. | | |
| MARSHALL_VIRAL_INFECTION_RESPONSE_DN | Genes down-regulated in the influenza-specific | 29 | −2.977 |
| | CD8+ [GeneID = 925] T lymphocytes from | | |
| | bronchoalveolar lavage (BAL) compared to those | | |
| | from spleen. | | |
| EHRLICH_ICF_SYNDROM_UP | Up-regulated in B lymphocytes from patients with | 13 | −2.981 |
| | ICF syndrome caused by mutations in DNMT3B | | |
| | [GeneID = 1789] compared to normals. | | |
| CHIARADONNA_NEOPLASTIC_TRANSFORMATION_CDC25_UP | Genes up-regulated in reverted NIH3T3 cells | 117 | −2.983 |
| | (fibroblasts transformed by activated KRAS | | |
| | [GeneID = 3845] which then reverted to normal | | |
| | cells upon stable over-expression of a dominant | | |
| | negative form of CDC25 [GeneID = 5923]) vs | | |
| | normal fibroblasts. | | |
| WANG_IMMORTALIZED_BY_HOXA9_AND_MEIS1_UP | Up-regulated genes in myeloid progenitors | 27 | −2.993 |
| | immortalized by HOXA9 [GeneID = 3205] vs | | |
| | those immortalized by HOXA9 and MEIS1 | | |
| | [GeneID = 4211]. | | |
| GHANDHI_BYSTANDER_IRRADIATION_UP | Genes significantly (FDR &lt; 10%) up-regulated | 81 | −2.993 |
| | in IMR-90 cells (fibroblast) in response to | | |
| | bystander irradiation. | | |
| HUANG_DASATINIB_RESISTANCE_UP | Genes whose expression positively correlated | 78 | −2.998 |
| | with sensitivity of breast cancer cell lines to | | |
| | dasatinib [PubChem = 3062316]. | | |
| SHIN_B_CELL_LYMPHOMA_CLUSTER_8 | Cluster 8 of genes distinguishing among different | 35 | −3.001 |
| | B lymphocyte neoplasms. | | |
| ALONSO_METASTASIS_DN | Down-regulated genes in melanoma tumors that | 25 | −3.023 |
| | developed metastatic disease compared to primary | | |
| | melanoma that did not. | | |
| PLASARI_TGFB1_SIGNALING_VIA_NFIC_1HR_DN | Genes down-regulated after 1 h of TGFB1 | 105 | −3.029 |
| | [GeneID = 7040] stimulation in MEF cells | | |
| | (embryonic fibroblast) with NFIC | | |
| | [GeneID = 4782] knockout vs wild type MEFs. | | |
| GAVIN_FOXP3_TARGETS_CLUSTER_T4 | Cluster T4 of genes with similar expression | 91 | −3.046 |
| | profiles in thymic T lymphocytes after FOXP3 | | |
| | [GeneID = 50943] loss of function (LOF). | | |
| GREGORY_SYNTHETIC_LETHAL_WITH_IMATINIB | Genes identified as synthetic lethal with imatinib | 135 | −3.054 |
| | [PubChemID = 5291] in RNAi screen in K562 cells | | |
| | (CML, chronic myelogenous leukemia). | | |
| BHATI_G2M_ARREST_BY_2METHOXYESTRADIOL_UP | Up-regulated genes in MDA-MB-435 cells (breast | 114 | −3.062 |
| | cancer) undergoing G2/M arrest after treatment | | |
| | with 2-methoxyestradiol | | |
| | (2ME2)[PubChem = 1573]. | | |
| SASSON_RESPONSE_TO_GONADOTROPHINS_DN | Genes down-regulated in primary granulosa cells | 84 | −3.065 |
| | after stimulation with LH or FSH gonadotrophic | | |
| | hormones for 24 h. | | |
| LIU_TARGETS_OF_VMYB_VS_CMYB_DN | Gene regulated in the opposite directions by v- | 42 | −3.08 |
| | MYB (DN) and c-MYB (UP) variants of CMYB | | |
| | [GeneID = 4602] overexpressed in primary | | |
| | monocyte cultures off adenoviral vectors. | | |
| SANA_TNF_SIGNALING_UP | Genes up-regulated in five primary endothelial | 83 | −3.091 |
| | cell types (lung, aortic, iliac, dermal, and colon) | | |
| | by TNF [GeneID = 7124]. | | |
| SHIN_B_CELL_LYMPHOMA_CLUSTER_3 | Cluster 3 of genes distinguishing among different | 27 | −3.093 |
| | B lymphocyte neoplasms. | | |
| LU_TUMOR_ENDOTHELIAL_MARKERS_UP | Genes specifically up-regulated in tumor | 22 | −3.118 |
| | endothelium. | | |
| XU_AKT1_TARGETS_6HR | Genes up-regulated in DU-145 cells (prostate | 27 | −3.12 |
| | cancer) expressing a dominant negative form of | | |
| | AKT1 [GeneID = 207] upon sham-treatment for 6 h | | |
| | (as a control for the HGF [GeneID = 3082] | | |
| | experiments). | | |
| CROONQUIST_NRAS_SIGNALING_UP | Genes up-regulated in ANBL-6 cell line (multiple | 39 | −3.127 |
| | myeloma, MM) expressing a constantly active | | |
| | form of NRAS [GeneID = 4893] off a plasmid | | |
| | vector compared to those grown in the presence of | | |
| | IL6 [GeneID = 3569]. | | |
| DAUER_STAT3_TARGETS_UP | Top 50 genes up-regulated in A549 cells (lung | 47 | −3.127 |
| | cancer) expressing STAT3 [GeneID = 6774] off an | | |
| | adenovirus vector. | | |
| AKL_HTLV1_INFECTION_DN | Genes down-regulated in WE17/10 cells (CD4+ | 66 | −3.168 |
| | [GeneID = 920] T lymphocytes) infected by | | |
| | HTLV1 (and thus displaying low CD7 | | |
| | [GeneID = 924]) compared to the uninfected (i.e., | | |
| | CD7+) cells. | | |
| MARSON_FOXP3_CORE_DIREC_TARGETS | Direct FOXP3 [GeneID = 50943] targets that | 18 | −3.18 |
| | exhibit consistent transcriptional behavior in | | |
| | hybridoma and in ex vivo T lymphocytes. | | |
| FLOTHO_PEDIATRIC_ALL_THERAPY_RESPONSE_DN | Down-regulated genes significantly associated | 29 | −3.19 |
| | with positive minimal residual disease (MRD) on | | |
| | day 46 after chemotherapy treatment of children | | |
| | with acute lymphoblastic leukemia (ALL). | | |
| WONG_ENDMETRIUM_CANCER_DN | Genes down-regulated in cancer endometrium | 80 | −3.194 |
| | samples compared to the normal endometrium. | | |
| SCHEIDEREIT_IKK_INTERACTING_PROTEINS | Genes encoding IkappaB kinase (IKK) interacting | 58 | −3.208 |
| | proteins. | | |
| BORCZUK_MALIGNANT_MESOTHELIOMA_DN | Genes down-regulated in biphasic (mixed) vs | 100 | −3.212 |
| | epithelial subtypes of malignant peritoneal | | |
| | mesothelioma. | | |
| RUIZ_TNC_TARGETS_UP | Genes up-regulated in T98G cells (glioblastoma) | 149 | −3.218 |
| | by TNC [GeneID = 3371]. | | |
| PEDERSEN_METASTASIS_BY_ERBB2_ISOFORM_3 | Genes regulated in MCF7 cells (breast cancer) by | 19 | −3.238 |
| | expression of the truncated (611-CTF) form of | | |
| | ERBB2 [GeneID = 2064] at both 15 h and 60 h | | |
| | time points. | | |
| GOUYER_TATI_TARGETS_UP | Genes up-regulated in constitutively invasive HT- | 10 | −3.324 |
| | 29 5M21 cells (colon cancer) vs those expressing | | |
| | functionally inactive TATI [GeneID = 6690]. | | |
| ZHAN_MULTIPLE_MYELOMA_DN | Genes most significantly down-regulated in | 39 | −3.337 |
| | multiple myeloma samples, compared to normal | | |
| | bone marrow plasma cells. | | |
| PUIFFE_INVASION_INHIBITED_BY_ASCITES_DN | Genes down-regulated in OV-90 cells (ovarian | 143 | −3.342 |
| | cancer) exposed to ascites which inhibited | | |
| | invasion. | | |
| TONKS_TARGETS_OF_RUNX1_RUNX1T1_FUSION_GRANULOCYTE_DN | Genes down-regulated in granulocytes by | 16 | −3.343 |
| | RUNX1-RUNX1T1 [GeneID = 861; 862] fusion. | | |
| MARSON_FOXP3_TARGETS_UP | Genes up-regulated by FOXP3 [GeneID = 50943] | 65 | −3.343 |
| | in both ex vivo and hybridoma cells. | | |
| TING_SILENCED_BY_DICER | Epigenetically silenced genes up-regulated in | 30 | −3.355 |
| | HCT116 cells (colon cancer) hypomorphic for | | |
| | DICER1 [GeneID = 23405]. | | |
| HINATA_NFKB_TARGETS_KERATINOCYTE_UP | Genes up-regulated in primary keratinocytes by | 90 | −3.364 |
| | expression of p50 (NFKB1) and p65 (RELA) | | |
| | [GeneID = 4790; 5970] components of NFKB. | | |
| WORSCHECH_TUMOR_EVASION_AND_TOLEROGENICITY_DN | Selected genes with immunologic function which | 13 | −3.404 |
| | were reciprocally changed in evasion and | | |
| | tolerogenic tumor models. | | |
| MARSON_FOXP3_TARGETS_STIMULATED_UP | Genes with promoters bound by FOXP3 | 29 | −3.414 |
| | [GeneID = 50943], dependent on it, and up- | | |
| | regulated in hybridoma cells stimulated by PMA | | |
| | [PubChem = 4792] and ionomycin | | |
| | [PubChem = 3733]. | | |
| BUCKANOVICH_T_LYMPHOCYTE_HOMING_ON_TUMOR_UP | Genes up-regulated in microdissected endothelial | 23 | −3.433 |
| | samples from ovarian cancer tumors with tumor- | | |
| | infiltrating lymphocytes (TIL) vs those without | | |
| | TILs. | | |
| LU_TUMOR_ANGIOGENESIS_UP | Up-regulated genes of putative pathways | 25 | −3.444 |
| | stimulated in tumor endothelial cells by papillary | | |
| | serous ovarian epithelial tumor cells. | | |
| BERENJENO_ROCK_SIGNALING_NOT_VIA_RHOA_DN | Genes down-regulated in NIH3T3 cells | 48 | −3.445 |
| | (fibroblasts) after treatment with Y27632 | | |
| | [PubChem = 123862], an inhibitor of ROCK | | |
| | proteins; the changes did not depend on | | |
| | expression of constitutively active (Q63L) form of | | |
| | RHOA [GeneID = 387]. | | |
| HOOI_ST7_TARGETS_UP | Genes up-regulated in PC-3 cells (prostate cancer) | 91 | −3.447 |
| | stably expressing ST7 [GeneID = 7982] off a | | |
| | plasmid vector. | | |
| DING_LUNG_CANCER_EXPRESSION_BY_COPY_NUMBER | The lung adenocarcinoma TSP (tumor sequencing | 100 | −3.46 |
| | project) genes showing strong correlation between | | |
| | DNA copy number variation and gene expression. | | |
| CHIANG_LIVER_CANCER_SUBCLASS_PROLIFERATION_UP | Top 200 marker genes up-regulated in the | 175 | −3.481 |
| | ‘proliferation’ subclass of hepatocellular | | |
| | carcinoma (HCC); characterized by increased | | |
| | proliferation, high levels of serum AFP | | |
| | [GeneID = 174], and chromosomal instability. | | |
| BASSO_CD40_SIGNALING_UP | Gene up-regulated by CD40 [GeneID = 958] | 97 | −3.489 |
| | signaling in Ramos cells (EBV negative Burkitt | | |
| | lymphoma). | | |
| JISON_SICKLE_CELL_DISEASE_UP | Genes up-regulated in peripheral blood | 176 | −3.5 |
| | mononuclear cells (PBMC) from sickle cell | | |
| | disease patients compared to those from healthy | | |
| | subjects. | | |
| PEDERSEN_TARGETS_OF_611CTF_ISOFORM_OF_ERBB2 | Genes up-regulated in MCF7 cells (breast cancer) | 73 | −3.533 |
| | more than three-fold by the truncated form 611- | | |
| | CTF of ERBB2 [GeneID = 2064] and less than | | |
| | two-fold by the full-length ERBB2 | | |
| | [GeneID = 2064]. | | |
| WAMUNYOKOLI_OVARIAN_CANCER_LMP_DN | Genes down-regulated in mucinous ovarian | 192 | −3.534 |
| | carcinoma tumors of low malignant potential | | |
| | (LMP) compared to normal ovarian surface | | |
| | epithelium tissue. | | |
| TONKS_TARGETS_OF_RUNX1_RUNX1T1_FUSION_MONOCYTE_DN | Genes down-regulated in monocytes by RUNX1- | 53 | −3.537 |
| | RUNX1T1 [GeneID = 861; 862] fusion. | | |
| MARSON_FOXP3_TARGETS_DN | Genes down-regulated by FOXP3 | 51 | −3.54 |
| | [GeneID = 50943] in both ex vivo and hybridoma | | |
| | cells. | | |
| NIKOLSKY_BREAST_CANCER_19Q13.1_AMPLICON | Genes within amplicon 19q13.1 identified in a | 21 | −3.543 |
| | copy number alterations study of 191 breast tumor | | |
| | samples. | | |
| HESS_TARGETS_OF_HOXA9_AND_MEIS1_DN | Genes down-regulated in hematopoietic precursor | 73 | −3.554 |
| | cells conditionally expressing HOXA9 and | | |
| | MEIS1 [GeneID = 3205; 4211]. | | |
| GINESTIER_BREAST_CANCER_20Q13_AMPLIFICATION_DN | Genes down-regulated in metastatic breast cancer | 171 | −3.565 |
| | tumors having type 2 amplification in the 20q13 | | |
| | region; involves MYBL2, STK6 and ZNF217 | | |
| | [GeneID = 4605; 6790; 7764] | | |
| LENAOUR_DENDRITIC_CELL_MATURATION_DN | Genes down-regulated during in vitro maturation | 125 | −3.586 |
| | of CD14+ [GeneID = 929] monocytes (day 0) into | | |
| | immature (day 7) and mature dendritic cells (day | | |
| | 14). | | |
| LIU_SOX4_TARGETS_UP | Genes up-regulated in LNCaP cells (prostate | 134 | −3.611 |
| | cancer) by overexpression of SOX4 | | |
| | [GeneID = 6659] and down-regulated by its RNAi | | |
| | knockdown. | | |
| VERHAAK_GLIOBLASTOMA_NEURAL | Genes correlated with neural type of glioblastoma | 125 | −3.632 |
| | multiforme tumors. | | |
| ONO_AML1_TARGETS_UP | Genes up-regulated in CD4+ [GeneID = 920] T | 24 | −3.633 |
| | lymphocytes by expression of AML1 | | |
| | [GeneID = 861] off a viral vector. | | |
| FERRANDO_TAL1_NEIGHBORS | Nearest neighbors of TAL1 [GeneID = 6886], | 21 | −3.645 |
| | based on the close agreement of their expression | | |
| | profiles with that of TAL1 in pediatric T cell acute | | |
| | lymphoblastic leukemia (T-ALL) | | |
| ZHENG_IL22_SIGNALING_UP | Genes up-regulated in ex-vivo colonic tissue after | 53 | −3.65 |
| | treatment with IL22 [GeneID = 50616]. | | |
| XU_RESPONSE_TO_TRETINOIN_AND_NSC682994_UP | Genes up-regulated synergistically in NB4 cells | 17 | −3.67 |
| | (acute promyelocytic leukemia, APL) by tretinoin | | |
| | and NSC682994 [PubChem = 444795; 388304]. | | |
| BROWNE_HCMV_INFECTION_4HR_UP | Genes up-regulated in primary fibroblast cell | 54 | −3.67 |
| | culture point after infection with HCMV (AD169 | | |
| | strain) at 4 h time point that were not up-regulated | | |
| | at the previous time point, 2 h. | | |
| ZHAN_EARLY_DIFFERENTIATION_GENES_DN | B lymphocyte early differentiation genes (EDG): | 42 | −3.679 |
| | top genes down-regulated in tonsil B lymphocytes | | |
| | (TBC) compared to the tonsil plasma cells (TPC). | | |
| GRANDVAUX_IRF3_TARGETS_UP | Genes up-regulated in Jurkat cells (T lymphocyte) | 15 | −3.682 |
| | by expression of a constitutively active form of | | |
| | IRF3 [GeneID = 3661]. | | |
| HADDAD_T_LYMPHOCYTE_AND_NK_PROGENITOR_DN | Genes down-regulated in hematopoietic | 61 | −3.692 |
| | progenitor cells (HPC) of T lymphocyte and NK | | |
| | (natural killer) lineage. | | |
| BOYLAN_MULTIPLE_MYELOMA_D_DN | Genes down-regulated in group D of tumors | 76 | −3.723 |
| | arising from overexpression of BCL2L1 and | | |
| | MYC [GeneID = 598; 4609] in plasma cells. | | |
| ONO_AML1_TARGETS_DN | Genes down-regulated in CD4+ [GeneID = 920] T | 39 | −3.73 |
| | lymphocytes by expression of AML1 | | |
| | [GeneID = 861] off a viral vector. | | |
| MARZEC_IL2_SIGNALING_DN | Genes down-regulated by IL2 [GeneID = 3558] in | 34 | −3.746 |
| | cells derived from CD4+ [GeneID = 920] | | |
| | cutaneous T-cell lymphoma (CTCL). | | |
| BILBAN_B_CLL_LPL_DN | Genes down-regulated in B-CLL (B-cell chronic | 39 | −3.749 |
| | leukemia) samples expressing high levels of LPL | | |
| | [GeneID = 4023] compared with those expressing | | |
| | low levels of the gene. | | |
| RODRIGUES_THYROID_CARCINOMA_DN | Genes down-regulated in poorly differentiated | 76 | −3.757 |
| | thyroid carcinoma (PDTC) compared to anaplastic | | |
| | thyroid carcinoma (ATC). | | |
| MARTINELLI_IMMATURE_NEUTROPHIL_DN | Neutrophil-specific genes down-regulated in | 11 | −3.76 |
| | comparison of immature with mature neutrophils. | | |
| MORI_PLASMA_CELL_DN | Down-regulated genes in the B lymphocyte | 33 | −3.763 |
| | developmental signature, based on expression | | |
| | profiling of lymphomas from the Emu-myc | | |
| | transgenic mice: plasma cell. | | |
| HOEBEKE_LYMPHOID_STEM_CELL_UP | Genes up-regulated in the common lymphoid | 91 | −3.767 |
| | progenitor (CLP, defined as CD34+CD38−CD7+ | | |
| | [GeneID = 947; 952; 924]) compared to a | | |
| | multipotent cord blood cell (defined as | | |
| | CD34+CD38+CD7−). | | |
| STAMBOLSKY_TARGETS_OF_MUTATED_TP53_DN | Genes repressed in SKBR3 cells (breast cancer) | 49 | −3.768 |
| | by mutated TP53 [GeneID = 7157]. | | |
| DER_IFN_GAMMA_RESPONSE_UP | Genes up-regulated in HT1080 (fibrosarcoma) | 69 | −3.782 |
| | cells by treatment with interferon gamma for 6 h. | | |
| TAVOR_CEBPA_TARGETS_UP | Genes up-regulated in KCL22 cells (chronic | 47 | −3.824 |
| | myelogenous leukemia, CML, with BCR-ABL1 | | |
| | [GeneID = 613; 25] fusion) by expression of | | |
| | CEBPA [GeneID = 1050]. | | |
| FOSTER_TOLERANT_MACROPHAGE_UP | Class T (tolerizeable) genes: induced during the | 149 | −3.841 |
| | first LPS stimulation and either not re-induced or | | |
| | induced to a much lesser degree in tolerant | | |
| | macrophages. | | |
| NEMETH_INFLAMMATORY_RESPONSE_LPS_UP | Genes up-regulated in RAW 264.7 cells | 83 | −3.858 |
| | (macrophage) 3 hr after stimulation with bacterial | | |
| | lipopolysaccharide (LPS). | | |
| SEKI_INFLAMMATORY_RESPONSE_LPS_UP | Genes up-regulated in hepatic stellar cells after | 75 | −3.877 |
| | stimulation with bacterial lipopolysacharide | | |
| | (LPS). | | |
| ANASTASSIOU_CANCER_MESENCHYMAL_TRANSITION_SIGNATURE | Genes in the ‘mesenchymal transition signature’ | 64 | −3.881 |
| | common to all invasive cancer types. | | |
| GEISS_RESPONSE_TO_DSRNA_UP | Genes up-pregulated by dsRNA in GRE cells | 37 | −3.905 |
| | (glioma; no interferon system). | | |
| DUTTA_APOPTOSIS_VIA_NFKB | NF-kB target genes involved in the regulation of | 33 | −3.925 |
| | programmed cell death. | | |
| DACOSTA_UV_RESPONSE_VIA_ERCC3_TTD_DN | Genes exclusively down-regulated in fibroblasts | 84 | −3.945 |
| | expressing the TTD mutant form of ERCC3 | | |
| | [GeneID = 2071], after UVC irradiation. | | |
| GAZDA_DIAMOND_BLACKFAN_ANEMIA_PROGENITOR_UP | Genes up-regulated in common hematopoietic | 39 | −3.999 |
| | progenitor cells isolated from bone marrow of | | |
| | patients with Diamond-Blackfan anemia (DBA) | | |
| | and mutated RPS19 [GeneID = 6223]. | | |
| GENTLES_LEUKEMIC_STEM_CELL_UP | Genes up-regulated in LSC (leukemic stem) cells | 29 | −4.016 |
| | compared to LPC (leukemia progenitor) cells | | |
| | from AML (acute myeloid leukemia) tumor | | |
| | samples. | | |
| VALK_AML_CLUSTER_5 | Top 40 genes from cluster 5 of acute myeloid | 32 | −4.024 |
| | leukemia (AML) expression profile; 96% of the | | |
| | samples are FAB M4 or M5 subtype. | | |
| GAVIN_PDE3B_TARGETS | Genes changed in peripheral regulatory T | 21 | −4.046 |
| | lymphocytes that depend on PDE3B | | |
| | [GeneID = 5140]. | | |
| SU_THYMUS | Genes up-regulated specifically in human thymus. | 20 | −4.093 |
| BENNETT_SYSTEMIC_LUPUS_ERYTHEMATOSUS | Genes significantly up-regulated in the blood | 28 | −4.116 |
| | mononuclear cells from patients with systemic | | |
| | lupus erythematosus compared to those from | | |
| | healthy persons. | | |
| TAKEDA_TARGETS_OF_NUP98_HOXA9_FUSION_10D_UP | Genes up-regulated in CD34+ [GeneID = 947] | 188 | −4.128 |
| | hematopoetic cells by expression of NUP98- | | |
| | HOXA9 fusion [GeneID = 4928; 3205] off a | | |
| | retroviral vector at 10 days after transduction. | | |
| WUNDER_INFLAMMATORY_RESPONSE_AND_CHOLESTEROL_UP | Genes up-regulated in gastric mucosal tissue of | 56 | −4.145 |
| | mice on 2% cholesterol [PubChem = 5997] diet and | | |
| | infected with H. pylori vs those infected with H. pylori | | |
| | while on 0% cholesterol diet. | | |
| IKEDA_MIR30_TARGETS_UP | Genes up-regulated in hypertrophic hearts (due to | 115 | −4.171 |
| | expression of constitutively active form of | | |
| | PPP3CA [GeneID = 5530]) and predicted to be | | |
| | targets of miR-30 microRNA. | | |
| CHIANG_LIVER_CANCER_SUBCLASS_CTNNB1_DN | Top 200 marker genes down-regulated in the | 168 | −4.178 |
| | ‘CTNNB1’ subclass of hepatocellular carcinoma | | |
| | (HCC); characterized by activated CTNNB1 | | |
| | [GeneID = 1499]. | | |
| TAKEDA_TARGETS_OF_NUP98_HOXA9_FUSION_16D_UP | Genes up-regulated in CD34+ [GeneID = 947] | 167 | −4.179 |
| | hematopoetic cells by expression of NUP98- | | |
| | HOXA9 fusion [GeneID = 4928; 3205] off a | | |
| | retroviral vector at 16 days after transduction. | | |
| NAKAJIMA_EOSINOPHIL | Top 30 increased eosinophil specific transcripts. | 29 | −4.191 |
| WANG_CLIM2_TARGETS_DN | Genes down-regulated in MCF7 cells (breast | 177 | −4.192 |
| | cancer) engineered to conditionally express a | | |
| | dominant negative form of CLIM2 | | |
| | [GeneID = 8861] by a Tet Off system. | | |
| MORI_LARGE_PRE_BII_LYMPHOCYTE_DN | Down-regulated genes in the B lymphocyte | 58 | −4.196 |
| | developmental signature, based on expression | | |
| | profiling of lymphomas from the Emu-myc | | |
| | transgenic mice: the Large Pre-BII stage. | | |
| GRAHAM_CML_DIVIDING_VS_NORMAL_QUIESCENT_DN | Genes down-regulated in quiescent CD34+ | 93 | −4.204 |
| | [GeneID = 8842] cells isolated from peripheral | | |
| | blood of normal donors compared to the dividing | | |
| | cells from CML (chronic myeloid leukemia) | | |
| | patients. | | |
| GRAHAM_NORMAL_QUIESCENT_VS_NORMAL_DIVIDING_UP | Genes up-regulated in quiescent vs dividing | 65 | −4.215 |
| | CD34+ [GeneID = 8842] cells isolated from | | |
| | peripheral blood of normal donors. | | |
| KIM_GLIS2_TARGETS_UP | Partial list of genes up-regulated in the kidney of | 82 | −4.247 |
| | GLIS2 [GeneID = 84662] knockout mice compared | | |
| | to the wild type. | | |
| LINDSTEDT_DENDRITIC_CELL_MATURATION_C | Maturation of monocyte-derived dendritic cells | 68 | −4.252 |
| | (DC) in response to inflammatory stimuli: genes | | |
| | up-regulated only at 48 hr after the stimulation | | |
| | (cluster C). | | |
| GAURNIER_PSMD4_TARGETS | Inflammatory cytokines, chemokines and their | 67 | −4.255 |
| | cognate receptors up-regulated in THP-1 cells | | |
| | (monocyte) after treatment with PSMD4 | | |
| | [GeneID = 5710]. | | |
| RADAEVA_RESPONSE_TO_IFNA1_UP | Genes up-regulated in primary hepatocytes and | 51 | −4.259 |
| | Hep3B (hepatocyte) cells in response to IFNA | | |
| | [GeneID = 3439]. | | |
| GAVIN_FOXP3_TARGETS_CLUSTER_P3 | Cluster P3 of genes with similar expression | 154 | −4.291 |
| | profiles in peripheral T lymphocytes after FOXP3 | | |
| | [GeneID = 50943] loss of function (LOF). | | |
| DAZARD_UV_RESPONSE_CLUSTER_G6 | Cluster G6: genes increasingly down-regulated in | 151 | −4.326 |
| | NHEK cells (normal keratinocyte) after UV-B | | |
| | irradiation. | | |
| CASORELLI_APL_SECONDARY_VS_DE_NOVO_UP | Genes up-regulated in secondary APL (acute | 39 | −4.348 |
| | promyelocytic leukemia) compared to the de novo | | |
| | tumors. | | |
| BOHN_PRIMARY_IMMUNODEFICIENCY_SYNDROM_DN | Genes down-regulated in B lymphocytes from | 39 | −4.362 |
| | patients with primary immunodefiency syndrome. | | |
| TIEN_INTESTINE_PROBIOTICS_2HR_DN | Genes down-regulated in Caco-2 cells (intestinal | 86 | −4.38 |
| | epithelium) after coculture with the probiotic | | |
| | bacteria L. casei for 2 h. | | |
| SWEET_KRAS_TARGETS_UP | Genes upregulated in KRAS [GeneID = 3845] | 81 | −4.407 |
| | knockdown vs control in a human cell line. | | |
| WORSCHECH_TUMOR_REJECTION_UP | Up-regulated genes defining rejection of | 55 | −4.409 |
| | mammary carcinoma (MMC) tumors. | | |
| LEE_NAIVE_T_LYMPHOCYTE | Genes enriched in the naive circulating T | 18 | −4.445 |
| | lymphocytes compared to the earlier | | |
| | differentiation stages. | | |
| DUNNE_TARGETS_OF_AML1_MTG8_FUSION_UP | Genes up-regulated in Kasumi-1 cells (acute | 48 | −4.447 |
| | myeloid leukaemia (AML) with the t(8; 21) | | |
| | translocation) after knockdown of the AML1 | | |
| | MTG8 fusion [GeneID = 861; 862] by RNAi. | | |
| RAGHAVACHARI_PLATELET_SPECIFIC_GENES | Genes in this set correspond to the most abuntant | 68 | −4.459 |
| | transcripts that are also specific to platelets. | | |
| RHEIN_ALL_GLUCOCORTICOID_THERAPY_UP | Genes up-regulated in ALL (acute lymphoblastic | 75 | −4.463 |
| | leukemia) blasts after 1 week of treatment with | | |
| | glucocorticoids. | | |
| BILD_CTNNB1_ONCOGENIC_SIGNATURE | Genes selected in supervised analyses to | 80 | −4.496 |
| | discriminate cells expressing activated beta- | | |
| | catenin (CTNNB1) [GeneID = 1499] oncogene | | |
| | from control cells expressing GFP. | | |
| KAMIKUBO_MYELOID_CEBPA_NETWORK | Network of differentially expressed myeloid genes | 28 | −4.511 |
| | centered around CEBPA [GeneID = 1050]. | | |
| FINETTI_BREAST_CANCER_KINOME_GREEN | Genes in the green cluster of protein kinases | 16 | −4.52 |
| | distinguishing between luminal A and basal breast | | |
| | cancer subtypes. | | |
| EINAV_INTERFERON_SIGNATURE_IN_CANCER | A gene expression signature found in a subset of | 27 | −4.532 |
| | cancer patients suggestive of a deregulated | | |
| | immune or inflammatory response. | | |
| SEITZ_NEOPLASTIC_TRANSFORMATION_BY_8P_DELETION_UP | Genes up-regulated in CT60/4 cells (breast cancer | 68 | −4.55 |
| | reverted to normal by transfer of chromosome 8p | | |
| | region) vs parental MDA-MB-231 cells (deleted | | |
| | chromosome 8p). | | |
| TIEN_INTESTINE_PROBIOTICS_6HR_DN | Genes down-regulated in Caco-2 cells (intestinal | 161 | −4.565 |
| | epithelium) after coculture with the probiotic | | |
| | bacteria L. casei for 6 h. | | |
| SENGUPTA_EBNA1_ANTICORRELATED | Genes whose reduced expression in | 161 | −4.565 |
| | nasopharyngeal carinoma (NPC) correlated most | | |
| | with the increased expression of EBNA1 | | |
| | [GeneID = 3783774], a latent gene of Epstein-Barr | | |
| | virus (EBV). | | |
| ZHU_CMV_8_HR_UP | Up-regulated at 8 h following infection of primary | 46 | −4.566 |
| | human foreskin fibroblasts with CMV | | |
| KUROZUMI_RESPONSE_TO_ONCOCYTIC_VIRUS_AND_CYCLIC_RGD | Inflammatory cytokines and their receptors | 21 | −4.577 |
| | moduated in brain tumors in response to treatment | | |
| | with cyclic RGD peptide prior to the oncocytic | | |
| | virus therapy. | | |
| MORI_PRE_BI_LYMPHOCYTE_DN | Down-regulated genes in the B lymphocyte | 76 | −4.592 |
| | developmental signature, based on expression | | |
| | profiling of lymphomas from the Emu-myc | | |
| | transgenic mice: the Pre-BI stage. | | |
| WIELAND_UP_BY_HBV_INFECTION | Genes induced in the liver during hepatitis B | 96 | −4.641 |
| | (HBV) viral clearance in chimpanzees. | | |
| LIU_VAV3_PROSTATE_CARCINOGENESIS_UP | Selected genes up-regulated in prostate tumors | 88 | −4.649 |
| | developed by transgenic mice overexpressing | | |
| | VAV3 [GeneID = 10451] in prostate epithelium. | | |
| TONKS_TARGETS_OF_RUNX1_RUNX1T1_FUSION_HSC_DN | Genes down-regulated in normal hematopoietic | 181 | −4.692 |
| | progenitors by RUNX1-RUNX1T1 | | |
| | [GeneID = 861; 862] fusion. | | |
| ODONNELL_TARGETS_OF_MYC_AND_TFRC_UP | Genes down-regulated in P493-6 cells (B | 82 | −4.732 |
| | lymphocyte, Burkitt's lymphoma model) by MYC | | |
| | [GeneID = 4609] and up-regulated by RNAi | | |
| | knockdown of TFRC [GeneID = 7037]. | | |
| KRASNOSELSKAYA_ILF3_TARGETS_UP | Up-regulated in GHOST(3)CXCR4 cells | 38 | −4.82 |
| | (osteosarcoma) upon ectopic expression of ILF3 | | |
| | [GeneID = 3609]. | | |
| GAVIN_FOXP3_TARGETS_CLUSTER_P4 | Cluster P4 of genes with similar expression | 99 | −4.832 |
| | profiles in peripheral T lymphocytes after FOXP3 | | |
| | [GeneID = 50943] loss of function (LOF). | | |
| DAZARD_RESPONSE_TO_UV_SCC_DN | Genes down-regulated in SCC12B2 cells | 121 | −4.833 |
| | (squamous cell carcinoma) by UV-B irradiation. | | |
| LINDSTEDT_DENDRITIC_CELL_MATURATION_B | Maturation of monocyte-derived dendritic cells | 50 | −4.835 |
| | (DC) in response to inflammatory stimuli: genes | | |
| | up-regulated both at 8 hr and 48 hr after the | | |
| | stimulation (cluster B). | | |
| MORI_MATURE_B_LYMPHOCYTE_UP | Up-regulated genes in the B lymphocyte | 88 | −4.87 |
| | developmental signature, based on expression | | |
| | profiling of lymphomas from the Emu-myc | | |
| | transgenic mice: the mature B | | |
| NAKAYAMA_SOFT_TISSUE_TUMORS_PCA1_UP | Top 100 probe sets contributing to the positive | 70 | −4.902 |
| | side of the 1st principal component; | | |
| | predominantly associated with spindle cell and | | |
| | pleomorphic sarcoma samples. | | |
| ICHIBA_GRAFT_VERSUS_HOST_DISEASE_35D_UP | Hepatic graft versus host disease (GVHD), day | 127 | −4.903 |
| | 35: genes up-regulated in allogeneic vs syngeneic | | |
| | bone marrow transplant. | | |
| BERTUCCI_MEDULLARY_VS_DUCTAL_BREAST_CANCER_UP | Genes up-regulated in medullary breast cancer | 197 | −4.942 |
| | (MBC) relative to ductal breast cancer (DBD). | | |
| TURASHVILI_BREAST_LOBULAR_CARCINOMA_VS_LOBULAR_NORMAL_DN | Genes down-regulated in lobular carcinoma vs | 73 | −4.994 |
| | normal lobular breast cells. | | |
| DER_IFN_BETA_RESPONSE_UP | Genes up-regulated in HT1080 (fibrosarcoma) | 100 | −5.009 |
| | cells by treatment with interferon beta for 6 h. | | |
| CHAN_INTERFERON_PRODUCING_DENDRITIC_CELL | Genes up-regulated in spleen interferon-producing | 10 | −5.027 |
| | dendritic cells (IKDC) compared to plasmacytoid | | |
| | dendritic cells (PDC) and conventional dendritic | | |
| | cells (cDC). | | |
| KIM_GERMINAL_CENTER_T_HELPER_UP | Genes up-regulated in germinal center T helper | 65 | −5.041 |
| | cells compared to other CD4+ [GeneID = 920] T | | |
| | lymphocyte types. | | |
| HAHTOLA_SEZARY_SYNDROM_UP | Genes up-regulated in monocytes isolated from | 96 | −5.05 |
| | peripheral blood samples of Sezary syndrome | | |
| | patients compared to those from healthy normal | | |
| | donors. | | |
| BOYLAN_MULTIPLE_MYELOMA_PCA1_UP | Top up-regulated genes from principal component | 98 | −5.089 |
| | 1 (PCA1) which captures variation between | | |
| | normal plasma cells and tumors arising from | | |
| | aberrant expression of BCL2L1 and MYC | | |
| | [GeneID = 598; 4609]. | | |
| GAVIN_FOXP3_TARGETS_CLUSTER_P7 | Cluster P7 of genes with similar expression | 89 | −5.099 |
| | profiles in peripheral T lymphocytes after FOXP3 | | |
| | [GeneID = 50943] loss of function (LOF). | | |
| ZHANG_INTERFERON_RESPONSE | Interferon-inducible genes up-regulated in A549 | 22 | −5.1 |
| | cells (lung cancer) infected with a respiratory | | |
| | syncytial virus (RSV) that had its NS1 | | |
| | [GeneID = 1494468] gene knocked down by RNAi. | | |
| LU_IL4_SIGNALING | Genes up-regulated in peripheral B lymphocytes | 91 | −5.159 |
| | after incubation with IL4 [GeneID = 3565] for 4 h. | | |
| HAHTOLA_CTCL_PATHOGENESIS | Differentially expressed genes relevant to | 16 | −5.221 |
| | pathogenesis of cutaneous T cell lymphoma | | |
| | (CTCL). | | |
| DIAZ_CHRONIC_MEYLOGENOUS_LEUKEMIA_DN | Genes down-regulated in CD34+ [GeneID = 947] | 106 | −5.234 |
| | cells isolated from bone marrow of CML (chronic | | |
| | myelogenous leukemia) patients, compared to | | |
| | those from normal donors. | | |
| ONO_FOXP3_TARGETS_DN | Genes down-regulated in CD4+ [GeneID = 920] T | 41 | −5.235 |
| | lymphocytes transduced with FOXP3 | | |
| | [GeneID = 50943]. | | |
| TURASHVILI_BREAST_LOBULAR_CARCINOMA_VS_DUCTAL_NORMAL_UP | Genes up-regulated in lobular carcinoma vs | 68 | −5.314 |
| | normal ductal breast cells. | | |
| ZHENG_FOXP3_TARGETS_IN_T_LYMPHOCYTE_DN | Genes with promoters bound by FOXP3 | 36 | −5.329 |
| | [GeneID = 50943] and which are down-regulated | | |
| | only in mature (peripheral blood) regulatory | | |
| | CD4+ [GeneID = 920] T lymphocytes. | | |
| IKEDA_MIR133_TARGETS_UP | Genes up-regulated in hypertrophic hearts (due to | 43 | −5.33 |
| | expression of constitutively active form of | | |
| | PPP3CA [GeneID = 5530]) and predicted to be | | |
| | targets of miR-133 microRNA. | | |
| HUANG_GATA2_TARGETS_UP | Genes up-regulated in G1ME cells | 141 | −5.337 |
| | (megakaryocyte/erythroid progenitor lacking | | |
| | GATA1 [GeneID = 2623]) upon knockdown of | | |
| | GATA2 [GeneID = 2624] by RNAi. | | |
| VECCHI_GASTRIC_CANCER_ADVANCED_VS_EARLY_UP | Up-regulated genes distinguishing between two | 172 | −5.379 |
| | subtypes of gastric cancer: advanced (AGC) and | | |
| | early (EGC). | | |
| BOWIE_RESPONSE_TO_TAMOXIFEN | Genes up-regulated by tamoxifen | 18 | −5.393 |
| | [PubChem = 5376] in HMEC-E6 cells (mammary | | |
| | epithelial cells damaged by expression of HPV-16 | | |
| | E6 [GeneID = 1489078]). | | |
| TAKEDA_TARGETS_OF_NUP98_HOXA9_FUSION_3D_UP | Genes up-regulated in CD34+ [GeneID = 947] | 178 | −5.46 |
| | hematopoetic cells by expression of NUP98- | | |
| | HOXA9 fusion [GeneID = 4928; 3205] off a | | |
| | retroviral vector at 3 days after transduction. | | |
| SMIRNOV_RESPONSE_TO_IR_6HR_DN | Genes down-regulated in B lymphocytes at 6 h | 113 | −5.489 |
| | after exprosure to 10 Gy dose of ionizing | | |
| | radiation. | | |
| WINTER_HYPOXIA_DN | Genes down-regulated in head and neck tumor | 52 | −5.505 |
| | samples which clustered around known hypoxia | | |
| | genes. | | |
| HAHTOLA_SEZARY_SYNDROM_DN | Genes down-regulated in monocytes isolated from | 38 | −5.539 |
| | peripheral blood samples of Sezary syndrome | | |
| | patients compared to those from healthy normal | | |
| | donors. | | |
| ROSS_AML_WITH_CBFB_MYH11_FUSION | Top 63 probe sets for pediatric acute myeloid | 50 | −5.565 |
| | leukemia (AML) subtype inv(16); has a CBFB- | | |
| | MYH11 fusion [GeneID = 865; 4629]. | | |
| WATANABE_ULCERATIVE_COLITIS_WITH_CANCER_DN | Genes down-regulated in non-neoplastic rectal | 14 | −5.575 |
| | mucosa samples from patients having cancer | | |
| | associated with ulcerative collitis, compared to | | |
| | those who did not have the cancer. | | |
| BOSCO_ALLERGEN_INDUCED_TH2_ASSOCIATED_MODULE | Genes representing a co-expression network in | 143 | −5.688 |
| | atopic CD4 [GeneID = 920] T lymphocyte | | |
| | responses. | | |
| DER_IFN_ALPHA_RESPONSE_UP | Genes up-regulated in HT1080 cells | 73 | −5.851 |
| | (fibrosarcoma) by treatment with interferon alpha | | |
| | for 6 h. | | |
| DAUER_STAT3_TARGETS_DN | Top 50 genes down-regulated in A549 cells (lung | 49 | −5.852 |
| | cancer) expressing STAT3 [GeneID = 6774] off an | | |
| | adenovirus vector. | | |
| UROSEVIC_RESPONSE_TO_IMIQUIMOD | Interferon cluster genes up-regulated in skin | 23 | −5.914 |
| | tumors treated with imiquimod | | |
| | [PubChem = 57469]. | | |
| ICHIBA_GRAFT_VERSUS_HOST_DISEASE_D7_UP | Hepatic graft versus host disease (GVHD), day 7: | 101 | −6.048 |
| | up-regulated in allogeneic vs syngeneic bone | | |
| | marrow transplant. | | |
| LIANG_SILENCED_BY_METHYLATION_2 | Genes up-regulated in T24 cells (bladder | 51 | −6.1 |
| | carcinoma) after treatment with decitabine (5-aza- | | |
| | 2′-deoxycytidine) [PubChem = 451668]. | | |
| ZHAN_MULTIPLE_MYELOMA_LB_DN | Top 50 down-regulated genes in cluster LB of | 39 | −6.128 |
| | multiple myeloma samples belonging to the low | | |
| | bone disease group. | | |
| SMIRNOV_RESPONSE_TO_IR_2HR_DN | Genes down-regulated in B lymphocytes at 2 h | 55 | −6.131 |
| | after exprosure to 10 Gy dose of ionizing | | |
| | radiation. | | |
| PASQUALUCCI_LYMPHOMA_BY_GC_STAGE_DN | Genes down-regulated in post-GC, BCL6 | 161 | −6.177 |
| | [GeneID = 604] dependent B cell non-Hodgkin's | | |
| | lymphoma (B-NHL) vs MYC [GeneID = 4609] | | |
| | driven pre-GC lymphoma. | | |
| TARTE_PLASMA_CELL_VS_B_LYMPHOCYTE_DN | Genes down-regulated in plasma cells compared | 38 | −6.207 |
| | with B lymphocytes. | | |
| BOWIE_RESPONSE_TO_EXTRACELLULAR_MATRIX | Genes up-regulated by growing HMEC-E6 cells | 17 | −6.209 |
| | (mammary epithelial cells damaged by expression | | |
| | of HPV-16 E6 [GeneID = 1489078]) in | | |
| | extracellular matrix (ECM). | | |
| SMIRNOV_CIRCULATING_ENDOTHELIOCYTES_IN_CANCER_UP | Genes up-regulated in circulating endothelial cells | 155 | −6.213 |
| | (CEC) from cancer patients compared to those | | |
| | from healthy donors. | | |
| GRANDVAUX_IFN_RESPONSE_NOT_VIA_IRF3 | Genes up-regulated in Jurkat cells (T lymphocyte) | 13 | −6.218 |
| | by IFN1@, and IFNB1 [GeneID = 3438; 3456] but | | |
| | not by overexpression of a constitutively active | | |
| | form of IRF3 [GeneID = 3661]. | | |
| KUROZUMI_RESPONSE_TO_ONCOCYTIC_VIRUS | Inflammatory cytokines and their receptors | 43 | −6.354 |
| | modulated in brain tumors after treatment with an | | |
| | oncocytic virus, a potential anticancer therapy. | | |
| TAKEDA_TARGETS_OF_NUP98_HOXA9_FUSION_16D_DN | Genes down-regulated in CD34+ [GeneID = 947] | 130 | −6.365 |
| | hematopoetic cells by expression of NUP98- | | |
| | HOXA9 fusion [GeneID = 4928; 3205] off a | | |
| | retroviral vector at 16 days after transduction. | | |
| BROWN_MYELOID_CELL_DEVELOPMENT_UP | Genes defining differentiation potential of the | 158 | −6.503 |
| | bipotential myeloid cell line FDB. | | |
| TAKEDA_TARGETS_OF_NUP98_HOXA9_FUSION_10D_DN | Genes down-regulated in CD34+ [GeneID = 947] | 139 | −6.513 |
| | hematopoetic cells by expression of NUP98- | | |
| | HOXA9 fusion [GeneID = 4928; 3205] off a | | |
| | retroviral vector at 10 days after transduction. | | |
| VILIMAS_NOTCH1_TARGETS_UP | Genes up-regulated in bone marrow progenitors | 51 | −6.594 |
| | by constitutively active NOTCH1 | | |
| | [GeneID = 4851]. | | |
| SANA_RESPONSE_TO_IFNG_UP | Genes up-regulated in five primary endothelial | 75 | −6.598 |
| | cell types (lung, aortic, iliac, dermal, and colon) | | |
| | by IFNG [GeneID = 3458]. | | |
| BOSCO_INTERFERON_INDUCED_ANTIVIRAL_MODULE | Genes representing interferon-induced antiviral | 75 | −6.911 |
| | module in sputum during asthma exacerbations. | | |
| BOSCO_TH1_CYTOTOXIC_MODULE | Genes representing Th1/cytotoxic module in | 107 | −6.961 |
| | sputum during asthma exacerbations. | | |
| CHUNG_BLISTER_CYTOTOXICITY_DN | Genes down-regulated in blister cells from | 44 | −7.116 |
| | patients with adverse drug reactions (ADR). | | |
| FARMER_BREAST_CANCER_CLUSTER_1 | Cluster 1: interferon, T and B lymphocyte genes | 42 | −7.281 |
| | clustered together across breast cancer samples. | | |
| MOSERLE_IFNA_RESPONSE | Top 50 genes up-regulated in ovarian cancer | 30 | −7.46 |
| | progenitor cells (also known as side population, | | |
| | SP, cells) in response to interferon alpha (IFNA). | | |
| ZHENG_FOXP3_TARGETS_IN_THYMUS_UP | Genes with promoters bound by FOXP3 | 187 | −7.485 |
| | [GeneID = 50943] and which are up-regulated only | | |
| | in developing (located in the thymus) regulatory | | |
| | CD4+ [GeneID = 920] T lymphocytes. | | |
| ALTEMEIER_RESPONSE_TO_LPS_WITH_MECHANICAL_VENTILATION | Genes up-regulated in lung tissue upon LPS | 117 | −7.638 |
| | aspiration with mechanical ventilation (MV) | | |
| | compared to control (PBS aspiration without | | |
| | MV). | | |
| FLECHNER_BIOPSY_KIDNEY_TRANSPLANT_REJECTED_VS_OK_UP | Genes up-regulated in kidney biopsies from | 83 | −7.809 |
| | patients with acute transplant rejection compared | | |
| | to the biopsies from patients with well functioning | | |
| | kidneys more than 1-year post transplant. | | |
| HECKER_IFNB1_TARGETS | Genes transcriptionally modulated in the blood of | 92 | −7.956 |
| | multiple sclerosis patients in response to | | |
| | subcutaneous treatment with recombinant IFNB1 | | |
| | [GeneID = 3456]. | | |
| TAKEDA_TARGETS_OF_NUP98_HOXA9_FUSION_8D_DN | Genes down-regulated in CD34+ [GeneID = 947] | 198 | −9.058 |
| | hematopoetic cells by expression of NUP98- | | |
| | HOXA9 fusion [GeneID = 4928; 3205] off a | | |
| | retroviral vector at 8 days after transduction. | | |
| BROWNE_INTERFERON_RESPONSIVE_GENES | Genes up-regulated in primary fibroblast culture | 67 | −9.279 |
| | after treatment with interferon alpha for 6 h. | | |
| LEE_EARLY_T_LYMPHOCYTE_DN | Genes down-regulated at early stages of | 55 | −9.974 |
| | progenitor T lymphocyte maturation compared to | | |
| | the late stages. | | |
| LEE_DIFFERENTIATING_T_LYMPHOCYTE | Genes enriched at every T lymphocyte | 192 | −10.833 |
| | differentiation stage compared to the early | | |
| | passage fetal thymic stromal cultures (TSC). | | |

Table 8 shows the Number of Data Sets

TABLE 8
Number of Data Sets

| Age-group | No. of datasets | No. of samples |
|---|---|---|
| 11-12 | 13 | 47 |
| 12-13 | 9 | 35 |
| 13-14 | 8 | 29 |
| 14-15 | 8 | 27 |
| 15-16 | 10 | 27 |
| 16-17 | 10 | 51 |
| 17-18 | 14 | 74 |
| 18-19 | 17 | 56 |
| 19-20 | 13 | 33 |
| 20-21 | 14 | 40 |
| 21-22 | 16 | 49 |
| 22-23 | 17 | 43 |
| 23-24 | 14 | 49 |
| 24-25 | 19 | 61 |
| 25-26 | 19 | 51 |
| 26-27 | 18 | 55 |
| 27-28 | 17 | 61 |
| 28-29 | 13 | 53 |
| 29-30 | 15 | 50 |
| 30-31 | 18 | 49 |
| 31-32 | 17 | 48 |
| 32-33 | 17 | 60 |
| 33-34 | 18 | 73 |
| 34-35 | 17 | 64 |
| 35-36 | 20 | 73 |
| 36-37 | 21 | 78 |
| 37-38 | 20 | 57 |
| 38-39 | 17 | 46 |
| 39-40 | 20 | 65 |
| 40-41 | 22 | 94 |
| 41-42 | 18 | 79 |
| 42-43 | 19 | 68 |
| 43-44 | 17 | 69 |
| 44-45 | 23 | 63 |
| 45-46 | 20 | 63 |
| 46-47 | 17 | 55 |
| 47-48 | 20 | 67 |
| 48-49 | 22 | 82 |
| 49-50 | 26 | 89 |

Thus, in certain embodiments, as shown herein, samples are sorted by age, and then hierarchical clustering can be performed to identify sets of genes that are most correlated with older and thus deleterious effects, and younger. These genes are candidate biomarkers for reproductive status.

In certain embodiments, predictors of reproductive success can be verified as follows: The genes most associated with pregnancy outcome (both positive and negative) can be tested on a separate sample set prior to IVF treatment to verify predictive power of the gene set. Two approaches can be used: one with individual qRT-PCR primers for each gene, and a second with the entire set of primers.

In a non-limiting Example, the diagnostic application can be implemented as follows: A blood sample from a patient is collected, RT-PCR performed using the diagnostic primer set, and the profile results matched using Pearson correlation to the reproductive age profile. For example, a 32-year old patient might have a blood profile that best matches that of a 38 year old, indicating that she is reproductively aged relative to her chronological age, and thus might not want to delay childbearing much longer. Under current guidelines, older patients might be denied IVF or other ART treatment, but the tests herein could show that a chronologically 43-year old patient might best match that of an average 38 year old, and thus would still be a viable candidate for IVF or other assisted reproductive techniques. Thus, a diagnostic that easily and accurately correlates a key set of biomarkers with reproductive capacity can be useful for several applications.

In certain embodiments, the methods herein may be directed to the measure or determination of oocyte quality based on a combination of two or more any of the markers discussed herein. For example, a determination of characteristics of two or more of the genes or pathways discussed herein can, in certain embodiments, provide a more accurate set of data regarding a subject's oocyte quality and thus her likelihood of conceiving, than would be the case with only a single gene or pathway.

In certain embodiments, the technology herein contemplates methods or kits that comprise a binding molecule, for example, a binding composition that specifically binds to any protein produced by a biomarker gene discussed herein and may be conjugated to another molecule, for example, an enzyme or a molecule that provides a visual indication of oocyte quality of some other detected characteristic of the cell.

Although the present technology has been described in relation to particular embodiments thereof, these embodiments and examples are merely exemplary and not intended to be limiting. Many other variations and modifications and other uses will become apparent to those skilled in the art. The present technology should, therefore, not be limited by the specific disclosure herein, and may be embodied in other forms not explicitly described here, without departing from the spirit thereof.

I claim:

1. A method of determining the quality of an oocyte in the body of a human without disturbing or destroying the oocyte, the method comprising:
(a) obtaining a cell sample from a female subject, wherein the cell sample does not include the oocyte;
(b) measuring a characteristic of a gene or pathway indicative of oocyte quality in the cell sample; and
(c) predicting or determining the quality of the oocyte based on the characteristic of the gene or pathway.

2. The method of claim 1, wherein step (b) comprises measuring a gene expression value of the cell sample through an RT-PCR assay, an ELISA assay or a colorometric test.

3. The method of claim 2, wherein step (b) further comprises comparing the measured gene expression values to a known gene expression value of an oocyte with Pearson correlation, or by matching the measured gene expression values to a known gene expression profile from a library of genes as markers of oocyte quality.

4. The method of claim 1, wherein the cell sample is extracted from blood, skin, hair, urine, saliva, sweat or vaginal secretion.

5. The method of claim 1, wherein the gene or pathway is chosen from SERPINB2 (serpin peptidase inhibitor, Glade B, member 2, also known as PAI-2); IGFIR (insulin-like growth factor 1 receptor); PIK3CB (phosphoinositide-3-kinase, catalytic, beta polypeptide), IRS2 (insulin receptor substrate 2), HSPA8, HSPD1, HSP60, TGF-β and insulin/IGF-1 (IIS) signaling pathway.

6. A method of predicting the quality of an oocyte in the body of a mammal without disturbing or destroying the oocyte, the method comprising the steps of:
(a) obtaining a cell sample from the mammal, wherein the cell sample does not include the oocyte;
(b) conducting an RT-PCR assay or an ELISA assay on the cell sample using a primer for a gene known to be correlated with aging, and comparing the result with a known value obtained from a library of genes known to be correlated with decreased oocyte quality; and
(d) predicting the likelihood of oocyte viability based on (b).

7. A kit for predicting a woman's oocyte quality without the need for disturbing or destroying an oocyte, the kit comprising:
(a) a collection container for collecting a cell sample obtained from the woman's body, wherein the cell sample does not include an oocyte;
(b) a testing assay comprising RT-PCR or ELISA, wherein the testing assay measures a characteristic of a gene, pathway or transcriptional profile characteristic of the cell sample, and wherein the characteristic indicates the likely quality of an oocyte; and
(c) a visual indicator visible to the woman, the visual indicator providing information regarding the predicted quality of the oocyte.

8. The kit of claim 7, wherein the gene or pathway is chosen from SERPINB2 (serpin peptidase inhibitor, Glade B, member 2, also known as PAI-2); IGFIR (insulin-like growth factor 1 receptor); PIK3CB (phosphoinositide-3-kinase, catalytic, beta polypeptide), IRS2 (insulin receptor substrate 2), HSPA8, HSPD1, HSP60, TGF-β and insulin/IGF-1 (IIS) signaling pathway.

9. A method of producing a library of genes as markers of oocyte quality, the method comprising the steps of:
(a) gathering expression data from cells of women in a particular age range;
(b) calculating an average gene expression for each gene at each age in the range by averaging the expression for that gene in a window of a given period of time;
(c) comparing the average gene expression of (b) to an “age vector” to indicate which genes change most with age; and
(d) calculating a FisherZ score, thereby identifying the genes at the tail ends of the distribution as indicators of biological age.

10. The method of claim 9, wherein for one or more of the genes identified in step (d), the Spearmann correlation of the average gene expression to the age vector was determined, and then sorted by the FisherZ score.

11. The method of claim 10, wherein the genes for which a score above 2 (top 5%) was calculated were added to a set of significantly changed age-dependent genes to comprise the library of genes.

12. The method of claim 9, further comprising any of the following steps: correlating a test gene with a quantitative and measured characteristic of oocyte quality; listing the correlation in the library; comparing a measured characteristic of a gene provided by a patient with that listed in the library; and determining the quality of an oocyte of a patient based on the comparison.

13. A method of developing a reproductive aging gene expression profile and one or more candidate markers of reproductive success or oocyte quality, the method comprising the steps of claim 9.

14. The method of claim 1, wherein the characteristic measured in the cell sample is assigned a score that conveys the expected oocyte viability.

15. The method of claim 14, wherein the score conveys the expected oocyte viability compared to an average for women of the same age as the female subject.
